(19) United States
(12) Patent Application Publication
(10) Pub. No.: US 2024/0408241 A1
(43) Pub. Date: Dec. 12, 2024

(54) NOVEL ANELLOVECTOR COMPOSITIONS AND METHODS

(71) Applicant: FLAGSHIP PIONEERING INNOVATION V, INC., Cambridge, MA (US)

(72) Inventors: Nathan Lawrence Yozwiak, Newton, MA (US); Cesar A. Arze, Boston, MA (US); Dhananjay Maniklal Nawandar, Waltham, MA (US); Simon Delagrave, Sudbury, MA (US); Roger Joseph Hajjar, Lexington, MA (US); Tuyen Ong, Weston, MA (US); Lianna Swanson, West Newton, MA (US)

(21) Appl. No.: 18/700,524

(22) PCT Filed: Oct. 11, 2022
(86) PCT No.: PCT/US2022/077923

§ 371 Date: Apr. 11, 2024

Related U.S. Application Data
(60) Provisional application No. 63/320,515, filed on Mar. 16, 2022.
(60) Provisional application No. 63/254,854, filed on Oct. 12, 2021.

(51) Int. Cl. A61K 48/00; C07K 14/005; C12N 15/86

(52) U.S. Cl. CPC A61K 48/0058; C07K 14/005; C12N 15/86; C12N 2750/00022; C12N 2750/00043; C12N 2750/00045; C12N 2750/00052

(57) ABSTRACT

This invention relates generally to Anelloviridae family vectors (e.g., anellovectors) and compositions and uses thereof.

### BACKGROUND

There is an ongoing need to develop suitable vectors to deliver therapeutic genetic material to patients.

### SUMMARY

The present disclosure provides an Anelloviridae family vector (e.g., anellovector), e.g., a synthetic Anelloviridae family vector (e.g., anellovector), that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). In some embodiments, an Anelloviridae family vector (e.g., anellovector) (e.g., particle, e.g., a viral particle, e.g., an Anellovirus particle) comprises a genetic element (e.g., a genetic element comprising a therapeutic DNA sequence) encapsulated in a proteinaceous exterior (e.g., a proteinaceous exterior comprising an Anelloviridae family virus capsid protein (e.g., an Anellovirus capsid protein, e.g., an Anellovirus ORF1 protein or a polypeptide encoded by an Anellovirus ORF1 nucleic acid; or a chicken anemia virus (CAV) VP1 protein or a polypeptide encoded by a CAV VP1 nucleic acid, e.g., as described herein), which is capable of introducing the genetic element into a cell (e.g., a mammalian cell, e.g., a human cell). In some embodiments, the Anelloviridae family vector (e.g., anellovector) is a particle comprising a proteinaceous exterior comprising a polypeptide encoded by an Anellovirus ORF1 nucleic acid (e.g., an ORF1 nucleic acid of an Alphatorquevirus, Betatorquevirus, or Gammatorquevirus, e.g., as described herein) or a polypeptide encoded by a CAV VP1 nucleic acid (e.g., as described herein). The genetic element of an Anelloviridae family vector (e.g., anellovector) of the present disclosure is typically a circular and/or single-stranded DNA molecule (e.g., circular and single stranded), and generally includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some instances, the genetic element is circular or linear. In some instances, the genetic element comprises or encodes an effector (e.g., a nucleic acid effector, such as a non-coding RNA, or a polypeptide effector, e.g., a protein), e.g., which can be expressed in the cell. In some embodiments, the effector is a therapeutic agent or a therapeutic effector, e.g., as described herein. In some instances, the effector is an endogenous effector or an exogenous effector, e.g., to a wild-type Anellovirus or a target cell. In some embodiments, the effector is exogenous to a wild-type Anellovirus or a target cell. In some embodiments, the Anelloviridae family vector (e.g., anellovector) can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the Anelloviridae family vector (e.g., anellovector)). In other instances, the effector is an exogenous effector. The effector can, in some instances, modulate a function of the cell or modulate an activity or level of a target molecule in the cell. For example, the effector can decrease levels of a target protein in the cell (e.g., as described in Examples 3 and 4). In another example, the Anelloviridae family vector (e.g., anellovector) can deliver and express an effector, e.g., an exogenous protein, in vivo (e.g., as described in Examples 19 and 28). Anelloviridae family vectors (e.g., anellovectors) can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject.

The invention further provides synthetic Anelloviridae family vectors (e.g., anellovectors). A synthetic Anelloviridae family vector (e.g., anellovector) has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic Anelloviridae family vectors (e.g., anellovectors) include an exogenous genetic element enclosed within a proteinaceous exterior, which can be used for delivering the genetic element, or an effector (e.g., an exogenous effector or an endogenous effector) encoded therein (e.g., a polypeptide or nucleic acid effector), into eukaryotic (e.g., human) cells. In some embodiments, the Anelloviridae family vector (e.g., anellovector) does not cause a detectable and/or an unwanted immune or inflammarory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the Anelloviridae family vector (e.g., anellovector) may be substantially non-immunogenic to the target cell, tissue or subject.

In an aspect, the invention features an Anelloviridae family vector (e.g., anellovector) comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid); and wherein the Anelloviridae family vector (e.g., anellovector) is capable of delivering the genetic element into a eukaryotic (e.g., mammalian, e.g., human) cell. In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined as described in Wang et al. (2004, Gene Therapy 11: 711-721, incorporated herein by reference in its entirety). In some embodiments, the genetic element is enclosed within the proteinaceous exterior. In some embodiments, the Anelloviridae family vector (e.g., anellovector) is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), wild-type TTMDV sequence, or wild-type CAV, e.g., a wild-type Anellovirus sequence as listed in Table N1-N4). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anelloviridae family virus (e.g., a wild-type Anellovirus or CAV sequence as described herein, e.g., as listed in Table N1-N4). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In an aspect, the invention features an infectious (to a human cell) particle comprising an Anelloviridae family virus capsid, e.g., an Anellovirus capsid (e.g., a capsid comprising an Anellovirus ORF, e.g., ORF1 polypeptide) or a CAV capsid (e.g., a capsid comprising a CAV VP1 polypeptide) encapsulating a genetic element comprising a protein binding sequence that binds to the capsid and a heterologous (to the Anellovirus) sequence encoding a therapeutic effector. In some embodiments, the particle is capable of delivering the genetic element into a mammalian, e.g., human, cell. In some embodiments, the genetic element has less than about 6% (e.g., less than 6%, 5.5%, 5%, 4.5%, 4%, 3.5%, 3%, 2.5%, 2%, 1.5%, or less) identity to a wild type Anellovirus or CAV. In some embodiments, the genetic element has no more than 1.5%, 2%, 2.5%, 3%, 3.5%, 4%, 4.5%, 5%, 5.5% or 6% identity to a wild type Anellovirus or CAV. In some embodiments, the genetic element has at least about 2% to at least about 5.5% (e.g., 2 to 5%, 3% to 5%, 4% to 5%) identity to a wild type Anellovirus or CAV. In some embodiments, the genetic element has greater than about 2000, 3000, 4000, 4500, or 5000 nucleotides of non-viral sequence (e.g., non Anellovirus genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non Anellovirus genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety).

Also described herein are viral vectors and viral particles based on Anelloviridae family viruses (e.g., Anelloviruses or CAV), which can be used to deliver an agent (e.g., an exogenous effector or an endogenous effector, e.g., a therapeutic effector) to a cell (e.g., a cell in a subject to be treated therapeutically). In some embodiments, Anelloviridae family viruses (e.g., Anelloviruses or CAV) can be used as effective delivery vehicles for introducing an agent, such as an effector described herein, to a target cell, e.g., a target cell in a subject to be treated therapeutically or prophylactically.

In an aspect, the invention features a polypeptide (e.g., a synthetic polypeptide, e.g., an ORF1 molecule or a VP1 molecule) comprising (e.g., in series):
(i) a first region comprising an arginine-rich region, e.g., amino acid sequence having at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof),
(ii) a second region comprising a jelly-roll domain, e.g., an amino acid sequence having at least 30% (e.g., at least about 30, 35, 40, 50, 60, 70, 80, 90, 95, 96, 97, 98, 99, or 100%) sequence identity to a jelly-roll region sequence described herein or a sequence comprising at least 6 beta strands,
(iii) a third region comprising an amino acid sequence having at least 30% (e.g., at least about 30, 35, 40, 50, 60, 70, 80, 90, 95, 96, 97, 98, 99, or 100%) sequence identity to an N22 domain sequence described herein,
(iv) a fourth region comprising an amino acid sequence having at least 70% (e.g., at least about 70, 80, 90, 95, 96, 97, 98, 99, or 100%) sequence identity to an Anellovirus ORF1 or CAV VP1 C-terminal domain (CTD) sequence described herein, and
(v) optionally wherein the polypeptide has an amino acid sequence having less than 100%, 99%, 98%, 95%, 90%, 85%, 80% sequence identity to a wild type Anellovirus ORF1 or CAV VP1 protein described herein.

In some embodiments, the invention features a polypeptide (e.g., a synthetic polypeptide, e.g., an VP1 molecule) comprising (e.g., in series):
(i) a first region comprising an arginine-rich region, e.g., a sequence of at least about 40 amino acids comprising at least 60%, 70%, or 80% basic residues (e.g., arginine, lysine, or a combination thereof),
(ii) a second region comprising a jelly-roll domain, e.g., a sequence comprising at least 6 beta strands, e.g., 6, 7 or 8 beta strands arranged in two antiparallel beta sheets which pack together across a hydrophobic interface, and
(iii) optionally wherein the polypeptide has an amino acid sequence having less than 100%, 99%, 98%, 95%, 90%, 85%, 80% sequence identity to a wild type CAV VP1 protein, e.g., as described herein.

In some embodiments, the polypeptide comprises at least about 70, 80, 90, 95, 96, 97, 98, 99, or 100% sequence identity to an Anellovirus ORF1 molecule or CAV VP1 molecule as described herein (e.g., as listed in any of Tables A1-A3). In some embodiments, the polypeptide comprises at least about 70, 80, 90, 95, 96, 97, 98, 99, or 100% sequence identity to a subsequence (e.g., an arginine (Arg)-rich domain, a jelly-roll domain, a hypervariable region (HVR), an N22 domain, or a C-terminal domain (CTD)) of an Anellovirus ORF1 or CAV VP1 molecule as described herein (e.g., as listed in any of Tables A1-A3). In one embodiment, the amino acid sequences of the (i), (ii), (iii), and (iv) region have at least 90% sequence identity to their respective references and wherein the polypeptide has an amino acid sequence having less than 100%, 99%, 98%, 95%, 90%, 85%, 80% sequence identity to a wild type Anellovirus ORF1 or CAV VP1 protein described herein.

In an aspect, the invention features a complex comprising a polypeptide as described herein (e.g., an Anellovirus ORF1 molecule or CAV VP1 molecule as described herein) and a genetic element comprising a promoter element and a nucleic acid sequence (e.g., a DNA sequence) encoding an effector (e.g., an exogenous effector or an endogenous effector), and a protein binding sequence.

The present disclosure further provides nucleic acid molecules (e.g., a nucleic acid molecule that includes a genetic element as described herein, or a nucleic acid molecule that includes a sequence encoding a proteinaceous exterior protein as described herein). A nucleic acid molecule of the invention may include one or both of (a) a genetic element as described herein, and (b) a nucleic acid sequence encoding a proteinaceous exterior protein as described herein.

In an aspect, the invention features an isolated nucleic acid molecule comprising a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an Anellovirus or CAV, as disclosed herein. In some embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2. In embodiments, the promoter element is capable of directing expression of the effector in a eukaryotic (e.g., mammalian, e.g., human) cell.

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an ORF1 molecule (e.g., an Anellovirus ORF1 protein, e.g., as described herein). In some embodiments, the nucleic acid molecule comprises a sequence encoding an ORF2 molecule (e.g., an Anellovirus ORF2 protein, e.g., as described herein). In some embodiments, the nucleic acid molecule comprises a sequence encoding an ORF3 molecule (e.g., an Anellovirus ORF3 protein, e.g., as described herein). In some embodiments, the nucleic acid molecule comprises a sequence encoding a VP1 molecule (e.g., an CAV VP1 protein, e.g., as described herein). In an aspect, the invention features a genetic element comprising one, two, or three of: (i) a promoter element and a sequence encoding an effector, e.g., an exogenous or endogenous effector; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus or CAV sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus or CAV sequence; and (iii) a protein binding sequence, e.g., an exterior protein binding sequence, and wherein the nucleic acid construct is a single-stranded DNA; and wherein the nucleic acid construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, a genetic element described herein comprises one or more modified nucleotides (e.g., a base modification, sugar modification, or backbone modification). In some embodiments, the genetic element comprises a sequence encoding an ORF1 molecule (e.g., an Anellovirus ORF1 protein, e.g., as described herein). In some embodiments, the genetic element comprises a sequence encoding an ORF2 molecule (e.g., an Anellovirus ORF2 protein, e.g., as described herein). In some embodiments, the genetic element comprises a sequence encoding an ORF3 molecule (e.g., an Anellovirus ORF3 protein, e.g., as described herein). In some embodiments, the genetic element comprises a sequence encoding a VP1 molecule (e.g., a CAV VP1 protein, e.g., as described herein).

In an aspect, the invention features a host cell or helper cell comprising: (a) a nucleic acid comprising a sequence encoding one or more of an ORF1 molecule, an ORF2 molecule, an ORF3, a VP1 molecule, a VP2 molecule, or a VP3 molecule (e.g, a sequence encoding an Anellovirus ORF1 polypeptide or CAV VP1 polypeptide described herein), wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode an ORF1 or VP1 polypeptide (e.g., an ORF1 protein or a VP1 protein). For example, the host cell or helper cell comprises (a) and (b) either in cis (both part of the same nucleic acid molecule) or in trans (each part of a different nucleic acid molecule). In embodiments, the genetic element of (b) is circular, single-stranded DNA. In some embodiments, the host cell is a manufacturing cell line. In some embodiments, the host cell or helper cell is adherent or in suspension, or both. In some embodiments, the host cell or helper cell is grown in a microcarrier. In some mbodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of anellovectors by the host cell or helper cell.

In an aspect, the invention features a pharmaceutical composition comprising an Anelloviridae family vector (e.g., anellovector) (e.g., a synthetic Anelloviridae family vector (e.g., anellovector)) as described herein. In embodiments, the pharmaceutical composition further comprises a pharmaceutically acceptable carrier or excipient. In embodiments, the pharmaceutical composition comprises a unit dose comprising about 10⁵-10¹⁴ genome equivalents of the Anelloviridae family vector (e.g., anellovector) per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of Anelloviridae family vector (e.g., anellovector) genomes or genomic equivalents (e.g., as defined by number of genomes per volume).

In an aspect, the invention features a method of treating a disease or disorder in a subject, the method comprising administering to the subject an Anelloviridae family vector (e.g., anellovector), e.g., a synthetic Anelloviridae family vector (e.g., anellovector), e.g., as described herein. In an aspect, the invention features a method of treating a disease or disorder in a subject, the method comprising administering to the eye of the subject an Anelloviridae family vector (e.g., anellovector), e.g., a synthetic Anelloviridae family vector (e.g., anellovector), e.g., as described herein.

In an aspect, the invention features a method of delivering an effector or payload (e.g., an endogenous or exogenous effector) to a cell, tissue or subject, the method comprising administering to the subject an Anelloviridae family vector (e.g., anellovector), e.g., a synthetic Anelloviridae family vector (e.g., anellovector), e.g., as described herein, wherein the anellovector comprises a nucleic acid sequence encoding the effector. In embodiments, the payload is a nucleic acid. In embodiments, the payload is a polypeptide. In some embodiments, the cell is a cell of the eye. In certain embodiments, the cell of the eye is a photoreceptor cell, a retinal cell, a cell of the posterior eye cup (PEC), a cell of the optic nerve, a cell of the optic nerve head, retinal ganglion cell, or a retinal pigmented epithelium (RPE) cell. In some embodiments, the tissue is a tissue of the eye. In certain embodiments, the tissue of the eye is the retina, posterior eye cup, retinal ganglion, retinal pigmented epithelium, optical nerve, optic nerve head, subretinal space, or intravitreal space.

In an aspect, the invention features a method of delivering an Anelloviridae family vector (e.g., anellovector) to a cell, comprising contacting the Anelloviridae family vector (e.g., anellovector), e.g., a synthetic Anelloviridae family vector (e.g., anellovector), e.g., as described herein, with a cell, e.g., a eukaryotic cell, e.g., a mammalian cell, e.g., in vivo or ex vivo. In some embodiments, the cell is a cell of the eye. In certain embodiments, the cell of the eye is a photoreceptor cell, a retinal cell, a cell of the posterior eye cup (PEC), a cell of the optic nerve, a cell of the optic nerve head, retinal ganglion cell, or a retinal pigmented epithelium (RPE) cell.

In an aspect, the invention features a method of making an Anelloviridae family vector (e.g., anellovector), e.g., a synthetic anellovector. The method includes:
a) providing a host cell comprising:
(i) a first nucleic acid molecule comprising the nucleic acid sequence of a genetic element of an anellovector, e.g., a synthetic anellovector, as described herein, and
(ii) the first nucleic acid or a second nucleic acid molecule encoding one or more of an amino acid sequence chosen from ORF1, ORF2, ORF2/2, ORF2/3, ORF1/1, ORF1/2, VP1, VP2, or VP3, e.g., as listed in Table A1-A3, or an amino acid sequence having at least 70% (e.g., at least 70%, 75%, 80%, 85%, 90%, 95%, 96%, 97%, 98%, 99%, or 100%) sequence identity thereto; and
b) incubating the host cell under conditions suitable to make the Anelloviridae family vector (e.g., anellovector).

In some embodiments, the method further includes, prior to step (a), introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is a helper (e.g., a helper plasmid or the genome of a helper virus).

In another aspect, the invention features a method of manufacturing an Anelloviridae family vector (e.g., anellovector) composition, comprising:
a) providing a host cell comprising, e.g., expressing one or more components (e.g., all of the components) of an Anelloviridae family vector (e.g., anellovector), e.g., a synthetic Anelloviridae family vector (e.g., anellovector), e.g., as described herein. For example, the host cell comprises (a) a nucleic acid comprising a sequence encoding an Anellovirus ORF1 or CAV VP1 polypeptide described herein, wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g, packaging sequence) that binds the polypeptide of (a), wherein the host cell or helper cell comprises (a) and (b) either in cis or in trans. In embodiments, the genetic element of (b) is circular, single-stranded DNA. In some embodiments, the host cell is a manufacturing cell line;
b) culturing the host cell under conditions suitable for producing a preparation of Anelloviridae family vector (e.g., anellovector) from the host cell, wherein the Anelloviridae family vector (e.g., anellovector) of the preparation comprise a proteinaceous exterior (e.g., comprising an ORF1 molecule) encapsulating the genetic element (e.g., as described herein), thereby making a preparation of Anelloviridae family vector (e.g., anellovector); and
optionally, c) formulating the preparation of Anelloviridae family vector (e.g., anellovector), e.g., as a pharmaceutical composition suitable for administration to a subject.

In some embodiments, the components of the Anelloviridae family vector (e.g., anellovector) are introduced into the host cell at the time of production (e.g., by transient transfection). In some embodiments, the host cell stably expresses the components of the Anelloviridae family vector (e.g., anellovector) (e.g., wherein one or more nucleic acids encoding the components of the Anelloviridae family vector (e.g., anellovector) are introduced into the host cell, or a progenitor thereof, e.g., by stable transfection).

In some embodiments, the method further comprises one or more purification steps (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

In an aspect, the invention features a method of manufacturing an Anelloviridae family vector (e.g., anellovector) composition, comprising: a) providing a plurality of Anelloviridae family vectors (e.g., anellovectors) described herein, or a preparation of Anelloviridae family vectors (e.g., anellovectors) described herein; and b) formulating the Anelloviridae family vectors (e.g., anellovectors) or preparation thereof, e.g., as a pharmaceutical composition suitable for administration to a subject.

In an aspect, the invention features a method of making a host cell, e.g., a first host cell or a producer cell (e.g., as shown in FIG. 12), e.g., a population of first host cells, comprising an Anelloviridae family vector (e.g., anellovector), the method comprising introducing a genetic element, e.g., as described herein, to a host cell and culturing the host cell under conditions suitable for production of the Anelloviridae family vector (e.g., anellovector). In some embodiments, the method further comprises introducing a helper, e.g., a helper virus, to the host cell. In some embodiments, the introducing comprises transfection (e.g., chemical transfection) or electroporation of the host cell with the Anelloviridae family vector (e.g., anellovector).

In an aspect, the invention features a method of making an Anelloviridae family vector (e.g., anellovector), comprising providing a host cell, e.g., a first host cell or producer cell (e.g., as shown in FIG. 12), comprising an Anelloviridae family vector (e.g., anellovector), e.g., as described herein, and purifying the Anelloviridae family vector (e.g., anellovector) from the host cell. In some embodiments, the method further comprises, prior to the providing step, contacting the host cell with an Anelloviridae family vector (e.g., anellovector), e.g., as described herein, and incubating the host cell under conditions suitable for production of the Anelloviridae family vector (e.g., anellovector). In some embodiments, the host cell is the first host cell or producer cell described in the above method of making a host cell. In some embodiments, purifying the Anelloviridae family vector (e.g., anellovector) from the host cell comprises lysing the host cell.

In some embodiments, the method further comprises a second step of contacting the Anelloviridae family vector (e.g., anellovector) produced by the first host cell or producer cell with a second host cell, e.g., a permissive cell (e.g., as shown in FIG. 12), e.g., a population of second host cells. In some embodiments, the method further comprises incubating the second host cell inder conditions suitable for production of the Anelloviridae family vector (e.g., anellovector). In some embodiments, the method further comprises purifying an Anelloviridae family vector (e.g., anellovector) from the second host cell, e.g., thereby producing an Anelloviridae family vector (e.g., anellovector) seed population. In some embodiments, at least about 2-100-fold more of the Anelloviridae family vector (e.g., anellovector) is produced from the population of second host cells than from the population of first host cells. In some embodiments, purifying the Anelloviridae family vector (e.g., anellovector) from the second host cell comprises lysing the second host cell. In some embodiments, the method further comprises a second step of contacting the Anelloviridae family vector (e.g., anellovector) produced by the second host cell with a third host cell, e.g., permissive cells (e.g., as shown in FIG. 12), e.g., a population of third host cells. In some embodiments, the method further comprises incubating the third host cell inder conditions suitable for production of the Anelloviridae family vector (e.g., anellovector). In some embodiments, the method further comprises purifying an Anelloviridae family vector (e.g., anellovector) from the third host cell, e.g., thereby producing an Anelloviridae family vector (e.g., anellovector) stock population. In some embodiments, purifying the Anelloviridae family vector (e.g., anellovector) from the third host cell comprises lysing the third host cell. In some embodiments, at least about 2-100-fold more of the Anelloviridae family vector (e.g., anellovector) is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of Anelloviridae family vectors (e.g., anellovectors) by the host cell. In some embodiments, Anelloviridae family vector (e.g., anellovectors) produced by a host cell separated from the host cell (e.g., by lysing the host cell) prior to contact with a second host cell. In some embodiments, Anelloviridae family vectors (e.g., anellovectors) produced by a host cell are contacted with a second host cell without an intervening purification step.

In an aspect, the invention features a method of making a pharmaceutical Anelloviridae family vector (e.g., anellovector) preparation. The method comprises (a) making an Anelloviridae family vector (e.g., anellovector) preparation as described herein, (b) evaluating the preparation (e.g., a pharmaceutical Anelloviridae family vector (e.g., anellovector) preparation, Anelloviridae family vector (e.g., anellovector) seed population or the Anelloviridae family vector (e.g., anellovector) stock population) for one or more pharmaceutical quality control parameters, e.g., identity, purity, titer, potency (e.g., in genomic equivalents per Anelloviridae family vector (e.g., anellovector) particle), and/or the nucleic acid sequence, e.g., from the genetic element comprised by the Anelloviridae family vector (e.g., anellovector), and (c) formulating the preparation for pharmaceutical use of the evaluation meets a predetermined criterion, e.g, meets a pharmaceutical specification. In some embodiments, evaluating identity comprises evaluating (e.g., confirming) the sequence of the genetic element of the Anelloviridae family vector (e.g., anellovector), e.g., the sequence encoding the effector. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted Anelloviridae family vectors (e.g., anellovectors) (e.g., an Anelloviridae family vector (e.g., anellovector) other than the desired Anelloviridae family vector (e.g., anellovector), e.g., a synthetic Anelloviridae family vector (e.g., anellovector) as described herein), free viral capsid protein, adventitious agents, and aggregates. In some embodiments, evalating titer comprises evaluating the ratio of functional versus nonfunctional (e.g., infectious vs non-infectious) Anelloviridae family vectors (e.g., anellovectors) in the preparation (e.g., as evaluated by HPLC). In some embodiments, evaluating potency comprises evaluating the level of Anelloviridae family vector (e.g., anellovector) function (e.g., expression and/or function of an effector encoded therein or genomic equivalents) detectable in the preparation.

In some embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple Anelloviridae family vectors (e.g., anellovectors) can be produced in a single batch. In some embodiments, the levels of the Anelloviridae family vectors (e.g., anellovectors) produced in the batch can be evaluated (e.g., individually or together).

In an aspect, the invention features a host cell comprising:
(i) a first nucleic acid molecule comprising the nucleic acid sequence of a genetic element of an Anelloviridae family vector (e.g., anellovector) as described herein, and
(ii) optionally, a second nucleic acid molecule encoding one or more of an amino acid sequence chosen from ORF1, ORF2, ORF2/2, ORF2/3, ORF1/1, ORF1/2, VP1, VP2, or VP3 as listed in Table A1-A3, or an amino acid sequence having at least about 70% (e.g., at least about 70, 80, 90, 95, 96, 97, 98, 99, or 100%) sequence identity thereto.

In an aspect, the invention features a reaction mixture comprising an Anelloviridae family vector (e.g., anellovector) described herein and a helper virus, wherein the helper virus comprises a polynucleotide, e.g., a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, an Anelloviridae family vector (e.g., anellovector) (e.g., a synthetic Anelloviridae family vector (e.g., anellovector)) is isolated, e.g., isolated from a host cell and/or isolated from other constituents in a solution (e.g., a supernatant). In some embodiments, an Anelloviridae family vector (e.g., anellovector) (e.g., a synthetic Anelloviridae family vector (e.g., anellovector)) is purified, e.g., from a solution (e.g., a supernatant). In some embodiments, an Anelloviridae family vector (e.g., anellovector) is enriched in a solution relative to other constituents in the solution.

In some embodiments of any of the aforesaid Anelloviridae family vectors (e.g., anellovectors), compositions or methods, providing an Anelloviridae family vector (e.g., anellovector) comprises separating (e.g., harvesting) an Anelloviridae family vector (e.g., anellovector) from a composition comprising an Anelloviridae family vector (e.g., anellovector)-producing cell, e.g., as described herein. In other embodiments, providing an Anelloviridae family vector (e.g., anellovector) comprises obtaining an Anelloviridae family vector (e.g., anellovector) or a preparation thereof, e.g., from a third party.

In some embodiments of any of the aforesaid Anelloviridae family vectors (e.g., anellovectors), compositions or methods, the genetic element comprises an Anelloviridae family vector (e.g., anellovector) genome, e.g., as identified according to the method described in Example 9. In embodiments, the Anelloviridae family vector (e.g., anellovector) genome is an Anelloviridae family vector (e.g., anellovector) genome capable of self-replication and/or self-amplification. In some embodiments, the Anelloviridae family vector (e.g., anellovector) genome is not capable of self-replication and/or self-amplification. In some embodiments, the Anelloviridae family vector (e.g., anellovector) genome is capable of replicating and/or being amplified in trans, e.g., in the presence of a helper, e.g., a helper virus.

It is understood that applicable embodiments described herein with respect to anellovectors may also be applied to Anelloviridae family vectors (e.g., a vector based on or derived from a chicken anemia virus (CAV), e.g., as described herein).

Additional features of any of the aforesaid Anelloviridae family vectors (e.g., anellovectors), compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. An Anelloviridae family vector (e.g., an anellovector) comprising:
(i) a proteinaceous exterior comprising an Anellovirus ORF1 protein as listed in Table A1 or A2 or a CAV VP1 protein as listed in Table A3, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.

2. An Anelloviridae family vector (e.g., an anellovector) comprising:
(i) a proteinaceous exterior comprising an Anellovirus ORF1 protein as listed in Table A1 or A2 or a CAV VP1 protein as listed in Table A3, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively or relative to a wild-type CAV VP1 protein and/or wild-type CAV genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

3. An Anelloviridae family vector (e.g., an anellovector) comprising:
(i) a proteinaceous exterior comprising a polypeptide encoded by an Anellovirus ORF1 nucleic acid sequence as listed in any of Tables N1-N2 or by a CAV VP1 nucleic acid sequence of Table N3 or N4, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence or the CAV VP1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.

4. An Anelloviridae family vector (e.g., an anellovector) comprising:
(i) a proteinaceous exterior comprising a polypeptide encoded by an Anellovirus ORF1 nucleic acid sequence as listed in any of Tables N1-N2 or by a CAV VP1 nucleic acid sequence of Table N3 or N4, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence or the CAV nucleic acid sequence, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively, or a wild-type CAV VP1 protein and/or wild-type CAV genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

5. An Anelloviridae family vector (e.g., an anellovector) comprising:
(i) a proteinaceous exterior (e.g., comprising an Anellovirus ORF1 molecule or VP1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in any of Tables N1-N4, or a nucleic acid sequence having at least 70%, 75%, 80%, 85%, 90%, 95%, 96%, 97%, 98%, 99%, or 100% sequence identity thereto, or a complement thereof, and (b) a promoter element operably linked to a nucleic acid sequence (e.g., a DNA sequence) encoding an exogenous effector.

6. An Anelloviridae family vector (e.g., an anellovector) comprising:
(i) a proteinaceous exterior (e.g., comprising an Anellovirus ORF1 molecule or VP1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in any of Tables N1-N4, or a nucleic acid sequence having at least 70%, 75%, 80%, 85%, 90%, 95%, 96%, 97%, 98%, 99%, or 100% sequence identity thereto, or a complement thereof, and (b) a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively or a wild-type CAV VP1 protein and/or wild-type CAV VP1 genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

7. An Anelloviridae family vector (e.g., an anellovector) comprising:
(i) a proteinaceous exterior (e.g., comprising an Anelloviridae family capsid protein, e.g., an Anellovirus ORF1 molecule or CAV VP1 protein as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an exogenous effector, and wherein the genetic element has at least about 70%, 75%, 80%, 85%, 90%, 95%, 96%, 97%, 98%, 99%, or 100% sequence identity to an Anelloviridae family virus genome sequence as listed in any of Tables N1-N4, or a complement thereof.

8. An Anelloviridae family vector (e.g., an anellovector) comprising:
(i) a proteinaceous exterior (e.g., comprising an Anelloviridae capsid protein, e.g., an Anellovirus ORF1 molecule or CAV VP1 molecule as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector), and wherein the genetic element has at least about 70%, 75%, 80%, 85%, 90%, 95%, 96%, 97%, 98%, 99%, or 100% sequence identity to an Anelloviridae family virus (e.g., Anellovirus or CAV) genome sequence as listed in any of Tables N1-N4, or a complement thereof;
wherein the proteinaceous exterior and/or the genetic element comprises at least one difference (e.g., a mutation, chemical modification, or epigenetic alteration) relative to a wild-type Anelloviridae family virus (e.g., Anellovirus or CAV) ORF1 protein and/or wild-type Anelloviridae family virus (e.g., Anellovirus or CAV) genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

9. The Anelloviridae family vector (e.g., anellovector) of any of the preceding embodiments, wherein the at least one difference relative to a wild-type Anelloviridae family virus (e.g., Anellovirus or CAV) ORF1 protein and/or wild-type Anelloviridae family virus (e.g., Anellovirus or CAV) genome comprises encoding an exogenous effector.

10. The Anelloviridae family vector (e.g., anellovector) of any of the preceding embodiments, wherein the proteinaceous exterior comprises the amino acid sequence YNPX²DXGX²N (SEQ ID NO: 829), wherein Xⁿ is a contiguous sequence of any n amino acids.

11. An isolated ORF1 molecule comprising the amino acid sequence of an ORF1 as listed in Table A1 or A2, or an amino acid sequence having at least about 70%, 75%, 80%, 85%, 90%, 95%, 96%, 97%, 98%, 99%, or 100% sequence identity thereto;
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

12. An isolated ORF1 molecule comprising the amino acid sequence of the jelly-roll domain of an ORF1 as listed in Table A1 or A2, or an amino acid sequence having at least about 70%, 75%, 80%, 85%, 90%, 95%, 96%, 97%, 98%, 99%, or 100% sequence identity thereto;
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

13. An isolated VP1 molecule comprising the amino acid sequence of an VP1 as listed in Table A3, or an amino acid sequence having at least about 70%, 75%, 80%, 85%, 90%, 95%, 96%, 97%, 98%, 99%, or 100% sequence identity thereto;
wherein the VP1 molecule comprises at least one difference (e.g., a mutation, chemical modification, or epigenetic alteration) relative to a wild-type VP1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

14. An isolated VP1 molecule comprising the amino acid sequence of the jelly-roll domain of an VP1 as listed in Table A3, or an amino acid sequence having at least about 70%, 75%, 80%, 85%, 90%, 95%, 96%, 97%, 98%, 99%, or 100% sequence identity thereto;
wherein the VP1 molecule comprises at least one difference (e.g., a mutation, chemical modification, or epigenetic alteration) relative to a wild-type VP1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

15. The ORF1 or VP1 molecule of any one of embodiments 13-14, wherein the ORF1 or VP1 molecule comprises the amino acid sequence YNPX²DXGX²N (SEQ ID NO: 829), wherein Xⁿ is a contiguous sequence of any n amino acids.

16. The ORF1 or VP1 molecule of embodiment 15, wherein the amino acid sequence YNPX²DXGX²N (SEQ ID NO: 829) is comprised in an N22 domain of the ORF1 or VP1 molecule.

17. The ORF1 or VP1 molecule of any one of embodiments 13-16, wherein the ORF1 or VP1 molecule comprises one or more (e.g., 1, 2, 3, 4, or all 5) of the following Anellovirus ORF1 or CAV VP1 subdomains: an arginine-rich region, a jelly-roll region, a hypervariable region, an N22 domain, a C-terminal domain (CTD) (e.g., as described herein), e.g., of an Anellovirus ORF1 protein as listed in Table A1 or A2 or a CAV VP1 protein as listed in Table A3 (or a sequence having at least about 70%, 75%, 80%, 85%, 90%, 95%, 96%, 97%, 98%, 99%, or 100% sequence identity thereto).

18. An isolated ORF2 molecule comprising the amino acid sequence of an ORF2 as listed in Table A1 or A2, or an amino acid sequence having at least about 70%, 75%, 80%, 85%, 90%, 95%, 96%, 97%, 98%, 99%, or 100% sequence identity thereto;
wherein the ORF2 molecule comprises at least one difference (e.g., a mutation, chemical modification, or epigenetic alteration) relative to a wild-type ORF2 protein (e.g., as described herein), e.g., an insertion, substitution, chemical or enzymatic modification, and/or deletion, e.g., a deletion of a domain.

19. The ORF2 molecule of embodiment 18, wherein the ORF2 molecule comprises the amino acid sequence [W/F]X⁷HX³CX¹CX⁵H (SEQ ID NO: 949), wherein Xⁿ is a contiguous sequence of any n amino acids.

20. An isolated nucleic acid molecule (e.g., a genetic element construct or a genetic element) comprising the nucleic acid sequence of a 5′ UTR conserved domain as listed in any of Tables N1-N4, or a nucleic acid sequence having at least 70%, 75%, 80%, 85%, 90%, 95%, 96%, 97%, 98%, or 99% sequence identity thereto, or a complement thereof.

21. An isolated nucleic acid molecule (e.g., a genetic element construct or a construct for providing an ORF1 molecule or VP1 molecule in trans, e.g., as described herein) comprising the nucleic acid sequence of an ORF1 gene or a VP1 gene as listed in any of Tables N1-N4, or a nucleic acid sequence having at least 70%, 75%, 80%, 85%, 90%, 95%, 96%, 97%, 98%, or 99% sequence identity thereto, or a complement thereof.

22. An isolated nucleic acid molecule (e.g., a genetic element construct or a construct for providing an ORF2 molecule in trans, e.g., as described herein) comprising the nucleic acid sequence of an ORF2 gene as listed in any of Tables N1-N2, or a nucleic acid sequence having at least 70%, 75%, 80%, 85%, 90%, 95%, 96%, 97%, 98%, or 99% sequence identity thereto, or a complement thereof.

23. An isolated nucleic acid molecule (e.g., a genetic element construct, a genetic element, or a construct for providing an ORF1, ORF2, VP1, or VP2 molecule in trans, e.g., as described herein) comprising an Anellovirus genome sequence as listed in any of Tables N1-N4, or a nucleic acid sequence having at least 50%, 60%, 70%, 75%, 80%, 85%, 90%, 95%, 96%, 97%, 98%, or 99% sequence identity thereto, or a complement thereof.

24. The isolated nucleic acid molecule of any of embodiments 20-23, wherein the isolated nucleic acid molecule comprises at least one difference (e.g., a mutation, chemical modification, or epigenetic alteration) relative to a wild-type Anellovirus genome sequence (e.g., as described herein)

25. The isolated nucleic acid molecule of embodiment 24, wherein the at least one difference comprises a deletion (e.g, lacks one or more of: a 5′ UTR conserved domain, an ORF1 gene, ORF2 gene, a VP1 gene, a VP2 gene, a GC-rich region, an ORF3 gene, a VP3 gene, or a functional fragment thereof).

26. The isolated nucleic acid molecule of any of embodiments 20-25, wherein the isolated nucleic acid molecule is substantially unable to be enclosed in an Anellovirus or CAV capsid (e.g., a proteinaceous exterior of an Anelloviridae family vector (e.g., anellovector) as described herein).

27. The isolated nucleic acid molecule of any of embodiment 20-26, wherein the isolated nucleic acid molecule encodes an effector (e.g., an exogenous effector or an endogenous effector).

28. A genetic element comprising:
(a) a 5′ UTR conserved domain as listed in any of Tables N1-N4, or a nucleic acid sequence having at least 70%, 75%, 80%, 85%, 90%, 95%, 96%, 97%, 98%, 99%, or 100% sequence identity thereto, or a complement thereof, and
(b) a promoter element operably linked to a nucleic acid sequence (e.g., a DNA sequence) encoding an exogenous effector.

29. A genetic element comprising (e.g., in 5′ to 3′ order):
(i) nucleotides 1-71 of SEQ ID NO: 1, or a nucleic acid sequence having at least 93%, 94%, 95%, 96%, 97%, 98%, or 99% sequence identity thereto;
(ii) a 5′ portion of an ORF2 nucleic acid sequence;
(iii) a promoter element;
(iv) a nucleic acid sequence encoding an exogenous effector (e.g., a therapeutic exogenous effector); and
(v) a 3′ portion of an ORF1 nucleic acid sequence;
or a complement of (i)-(v);
wherein the genetic element does not encode a full-length ORF1 polypeptide or a full-length ORF2 polypeptide.

30. The genetic element of embodiment 29, wherein the 3′ portion of the ORF1 nucleic acid sequence comprises nucleotides 4367-5358 of SEQ ID NO: 7, or a nucleic acid sequence having at least 75%, 80%, 85%, 90%, 95%, 96%, 97%, 98%, or 99% sequence identity thereto.

31. The genetic element of embodiment 29 or 30, wherein the 3′ portion of the ORF1 nucleic acid sequence comprises 0-100, 100-200, 200-300, 300-400, 400-500, 500-600, 600-700, 700-800, 800-900, or 900-1000 contiguous nucleotides of the sequence of nucleotides 283-2250 of SEQ ID NO: 1, or a nucleic acid sequence having at least 75%, 80%, 85%, 90%, 95%, 96%, 97%, 98%, or 99% sequence identity thereto.

32. The genetic element of any of embodiments 29-31, wherein the genetic element does not comprise 1-100, 100-200, 200-300, 300-400, 400-500, 500-600, 600-700, 700-800, 800-900, or 900-1000 contiguous nucleotides from the 5′ end of nucleotides 283-2250 of SEQ ID NO: 1, or a nucleic acid sequence having at least 75%, 80%, 85%, 90%, 95%, 96%, 97%, 98%, or 99% sequence identity thereto.

33. The genetic element of embodiment 29, wherein the 3′ portion of the ORF1 nucleic acid sequence comprises nucleotides 4890-5284 of SEQ ID NO: 11, or a nucleic acid sequence having at least 75%, 80%, 85%, 90%, 95%, 96%, 97%, 98%, or 99% sequence identity thereto.

34. The genetic element of embodiment 29 or 30, wherein the 3′ portion of the ORF1 nucleic acid sequence comprises 0-100, 100-200, 200-300, or 300-350, 350-360, 360-370, 370-380, 380-390, or 390-395 contiguous nucleotides of the sequence of nucleotides 4890-5284 of SEQ ID NO: 11, or a nucleic acid sequence having at least 75%, 80%, 85%, 90%, 95%, 96%, 97%, 98%, or 99% sequence identity thereto.

35. The genetic element of any of embodiments 29-34, wherein the ORF2 nucleic acid sequence comprises nucleotides 101-391 of SEQ ID NO: 1, or a nucleic acid sequence having at least 75%, 80%, 85%, 90%, 95%, 96%, 97%, 98%, or 99% sequence identity thereto.

36. The genetic element of any of embodiments 29-35, wherein the ORF2 nucleic acid sequence encodes an ORF2 molecule comprising SEQ ID NO: 3, or an amino acid sequence having at least 75%, 80%, 85%, 90%, 95%, 96%, 97%, 98%, or 99% sequence identity thereto.

37. The genetic element of any of embodiments 29-36, wherein the 5′ portion of the ORF2 nucleic acid sequence comprises nucleotides 3218-3385 of SEQ ID NO: 7, or a nucleic acid sequence having at least 75%, 80%, 85%, 90%, 95%, 96%, 97%, 98%, or 99% sequence identity thereto.

38. The genetic element of any of embodiments 29-37, wherein the 5′ portion of the ORF2 nucleic acid sequence comprises 0-50, 50-100, 100-150, 150-160, 160-165, or 165-168 contiguous nucleotides of the sequence of nucleotides 3218-3385 of SEQ ID NO: 7, or a nucleic acid sequence having at least 75%, 80%, 85%, 90%, 95%, 96%, 97%, 98%, or 99% sequence identity thereto.

39. The genetic element of any of embodiments 29-38, wherein the genetic element does not comprise 0-50, 50-100, 100-150, 150-160, 160-166, 166-170, 170-180, 180-190, 190-200, 200-225, 225-250, 250-275, 275-300, 300-310, 310-320, 320-330, 330-333, contiguous nucleotides from the 3′ end of nucleotides 59-391 of SEQ ID NO: 1, or a nucleic acid sequence having at least 75%, 80%, 85%, 90%, 95%, 96%, 97%, 98%, or 99% sequence identity thereto.

40. The genetic element of any of embodiments 29-39, which further comprises at least one nucleotide (e.g., 1-5, 5-10, 10-20, 20-30, 30-40, 40-50, 50-75, 75-100, 100-110, 110-120, 120-130, 130-132, 132-135, 135-139, 139-140, 140-150, 150-160, 160-170, 170-180, 180-190, or 190-200 nucleotides) between the 5′ portion of the ORF2 nucleic acid and the promoter.

41. The genetic element of any of claims 29-40, which further comprises at least one nucleotide (e.g., 1-5, 5-10, 10-20, 20-30, 30-40, 40-50, 50-75, 75-100, 100-110, 110-120, 120-130, 130-135, 135-139, 139-140, 140-150, 150-160, 160-170, 170-180, 180-190, 190-200, 200-250, 250-300, 300-310, 310-320, 320-323, 323-330, 330-340, 340-350, or 350-400 nucleotides) between the nucleic acid sequence encoding the exogenous effector and the 3′ portion of the ORF1 nucleic acid sequence.

42. The genetic element of any of embodiments 29-41, which further comprises a poly-A tail, e.g., positioned between the nucleic acid sequence encoding the exogenous effector amd the 3′ portion of the ORF1 nucleic acid sequence.

43. The genetic element of embodiment 42, which further comprises at least one nucleotide (e.g., 1-5, 5-10, 10-20, 20-30, 30-40, 40-50, 50-75, 75-100, 100-110, 110-120, 120-130, 130-135, 135-139, 139-140, 140-150, 150-160, 160-170, 170-180, 180-190, 190-200, 200-250, 250-300, 300-310, 310-320, 320-323, 323-330, 330-340, 340-350, or 350-400 nucleotides) between the poly-A tail and the 3′ portion of the ORF1 nucleic acid sequence.

44. A genetic element comprising (e.g., in 5′ to 3′ order):
(i) nucleotides 1-71 of SEQ ID NO: 1, or a nucleic acid sequence having at least 93%, 94%, 95%, 96%, 97%, 98%, or 99% sequence identity thereto;
(ii) a 5′ portion of an ORF1 nucleic acid sequence;
(iii) a promoter element;
(iv) a nucleic acid sequence encoding an exogenous effector (e.g., a therapeutic exogenous effector); and
(v) a 3′ portion of an ORF1 nucleic acid sequence;
or a complement of (i)-(v);
wherein the genetic element does not encode a full-length ORF1 polypeptide.

45. The genetic element of embodiment 44, wherein the 5′ portion of the ORF1 nucleic acid sequence comprises nucleotides 3400-3684 of SEQ ID NO: 8, or a nucleic acid sequence having at least 75%, 80%, 85%, 90%, 95%, 96%, 97%, 98%, or 99% sequence identity thereto.

46. The genetic element of any of embodiments 44-45, wherein the 5′ portion of the ORF1 nucleic acid sequence comprises 0-100, 100-200, 200-300, 250-260, 260-270, 270-280, 280-284, 284-290, or 290-300 contiguous nucleotides of the sequence of nucleotides 283-2250 of SEQ ID NO: 1, or a nucleic acid sequence having at least 75%, 80%, 85%, 90%, 95%, 96%, 97%, 98%, or 99% sequence identity thereto.

47. The genetic element of any of embodiments 44-46, wherein the 3′ portion of the ORF1 nucleic acid sequence comprises nucleotides 4663-5358 of SEQ ID NO: 8, or a nucleic acid sequence having at least 75%, 80%, 85%, 90%, 95%, 96%, 97%, 98%, or 99% sequence identity thereto.

48. The genetic element of any of embodiments 44-47, wherein the 3′ portion of the ORF1 nucleic acid sequence comprises 0-100, 100-200, 200-300, 300-400, 400-500, 500-600, or 600-700 contiguous nucleotides of the sequence of nucleotides 283-2250 of SEQ ID NO: 1, or a nucleic acid sequence having at least 75%, 80%, 85%, 90%, 95%, 96%, 97%, 98%, or 99% sequence identity thereto.

49. The genetic element of any of embodiments 44-48, wherein the genetic element does not comprise 1-100, 100-200, 200-300, 300-350, 350-400, 400-450, 450-500, 500-550, 550-600, 600-650, 650-700, 700-750, 750-800, 800-850, 850-900, 900-950, 950-960, 960-970, 970-980, 980-987, 987-990, or 990-1000 contiguous nucleotides from the portion of nucleotides 283-2250 of SEQ ID NO: 1 corresponding to the portion of SEQ ID NO: 8 replaced by an nLuc expression cassette, or a nucleic acid sequence having at least 75%, 80%, 85%, 90%, 95%, 96%, 97%, 98%, or 99% sequence identity thereto.

50. The genetic element of any of embodiments 44-49, wherein the nucleic acid sequences of (iii) and (iv) are comprised in the portion of nucleotides 283-2250 of SEQ ID NO: 1 corresponding to the portion of SEQ ID NO: 8 replaced by an nLuc expression cassette.

51. The genetic element of embodiment 44, wherein the 5′ portion of the ORF1 nucleic acid sequence comprises nucleotides 3400-3984 of SEQ ID NO: 9, or a nucleic acid sequence having at least 75%, 80%, 85%, 90%, 95%, 96%, 97%, 98%, or 99% sequence identity thereto.

52. The genetic element of embodiment 44 or 51, wherein the 5′ portion of the ORF1 nucleic acid sequence comprises 0-100, 100-200, 200-300, 300-400, 400-500, 500-600, 550-560, 560-570, 570-580, 580-584, 584-590, or 590-600 contiguous nucleotides of the sequence of nucleotides 283-2250 of SEQ ID NO: 1 or a nucleic acid sequence having at least 75%, 80%, 85%, 90%, 95%, 96%, 97%, 98%, or 99% sequence identity thereto.

53. The genetic element of any of embodiments 44 or 51-52, wherein the 3′ portion of the ORF1 nucleic acid sequence comprises nucleotides 4964-5358 of SEQ ID NO: 9, or a nucleic acid sequence having at least 75%, 80%, 85%, 90%, 95%, 96%, 97%, 98%, or 99% sequence identity thereto.

54. The genetic element of any of embodiments 44 or 51-53, wherein the 3′ portion of the ORF1 nucleic acid sequence comprises 0-100, 100-200, 200-300, 300-400, 350-360, 360-370, 370-380, 380-390, 390-394, or 394-400 contiguous nucleotides of the sequence of nucleotides 283-2250 of SEQ ID NO: 1, or a nucleic acid sequence having at least 75%, 80%, 85%, 90%, 95%, 96%, 97%, 98%, or 99% sequence identity thereto.

55. The genetic element of any of embodiments 44 or 51-54, wherein the genetic element does not comprise 1-100, 100-200, 200-300, 300-400, 400-500, 500-600, 600-700, 700-800, 800-900, or 900-1000 contiguous nucleotides from the portion of nucleotides 283-2250 of SEQ ID NO: 1 corresponding to the portion of SEQ ID NO: 9 replaced by an nLuc expression cassette, or a nucleic acid sequence having at least 75%, 80%, 85%, 90%, 95%, 96%, 97%, 98%, or 99% sequence identity thereto.

56. The genetic element of any of embodiments 44 or 51-55, wherein the nucleic acid sequences of (iii) and (iv) are comprised in the portion of nucleotides 283-2250 of SEQ ID NO: 1 corresponding to the portion of SEQ ID NO: 9 replaced by an nLuc expression cassette.

57. The genetic element of any of embodiments 44-56, which further comprises at least one nucleotide (e.g., 1-5, 5-10, 10-20, 20-30, 30-40, 40-50, 50-75, 75-100, 100-110, 110-120, 120-130, 130-135, 135-139, 139-140, 140-150, 150-160, 160-170, 170-180, 180-190, or 190-200 nucleotides) between the 5′ portion of the ORF1 nucleic acid and the promoter.

58. The genetic element of embodiment 57, which further comprises at least one nucleotide (e.g., 1-5, 5-10, 10-20, 20-30, 30-40, 40-50, 50-75, 75-100, 100-110, 110-120, 120-130, 130-135, 135-139, 139-140, 140-150, 150-160, 160-170, 170-180, 180-190, 190-200, 200-250, 250-300, 300-310, 310-320, 320-323, 323-330, 330-340, 340-350, or 350-400 nucleotides) between the nucleic acid sequence encoding the exogenous effector and the 3′ portion of the ORF1 nucleic acid sequence.

59. The genetic element of any of embodiments 44-58, which further comprises a poly-A tail, e.g., positioned between the nucleic acid sequence encoding the exogenous effector amd the 3′ portion of the ORF1 nucleic acid sequence.

60. The genetic element of embodiment 59, which further comprises at least one nucleotide (e.g., 1-5, 5-10, 10-20, 20-30, 30-40, 40-50, 50-75, 75-100, 100-110, 110-120, 120-130, 130-135, 135-139, 139-140, 140-150, 150-160, 160-170, 170-180, 180-190, 190-200, 200-250, 250-300, 300-310, 310-320, 320-323, 323-330, 330-340, 340-350, or 350-400 nucleotides) between the poly-A tail and the 3′ portion of the ORF1 nucleic acid sequence.

61. The genetic element of any of embodiments 44-60, which further comprises an ORF2 nucleic acid sequence.

62. The genetic element of embodiment 61, wherein the ORF2 nucleic acid sequence comprises nucleotides 101-391 of SEQ ID NO: 1, or a nucleic acid sequence having at least 75%, 80%, 85%, 90%, 95%, 96%, 97%, 98%, or 99% sequence identity thereto.

63. The genetic element of embodiment 61, wherein the ORF2 molecule comprises the amino acid sequence of SEQ ID NO: 3, or a sequence having at least 75%, 80%, 85%, 90%, 95%, 96%, 97%, 98%, or 99% sequence identity thereto.

64. The genetic element of any of the preceding embodiments, wherein the ORF1 nucleic acid sequence comprises nucleotides 283-2250 of SEQ ID NO: 1, or a nucleic acid sequence having at least 75%, 80%, 85%, 90%, 95%, 96%, 97%, 98%, or 99% sequence identity thereto.

65. The genetic element of embodiment 64, wherein the 5′ codon of the ORF1 nucleic acid sequence is an ATG.

66. The genetic element of embodiment 64, wherein the 5′ codon of the ORF1 nucleic acid sequence is not an ATG (e.g., wherein the 5′ codon of the ORF1 nucleic acid sequence is AAA).

67. The genetic element of any of the preceding embodiments, wherein the encoded ORF1 molecule comprises SEQ ID NO: 2, or an amino acid sequence having at least 75%, 80%, 85%, 90%, 95%, 96%, 97%, 98%, or 99% sequence identity thereto.

68. The genetic element of any of the preceding embodiments, which further comprises nucleotides 2277-2462 of SEQ ID NO: 1, or a nucleic acid sequence having at least 75%, 80%, 85%, 90%, 95%, 96%, 97%, 98%, or 99% sequence identity thereto.

69. The genetic element of any of the preceding embodiments, which further comprises a sequence encoding SEQ ID NO: 4, or an amino acid sequence having at least 75%, 80%, 85%, 90%, 95%, 96%, 97%, 98%, or 99% sequence identity thereto.

70. The genetic element of any of the preceding embodiments, which further comprises nucleotides 2515-2615 of SEQ ID NO: 1, or a nucleic acid sequence having at least 75%, 80%, 85%, 90%, 95%, 96%, 97%, 98%, or 99% sequence identity thereto.

71. The genetic element of any of the preceding embodiments, which further comprises a promoter.

72. The genetic element of embodiment 71, wherein the promoter comprises a CMV promoter, e.g., comprising the nucleic acid sequence of nucleotides 3525-3728 of SEQ ID NO: 8, or a nucleic acid sequence having at least 75%, 80%, 85%, 90%, 95%, 96%, 97%, 98%, or 99% sequence identity thereto.

73. The genetic element of embodiment 71, wherein the promoter comprises a hEF1a promoter (e.g., a minimal hEF1a promoter), a UbC promoter, an MSCV promoter, a SFFV promoter, a hPGK promoter, a CMV promoter (e.g., a minimal CMV promoter), an INS84 promoter, or a U1a promoter.

74. The genetic element of embodiment 71, wherein the promoter comprises an SV40 promoter, e.g., comprising the nucleic acid sequence of nucleotides 3417-3613 of SEQ ID NO: 11, or a nucleic acid sequence having at least 75%, 80%, 85%, 90%, 95%, 96%, 97%, 98%, or 99% sequence identity thereto.

75. The genetic element of any of the preceding embodiments, which further comprises a poly A sequence (e.g., an SV40 poly A sequence, e.g., comprising the nucleic acid sequence of nucleotides 4301-4349 of SEQ ID NO: 7, or a nucleic acid sequence having at least 75%, 80%, 85%, 90%, 95%, 96%, 97%, 98%, or 99% sequence identity thereto).

76. The genetic element of any of the preceding embodiments, wherein the 5′ codon of the ORF2 nucleic acid sequence is an ATG.

77. The genetic element of any of the preceding embodiments, wherein the 5′ codon of the ORF2 nucleic acid sequence is not an ATG (e.g., wherein the 5′ codon of the ORF2 nucleic acid sequence is AAA).

78. The genetic element of any of the preceding embodiments, wherein the 5′ codon of the ORF1 nucleic acid sequence is an ATG.

79. The genetic element of any of the preceding embodiments, wherein the 5′ codon of the ORF1 nucleic acid sequence is not an ATG (e.g., wherein the 5′ codon of the ORF1 nucleic acid sequence is AAA).

80. A nucleic acid molecule comprising (e.g., in 5′ to 3′ order):
(a) an Anellovirus genome sequence (e.g., comprising the nucleic acid sequence of SEQ ID NO: 1, or a nucleic acid sequence having at least 75%, 80%, 85%, 90%, 95%, 96%, 97%, 98%, or 99% sequence identity thereto; and
(b) the nucleic acid sequence of the genetic element of any of the preceding embodiments.

81. The nucleic acid molecule of embodiment 80, which is a plasmid.

82. An anellovector comprising:
(i) a proteinaceous exterior (e.g., comprising an Anellovirus ORF1 protein, e.g., as listed in Table A1, or a polypeptide comprising an amino acid sequence having at least about 70%, 75%, 80%, 85%, 90%, 95%, 96%, 97%, 98%, or 99% sequence identity thereto), and
(ii) the genetic element of any of the preceding embodiments;
wherein the genetic element is enclosed by the proteinaceous exterior.

83. A method of making an anellovector, the method comprising:
(a) providing a cell, e.g., a host cell as described herein;
(b) introducing a nucleic acid molecule encoding an ORF1 polypeptide (e.g., comprising the amino acid sequence of an ORF1 protein as listed in Table A1, or a sequence having at least about 70%, 75%, 80%, 85%, 90%, 95%, 96%, 97%, 98%, or 99% sequence identity thereto) into the cell;
(c) introducing the nucleic acid molecule of embodiment 80 or 81 into the cell (e.g., before, after, or simultaneously with (b)),
(d) incubating the cell under conditions that allow the cell to produce an anellovector; and
thereby making the anellovector.

84. A method of making an anellovector, the method comprising:
(a) providing a cell (e.g., a host cell as described herein) comprising a nucleic acid molecule encoding an ORF1 polypeptide (e.g., comprising the amino acid sequence of an ORF1 protein as listed in Table A1, or a sequence having at least about 70%, 75%, 80%, 85%, 90%, 95%, 96%, 97%, 98%, or 99% sequence identity thereto);
(b) introducing the nucleic acid molecule of embodiment 80 or 81 into the cell,
(c) incubating the cell under conditions that allow the cell to produce an anellovector; and
thereby making the anellovector.

85. The method of embodiment 83 or 84, further comprising formulating the anellovectors, e.g., as a pharmaceutical composition suitable for administration to a subject.

86. A pharmaceutical composition comprising the Anelloviridae family vector (e.g., anellovector), ORF1 molecule, ORF2 molecule, VP1 molecule, VP2 molecule, genetic element, or nucleic acid molecule of any of the preceding embodiments, and a pharmaceutically acceptable carrier and/or excipient.

87. The pharmaceutical composition of embodiment 86, wherein the pharmaceutical composition has one or more of the following characteristics:
a) the pharmaceutical composition meets a pharmaceutical or good manufacturing practices (GMP) standard;
b) the pharmaceutical composition was made according to good manufacturing practices (GMP);
c) the pharmaceutical composition has a pathogen level below a predetermined reference value, e.g., is substantially free of pathogens;
d) the pharmaceutical composition has a contaminant level below a predetermined reference value, e.g., is substantially free of contaminants;
e) the pharmaceutical composition has a predetermined level of non-infectious particles or a predetermined ratio of particles:infectious units (e.g., <300:1, <200:1, <100:1, or <50:1), or
f) the pharmaceutical composition has low immunogenicity or is substantially non-immunogenic, e.g., as described herein.

88. The pharmaceutical composition of any one of embodiments 86-87, wherein the pharmaceutical composition has a contaminant level below a predetermined reference value, e.g., is substantially free of contaminants.

89. The pharmaceutical composition of embodiment 88, wherein the contaminant is selected from the group consisting of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted Anelloviridae family vector (e.g., anellovector) (e.g., an Anelloviridae family vector other than the desired Anelloviridae family vector, e.g., a synthetic Anelloviridae family vector as described herein), free viral capsid protein, adventitious agents, and aggregates.

90. The pharmaceutical composition of embodiment 88, wherein the contaminant is host cell DNA and the threshold amount is about 10 ng of host cell DNA per dose of the pharmaceutical composition.

91. The pharmaceutical composition of any one of embodiments 86-90, wherein the pharmaceutical composition comprises less than 10% (e.g., less than about 10%, 5%, 4%, 3%, 2%, 1%, 0.5%, or 0.1%) contaminant by weight.

92. An ocular delivery system comprising an Anelloviridae family vector (e.g., an anellovector, e.g., as described herein).

93. An isolated cell, e.g., a host cell, comprising:
(a) a nucleic acid molecule encoding an ORF1 polypeptide and/or an ORF2 polypeptide or a VP1 polypeptide and/or a VP2 polypeptide of any of the preceding embodiments, wherein the nucleic acid is a plasmid, is a viral nucleic acid, or is integrated into a cell chromosome, and
(b) a genetic element construct comprising a promoter element and a nucleic acid sequence (e.g., a DNA sequence) encoding an effector (e.g., an exogenous effector or an endogenous effector), and a protein binding sequence,
wherein optionally the genetic element does not encode an ORF1 polypeptide (e.g., an ORF1 protein) or a VP1 polypeptide.

94. An isolated cell, e.g., a host cell, comprising:
(i) a first nucleic acid molecule comprising the nucleic acid sequence of a genetic element of an Anelloviridae family vector (e.g., anellovector) of any of the preceding embodiments (optionally wherein the genetic element does not encode an ORF1 molecule or VP1 molecule), and
(ii) a second nucleic acid molecule, encoding an amino acid sequence of an ORF1 or ORF2 as listed in Table A1 or A2, or an amino acid sequence of a VP1 or VP2 as listed in Table A3, or an amino acid sequence having at least 70% (e.g., at least 70%, 75%, 80%, 85%, 90%, 95%, 96%, 97%, 98%, 99%, or 100%) sequence identity thereto.

95. A method of manufacturing an Anelloviridae family vector (e.g., anellovector) composition, the method comprising:
(a) providing a cell, e.g., a host cell as described herein;
(b) introducing a genetic element construct encoding the genetic element of an Anelloviridae family vector (e.g., anellovector) of any of the preceding embodiments into the cell;
(c) incubating the cell under conditions that allow the cell to produce Anelloviridae family vector (e.g., anellovector), and
(d) formulating the anellovectors, e.g., as a pharmaceutical composition suitable for administration to a subject,
thereby making the Anelloviridae family vector (e.g., anellovector) composition.

96. A method of manufacturing an Anelloviridae family vector (e.g., anellovector) composition, the method comprising:
(a) providing a cell, e.g., a host cell as described herein;
(b) introducing a nucleic acid molecule encoding an ORF1 or ORF2 polypeptide as listed in Table A1 or A2, or a VP1 polypeptide as listed in Table A3 (or an amino acid sequence having at least about 70%, 75%, 80%, 85%, 90%, 95%, 96%, 97%, 98%, or 99% sequence identity thereto) into the cell;
(c) introducing a genetic element construct into the cell (e.g., before, after, or simultaneously with (b)),
(d) incubating the cell under conditions that allow the cell to produce Anelloviridae family vector (e.g., anellovector); and
(e) formulating the Anelloviridae family vector (e.g., anellovector), e.g., as a pharmaceutical composition suitable for administration to a subject,
thereby making the Anelloviridae family vector (e.g., anellovector) composition.

97. A method of manufacturing an Anelloviridae family vector (e.g., anellovector) composition, the method comprising:
(a) providing a cell, e.g., a host cell as described herein;
(b) introducing a nucleic acid molecule encoding an ORF1, ORF2, VP1 or VP2 polypeptide into the cell;
(c) introducing a genetic element construct into the cell as listed in any of Tables N1-N4 (or a nucleic acid sequence having at least about 70%, 75%, 80%, 85%, 90%, 95%, 96%, 97%, 98%, or 99% sequence identity thereto) (e.g., before, after, or simultaneously with (b)),
(d) incubating the cell under conditions that allow the cell to produce Anelloviridae family vector (e.g., anellovector); and
(e) formulating the Anelloviridae family vectors (e.g., anellovectors), e.g., as a pharmaceutical composition suitable for administration to a subject,
thereby making the Anelloviridae family vector (e.g., anellovector) composition.

98. A method of making an Anelloviridae family vector (e.g., anellovector), e.g., a synthetic Anelloviridae family vector (e.g., anellovector), comprising:
(a) providing a host cell comprising:
   (i) a nucleic acid molecule, e.g., a first nucleic acid molecule, comprising the nucleic acid sequence of a Anellovirus genome as listed in any of Tables N1-N4 (or a nucleic acid sequence having at least 70%, 75%, 80%, 85%, 90%, 95%, 96%, 97%, 98%, or 99% sequence identity thereto), and
   (ii) a nucleic acid molecule, e.g., a second nucleic acid molecule, encoding one or more of an amino acid sequence chosen from ORF1, ORF2, ORF2/2, ORF2/3, ORF1/1, ORF1/2, VP1, or VP2, e.g., as listed in Table A1-A3, or an amino acid sequence having at least 70% 75%, 80%, 85%, 90%, 95%, 96%, 97%, 98%, or 99% sequence identity thereto; and
(b) culturing the host cell under conditions suitable to make the Anelloviridae family vector (e.g., anellovector).

99. The method of embodiment 98, further comprising, prior to step (a), introducing the first nucleic acid molecule and/or the second nucleic acid molecule into the host cell.

100. The method of embodiment 98 or 99, wherein the second nucleic acid molecule is introduced into the host cell prior to, concurrently with, or after the first nucleic acid molecule.

101. The method of any of embodiments 95-100, further comprising separating the Anelloviridae family vector (e.g., anellovector) from the cell.

102. A method of manufacturing an ORF1 or VP1 molecule, the method comprising:
(a) providing a host cell (e.g., a host cell described herein) comprising a nucleic acid encoding the ORF1 polypeptide or VP1 polypeptide of any of the preceding embodiments, and
(b) maintaining the host cell under conditions that allow the cell to produce the polypeptide; thereby manufacturing the ORF1 or VP1 molecule.

103. The method of any of embodiments 95-102, wherein the method comprises purifying the Anelloviridae family vector using a CsCl gradient (e.g., as described in Example 20).

104. The method of any of embodiments 95-103, wherein the method comprises purifying the Anelloviridae family vector using an iodixanol linear gradient (e.g., as described in Example 20).

105. A method of delivering an effector (e.g., an exogenous effector or an endogenous effector, e.g., overexpressing an endogenous effector) to a subject (e.g., to an eye of the subject, e.g., to a photoreceptor, retina, posterior eye cup (PEC), retinal ganglion, optic nerve, optic nerve head, retinal pigmented epithelium (RPE), intravitreal space, or subretinal space of the subject), the method comprising administering to the subject (e.g., to the eye of the subject, e.g., to a photoreceptor, retina, posterior eye cup (PEC), retinal ganglion, optic nerve head, subretinal space, intravitreal space, or retinal pigmented epithelium (RPE) of the subject) an Anelloviridae family vector (e.g., anellovector) or pharmaceutical composition of any of the preceding embodiments.

106. A method of delivering an effector (e.g., an exogenous effector or an endogenous effector, e.g., overexpressing an endogenous effector) to a target cell (e.g., a cell of the eye, e.g., a photoreceptor cell, a retinal cell, a cell of the posterior eye cup (PEC), retinal ganglion cell, a cell of the optic nerve, a cell of the optic nerve head, or a retinal pigmented epithelium (RPE) cell), the method comprising contacting the target cell with an Anelloviridae family vector (e.g., anellovector) of any of the preceding embodiments.

107. A method of delivering an effector (e.g., an exogenous effector or an endogenous effector, e.g., overexpressing an endogenous effector) to a target cell ex vivo (e.g., a target cell isolated from a subject, e.g., a patient), the method comprising contacting the target cell with an Anelloviridae family vector (e.g., anellovector) of any of the preceding embodiments.

108. A method of modulating, e.g., enhancing or inhibiting, a biological function (e.g., as described herein) in a subject (e.g., in an eye of the subject, e.g., in a photoreceptor, retina, posterior eye cup (PEC), retinal ganglion, optic nerve, optic nerve head, subretinal space, intravitreal space, or retinal pigmented epithelium (RPE) of the subject), the method comprising administering the Anelloviridae family vector (e.g., anellovector) or the pharmaceutical composition of any of the preceding embodiments to the subject (e.g., to the eye of the subject, e.g., to a photoreceptor, retina, posterior eye cup (PEC), retinal ganglion, optic nerve, optic nerve head, subretinal space, intravitreal space, or retinal pigmented epithelium (RPE) of the subject).

109. A method of treating a disease or disorder (e.g., an eye disease or disorder) in a subject in need thereof, the method comprising administering to the subject (e.g., to an eye of the subject, e.g., to a photoreceptor, retina, posterior eye cup (PEC), retinal ganglion, optic nerve, optic nerve head, subretinal space, intravitreal space, or retinal pigmented epithelium (RPE) of the subject) an Anelloviridae family vector (e.g., anellovector) or pharmaceutical composition of any of the preceding embodiments.

110. Use of the Anelloviridae family vector (e.g., anellovector) or pharmaceutical composition of any the preceding embodiments for treating a disease or disorder (e.g., as described herein) in a subject, wherein optionally the disease or disorder is a disease or disorder of the eye.

111. The Anelloviridae family vector (e.g., anellovector) or pharmaceutical composition of any the preceding embodiments for use in treating a disease or disorder (e.g., as described herein) in a subject, wherein optionally the disease or disorder is a disease or disorder of the eye.

112. Use of the Anelloviridae family vector (e.g., anellovector) or pharmaceutical composition of any the preceding embodiments in the manufacture of a medicament for treating a disease or disorder (e.g., as described herein) in a subject, wherein optionally the disease or disorder is a disease or disorder of the eye.

113. A method of delivering an effector (e.g., an exogenous effector or an endogenous effector, e.g., overexpressing an endogenous effector) to an eye of the subject (e.g., to a photoreceptor, retina, posterior eye cup (PEC), retinal ganglion, optic nerve, optic nerve head, subretinal space, intravitreal space, or retinal pigmented epithelium (RPE) of the subject), the method comprising administering to the eye of the subject (e.g., to a photoreceptor, retina, posterior eye cup (PEC), retinal ganglion, optic nerve, optic nerve head, subretinal space, intravitreal space, or retinal pigmented epithelium (RPE) of the subject) an Anelloviridae family vector (e.g., an anellovector).

114. A method of delivering an effector (e.g., an exogenous effector or an endogenous effector, e.g., overexpressing an endogenous effector) to a cell of the eye (e.g., a photoreceptor cell, a retinal cell, a cell of the posterior eye cup (PEC), retinal ganglion cell, a cell of the optic nerve, a cell of the optic nerve head, or a retinal pigmented epithelium (RPE) cell), the method comprising contacting the cell of the eye with an Anelloviridae family vector (e.g., an anellovector) of any of the preceding embodiments.

115. A method of delivering an effector (e.g., an exogenous effector or an endogenous effector, e.g., overexpressing an endogenous effector) to a target eye cell ex vivo (e.g., a target eye cell isolated from a subject, e.g., a patient), the method comprising contacting the target eye cell with an Anelloviridae family vector (e.g., an anellovector) of any of the preceding embodiments.

116. A method of modulating, e.g., enhancing or inhibiting, a biological function (e.g., as described herein) in an eye of the subject (e.g., in a photoreceptor, retina, posterior eye cup (PEC), retinal ganglion, optic nerve, optic nerve head, subretinal space, intravitreal space, or retinal pigmented epithelium (RPE) of the subject), the method comprising administering the Anelloviridae family vector (e.g., the anellovector) or the pharmaceutical composition of any of the preceding embodiments to the eye of the subject (e.g., to a photoreceptor, retina, posterior eye cup (PEC), retinal ganglion, optic nerve, optic nerve head, subretinal space, intravitreal space, or retinal pigmented epithelium (RPE) of the subject).

117. The method of embodiment 116, wherein the biological function comprises one or more of: best corrected visual acuity (BCVA) retinal sensitivity to light (e.g., as measured by perimetry or microperimetry, e.g., in the dark and light-adapted states, full-field, multi-focal, focal or pattern electroretinography ERG), contrast sensitivity, reading speed, and/or color vision.

118. The method of embodiment 116 or 117, wherei the biological function is measured using clinical biomicroscopic examination, fundus photography, optical coherence tomography (OCT), fundus auto-fluorescence (FAF), infrared and/or multicolor imaging, fluorescein or ICG angiography, and/or adoptive optics.

119. A method of treating a disease or disorder (e.g., an eye disease or disorder) in a subject in need thereof, the method comprising administering to an eye of the subject (e.g., to a photoreceptor, retina, posterior eye cup (PEC), retinal ganglion, optic nerve, optic nerve head, subretinal space, intravitreal space, or retinal pigmented epithelium (RPE) of the subject) an Anelloviridae family vector (e.g., an anellovector) or pharmaceutical composition of any of the preceding embodiments.

120. Use of Anelloviridae family vector (e.g., anellovector) or pharmaceutical composition of any the preceding embodiments for treating a disease or disorder (e.g., as described herein) in a subject, wherein the disease or disorder is a disease or disorder of the eye.

121. The Anelloviridae family vector (e.g., anellovector) or pharmaceutical composition of any the preceding embodiments for use in treating a disease or disorder (e.g., as described herein) in a subject, wherein the disease or disorder is a disease or disorder of the eye.

122. Use of the Anelloviridae family vector (e.g., anellovector) or pharmaceutical composition of any the preceding embodiments in the manufacture of a medicament for treating a disease or disorder (e.g., as described herein) in a subject, wherein the disease or disorder is a disease or disorder of the eye.

123. The method, use, or Anelloviridae family vector (e.g., anellovector) or pharmaceutical composition or use of any of claims 109-122, wherein the disease or disorder is a monogenic disease.

124. The method, use, or Anelloviridae family vector (e.g., anellovector) or pharmaceutical composition or use of any of claims 109-123, wherein the disease or disorder is a polygenic disease (e.g., glaucoma).

125. The method, use, or Anelloviridae family vector (e.g., anellovector) or pharmaceutical composition or use of any of claims 109-124, wherein the disease or disorder is macular degeneration (e.g., age-related macular degeneration (AMD), Stargardt disease, or myopic macular degeneration).

126. The method, use, or Anelloviridae family vector (e.g., anellovector) or pharmaceutical composition or use of claim 125, wherein the macular degeneration is wet AMD.

127. The method, use, or Anelloviridae family vector (e.g., anellovector) or pharmaceutical composition or use of claim 125, wherein the macular degeneration is dry AMD (e.g., AMD with geographic atrophy).

128. The method, use, or Anelloviridae family vector (e.g., anellovector) or pharmaceutical composition or use of any of claims 109-127, wherein the disease or disorder is a retinal disease.

129. The method, use, or Anelloviridae family vector (e.g., anellovector) or pharmaceutical composition or use of claim 128, wherein the retinal disease is an inherited retinal disease (IRD), e.g., as described in Stone et al. (2017, Ophthalmology; incorporated herein by reference with respect to diseases and disorders described therein).

130. The method, use, or Anelloviridae family vector (e.g., anellovector) or pharmaceutical composition or use of claim 128, wherein the retinal disease is retinitis pigmentosa (e.g., X-linked retinitis pigmentosa (XLRP)).

131. The method, use, or Anelloviridae family vector (e.g., anellovector) or pharmaceutical composition or use of any of claims 109-130, wherein the disease or disorder is a VEGF-associated disorder (e.g., a cancer, e.g., as described herein; a macular edema; or a proliferative retinopathy).

132. The method, use, or Anelloviridae family vector (e.g., anellovector) or pharmaceutical composition or use of any of claims 109-131, wherein the disease or disorder is selected from the group consisting of: retinal leakage, Leber congenital amaurosis (LCA) (e.g., wherein the genetic element comprises a human RPE65 sequence, e.g., a sequence encoding a human RPE65 protein, or an amino acid sequence having at least 75%, 80%, 85%, 90%, 95%, 96%, 97%, 98%, or 99% sequence identity thereto), amaurosis congenita, cone rod dystrophy, choroideremia, vitelliform macular dystrophy, hyperferritinemia-cataract syndrome, optic atrophy, XLR retinoschisis, cytomegalovirus retinitis, achromatopsia, Leber hereditary optical neuropathy, keratitis, uveitis, Grave's opthalmolopathy, diabetic retinopathy, or diabetic macular edema.

133. The method, use, or Anelloviridae family vector (e.g., anellovector) or pharmaceutical composition or use of any of claims 109-132, wherein the Anelloviridae family vector is administered to the subject subretinally or into the subretinal space, intravitreally or into the intravitreal space, suprachoroidally or into the suprachoroidal space.

134. The method, use, or Anelloviridae family vector (e.g., anellovector) or pharmaceutical composition or use of any of claims 109-133, wherein the Anelloviridae family vector is administered to the subject subretinally or into the subretinal space.

135. The method, use, or Anelloviridae family vector (e.g., anellovector) or pharmaceutical composition or use of any of claims 109-134, wherein the Anelloviridae family vector is administered to the subject intravitreally or into the intravitreal space.

136. The method, use, or Anelloviridae family vector (e.g., anellovector) or pharmaceutical composition or use of any of claims 109-135, wherein the Anelloviridae family vector is administered to the subject suprachoroidally or into the suprachoroidal space.

137. The method, use, or Anelloviridae family vector (e.g., anellovector) or pharmaceutical composition or use of any of claims 109-136, wherein the Anelloviridae family vector is administered to the subject via an SCS microinjector, via a cannula, and/or via a needle.

138. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element is single-stranded.

139. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element is circular.

140. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises DNA.

141. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element is double-stranded.

142. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element is linear.

143. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises RNA.

144. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises a nucleic acid sequence encoding an Anelloviridae capsid protein, e.g., an Anellovirus ORF1 molecule or CAV VP1 molecule (e.g., an ORF1 or VP1 protein as listed in Table A1-A3 or an amino acid sequence having at least 70%, 75%, 80%, 85%, 90%, 95%, 96%, 97%, 98%, or 99% sequence identity thereto).

145. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element does not comprise a nucleic acid sequence encoding an Anelloviridae capsid protein, e.g., an Anellovirus ORF1 molecule or CAV VP1 molecule (e.g., an ORF1 or VP1 protein as listed in Table A1-A3 or an amino acid sequence having at least 70%, 75%, 80%, 85%, 90%, 95%, 96%, 97%, 98%, or 99% sequence identity thereto).

146. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises a nucleic acid sequence encoding an Anellovirus ORF2 molecule or a VP2 molecule (e.g., an ORF2 protein as listed in Table A1 or A2 or a VP2 molecule as listed in Table A3, or an amino acid sequence having at least 70%, 75%, 80%, 85%, 90%, 95%, 96%, 97%, 98%, or 99% sequence identity thereto).

147. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element does not comprise a nucleic acid sequence encoding an Anellovirus ORF2 molecule or a CAV VP2 molecule (e.g., an ORF2 protein or VP1 protein as listed in Table A1-A3 or an amino acid sequence having at least 70%, 75%, 80%, 85%, 90%, 95%, 96%, 97%, 98%, or 99% sequence identity thereto).

148. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises at least 20, 25, 30, 31, 32, 33, 34, 35, 36, 37, 38, 39, or 40 consecutive nucleotides having a GC content of at least 70%, 75%, 80%, 85%, 90%, 95%, or 99%.

149. The Anelloviridae family vector (e.g., anellovector), ORF1 molecule, ORF2 molecule, VP1 molecule, VP2 molecule, nucleic acid molecule, or method of any of the preceding embodiments, wherein the proteinaceous exterior comprises the amino acid sequence YNPX²DXGX²N (SEQ ID NO: 829), wherein Xⁿ is a contiguous sequence of any n amino acids.

150. The Anelloviridae family vector (e.g., anellovector), ORF1 molecule, ORF2 molecule, VP1 molecule, VP2 molecule, nucleic acid molecule, or method of embodiment 149, wherein the amino acid sequence YNPX²DXGX²N (SEQ ID NO: 829) is comprised in an N22 domain.

151. The Anelloviridae family vector (e.g., anellovector), ORF1 molecule, ORF2 molecule, VP1 molecule, VP2 molecule, nucleic acid molecule, or method of any of the preceding embodiments, wherein the ORF1 or VP1 molecule comprises an arginine-rich region (e.g., having at least 70%, 80%, 85%, 90%, 95%, 96%, 97%, 98%, 99%, or 100% identity to an arginine-rich region sequence of an ORF1 protein or VP1 protein listed in Table A1-A3).

152. The Anelloviridae family vector (e.g., anellovector), ORF1 molecule, ORF2 molecule, VP1 molecule, VP2 molecule, nucleic acid molecule, or method of any of the preceding embodiments, wherein the proteinaceous exterior comprises an amino acid sequence of at least 15, 20, 25, 26, 27, 28, 29, 30, 31, 32, 33, 34, 35, 36, 37, 38, 39, 40, 45, or 50 consecutive nucleotides comprising at least 40% (e.g., at least 40%, 41%, 42%, 43%, 44%, 45%, 46%, 47%, 48%, 49%, 50%, 55%, 60%, 65%, 66%, 67%, 68%, 69%, 70%, 75%, 80%, 85%, 90%, or 95%) arginine residues.

153. The Anelloviridae family vector (e.g., anellovector), ORF1 molecule, ORF2 molecule, VP1 molecule, VP2 molecule, nucleic acid molecule, or method of embodiment 151 or 152, wherein the arginine-rich region is located at the N-terminal or C-terminal end of the ORF1 or VP1 molecule.

154. The Anelloviridae family vector (e.g., anellovector), ORF1 molecule, ORF2 molecule, VP1 molecule, VP2 molecule, nucleic acid molecule, or method of any of the preceding embodiments, wherein the ORF1 or VP1 molecule comprises a jelly-roll domain having at least 70%, 80%, 85%, 90%, 95%, 96%, 97%, 98%, 99%, or 100% identity to a jelly-roll domain sequence of an ORF1 or VP1 protein listed in Table A1-A3.

155. The Anelloviridae family vector (e.g., anellovector), ORF1 molecule, ORF2 molecule, VP1 molecule, VP2 molecule, nucleic acid molecule, or method of any of the preceding embodiments, wherein the ORF1 or VP1 molecule comprises an N22 domain having at least 70%, 80%, 85%, 90%, 95%, 96%, 97%, 98%, 99%, or 100% identity to an N22 domain sequence of an ORF1 or VP1 protein listed in Table A1-A3.

156. The Anelloviridae family vector (e.g., anellovector), ORF1 molecule, ORF2 molecule, VP1 molecule, VP2 molecule, nucleic acid molecule, or method of any of the preceding embodiments, wherein the ORF1 or VP1 molecule comprises a C-terminal domain (CTD) having at least 70%, 80%, 85%, 90%, 95%, 96%, 97%, 98%, 99%, or 100% identity to a CTD domain sequence of an ORF1 or VP1 protein listed in Table A1-A3.

157. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises one or more of: a TATA box, an initiator element, a cap site, a transcriptional start site, an ORF1/1-encoding sequence, an ORF1/2-encoding sequence, an ORF2/2-encoding sequence, an ORF2/3-encoding sequence, an ORF2/3t-encoding sequence, a three open-reading frame region, a poly(A) signal, and/or a GC-rich region from an Anellovirus or CAV described herein (e.g., as listed in any of Tables N1-N4), or a sequence having at least 70%, 75%, 80%, 85%, 90%, 95%, 96%, 97%, 98%, 99%, or 100% sequence identity thereto.

158. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element comprises at least 75% (e.g., at least 75, 76, 77, 78, 79, 80, 85, 90, 91, 92, 93, 94, 95, 96, 97, 98, 99, or 100%) sequence identity to a 5′ UTR conserved domain sequence as listed in any of Tables N1-N4.

159. The Anelloviridae family vector (e.g., anellovector), ORF1 molecule, ORF2 molecule, VP1 molecule, VP2 molecule, nucleic acid molecule, or method of any of the preceding embodiments, wherein the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

160. The Anelloviridae family vector (e.g., anellovector), ORF1 molecule, ORF2 molecule, VP1 molecule, VP2 molecule, nucleic acid molecule, or method of any of the preceding embodiments, wherein the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, comprises one or more desired carbohydrates (e.g., glycosylations), is pH and temperature stable, is detergent resistant, and is non-immunogenic or non-pathogenic in a host.

161. The genetic element, Anelloviridae family vector (e.g., anellovector), ORF1 molecule, ORF2 molecule, VP1 molecule, VP2 molecule, nucleic acid molecule, or method of any of the preceding embodiments, wherein the promoter comprises an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc).

162. The genetic element, Anelloviridae family vector (e.g., anellovector), ORF1 molecule, ORF2 molecule, VP1 molecule, VP2 molecule, nucleic acid molecule, or method of any of the preceding embodiments, wherein the effector encodes a therapeutic agent, e.g., a therapeutic peptide or polypeptide or a therapeutic nucleic acid.

163. The genetic element, Anelloviridae family vector (e.g., anellovector), ORF1 molecule, ORF2 molecule, VP1 molecule, VP2 molecule, nucleic acid molecule, or method of any of the preceding embodiments, wherein the effector is an exogenous effector.

164. The Anelloviridae family vector (e.g., anellovector), ORF1 molecule, ORF2 molecule, VP1 molecule, VP2 molecule, nucleic acid molecule, or method of any of the preceding embodiments, wherein the effector is an endogenous effector (e.g., wherein the anellovector overexpresses the endogenous effector in a target cell).

165. The genetic element, Anelloviridae family vector (e.g., anellovector), ORF1 molecule, ORF2 molecule, VP1 molecule, VP2 molecule, nucleic acid molecule, or method of any of the preceding embodiments, wherein the effector comprises a regulatory nucleic acid, e.g., an miRNA, siRNA, mRNA, lncRNA, RNA, DNA, an antisense RNA, gRNA; a fluorescent tag or marker, an antigen, a peptide, a synthetic or analog peptide from a naturally-bioactive peptide, an agonist or antagonist peptide, an anti-microbial peptide, a pore-forming peptide, a bicyclic peptide, a targeting or cytotoxic peptide, a degradation or self-destruction peptide, a small molecule, an immune effector (e.g., influences susceptibility to an immune response/signal), a death protein (e.g., an inducer of apoptosis or necrosis), a non-lytic inhibitor of a tumor (e.g., an inhibitor of an oncoprotein), an epigenetic modifying agent, an epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand, an antibody, a receptor, or a CRISPR system or component.

166. The genetic element, Anelloviridae family vector (e.g., anellovector), ORF1 molecule, ORF2 molecule, VP1 molecule, VP2 molecule, nucleic acid molecule, or method of any of the preceding embodiments, wherein the effector comprises a miRNA, e.g., wherein the miRNA decreases expression of a target gene.

167. The genetic element, Anelloviridae family vector (e.g., anellovector), ORF1 molecule, ORF2 molecule, VP1 molecule, VP2 molecule, nucleic acid molecule, or method of any of the preceding embodiments, wherein the effector modulates expression or activity of a gene or protein, e.g., increases or decreases expression or activity of the gene or protein.

168. The Anelloviridae family vector (e.g., anellovector), ORF1 molecule, ORF2 molecule, VP1 molecule, VP2 molecule, nucleic acid molecule, or method of any of the preceding embodiments, wherein the Anelloviridae family vector is capable of replicating autonomously

169. The Anelloviridae family vector (e.g., anellovector), ORF1 molecule, ORF2 molecule, VP1 molecule, VP2 molecule, nucleic acid molecule, or method of any of the preceding embodiments, wherein the Anelloviridae family vector is replication-deficient (e.g., incapable of replicating autonomously).

170. The genetic element, Anelloviridae family vector (e.g., anellovector), ORF1 molecule, ORF2 molecule, VP1 molecule, VP2 molecule, nucleic acid molecule, or method of any of the preceding embodiments, wherein the genetic element integrates into the genome of a eukaryotic cell at a frequency of less than about 0.001%, 0.005%, 0.01%, 0.05%, 0.1%, 0.5%, 1%, 1.5%, or 2% of the genetic element that enters the cell.

171. The Anelloviridae family vector (e.g., anellovector), ORF1 molecule, ORF2 molecule, VP1 molecule, VP2 molecule, nucleic acid molecule, or method of any of the preceding embodiments, wherein the Anelloviridae family vector is substantially non-pathogenic, e.g., does not induce a detectable deleterious symptom in a subject (e.g., elevated cell death or toxicity, e.g., relative to a subject not exposed to the anellovector).

172. The Anelloviridae family vector (e.g., anellovector), ORF1 molecule, ORF2 molecule, VP1 molecule, VP2 moledule, nucleic acid molecule, or method of any of the preceding embodiments, wherein the Anelloviridae family vector is substantially non-immnuogenic, e.g., does not induce a detectable and/or unwanted immune response.

173. The Anelloviridae family vector (e.g., anellovector), ORF1 molecule, ORF2 molecule, VP1 molecule, VP2 molecule, nucleic acid molecule, or method of any of the preceding embodiments, wherein a population of at least 1000 of the Anelloviridae family vectors is capable of delivering at least about 100 copies (e.g., at least 1, 2, 3, 4, 5, 10, 20, 30, 40, 50, 100, 200, 300, 400, 500, 600, 700, 800, 900, or 1000 copies) of the genetic element into one or more eukaryotic cells (e.g., mammalian cells, e.g., human cells).

174. The Anelloviridae family vector (e.g., anellovector), ORF1 molecule, ORF2 molecule, VP1 molecule, VP2 moledule, nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering the genetic element into at least 10%, 20%, 30%, 40%, 50%, 60%, 70%, 80%, 90%, 95%, 99%, or more of a population of eukaryotic cells (e.g., mammalian cells, e.g., human cells).

175. The Anelloviridae family vector (e.g., anellovector), ORF1 molecule, ORF2 molecule, VP1 molecule, VP2 moledule, nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering at least 1, 2, 3, 4, 5, 6, 7, 8, 9, 10, 20, 50, 100, 200, 500, 1000, 2000, 5000, 8,000, 1×10⁴, 1×10⁵, 1×10⁶, 1×10⁷ or greater copies of the genetic element per cell to a population of eukaryotic cells (e.g., mammalian cells, e.g., human cells).

176. The Anelloviridae family vector (e.g., anellovector), ORF1 molecule, ORF2 molecule, VP1 molecule, VP2 moledule, nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering 1-3, 1-4, 1-5, 1-6, 1-7, 1-8, 1-9, 1-10, 5-10, 10-20, 20-50, 50-100, 100-1000, 1000-10⁴, 1×10⁴-1×10⁵, 1×10⁴-1×10⁶, 1×10⁴-1×10⁷, 1×10⁵-1×10⁶, 1×10⁵-1×10⁷, or 1×10⁶-1× 10⁷ copies of the genetic element per cell to a population of eukaryotic cells (e.g., mammalian cells, e.g., human cells).

177. The Anelloviridae family vector (e.g., anellovector), ORF1 molecule, ORF2 molecule, VP1 molecule, VP2 molecule, nucleic acid molecule, or method of any of the preceding embodiments, wherein the target cells into which the genetic element is delivered each receive at least 10, 50, 100, 500, 1000, 10,000, 50,000, 100,000, or more copies of the genetic element.

178. The Anelloviridae family vector (e.g., anellovector), ORF1 molecule, ORF2 molecule, VP1 molecule, VP2 molecule, nucleic acid molecule, or method of any of the preceding embodiments, wherein the Anelloviridae family vector is resistant to degradation by a detergent (e.g., a mild detergent, e.g., a biliary salt, e.g., sodium deoxycholate) relative to a viral particle comprising an external lipid bilayer, e.g., a retrovirus.

179. The Anelloviridae family vector (e.g., anellovector), ORF1 molecule, ORF2 molecule, VP1 molecule, VP2 molecule, nucleic acid molecule, or method of any of the preceding embodiments, wherein the genetic element enclosed by the proteinaceous exterior is resistant to degradation by a nuclease enzyme (e.g., a DNase).

180. The Anelloviridae family vector (e.g., anellovector), ORF1 molecule, ORF2 molecule, VP1 molecule, VP2 molecule, nucleic acid molecule, or method of any of the preceding embodiments, wherein the Anelloviridae family vector is capable of infecting mammalian cells, e.g., human cells, e.g., in vitro, in vivo, or ex vivo.

181. The Anelloviridae family vector (e.g., anellovector), ORF1 molecule, ORF2 molecule, VP1 molecule, VP2 molecule, nucleic acid molecule, or method of any of the preceding embodiments, wherein the Anelloviridae family vector selectively delivers the effector to, or is present at higher levels in (e.g., preferentially accumulates in), a desired cell type, tissue, or organ (e.g., bone marrow, blood, heart, GI, skin, photoreceptors in the retina, epithelial linings, or pancreas).

182. The genetic element, Anelloviridae family vector (e.g., anellovector), ORF1 molecule, ORF2 molecule, VP1 molecule, VP2 molecule, nucleic acid molecule, or method of any of the preceding embodiments, wherein genetic element or genetic element construct is capable of replicating (e.g., by rolling circle replication), e.g., capable of generating at least 1, 2, 3, 4, 5, 6, 7, 8, 9, 10, 20, 30, 40, 50, 60, 70, 80, 90, 10², 2×10², 5×10², 10³, 2×10³, 5×10³, or 10⁴ genomic equivalents of the genetic element per cell, e.g., as measured by a quantitative PCR assay.

183. The Anelloviridae family vector (e.g., anellovector), ORF1 molecule, ORF2 molecule, VP1 molecule, VP2 molecule, nucleic acid molecule, or method of any of the preceding embodiments, wherein the proteinaceous exterior is provided in cis relative to the genetic element.

184. The Anelloviridae family vector (e.g., anellovector), ORF1 molecule, ORF2 molecule, VP1 molecule, VP2 molecule, nucleic acid molecule, or method of any of the preceding embodiments, wherein the proteinaceous exterior is provided in trans relative to the genetic element.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings.

FIG. 1A is an illustration showing percent sequence similarity of amino acid regions of capsid protein sequences.

FIG. 1B is an illustration showing percent sequence similarity of capsid protein sequences.

FIG. 2 is an illustration showing one embodiment of an anellovector.

FIG. 3 depicts a schematic of a kanamycin vector encoding the LY1 strain of TTMiniV (“Anellovector 1”).

FIG. 4 depicts a schematic of a kanamycin vector encoding the LY2 strain of TTMiniV (“Anellovector 2”).

FIG. 5 depicts transfection efficiency of synthetic anellovectors in 293T and A549 cells.

FIGS. 6A and 6B depict quantitative PCR results that illustrate successful infection of 293T cells by synthetic anellovectors.

FIGS. 7A and 7B depict quantitative PCR results that illustrate successful infection of A549 cells by synthetic anellovectors.

FIGS. 8A and 8B depict quantitative PCR results that illustrate successful infection of Raji cells by synthetic anellovectors.

FIGS. 9A and 9B depict quantitative PCR results that illustrate successful infection of Jurkat cells by synthetic anellovectors.

FIGS. 10A and 10B depict quantitative PCR results that illustrate successful infection of Chang cells by synthetic anellovectors.

FIGS. 11A-11B are a series of graphs showing luciferase expression from cells transfected or infected with TTMV-LY2A574-1371, A1432-2210,2610::nLuc. Luminescence was observed in infected cells, indicating successful replication and packaging.

FIG. 11C is a diagram depicting the phylogenetic tree of Alphatorquevirus (Torque Teno Virus; TTV), with clades highlighted. At least 100 Anellovirus strains are represented. Exemplary sequences from several clades is provided herein.

FIG. 12 is a schematic showing an exemplary workflow for production of anellovectors (e.g., replication-competent or replication-deficient anellovectors as described herein).

FIG. 13 is a graph showing primer specificity for primer sets designed for quantification of TTV and TTMV genomic equivalents. Quantitative PCR based on SYBR green chemistry shows one distinct peak for each of the amplification products using TTMV or TTV specific primer sets, as indicated, on plasmids encoding the respective genomes.

FIG. 14 is a series of graphs showing PCR efficiencies in the quantification of TTV genome equivalents by qPCR. Increasing concentrations of primers and a fixed concentration of hydrolysis probe (250 nM) were used with two different commercial qPCR master mixes. Efficiencies of 90-110% resulted in minimal error propagation during quantification.

FIG. 15 is a graph showing an exemplary amplification plot for linear amplification of TTMV (Target 1) or TTV (Target 2) over a 7 log 10 of genome equivalent concentrations. Genome equivalents were quantified over 7 10-fold dilutions with high PCR efficiencies and linearity (R² TTMV: 0.996; R² TTV: 0.997).

FIGS. 16A-16B are a series of graphs showing quantification of TTMV genome equivalents in an anellovector stock. (A) Amplification plot of two stocks, each diluted 1:10 and run in duplicate. (B) The same two samples as shown in panel A, here shown in the context of the linear range. Shown are the upper and lower limits in the two representative samples. PCR Efficiency: 99.58%, R²: 0988.

FIG. 17 is a graph showing fold change in miR-625 expression in HEK293T cells transfected with the indicated plasmid.

FIG. 18 is a diagram showing pairwise identity for alignments of representative sequences from each Alphatorquevirus clade. DNA sequences for TTV-CT30F, TTV-P13-1, TTV-tth8, TTV-HD20a, TTV-16, TTV-TJN02, and TTV-HD16d were aligned. Pairwise percent identity across a 50-bp sliding window is shown along the length of the alignment. Brackets above indicate non-coding and coding regions with pairwise identities are indicated. Brackets below indicate regions of high or low sequence conservation.

FIG. 19 is a diagram showing pairwise identity for amino acid alignments for putative proteins across the seven Alphatorquevirus clades. Amino acid sequences for putative proteins from TTV-CT30F, TTV-P13-1, TTV-tth8, TTV-HD20a, TTV-16, TTV-TJN02, and TTV-HD16d were aligned. Pairwise percent identity across a 15-aa sliding window is shown along the length of each alignment. Pairwise identity for both open reading frame DNA sequence and protein amino acid sequence is indicated. (*) Putative ORF2t/3 amino acid sequences were aligned for TTV-CT30F, TTV-tth8, TTV-16, and TTV-TJN02.

FIG. 20 is a diagram showing that a domain within the 5′ UTR is highly conserved across the seven Alphatorquevirus clades (SEQ ID NOS 810-817, respectively, in order of appearance). The 71-bp 5′UTR conserved domain sequences for each representative Alphatorquevirus were aligned. The sequence has 95.2% pairwise identity between the seven clades.

FIG. 21 is a diagram showing an alignment of the GC-rich domains from the seven Alphatorquevirus clades. Each Anellovirus has a region downstream of the ORFs with greater than 70% GC content. Shown is an alignment of the GC-rich regions from TTV-CT30F, TTV-P13-1, TTV-tth8, TTV-HD20a, TTV-16, TTV-TJN02, and TTV-HD16d. The regions vary in length, but where they do align they have 75.4% pairwise identity.

FIG. 22 is a diagram showing infection of Raji B cells with anellovectors encoding a miRNA targeting n-myc interacting protein (NMI). Shown is quantification of genome equivalents of anellovectors detected after infection of Raji B cells (arrow) or control cells with NMI miRNA-encoding anellovectors.

FIG. 23 is a diagram showing infection of Raji B cells with anellovectors encoding a miRNA targeting n-myc interacting protein (NMI). The Western blot shows that anellovectors encoding the miRNA against NMI reduced NMI protein expression in Raji B cells, whereas Raji B cells infected with anellovectors lacking the miRNA showed comparable NMI protein expression to controls.

FIG. 24 is a series of graphs showing quantification of anellovector particles generated in host cells after infection with an anellovector comprising an endogenous miRNA-encoding sequence and a corresponding anellovector in which the endogenous miRNA-encoding sequence was deleted.

FIGS. 25A-25C are a series of diagrams showing intracellular localization of ORFs from TTMV-LY2 fused to nano-luciferase. (A) In Vero cells, ORF2 (top row) appeared to localize to the cytoplasm while ORF1/1 (bottom row) appeared to localize to the nucleus. (B) In HEK293 cells, ORF2 (top row) appeared to localize to the cytoplasm while ORF1/1 (bottom row) appeared to localize to the nucleus. (C) Localization patterns for ORF1/2 and ORF2/2 in cells.

FIG. 26 is a series of diagrams showing sequential deletion controls in the 3′ non-coding region (NCR) of TTV-tth8. The top row shows the structure of the wild-type TTV-tth8 Anellovirus. The second row shows TTV-tth8 with a deletion of 36 nucleotides in the GC-rich region of the 3′ NCR (Δ36 nt (GC)). The third row shows TTV-tth8 with the 36 nucleotide deletion and an additional deletion of the miRNA sequence, resulting in a total deletion of 78 nucleotides (Δ36 nt (GC) ΔmiR). The fourth row shows TTV-tth8 with a deletion of 171 nucleotides from the 3′ NCR, which includes both the 36 nucleotide deletion region and the miRNA sequence (Δ3′ NCR).

FIGS. 27A-27D are a series of diagrams showing that sequential deletions in the 3′ NCR of TTV-tth8 have significant effects on Anellovirus ORF transcript levels. Shown are expression of ORF1 and ORF2 at day 2 (A), ORF1/1 and ORF2/2 at day 2 (B), ORF1/2 and ORF2/3 at day 2 (C), and ORF2t3 at day 2 (D).

FIGS. 28A-28B are a series of diagrams showing constructs used to produce anellovectors expressing nano-luciferase (A) and a series of anellovector/plasmid combinations used to transfect cells (B).

FIGS. 29A-29C are a series of diagrams showing nano-luciferase expression in mice injected with anellovectors. (A) Nano-luciferase expression in mice at days 0-9 after injection. (B) Nano-luciferase expression in mice injected with various anellovector/plasmid construct combinations, as indicated. (C) Quantification of nano-luciferase luminescence detected in mice after injection. Group A received a TTMV-LY2 vector±nano-luciferase. Group B received a nano-luciferase protein and TTMV-LY2 ORFs.

FIGS. 29D-1 to 29D-2 are a schematic of the genomic organization of representative anellos from seven different Alphatorquevirus clades. Sequences for TTV-CT30F, TTV-P13-1, TTV-tth8, TTV-HD20a, TTV-16, TTV-TJN02, and TTV-HD16d were aligned, with key regions annotated. Putative open reading frames (ORFs) are represented in light gray, TATA boxes are represented in dark gray, and key putative regulatory regions are represented in medium gray, including the initiator element, the 5′UTR conserved domain, and the GC-rich region (e.g., as indicated).

FIG. 30 is a schematic showing an exemplary workflow for determining the endogenous target of Anellovirus pre-miRNAs.

FIGS. 31A-31B are a series of diagrams showing that a tandem Anellovirus plasmid can increase anellovirus or anellovector production. (A) Plasmid map for an exemplary tandem Anellovirus plasmid. (B) Transfection of HEK293T cells with a tandem Anellovirus plasmid resulted in production of four times the number of viral genomes compared to single-copy harboring plasmids.

FIG. 31C is a gel electrophoresis image showing circularization of TTMV-LY2 plasmids pVL46-063 and pVL46-240.

FIG. 31D is a chromatogram showing copy numbers for linear and circular TTMV-LY2 constructs, as determined by size exclusion chromatography (SEC).

FIG. 32 is a diagram showing an alignment of 36-nucleotide GC-rich regions from nine Anellovirus genome sequences, and a consensus sequence based thereon (SEQ ID NOS 818-827, respectively, in order of appearance).

FIG. 33 is a series of diagrams showing ORF1 structures from Anellovirus strains LY2 and CBD203. Putative domains are labeled: arginine-rich region (arg-rich), core region comprising a jelly-roll domain, hypervariable region (HVR), N22 region, and C-terminal domain (CTD), as indicated.

FIG. 34 is a diagram showing an ORF1 structure from Betatorquevirus strain CBS203. Residues showing high similarity among a set of 110 betatorqueviruses are indicated. Indicated are residues of 60-79.9% similarity, residues of 80-99.9% similarity, and residues of 100% similarity among all strains evaluated.

FIG. 35 is a diagram showing the consensus sequence (SEQ ID NO: 828) from alignment of 258 sequences of Alphatorqueviruses with residues with high similarity scores highlighted dark gray (100%), medium gray (80-99.9%), light gray (60-80%). Putative domains are indicated in boxes. Percent identity is also indicated by the box graph below the consensus sequence, with medium-gray boxes indicating 100% identity, light gray boxes indicating 30-99% identity, and dark gray boxes indicating below 30% identity.

FIG. 36 is a schematic showing the domains of an Anellovirus ORF1 molecule and the hypervariable region to be replaced with a hypervariable domain from a different Anellovirus.

FIG. 37 is a schematic showing the domains of ORF1 and the hypervariable region that will be replaced with a protein or peptide of interest (POI) from a non-anellovirus source.

FIG. 38 is a series of diagrams showing the design of an exemplary anellovector genetic element based on an Anellovirus genome. The protein-coding region was deleted from the anellovirus genome (left), leaving the anelloviral non-coding region (NCR), including the viral promoter, 5′UTR conserved domain (5CD), and GC-rich region. Payload DNA was inserted into the non-coding region at the protein-coding locus (right). The resulting anellovector harbored the payload DNA (including open reading frames, genes, non-coding RNAs, etc.) and the essential anellovirus cis replication and packaging elements, but lacked the essential protein elements for replication and packaging.

FIG. 39 is a bar graph showing that anellovectors comprising a genetic element encoding an exogenous human immunoadhesin successfully transduced the human lung-derived cell line EKVX.

FIG. 40 is a graph showing that anellovectors based on tth8 or LY2, engineered to contain a sequence encoding human erythropoietin (hEpo), could deliver a functional transgene to mammalian cells.

FIGS. 41A and 41B are a series of graphs showing that engineered anellovectors administered to mice were detectable seven days after intravenous injection.

FIG. 42 is a graph showing that hGH mRNA was detected in the cellular fraction of whole blood seven days after intravenous administration of an engineered anellovector encoding hGH.

FIGS. 43A-43D are a series of diagrams illustrating a highly conserved motif in Anellovirus ORF2. FIG. 43 discloses SEQ ID NO: 949.

FIGS. 44A and 44B are a series of diagrams showing evidence of full-length ORF1 mRNA expression in human tissues.

FIG. 45 is a graph showing the ability of an in vitro circularized (IVC) TTV-tth8 genome (IVC TTV-tth8) compared to a TTV-tth8 genome in a plasmid to yield TTV-tth8 genome copies at the expected density in HEK293T cells.

FIG. 46 is a series of graphs showing the ability of an in vitro circularized (IVC) LY2 genome (WT LY2 IVC) and a wild-type LY2 genome in plasmid (WT LY2 Plasmid) to yield LY2 genome copies at the expected density in Jurkat cells.

FIG. 47 is a diagram showing an alignment of secondary structure of the jelly roll domain of Anellovirus ORF1 proteins from Alphatorquevirus, Betatorquevirus, and Gammatorquevirus (SEQ ID NOs: 950-975). These secondary structural elements are highly conserved.

FIG. 48 is a disgram showing the conserved sequence and secondary structure of the ORF1 motif located in the N22 domain (SEQ ID NOS 976-1000 and 851, respectively, in order of appearance). The conserved YNPXXDXGXXN (SEQ ID NO: 829) motif of human TTV ORF1 has a conserved secondary structure. In particular, the tyrosine in the motif breaks a beta strand, and a second beta strand starts on the terminal asparagine of the motif.

FIG. 49 is a diagram showing the production of Ring 19 anellovectors in human cells.

FIG. 50A is a schematic of the single-stranded, circular DNA genome of an anellovirus, alternatively spliced to generate three different mRNAs encoding seven putative proteins of varying molecular weight.

FIG. 50B depicts RT-qPCR data from MOLT-4 cells transfected with a plasmid encoding two copies of the RING2 genome in tandem. Untransfected MOLT4 cells (control) were used as negative control and GAPDH mRNA was used as a housekeeping gene for normalization.

FIG. 50C depicts Western blotting data performed at indicated time points post-transfection of a plasmid encoding two copies of the RING2 genome in tandem to study the kinetics of the anellovirus proteins ORF1 and ORF2 over time. GAPDH protein was used as a loading control.

FIG. 51 is a Southern blot of digested samples from MOLT-4 cells transfected with either a plasmid encoding a single copy of the RING2 genome (Sample #4) or a plasmid encoding two copies of the RING2 genome in tandem (Sample #5). Samples #1, 2, and 3 are in vitro circularized RING2 genome, a plasmid containing a single copy of the RING2 genome, and a plasmid containing two copies of the RING2 genome in tandem, respectively, which acted as controls.

FIG. 52 is a graph plotting density (plotted in gray) and viral titer (plotted in black) of clarified lysate subjected to isopycnic centrifugation using CsCl linear gradient.

FIG. 53 is a graph depicting the results of DNase protected qPCR from MOLT-4 cell samples transfected with plasmid encoding two copies of the RING2 genome in tandem (WT RING2 tandem), an in vitro circularized genome of RING2 in which the expression of all ORF1 variants has been knocked out (ORF1 KO IVC), an in vitro circularized genome of RING2 in which the expression of all ORF2 variants has been knocked out (ORF2 KO IVC), or were co-transfected with both ORF1 KO IVC and ORF2 KO IVC.

FIG. 54A is a schematic of the production and purification of RING2 particles form MOLT-4 cells.

FIG. 54B is a set of graphs depicting the density and viral titer for each fraction after a CsCl gradient.

FIG. 54C is a graph depicting viral titers in the pooled material (input), concentrated material, and flow through (FT).

FIG. 54D is Western blotting analysis to detect capsid protein ORF1 in the pooled material (input), concentrated material, and flow through.

FIG. 54E is a set of representative transmission electron microscopy images of concentrated RING2 particles.

FIG. 55A is a schematic of the fully annotated, circularized genome, RING19, recovered from a dissected RPE tissues. ORF1 and ORF2 were all computationally annotated while ORF2/2 and ORF2/3 were manually curated.

FIG. 55B is a schematic of the production and purification of RING19 particles from MOLT-4 cells.

FIG. 55C is a graph depicting DNase protected qPCR assay of fractions from SEC of purified RING19.

FIGS. 55D-55E are representative transmission electron microscopy images of concentrated RING19 particles.

FIGS. 56A-56B are a series of diagrams showing RING19 infectivity in the murine retina and posterior eye cup. (A) Table describing various groups, treatment, virus/vector dose, routes of administration, number of animals per group and time point for the in vivo study. Bottom panel shows a schematic of the anatomy of a mouse eye as well as study design. (B) Vector/virus genome copies present in the neuroretina or posterior eye cup (PEC), as assessed by qPCR in the harvest DNA of mice eye's injected intravitreally (IVT) or subretinally (SR) once with either PBS, 6.6E+5 vg of Ring 19, or dose matched AAV2.mCherry. N=5-6 eyes/group. Abbreviations: AAV=adeno-associated virus, DNA=deoxyribonucleic acid, IVT=intravitreal, PBS=phosphate-buffered saline, PEC=posterior eye cup, SR=subretinal.

FIG. 57 is a series of graphs showing Ring2 infectivity in the retina and PEC of mice following subretinal and intravitreal injection of anellovirus. Vector genome (vg) copies present in the eye of mice injected either intravitreally or subretinally once with PBS, 1.6E6 vg of Ring 2, or dose-matched AAV2.mCherry. At day 7 or 21, three eyes from each group were harvested and the retinas and PEC's were analyzed separately by qPCR analyses using probes either targeting the Ring 2 genome or the mCherry transgene. Abbreviations: AAV=adeno-associated virus, DNA=deoxyribonucleic acid, IVT=intravitreal, PBS=phosphate-buffered saline, PEC=posterior eye cup, SR=subretinal, VG=vector genomes.

FIG. 58 is a series of graphs showing CAV infectivity in the retina and PEC of mice following subretinal and intravitreal injection. DNA vector genome copies or mRNA transgene copies detected in the eyes of mice injected subretinally once with PBS, 9.4E5 vg of CAV, dose-matched AAV2.nLuc or 1E+9 vg AAV2.nLuc. At day 14, 5-6 eyes from each group were harvested and the retinas and PEC's were analyzed separately by qPCR (DNA) or RT-qPCR (mRNA) using probes for the nLuc transgene. Abbreviations: AAV=adeno-associated virus, DNA=deoxyribonucleic acid, IVT=intravitreal, PBS=phosphate-buffered saline, PEC=posterior eye cup, SR=subretinal, nLuc=nanoluc luciferase, mRNA=messenger ribonucleic acid.

FIG. 59A is a schematic showing three exemplary Ring19 tandem vector constructs. In each construct, a CMV_nLuc cassette is inserted into the second copy of the Ring19 genome in the tandem construct at the indicated position, replacing the corresponding nucleotides of the Ring19 genome sequence. In the first exemplary construct (referred to herein as the CMV_nLuc3 construct), the CMV_nLuc cassette replaces a C-terminal portion of the ORF2 gene as well as an N-terminal portion of the ORF1 gene. In the second exemplary construct (referred to herein as the CMV_nLuc4 construct), the CMV_nLuc cassette replaces an internal portion of the ORF1 gene. In the third exemplary construct (referred to herein as the CMV_nLuc5 construct), the CMV_nLuc cassette replaces an internal portion of the ORF1 gene that is more C-terminal relative to the position replaced in the CMV_nLuc4 construct.

FIG. 59B is a diagram showing an exemplary workflow for producing Ring19 anellovector particles.

FIG. 60 is a graph showing recovery of the indicated Ring19 anellovectors using the tandem vector-based workflow shown in FIG. 59B. Shown are levels of DNase-protected nLuc-containing viral genomes after production of the indicated anellovectors.

FIG. 61 is a diagram showing an exemplary tandem nucleic acid construct for producing a Ring19 anellovector. The tandem construct comprises a first region (or first copy) comprising a Ring19 Anellovirus genome (including the 5′ UTR, ORF2 coding sequence, ORF1 coding sequence, ORF3 coding sequence, and GC-rich region of Ring19, as described herein) and a second region (or second copy) comprising a Ring19-based anellovector genome (including the 5′ UTR, at least a portion of an ORF2 nucleic acid sequence, a transgene sequence encoding a payload polypeptide of interest, a C-terminal portion of an ORF1 nucleic acid sequence, at least a portion of an ORF3 nucleic acid sequence, and a GC rich region).

FIGS. 62A-62B are a series of graphs showing qPCR titer for eGFP or mCherry amplicons after production of Ring19 anellovectors carrying the indicated transgene under the control of various promoters (as listed in the x-axes).

FIGS. 63A-63D are a series of graphs showing qPCR titer for hGH, gLuc, iCre, or hEpo amplicons after production of Ring19 anellovectors carrying the indicated transgene under the control of various promoters (as listed in the x-axes).

FIGS. 64A-64B are a series of graphs showing qPCR titer for wild-type Ring19 amplicons after production of Ring19 anellovectors under the control of various promoters (as listed in the x-axes).

FIGS. 65A-65D are a series of graphs showing qPCR titer for wild-type Ring19 amplicons after production of Ring19 anellovectors under the control of various promoters (as listed in the x-axes).

### DETAILED DESCRIPTION OF CERTAIN EMBODIMENTS

### Definitions

The present invention will be described with respect to particular embodiments and with reference to certain figures, but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

As used herein, the term “Anelloviridae family vector” refers to a vehicle derived from or similar to a virus of the Anelloviridae family (e.g., an Alphatorquevirus, Betatorquevirus, Gammatorquevirus, or chicken anemia virus), wherein the vehicle comprises a genetic element enclosed in a proteinaceous exterior (e.g, the genetic element is substantially protected from digestion with DNAse I by a proteinaceous exterior). In some embodiments, an Anelloviridae family vector comprises a genetic element derived from or highly similar to (e.g., at least 85%, 90%, 95%, 96%, 97%, 98%, 99%, or 100% identical to) that of an Alphatorquevirus, Betatorquevirus, Gammatorquevirus, or chicken anemia virus (CAV). In some embodiments, an Anelloviridae family vector comprises a proteinaceous exterior comprising a protein derived from or similar to (e.g., at least 50%, 60%, 70%, 75%, 80%, 85%, 90%, 95%, 96%, 97%, 98%, 99%, or 100% identical to) a capsid protein of an Alphatorquevirus, Betatorquevirus, Gammatorquevirus, or chicken anemia virus (e.g., an Alphatorquevirus ORF1, Betatorquevirus ORF1, Gammatorquevirus ORF1, or CAV VP1). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with DNAse I, e.g., prior to entry into a host cell. In some embodiments, the Anelloviridae family vector is purified, e.g., it is separated from its original source and/or substantially free (>50%, >60%, >70%, >80%, >90%) of other components. In some embodiments, the Anelloviridae family vector is capable of introducing the genetic element into a target cell (e.g., via infection). In some embodiments, the Anelloviridae family vector is an infective synthetic viral particle.

As used herein, the term “anellovector” refers to a vehicle comprising a genetic element, e.g., an episome, e.g., circular DNA, enclosed in a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior, e.g., prior to entry into a host cell. In some embodiments, the anellovector is purified, e.g., it is separated from its original source and/or substantially free (>50%, >60%, >70%, >80%, >90%) of other components.

An anellovector may, in some embodiments, comprise a nucleic acid vector that comprises sufficient nucleic acid sequence derived from or highly similar to (e.g., at least 85%, 90%, 95%, 96%, 97%, 98%, 99%, or 100% identical to) an Anellovirus genome sequence or a contiguous portion thereof to allow packaging into a proteinaceous exterior (e.g., a capsid), and further comprises a heterologous sequence. In some embodiments, the nucleic acid vector is a viral vector or a naked nucleic acid. In some embodiments, the nucleic acid vector comprises at least about 50, 60, 70, 71, 72, 73, 74, 75, 80, 90, 100, 150, 200, 300, 400, 500, 600, 700, 800, 900, 1000, 1100, 1200, 1300, 1400, 1500, 1600, 1700, 1800, 1900, 2000, 2500, 3000, or 3500 consecutive nucleotides of a native Anellovirus sequence or a sequence highly similar (e.g., at least 85%, 90%, 95%, 96%, 97%, 98%, 99%, or 100% identical) thereto. In some embodiments, the anellovector further comprises one or more of an Anellovirus ORF1, ORF2, or ORF3. In some embodiments, the heterologous sequence comprises a multiple cloning site, comprises a heterologous promoter, comprises a coding region for a therapeutic protein, or encodes a therapeutic nucleic acid. In some embodiments, the capsid is a wild-type Anellovirus capsid. In embodiments, an anellovector comprises a genetic element described herein, e.g., comprises a genetic element comprising a promoter, a sequence encoding a therapeutic effector, and a capsid binding sequence.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

As used herein, a nucleic acid “encoding” refers to a nucleic acid sequence encoding an amino acid sequence or a functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid sequence, generally in an anellovector. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

As used herein, the term “ORF1 molecule” refers to a polypeptide having an activity and/or a structural feature of an Anellovirus ORF1 protein (e.g., an Anellovirus ORF1 protein as described herein, e.g., as listed in Table A1 or A2), or a functional fragment thereof. An ORF1 molecule may, in some instances, comprise one or more of (e.g., 1, 2, 3 or 4 of): a first region comprising at least 60% basic residues (e.g., at least 60% arginine residues), a second region comprising at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid (e.g., as listed in any of Tables N1-N2). An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein), e.g., an ORF1 protein having the amino acid sequence as listed in Table A1 or A2, or as encoded by the ORF1 gene as listed in any of Tables N1-N2.

As used herein, the term “ORF2 molecule” refers to a polypeptide having an activity and/or a structural feature of an Anellovirus ORF2 protein (e.g., an Anellovirus ORF2 protein as described herein, e.g., as listed in Table A1 or A2), or a functional fragment thereof. An “Anellovirus ORF2 protein,” as used herein, refers to an ORF2 protein encoded by an Anellovirus genome (e.g., a wild-type Anellovirus genome, e.g., as described herein), e.g., an ORF2 protein having the amino acid sequence as listed in Table A1 or A2, or as encoded by the ORF2 gene as listed in any of Tables N1-N2.

As used herein, the term “VP1 molecule” refers to a polypeptide having an activity and/or a structural feature of a CAV VP1 protein (e.g., a CAV VP1 protein as described herein, or a functional fragment thereof. A VP1 molecule may, in some instances, comprise a polypeptide encoded by a CAV VP1 nucleic acid. A VP1 molecule may, in some instances, further comprise a heterologous sequence, e.g., from a CAV VP1 protein, e.g., as described herein. In some embodiments, a VP1 molecule is encoded by a CAV genome (e.g., a wild-type CAV genome, e.g., as described herein). In some embodiments, a VP1 molecule is a polypeptide encoded by a CAV VP1 nucleic acid (e.g., a VP1 gene, e.g., as described herein). In some embodiments, a VP1 molecule is a splice variant or comprises a post-translational modification.

As used herein, the term “VP2 molecule” refers to a polypeptide having an activity and/or a structural feature of a CAV VP2 protein (e.g., a CAV VP2 protein as described herein, or a functional fragment thereof. In some embodiments, a VP2 molecule is encoded by a CAV genome (e.g., a wild-type CAV genome, e.g., as described herein). In some embodiments, a VP2 molecule is a polypeptide encoded by a CAV VP2 nucleic acid (e.g., a VP2 gene, e.g., as described herein). In some embodiments, a VP2 molecule is a splice variant or comprises a post-translational modification.

As used herein, the term “Apoptin molecule” and “VP3 molecule” are used interchangeably and refer to a polypeptide having an activity and/or a structural feature of a CAV Apoptin protein (e.g., a CAV Apoptin protein as described herein, or a functional fragment thereof. In some embodiments, an Apoptin molecule is encoded by a CAV genome (e.g., a wild-type CAV genome, e.g., as described herein). In some embodiments, an Apoptin molecule is a polypeptide encoded by a CAV Apoptin nucleic acid (e.g., an Apoptin gene). In some embodiments, an Apoptin molecule is a splice variant or comprises a post-translational modification.

As used herein, the term “CAV capsid polypeptide” refers to a polypeptide present in the capsid of a wild-type CAV, or a polypeptide having an activity and/or a structural feature of said polypeptide. In some embodiments, the CAV capsid polypeptide is a VP1 molecule.

As used herein, the term “VP1 nucleic acid” refers to a nucleic acid that encodes a VP1 molecule, or the reverse complement thereof. The nucleic acid may be single stranded or double stranded. In some embodiments, the VP1 nucleic acid comprises a CAV VP1 gene, e.g., as described herein. A “VP1 gene” generally refers to a nucleic acid sequence encoding a wild-type VP1 molecule, or the reverse complement thereof. In some embodiments, a VP1 gene comprises a sense strand. In some embodiments, a VP1 gene comprises an antisense strand. In some embodiments, a VP1 gene is double-stranded.

As used herein, the term “VP2 nucleic acid” refers to a nucleic acid that encodes a VP2 molecule, or the reverse complement thereof. The nucleic acid may be single stranded or double stranded. In some embodiments, the VP2 nucleic acid comprises a CAV VP2 gene, e.g., as described herein. A “VP2 gene” generally refers to a nucleic acid sequence encoding a wild-type VP2 molecule, or the reverse complement thereof. In some embodiments, a VP2 gene comprises a sense strand. In some embodiments, a VP2 gene comprises an antisense strand. In some embodiments, a VP2 gene is double-stranded.

As used herein, the term “Apoptin nucleic acid” and “VP3 nucleic acid” are used interchangeably, and refer to a nucleic acid that encodes a Apoptin molecule, or the reverse complement thereof. The nucleic acid may be single stranded or double stranded. In some embodiments, the Apoptin nucleic acid comprises a CAV Apoptin gene, e.g., as described herein. An “Apoptin gene” or “VP3 gene” generally refers to a nucleic acid sequence encoding a wild-type Apoptin molecule, or the reverse complement thereof. In some embodiments, an Apoptin gene comprises a sense strand. In some embodiments, an Apoptin gene comprises an antisense strand. In some embodiments, an Apoptin gene is double-stranded.

As used herein, the term “CAV genome sequence” refers to a nucleic acid sequence comprising a full-length genome sequence from a wild-type CAV, e.g., as described herein, or a sequence having at least 70%, 75%, 80%, 85%, 90%, 95%, 96%, 97%, 98%, 99%, or 100% sequence identity thereto. In some embodiments, a CAV genome comprises a CAV genome sequence as described herein (e.g., a wild-type CAV genome sequence, e.g., as listed in any of Tables N3-N4).

As used herein, the term “CAV UTR” refers to a nucleic acid sequence comprising an untranslated region (UTR) sequence (e.g., the sequence of a 5′ UTR or a 3′ UTR) from a CAV (e.g., a wild-type CAV, e.g., as described herein, e.g., as listed in Table N3-N4), or a sequence having at least 70%, 75%, 80%, 85%, 90%, 95%, 96%, 97%, 98%, 99%, or 100% identity thereto.

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “replication protein” refers to a protein, e.g., a viral protein, that is utilized during infection, viral genome replication/expression, viral protein synthesis, and/or assembly of the viral components.

As used herein, a “substantially non-pathogenic” organism, particle, or component, refers to an organism, particle (e.g., a virus or an anellovector, e.g., as described herein), or component thereof that does not cause or induce a detectable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce a detectable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization.

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In some embodiments, the substantially non-immunogenic organism, particle, or component does not produce a detectable immune response. In some embodiments, the substantially non-immunogenic anellovector does not produce a detectable immune response against a protein comprising an amino acid sequence or encoded by a nucleic acid sequence shown in any of Tables N1-N4. In some embodiments, an immune response (e.g., an undesired or untargeted immune response) is detected by assaying antibody presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subseqence comprising an exogenous sequence or a sequence heterologous to the remainder of the larger sequence, e.g., a corresponding subsequence from a different Anellovirus).

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

As used herein, the term “virome” refers to viruses in a particular environment, e.g., a part of a body, e.g., in an organism, e.g. in a cell, e.g. in a tissue.

This invention relates generally to Anelloviridae family vectors (e.g., anellovectors), e.g., synthetic Anelloviridae family vectors (e.g., anellovectors), and uses thereof. The present disclosure provides Anelloviridae family vectors (e.g., anellovectors), compositions comprising Anelloviridae family vectors (e.g., anellovectors), and methods of making or using Anelloviridae family vectors (e.g., anellovectors). Anelloviridae family vectors (e.g., anellovectors) are generally useful as delivery vehicles, e.g., for delivering a therapeutic agent to a eukaryotic cell. Generally, an Anelloviridae family vector (e.g., anellovector) will include a genetic element comprising a nucleic acid sequence (e.g., encoding an effector, e.g., an exogenous effector or an endogenous effector) enclosed within a proteinaceous exterior. An Anelloviridae family vector (e.g., anellovector) may include one or more deletions of sequences (e.g., regions or domains as described herein) relative to an Anellovirus sequence (e.g., as described herein). Anelloviridae family vectors (e.g., anellovectors) can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

### Table of Contents

I. Anelloviridae Family Vectors (e.g., Anellovectors)
   A. Anelloviridae Family Viruses (e.g., Anelloviruses and CAVs)
   B. Capsid Proteins (e.g., ORF1 molecules and VP1 molecules)
   C. ORF2 molecules
   D. Genetic elements
   E. Protein binding sequences
   F. 5′ UTR Regions
   G. GC-rich regions
   H. Effectors
   I. Proteinaceous exterior
II. Compositions and Methods for Making Anelloviridae Family Vectors
   A. Components and Assembly of Anelloviridae Family Vectors
      i. Capsid proteins (e.g., ORF1 molecules and VP1 molecules) for assembly of anellovectors
      ii. ORF2 molecules for assembly of anellovectors
      iii. Production of protein components
   B. Genetic Element Constructs
      i. Plasmids
      ii. Circular nucleic acid constructs
      iii. In vitro circularization
      iv. Tandem constructs
      v. Cis/trans constructs
      vi. Expression cassettes
      vii. Design and production of a genetic element construct
   C. Effectors
   D. Host Cells
      i. Introduction of genetic elements into host cells
      ii. Methods for providing protein(s) in cis or trans
      iii. Exemplary cell types
   E. Culture Conditions
   F. Harvest
   G. In vitro assembly methods
   H. Enrichment and Purification
III. Vectors
IV. Compositions
V. Host cells
VI. Methods of use
VII. Methods of production
VIII. Administration/Delivery

### I. Anelloviridae Family Vectors (e.g., Anellovectors)

In some aspects, the invention described herein comprises compositions and methods of using and making an Anelloviridae family vector (e.g., anellovector), Anelloviridae family vector (e.g., anellovector) preparations, and therapeutic compositions. In some embodiments, the anellovector has a sequence, structure, and/or function that is based on an Anelloviridae virus (e.g., an Anellovirus as described herein or a CAV). It is understood that applicable embodiments described herein with respect to anellovectors may also be applied to Anelloviridae family vectors (e.g., a vector based on or derived from a chicken anemia virus (CAV), e.g., as described herein). In some embodiments, the Anelloviridae family vector (e.g., anellovector) comprises a nucleic acid or polypeptide comprising a sequence as shown in Table A1-A3 (e.g., Table A1, A1.1, A2, or A3); or Table N1-N4 (e.g., Table N1, N1.1, N2, N3, or N4), or fragments or portions thereof, or other substantially non-pathogenic virus, e.g., a symbiotic virus, commensal virus, native virus. In some embodiments, an Anelloviridae family virus-based vector comprises at least one element exogenous to that Anelloviridae family virus, e.g., an exogenous effector or a nucleic acid sequence encoding an exogenous effector disposed within a genetic element of the vector. In some embodiments, an Anelloviridae family virus-based vector comprises at least one element heterologous to another element from that Anelloviridae family virus, e.g., an effector-encoding nucleic acid sequence that is heterologous to another linked nucleic acid sequence, such as a promoter element. In some embodiments, an Anelloviridae family vector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An Anelloviridae family vector may be a delivery vehicle (e.g., a substantially non-pathogenic delivery vehicle) for a payload into a host, e.g., a human. In some embodiments, the Anelloviridae family vector is capable of replicating in a eukaryotic cell, e.g., a mammalian cell, e.g., a human cell. In some embodiments, the Anelloviridae family vector is substantially non-pathogenic and/or substantially non-integrating in the mammalian (e.g., human) cell. In some embodiments, the Anelloviridae family vector is substantially non-immunogenic in a mammal, e.g., a human. In some embodiments, the Anelloviridae family vector is replication-deficient. In some embodiments, the Anelloviridae family vector is replication-competent.

In some embodiments the Anelloviridae family vector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety.

In an aspect, the invention includes an Anelloviridae family vector (e.g., an anellovector) comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the Anelloviridae family vector (e.g. anellovector) is capable of delivering the genetic element into a eukaryotic cell.

In some embodiments of the Anelloviridae family vector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the Anelloviridae family vectors (e.g. anellovectors) administered to a subject will integrate into the genome of one or more host cells in the subject. In some embodiments, the genetic elements of a population of Anelloviridae family vectors (e.g. anellovectors), e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an Anelloviridae family vector (e.g. anellovector) comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anelloviridae family virus (e.g., Anellovirus or CAV) sequence (e.g., a wild-type Torque Teno virus (TTV), Torque Teno mini virus (TTMV), TTMDV, or CAV sequence, e.g., a wild-type Anelloviridae family virus (e.g., Anellovirus or CAV) sequence as listed in any of Tables N1-N4, e.g., Table N1, N1.1, N2, N3, or N4); and (ii) a proteinaceous exterior; wherein the genetic element is enclosed within the proteinaceous exterior; and wherein the Anelloviridae family vector is capable of delivering the genetic element into a eukaryotic cell.

In one aspect, the invention includes an Anelloviridae family vector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops or encloses, the genetic element.

In some embodiments, the Anelloviridae family vector (e.g. anellovector) includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the Anelloviridae family vector (e.g. anellovector) modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In some embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In some embodiments, the promoter element comprises a TATA box. In some embodiments, the promoter element is endogenous to a wild-type Anelloviridae family virus (e.g., Anellovirus or CAV), e.g., as described herein.

In some embodiments, the genetic element comprises one or more of the following characteristics: single-stranded, circular, negative strand, and/or DNA. In some embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The Anelloviridae family vectors (e.g. anellovectors), compositions comprising Anelloviridae family vectors (e.g. anellovectors), methods using such Anelloviridae family vectors (e.g. anellovectors), etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce Anelloviridae family vectors which can then be used to deliver an effector to cells (e.g., animal cells, e.g., human cells or non-human animal cells such as pig or mouse cells). In embodiments, the effector can silence expression of a factor such as an interferon. The examples further describe how Anelloviridae family vectors can be made by inserting effectors into sequences derived, e.g., from an Anelloviridae family virus (e.g., Anellovirus or CAV). It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specific Anelloviridae family virus (e.g., Anellovirus or CAV) sequences described in the examples may also be replaced by the Anelloviridae family virus (e.g., Anellovirus or CAV) sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an Anelloviridae family vector (e.g. anellovector), or the genetic element comprised in the Anelloviridae family vector (e.g. anellovector), is introduced into a cell (e.g., a human cell). In some embodiments, the effector (e.g., an RNA, e.g., an miRNA), e.g., encoded by the genetic element of an Anelloviridae family vector (e.g. anellovector), is expressed in a cell (e.g., a human cell), e.g., once the Anelloviridae family vector (e.g. anellovector) or the genetic element has been introduced into the cell. In some embodiments, introduction of the Anelloviridae family vector (e.g. anellovector), or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, introduction of the Anelloviridae family vector (e.g. anellovector), or genetic element comprised therein, decreases level of interferon produced by the cell. In some embodiments, introduction of the Anelloviridae family vector (e.g. anellovector), or genetic element comprised therein, into a cell modulates (e.g., increases or decreases) a function of the cell. In some embodiments, introduction of the Anelloviridae family vector (e.g. anellovector), or genetic element comprised therein, into a cell modulates (e.g., increases or decreases) the viability of the cell. In some embodiments, introduction of the Anelloviridae family vector (e.g. anellovector), or genetic element comprised therein, into a cell decreases viability of a cell (e.g., a cancer cell).

In some embodiments, an Anelloviridae family vector (e.g. anellovector) (e.g., a synthetic anellovector) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anelloviridae family virus (e.g., Anellovirus or CAV) (e.g., as described herein), or an Anelloviridae family vector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anelloviridae family virus (e.g., Anellovirus or CAV) or an Anelloviridae family vector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, ORF2t/3, VP1, VP2, and/or VP3 e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1 or VP1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anelloviridae family virus (e.g., Anellovirus or CAV) (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, ORF2t/3, VP1, VP2, or VP3) that would permit packaging of the genetic element of a wild-type Anelloviridae family virus (e.g., Anellovirus or CAV) (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anelloviridae family virus (e.g., Anellovirus or CAV) (e.g., as described herein), even in the presence of factors (e.g., ORF1, ORF1/1, ORF1/2, ORF2, ORF2/2, ORF2/3, ORF2t/3, VP1, VP2, or VP3) that would permit packaging of the genetic element of a wild-type Anelloviridae family virus (e.g., Anellovirus or CAV) (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1 or VP1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anelloviridae family virus (e.g., Anellovirus or CAV) (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, ORF2t/3, VP1, VP2, or VP3) that would permit packaging of the genetic element of a wild-type Anelloviridae family virus (e.g., Anellovirus or CAV) (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anelloviridae family virus (e.g., Anellovirus or CAV) (e.g., as described herein) in the presence of factors (e.g., ORF1, ORF1/1, ORF1/2, ORF2, ORF2/2, ORF2/3, ORF2t/3, VP1, VP2, or VP3) that would permit packaging of the genetic element of a wild-type Anelloviridae family virus (e.g., Anellovirus or CAV) (e.g., as described herein).

### Anelloviridae Family Viruses (e.g., Anelloviruses and CAVs)

In some embodiments, an Anelloviridae family vector, e.g., as described herein, comprises sequences or expression products derived from an Anellovirus. In some embodiments, an Anelloviridae family vector includes one or more sequences or expression products that are exogenous relative to the Anellovirus. In some embodiments, an Anelloviridae family vector includes one or more sequences or expression products that are endogenous relative to the Anellovirus. In some embodiments, an Anelloviridae family vector includes one or more sequences or expression products that are heterologous relative to one or more other sequences or expression products in the Anelloviridae family vector. Anelloviridae family viruses (e.g., Anellovirus or CAV) generally have single-stranded circular DNA genomes with negative polarity. Anelloviruses have not generally been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anelloviruses are generally transmitted by oronasal or fecal-oral infection, mother-to-infant and/or in utero transmission (Gerner et al., Ped. Infect. Dis. J. (2000) 19:1074-1077). Infected persons can, in some instances, be characterized by a prolonged (months to years) Anellovirus viremia. Humans may be co-infected with more than one genogroup or strain (Saback, et al., Scad. J. Infect. Dis. (2001) 33:121-125). There is a suggestion that these genogroups can recombine within infected humans (Rey et al., Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-lnigo et al., Am. J. Pathol. (2000) 156:1227-1234).

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an Anelloviridae family vector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the Anelloviridae family vector comprises a nucleic acid sequence selected from a sequence as shown in any of Tables N1-N4, or a sequence having at least 70%, 75%, 80%, 85%, 90%, 95%, 96%, 97%, 98%, 99%, or 100% sequence identity thereto. In embodiments, the Anelloviridae family vector comprises a polypeptide comprising a sequence as shown in Table A1-A3, or a sequence having at least 70%, 75%, 80%, 85%, 90%, 95%, 96%, 97%, 98%, 99%, or 100% sequence identity thereto.

In some embodiments, an Anelloviridae family vector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, VP1, VP2, VP3 (apoptin), three open-reading frame region, poly(A) signal, GC-rich region, or any combination thereof, of any of the Anelloviridae family viruses (e.g., Anellovirus or CAV) described herein (e.g., an Anelloviridae family virus (e.g., Anellovirus or CAV) sequence as annotated, or as encoded by a sequence listed, in any of Tables N1-N4. In some embodiments, the nucleic acid molecule comprises a sequence encoding a capsid protein, e.g., an ORF1, ORF1/1, ORF1/2, ORF2, ORF2/2, ORF2/3, ORF2t/3, or VP1 sequence of any of the Anelloviruses described herein (e.g., an Anelloviridae family virus (e.g., Anellovirus or CAV) sequence as annotated, or as encoded by a sequence listed, in any of Tables N1-N4). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anelloviridae family virus (e.g., Anellovirus or CAV) ORF1 ORF2, VP1, VP2, or apoptin protein (e.g., an ORF1, ORF2, VP1, VP2, or apoptin amino acid sequence as shown in Table A1-A3, or an ORF1, ORF2, VP1, VP2, or apoptin amino acid sequence encoded by a nucleic acid sequence as shown in any of Tables N1-N4). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anelloviridae family virus (e.g., Anellovirus or CAV) ORF1 or VP1 protein (e.g., an ORF1 or VP1 amino acid sequence as shown in Table A1-A3, or an ORF1 or VP1 amino acid sequence encoded by a nucleic acid sequence as shown in any of Tables N1-N4).

### Nucleic Acid Sequences

In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus or CAV) ORF1 or VP1 nucleotide sequence of any of Tables N1-N4. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus or CAV) ORF2 or VP2 nucleotide sequence of any of Tables N1-N4. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus or CAV) ORF3 or VP3 nucleotide sequence of any of Tables N1-N4. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus or CAV) GC-rich region nucleotide sequence of any of Tables N1-N4. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus or CAV) 5′ UTR conserved domain nucleotide sequence of any of Tables N1-N4.

### Amino Acid Sequences Encoded by Nucleic Acid Sequences

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF1 or VP1 amino acid sequence of Table A1 or A2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF2 or VP2 amino acid sequence of Table A1 or A2. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF3 or VP3 amino acid sequence of Table A1 or A2.

### Proteins Comprising Amino Acid Sequences

In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF1 or VP1 amino acid sequence of Table A1-A3. In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF2 or VP2 amino acid sequence of Table A1 or A2. In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF3 or VP3 amino acid sequence of Table A1 or A2. In some embodiments, an ORF1 or VP1 molecule (e.g., comprised in the Anelloviridae family vector) comprises a polypeptide encoded by the Anelloviridae family virus (e.g., Anellovirus or CAV) ORF1 or VP1 nucleic acid sequence of any of Tables N1-N4. In some embodiments, the ORF1 or VP1 molecule (e.g., comprised in the Anelloviridae family vector) comprises an Anelloviridae family virus (e.g., Anellovirus or CAV) ORF1 or VP1 protein of Table A1-A3 or a splice variant or post-translationally processed (e.g., proteolytically processed) variant thereof. In some embodiments, an ORF2 or VP2 molecule (e.g., comprised in the Anelloviridae family vector) comprises a polypeptide encoded by the Anelloviridae family virus (e.g., Anellovirus or CAV) ORF2 or VP2 nucleic acid sequence of any of Tables N1-N4. In some embodiments, the ORF2 or VP2 molecule (e.g., comprised in the Anelloviridae family vector) comprises an Anelloviridae family virus (e.g., Anellovirus or CAV) ORF2 or VP2 protein of Table A1-A3 or a splice variant or post-translationally processed (e.g., proteolytically processed) variant thereof. In some embodiments, an ORF3 or VP3 molecule (e.g., comprised in the Anelloviridae family vector) comprises a polypeptide encoded by the Anelloviridae family virus (e.g., Anellovirus or CAV) ORF3 or VP3 nucleic acid sequence of any of Tables N1-N4. In some embodiments, the ORF3 or VP3 molecule (e.g., comprised in the Anelloviridae family vector) comprises an Anelloviridae family virus (e.g., Anellovirus or CAV) ORF3 or VP3 protein of Table A1-A3 or a splice variant or post-translationally processed (e.g., proteolytically processed) variant thereof.

### Polypeptides Comprising Amino Acid Sequences

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus or CAV) ORF1 or VP1 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF1 or VP1 amino acid sequence of Table A1-A3.

In some embodiments, the polypeptide described herein comprises an amino acid sequence having at least about 70%, 75%, 80%, 85%, 90%, 95%, 96%, 97%, 98%, 99%, or 100% sequence identity to an ORF1 or VP1 molecule encoded by an Anelloviridae family virus (e.g., Anellovirus or CAV) ORF1 or VP1 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF1 or VP1 molecule encoded by an Anelloviridae family virus (e.g., Anellovirus or CAV) ORF1 or VP1 nucleic acid as listed in Table N1-N4.

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus or CAV) ORF2 or VP2 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF2 or VP2 amino acid sequence of Table A1 or A2.

In some embodiments, the polypeptide described herein comprises an amino acid sequence having at least about 70%, 75%, 80%, 85%, 90%, 95%, 96%, 97%, 98%, 99%, or 100% sequence identity to an ORF2 or VP2 molecule encoded by an Anelloviridae family virus (e.g., Anellovirus or CAV) ORF2 or VP2 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF2 or VP2 molecule encoded by an Anelloviridae family virus (e.g., Anellovirus or CAV) ORF2 or VP2 nucleic acid as listed in Table N1-N4.

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus or CAV) ORF3 or VP3 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus or CAV) ORF3 or VP3 amino acid sequence of Table A1 or A2.

In some embodiments, the polypeptide described herein comprises an amino acid sequence having at least about 70%, 75%, 80%, 85%, 90%, 95%, 96%, 97%, 98%, 99%, or 100% sequence identity to an ORF3 or VP3 molecule encoded by an Anelloviridae family virus (e.g., Anellovirus or CAV) ORF3 or VP3 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF3 or VP3 molecule encoded by an Anelloviridae family virus (e.g., Anellovirus or CAV) ORF3 or VP3 nucleic acid as listed in Table N1-N4.

In some embodiments, the polypeptide comprises an amino acid sequence (e.g., an ORF1, ORF1/1, ORF1/2, ORF2, ORF2/2, ORF2/3, ORF2t/3, VP1, VP2, VP3 sequence) as shown in Table A1-A3, or a sequence having at least 70%, 75%, 80%, 85%, 90%, 95%, 96%, 97%, 98%, 99%, or 100% sequence identity thereto.

TABLE N1
Novel Anellovirus nucleic acid sequence (Betatorquevirus)

| | |
|---|---|
| Name | RING 19 |
| Genus/Clade | Betatorquevirus |
| Accession | N/A |

Full Sequence: 2876 bp
1 10 20 30 40 50
| | | | | |
CGGGAGCCGAAGGTGAGTGCAACCACCGTAGTCTAGGGGCAATTCGGGCT
AGTTCAGTATGGCGGAACGGGCAAGAAACTTAAATATTATTATTTTACAG
ATGCAAATACAACCACCTATTAGAACCTTCAAACAAACAATTTCAGATTG
GAAAAACTTAATTGTCCACGTTCACGACAACATTTGCAACTGCAATAAAC
CATTAGAACACACTATTGATACCTGTATCACCAATCCAGATGAATTAAGA
TTAAACAAATCTACTAAACAACAACTACAAAAATGCCTTGGTACCCCAGA
AGAAGATACCCAAGAAGACGTTATCGATGGCTTCGCAGATGGAGAGCTAG
ACGCCCTTTTCGCCCAAGATACAGAAGAAGATACTGGGTAAGAAACTATT
CTCGAAAGAGAAAACTATTTAAAATAACAACCAAAGAATGGCAACCAAAA
GTTATAAGAAAGACTCATGTAAAGGGCACCTATCCTTTGTTTCTTTGTAC
AAAGCACAGAATTAACAATAATATGATACAATATTTAGACTCTATAGCTC
CAGAACACTATTACGGAGGAGGAGGATTTTCAATAATGCAATTTTCCTTA
CAAGCCTTATATGAAGAATTTATAAAAGCAAAAAACTGGTGGACTAATAC
AAACTGCTTTTTACCACTTGTAAGATATATGGGTTGCTCATTCAAATTTT
ATAAAACTGAATTTTATGATTATATTGTACTAATTGAAAGATGTTATCCA
CTTGCTTGTACTGATGAAATGTACTTATCTACTCAACCTAGTATTATGAT
GCTTACAAGAAAATGTATTTTTGTACCATGCAAACAAAACAGCAAAGGTA
AAAAACCTTACAAAAAAGTTAGAGTAAGACCACCTTCACAAATGACTACA
GGATGGCATTTCTCACAAGACTTAGCAAACATGCCACTTGTAGTACTAAA
AACTTCAGTATGCAGCTTTGACAGATATTACACAGACAGTACAGCTAAAT
CAACCACAATAGGCTTTAAAACACTTAACACACAAACATTTAGATATCAT
GACTGGCAGGAACCACCTACAACAGGATACAAACCACAAAACCTACTATG
GTTTTATGGAGCAGAAAACGGATCACCAGTAGACCCCAACAACACAATAG
TATCAAACCTAATATACTTAGGAGGCACAGGACCTTATGAAAAAGGCACA
CCAATAAAAACAAACATAAGCAATTACTTTTCAGAGCCTAAACTGTGGGG
AAATATATTTCACGATGATTATACATCAGGAACATCACCCGTGTTTGTTA
CAAACAAATCACCATCAGAAATTAAAACCGCATGGAACACTATAAAAGAC
TTAACTGTTAAAGCTAGCGGTGTATTTACATTAAGAACAATTCCACTATG
GCTACCTTGCAGATACAACCCATTTGCAGACAAAGCAACCAACAACAAAA
TATGGCTAGTTTCTATACATTCAGACCACACAGAATGGAAACCAATAGAC
AATCCATTACTACAACGAACAGACCTTCCTTTATGGTTACTTGTATGGGG
TTGGCAAGATTGGCAGAAAAAAAACCAACAAACTTCACAACCTGATATTA
ATTATTTAACAGTAATATCTTCACCATATATATCATGCTACCCAAAATTA
GATTACTATGTGTTACTAGATGAAGGATTTTGGGAGGGTCACTCAACATA
CATAGAGTCAATTACAGACTCAGACAAAAAACACTGGTACCCTAAAAATA
GATTTCAAATAGAAACACTTAATCTAATAGCTAACACAGGTCCAGGAACT
GTAAAACTAAGAGAAAACCAAGCAGCAGAAGGTCACATGGTATATCGCTT
TAATTTTAAGCTTGGAGGATGTCCCGCACCGATGGAAAAAATATGTGACC
CTAGCAAACAATCCAAATATCCTATTCCCAATAACCAGCAACAAACAACT
TCGTTGCAGAGTCCAGAAAACCCAATTCAAACCTATCTCTACGACTTCGA
CGAAAGGAGGGGCCTACTTACAGAAAGAGCTACAAAAAGAATCAAACAAG
ATCACACATCTGAAAAAACTGTTTTGCCATTTACAGGAGCAGCAACAGAC
CTCCCCATACTCCAAACAACATCACAGGAGGAAAGCTCCTCGGAAGAAGA
AGAAGAGCAACAAGCGGAGAAGAAACTACTCCAGCTCCGAAGAAAGCAGC
ACCGACTCCGGGAGCGAATCCTCCAGCTATTAGACATACAAAATACATAA
TAAAACAAAGTACTGTAAAAATTGATATGTTTGGAGATACTCATGTACCT
AACCGTAGAATGACCCCAGAAGAATTTGAACAAGAACTAATTGTCGCTGG
TGTTTTTCGCAGACCTCCTTGTTACTATATAAAAGATAGACCTACTTATC
CTTATGTACCAAAACCTACTGATGAAAAATGTATGGTAAACTTTGACTTA
AACTTTCCTTAATAAACTACGCCTGCAAACTTTCACTCTCGGTGTCCATT
TATATAAGATAAAACTTAAATAAACATCCACCACTCTCCCAAATACGCAG
GCGCACAAGGGGGCTCCGCCCCCTTAAACCCCCAAGGGGGCTCCGCCCCC
TTAAACCCCCAAGGGGGCTCCGCCCCCTTACACCCCCTAATAAATATTCA
ACAGGAAAACCACCTAATTAGAATTGCCGACCACAAACCGTCACTTACTT
CTCCTTTTTGCACTTACTTCCTCTTTTACTTATTATTATTCATTACATTA
ATTAATAATCACTGTAATTCCGGGGAGGAGCTAACAATCTATATAACTAA
CTACACTTCCGAATGGCTGAGTTTATGCCGCCAGACGGAGACGGGATCAC
TTCAGTGACTCCAGGCTGAACTTGGG (SEQ ID NO: 1)
Annotations:

| Putative Domain | Base range |
|---|---|
| ORF1 | 283-2250 |
| ORF2 | 59-391 |
| ORF3 | 2277-2462 |
| GC-rich region, or a portion thereof | 2515-2615 |
| 5′ UTR Conserved Domain, or a portion thereof | 1-71 |

TABLE A1
Novel Anellovirus amino acid sequence (Betatorquevirus)
RING 19 (Betatorquevirus)

| | |
|---|---|
| ORF1 | MPWYPRRRYPRRRYRWLRRWRARRPFRPRYRRRYWVRNYSRKRKLFKITT |
| | KEWQPKVIRKTHVKGTYPLFLCTKHRINNNMIQYLDSIAPEHYYGGGGFS |
| | IMQFSLQALYEEFIKAKNWWTNTNCFLPLVRYMGCSFKFYKTEFYDYIVL |
| | IERCYPLACTDEMYLSTQPSIMMLTRKCIFVPCKQNSKGKKPYKKVRVRP |
| | PSQMTTGWHFSQDLANMPLVVLKTSVCSFDRYYTDSTAKSTTIGFKTLNT |
| | QTFRYHDWQEPPTTGYKPQNLLWFYGAENGSPVDPNNTIVSNLIYLGGTG |
| | PYEKGTPIKTNISNYFSEPKLWGNIFHDDYTSGTSPVFVTNKSPSEIKTA |
| | WNTIKDLTVKASGVFTLRTIPLWLPCRYNPFADKATNNKIWLVSIHSDHT |
| | EWKPIDNPLLQRTDLPLWLLVWGWQDWQKKNQQTSQPDINYLTVISSPYI |
| | SCYPKLDYYVLLDEGFWEGHSTYIESITDSDKKHWYPKNRFQIETLNLIA |
| | NTGPGTVKLRENQAAEGHMVYRFNFKLGGCPAPMEKICDPSKQSKYPIPN |
| | NQQQTTSLQSPENPIQTYLYDFDERRGLLTERATKRIKQDHTSEKTVLPF |
| | TGAATDLPILQTTSQEESSSEEEEEQQAEKKLLQLRRKQHRLRERILQLL |
| | DIQNT (SEQ ID NO: 2) |
| ORF2 | MAERARNLNIIILQMQIQPPIRTFKQTISDWKNLIVHVHDNICNCNKPLE |
| | HTIDTCITNPDELRLNKSTKQQLQKCLGTPEEDTQEDVIDGFADGELDAL |
| | FAQDTEEDTG (SEQ ID NO: 3) |
| ORF3 | MFGDTHVPNRRMTPEEFEQELIVAGVFRRPPCYYIKDRPTYPYVPKPTDE |
| | KCMVNFDLNFP (SEQ ID NO: 4) |

TABLE N1.1
Novel Anellovirus nucleic acid sequence (Betatorquevirus)

| | |
|---|---|
| Name | RING 19 alternate |
| Genus/Clade | Betatorquevirus |
| Accession | N/A |

Full Sequence: 2876 bp
1 10 20 30 40 50
| | | | | |
CGGGAGCCGAAGGTGAGTGCAACCACCGTAGTCTAGGGGCAATTCGGGCT
AGTTCAGTATGGCGGAACGGGCAAGAAACTTAAATATTATTATTTTACAG
ATGCAAATACAACCACCTATTAGAACCTTCAAACAAACAATTTCAGATTG
GAAAAACTTAATTGTCCACGTTCACGACAACATTTGCAACTGCAATAAAC
CATTAGAACACACTATTGATACCTGTATCACCAATCCAGATGAATTAAGA
TTAAACAAATCTACTAAACAACAACTACAAAAATGCCTTGGTACCCCAGA
AGAAGATACCCAAGAAGACGTTATCGATGGCTTCGCAGATGGAGAGCTAG
ACGCCCTTTTCGCCCAAGATACAGAAGAAGATACTGGGTAAGAAACTATT
CTCGAAAGAGAAAACTATTTAAAATAACAACCAAAGAATGGCAACCAAAA
GTTATAAGAAAGACTCATGTAAAGGGCACCTATCCTTTGTTTCTTTGTAC
AAAGCACAGAATTAACAATAATATGATACAATATTTAGACTCTATAGCTC
CAGAACACTATTACGGAGGAGGAGGATTTTCAATAATGCAATTTTCCTTA
CAAGCCTTATATGAAGAATTTATAAAAGCAAAAAACTGGTGGACTAATAC
AAACTGCTTTTTACCACTTGTAAGATATATGGGTTGCTCATTCAAATTTT
ATAAAACTGAATTTTATGATTATATTGTACTAATTGAAAGATGTTATCCA
CTTGCTTGTACTGATGAAATGTACTTATCTACTCAACCTAGTATTATGAT
GCTTACAAGAAAATGTATTTTTGTACCATGCAAACAAAACAGCAAAGGTA
AAAAACCTTACAAAAAAGTTAGAGTAAGACCACCTTCACAAATGACTACA
GGATGGCATTTCTCACAAGACTTAGCAAACATGCCACTTGTAGTACTAAA
AACTTCAGTATGCAGCTTTGACAGATATTACACAGACAGTACAGCTAAAT
CAACCACAATAGGCTTTAAAACACTTAACACACAAACATTTAGATATCAT
GACTGGCAGGAACCACCTACAACAGGATACAAACCACAAAACCTACTATG
GTTTTATGGAGCAGAAAACGGATCACCAGTAGACCCCAACAACACAATAG
TATCAAACCTAATATACTTAGGAGGCACAGGACCTTATGAAAAAGGCACA
CCAATAAAAACAAACATAAGCAATTACTTTTCAGAGCCTAAACTGTGGGG
AAATATATTTCACGATGATTATACATCAGGAACATCACCCGTGTTTGTTA
CAAACAAATCACCATCAGAAATTAAAACCGCATGGAACACTATAAAAGAC
TTAACTGTTAAAGCTAGCGGTGTATTTACATTAAGAACAATTCCACTATG
GCTACCTTGCAGATACAACCCATTTGCAGACAAAGCAACCAACAACAAAA
TATGGCTAGTTTCTATACATTCAGACCACACAGAATGGAAACCAATAGAC
AATCCATTACTACAACGAACAGACCTTCCTTTATGGTTACTTGTATGGGG
TTGGCAAGATTGGCAGAAAAAAAACCAACAAACTTCACAACCTGATATTA
ATTATTTAACAGTAATATCTTCACCATATATATCATGCTACCCAAAATTA
GATTACTATGTGTTACTAGATGAAGGATTTTGGGAGGGTCACTCAACATA
CATAGAGTCAATTACAGACTCAGACAAAAAACACTGGTACCCTAAAAATA
GATTTCAAATAGAAACACTTAATCTAATAGCTAACACAGGTCCAGGAACT
GTAAAACTAAGAGAAAACCAAGCAGCAGAAGGTCACATGGTATATCGCTT
TAATTTTAAGCTTGGAGGATGTCCCGCACCGATGGAAAAAATATGTGACC
CTAGCAAACAATCCAAATATCCTATTCCCAATAACCAGCAACAAACAACT
TCGTTGCAGAGTCCAGAAAACCCAATTCAAACCTATCTCTACGACTTCGA
CGAAAGGAGGGGCCTACTTACAGAAAGAGCTACAAAAAGAATCAAACAAG
ATCACACATCTGAAAAAACTGTTTTGCCATTTACAGGAGCAGCAACAGAC
CTCCCCATACTCCAAACAACATCACAGGAGGAAAGCTCCTCGGAAGAAGA
AGAAGAGCAACAAGCGGAGAAGAAACTACTCCAGCTCCGAAGAAAGCAGC
ACCGACTCCGGGAGCGAATCCTCCAGCTATTAGACATACAAAATACATAA
TAAAACAAAGTACTGTAAAAATTGATATGTTTGGAGATACTCATGTACCT
AACCGTAGAATGACCCCAGAAGAATTTGAACAAGAACTAATTGTCGCTGG
TGTTTTTCGCAGACCTCCTTGTTACTATATAAAAGATAGACCTACTTATC
CTTATGTACCAAAACCTACTGATGAAAAATGTATGGTAAACTTTGACTTA
AACTTTCCTTAATAAACTACGCCTGCAAACTTTCACTCTCGGTGTCCATT
TATATAAGATAAAACTTAAATAAACATCCACCACTCTCCCAAATACGCAG
GCGCACAAGGGGGCTCCGCCCCCTTAAACCCCCAAGGGGGCTCCGCCCCC
TTAAACCCCCAAGGGGGCTCCGCCCCCTTACACCCCCTAATAAATATTCA
ACAGGAAAACCACCTAATTAGAATTGCCGACCACAAACCGTCACTTACTT
CTCCTTTTTGCACTTACTTCCTCTTTTACTTATTATTATTCATTACATTA
ATTAATAATCACTGTAATTCCGGGGAGGAGCTAACAATCTATATAACTAA
CTACACTTCCGAATGGCTGAGTTTATGCCGCCAGACGGAGACGGGATCAC
TTCAGTGACTCCAGGCTGAACTTGGG (SEQ ID NO: 1)
Annotations:

| Putative Domain | Base range |
|---|---|
| ORF1 | 283-2250 |
| ORF2 | 101-391 |
| ORF3 | 2277-2462 |
| GC-rich region, or a portion thereof | 2515-2615 |
| 5′ UTR Conserved Domain, or a portion | 1-71 |
| thereof | |

TABLE A1.1
Novel Anellovirus amino acid sequence (Betatorquevirus)
RING 19 (Betatorquevirus)

| | |
|---|---|
| ORF1 | MPWYPRRRYPRRRYRWLRRWRARRPFRPRYRRRYWVRNYSRKRKLFKITT |
| | KEWQPKVIRKTHVKGTYPLFLCTKHRINNNMIQYLDSIAPEHYYGGGGFS |
| | IMQFSLQALYEEFIKAKNWWTNTNCFLPLVRYMGCSFKFYKTEFYDYIVL |
| | IERCYPLACTDEMYLSTQPSIMMLTRKCIFVPCKQNSKGKKPYKKVRVRP |
| | PSQMTTGWHFSQDLANMPLVVLKTSVCSFDRYYTDSTAKSTTIGFKTLNT |
| | QTFRYHDWQEPPTTGYKPQNLLWFYGAENGSPVDPNNTIVSNLIYLGGTG |
| | PYEKGTPIKTNISNYFSEPKLWGNIFHDDYTSGTSPVFVTNKSPSEIKTA |
| | WNTIKDLTVKASGVFTLRTIPLWLPCRYNPFADKATNNKIWLVSIHSDHT |
| | EWKPIDNPLLQRTDLPLWLLVWGWQDWQKKNQQTSQPDINYLTVISSPYI |
| | SCYPKLDYYVLLDEGFWEGHSTYIESITDSDKKHWYPKNRFQIETLNLIA |
| | NTGPGTVKLRENQAAEGHMVYRFNFKLGGCPAPMEKICDPSKQSKYPIPN |
| | NQQQTTSLQSPENPIQTYLYDFDERRGLLTERATKRIKQDHTSEKTVLPF |
| | TGAATDLPILQTTSQEESSSEEEEEQQAEKKLLQLRRKQHRLRERILQLL |
| | DIQNT (SEQ ID NO: 2) |
| ORF2 | MQIQPPIRTFKQTISDWKNLIVHVHDNICNCNKPLEHTIDTCITNPDELR |
| | LNKSTKQQLQKCLGTPEEDTQEDVIDGFADGELDALFAQDTEEDTG |
| | (SEQ ID NO: 173) |
| ORF3 | MFGDTHVPNRRMTPEEFEQELIVAGVFRRPPCYYIKDRPTYPYVPKPTDE |
| | KCMVNFDLNFP (SEQ ID NO: 4) |

TABLE N2
Exemplary Anellovirus nucleic acid sequence (Betatorquevirus)

| | |
|---|---|
| Name | Ring2 |
| Genus/Clade | Betatorquevirus |
| Accession Number | JX134045.1 |
| Full Sequence: 2797 bp | |

1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 54)
Annotations:

| Putative Domain | Base range |
|---|---|
| TATA Box | 237-243 |
| Cap Site | 260-267 |
| Transcriptional Start Site | 267 |
| 5′ UTR Conserved Domain | 323-393 |
| ORF2 | 424-723 |
| ORF2/2 | 424-719; 2274-2589 |
| ORF2/3 | 424-719; 2449-2812 |
| ORF1 | 612-2612 |
| ORF1/1 | 612-719; 2274-2612 |
| ORF1/2 | 612-719; 2449-2589 |
| Three open-reading frame region | 2441-2586 |
| Poly(A) Signal | 2808-2813 |
| GC-rich region | 2868-2929 |

TABLE A2
Exemplary Anellovirus amino acid sequences (Betatorquevirus)
Ring2 (Betatorquevirus)

| | |
|---|---|
| ORF2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEG (SEQ ID NO: 55) |
| ORF2/2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGFNIPYPVTSMKQLRY |
| | RVQGKPQNPSYTPSTIDTGTTQQQLCHELAKTGHLKTLFLKLQSQIDSNCSNKPSNA |
| | CKSRKKRRRKKKKKYSSSSATSDSSSSCTESE (SEQ ID NO: 56) |
| ORF2/3 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGARSTATAQTSPRMP |
| | ANLGRNAGEKRKRSTAAHQQPQTAAAAVQRANNIIIKGPITFNCVKKVKLFDDKPK |
| | NRRFTPEEFETELQIAKWLKRPPRSFVNDPPFYPWLPPEPVVNFKLNFTE (SEQ ID |
| | NO: 57) |
| ORF1 | MPYYYRRRRYNYRRPRWYGRGWIRRPFRRRFRRKRRVRPTYTTIPLKQWQPPYKR |
| | TCYIKGQDCLIYYSNLRLGMNSTMYEKSIVPVHWPGGGSFSVSMLTLDALYDIHKL |
| | CRNWWTSTNQDLPLVRYKGCKITFYQSTFTDYIVRIHTELPANSNKLTYPNTHPLM |
| | MMMSKYKHIIPSRQTRRKKKPYTKIFVKPPPQFENKWYFATDLYKIPLLQIHCTACN |
| | LQNPFVKPDKLSNNVTLWSLNTISIQNRNMSVDQGQSWPFKILGTQSFYFYFYTGA |
| | NLPGDTTQIPVADLLPLTNPRINRPGQSLNEAKITDHITFTEYKNKFTNYWGNPFNK |
| | HIQEHLDMILYSLKSPEAIKNEWTTENMKWNQLNNAGTMALTPFNEPIFTQIQYNP |
| | DRDTGEDTQLYLLSNATGTGWDPPGIPELILEGFPLWLIYWGFADFQKNLKKVTNID |
| | TNYMLVAKTKFTQKPGTFYLVILNDTFVEGNSPYEKQPLPEDNIKWYPQVQYQLEA |
| | QNKLLQTGPFTPNIQGQLSDNISMFYKFYFKWGGSPPKAINVENPAHQIQYPIPRNE |
| | HETTSLQSPGEAPESILYSFDYRHGNYTTTALSRISQDWALKDTVSKITEPDRQQLLK |
| | QALECLQISEETQEKKEKEVQQLISNLRQQQQLYRERIISLLKDQ (SEQ ID NO: 58) |
| ORF1/1 | MPYYYRRRRYNYRRPRWYGRGWIRRPFRRRFRRKRRIQYPIPRNEHETTSLQSPGE |
| | APESILYSFDYRHGNYTTTALSRISQDWALKDTVSKITEPDRQQLLKQALECLQISEE |
| | TQEKKEKEVQQLISNLRQQQQLYRERIISLLKDQ (SEQ ID NO: 59) |
| ORF1/2 | MPYYYRRRRYNYRRPRWYGRGWIRRPFRRRFRRKRRSQIDSNCSNKPSNACKSRK |
| | KRRRKKKKKYSSSSATSDSSSSCTESE (SEQ ID NO: 60) |

TABLE N3
Exemplary chicken anemia virus (CAV) nucleic acid sequence

| | |
|---|---|
| Name | CAV isolate Cuxhaven 1 |
| Genus/Clade | Gyrovirus |
| Accession Number | M55918 |
| Full Sequence: 2313 bp | |

| | |
|---|---|
| CGAGTGGTTA CTATTCCATC ACCATTCTAG CCTGTACACA GAAAGTCAAG ATGGACGAAT | 60 |
| CGCTCGACTT CGCTCGCGAT TCGTCGAAGG CGGGGGGCCG GAGGCCCCCC GGTGGCCCCC | 120 |
| CTCCAACGAG TGGAGCACGT ACAGGGGGGT ACGTCATCCG TACAGGGGGG TACGTCATCC | 180 |
| GTACAGGGGG GTACGTCACA AAGAGGCGTT CCCGTACAGG GGGGTACGTC ACGCGTACAG | 240 |
| GGGGGTACGT CACAGCCAAT CAAAAGCTGC CACGTTGCGA AAGTGACGTT TCGAAAATGG | 300 |
| GCGGCGCAAG CCTCTCTATA TATTGAGCGC ACATACCGGT CGGCAGTAGG TATACGCAAG | 360 |
| GCGGTCCGGG TGGATGCACG GGAACGGCGG ACAACCGGCC GCTGGGGGCA GTGAATCGGC | 420 |
| GCTTAGCCGA GAGGGGCAAC CTGGGCCCAG CGGAGCCGCG CAGGGGCAAG TAATTTCAAA | 480 |
| TGAACGCTCT CCAAGAAGAT ACTCCACCCG GACCATCAAC GGTGTTCAGG CCACCAACAA | 540 |
| GTTCACGGCC GTTGGAAACC CCTCACTGCA GAGAGATCCG GATTGGTATC GCTGGAATTA | 600 |
| CAATCACTCT ATCGCTGTGT GGCTGCGCGA ATGCTCGCGC TCCCACGCTA AGATCTGCAA | 660 |
| CTGCGGACAA TTCAGAAAGC ACTGGTTTCA AGAATGTGCC GGACTTGAGG ACCGATCAAC | 720 |
| CCAAGCCTCC CTCGAAGAAG CGATCCTGCG ACCCCTCCGA GTACAGGGTA AGCGAGCTAA | 780 |
| AAGAAAGCTT GATTACCACT ACTCCCAGCC GACCCCGAAC CGCAAAAAGG CGTATAAGAC | 840 |
| TGTAAGATGG CAAGACGAGC TCGCAGACCG AGAGGCCGAT TTTACTCCTT CAGAAGAGGA | 900 |
| CGGTGGCACC ACCTCAAGCG ACTTCGACGA AGATATAAAT TTCGACATCG GAGGAGACAG | 960 |
| CGGTATCGTA GACGAGCTTT TAGGAAGGCC TTTCACAACC CCCGCCCCGG TACGTATAGT | 1020 |
| GTGAGGCTGC CGAACCCCCA ATCTACTATG ACTATCCGCT TCCAAGGGGT CATCTTTCTC | 1080 |
| ACGGAAGGAC TCATTCTGCC TAAAAACAGC ACAGCGGGGG GCTATGCAGA CCACATGTAC | 1140 |
| GGGGCGAGAG TCGCCAAGAT CTCTGTGAAC CTGAAAGAGT TCCTGCTAGC CTCAATGAAC | 1200 |
| CTGACATACG TGAGCAAAAT CGGAGGCCCC ATCGCCGGTG AGTTGATTGC GGACGGGTCT | 1260 |
| AAATCACAAG CCGCGGACAA TTGGCCTAAT TGCTGGCTGC CGCTAGATAA TAACGTGCCC | 1320 |
| TCCGCTACAC CATCGGCATG GTGGAGATGG GCCTTAATGA TGATGCAGCC CACGGACTCT | 1380 |
| TGCCGGTTCT TTAATCACCC AAAGCAGATG ACCCTGCAAG ACATGGGTCG CATGTTTGGG | 1440 |
| GGCTGGCACC TGTTCCGACA CATTGAAACC CGCTTTCAGC TCCTTGCCAC TAAGAATGAG | 1500 |
| GGATCCTTCA GCCCCGTGGC GAGTCTTCTC TCCCAGGGAG AGTACCTCAC GCGTCGGGAC | 1560 |
| GATGTTAAGT ACAGCAGCGA TCACCAGAAC CGGTGGCAAA AAGGCGGACA ACCGATGACG | 1620 |
| GGGGGCATTG CTTATGCGAC CGGGAAAATG AGACCCGACG AGCAACAGTA CCCTGCTATG | 1680 |
| CCCCCAGACC CCCCGATCAT CACCGCTACT ACAGCGCAAG GCACGCAAGT CCGCTGCATG | 1740 |
| AATAGCACGC AAGCTTGGTG GTCATGGGAC ACATATATGA GCTTTGCAAC ACTCACAGCA | 1800 |
| CTCGGTGCAC AATGGTCTTT TCCTCCAGGG CAACGTTCAG TTTCTAGACG GTCCTTCAAC | 1860 |
| CACCACAAGG CGAGAGGAGC CGGGGACCCC AAGGGCCAGA GATGGCACAC GCTGGTGCCG | 1920 |
| CTCGGCACGG AGACCATCAC CGACAGCTAC ATGTCAGCAC CCGCATCAGA GCTGGACACT | 1980 |
| AATTTCTTTA CGCTTTACGT AGCGCAAGGC ACAAATAAGT CGCAACAGTA CAAGTTCGGC | 2040 |
| ACAGCTACAT ACGCGCTAAA GGAGCCGGTA ATGAAGAGCG ATGCATGGGC AGTGGTACGC | 2100 |
| GTCCAGTCGG TCTGGCAGCT GGGTAACAGG CAGAGGCCAT ACCCATGGGA CGTCAACTGG | 2160 |
| GCGAACAGCA CCATGTACTG GGGGACGCAG CCCTGAAAAG GGGGGGGGGC TAAAGCCCCC | 2220 |
| CCCCCTTAAA CCCCCCCCTG GGGGGGATTC CCCCCCAGAC CCCCCCTTTA TATAGCACTC | 2280 |
| AATAAACGCA GAAAATAGAT TTATCGCACT ATC | 2313 |

(SEQ ID NO: 5)
Annotations:

| Putative Domain | Base range |
|---|---|
| 5′ UTR | 1-374 |
| Repeat Region | 138-254 |
| CAAT Signal | 255-260 |
| TATA Box | 317-322 |
| VP2 | 374-1024 |
| VP3 (Apoptin) | 480-845 |
| VP1 | 847-2196 |
| 3′ UTR | 2197-2313 |
| GC-Rich Region | 2200-2266 |
| PolyA Signal Sequence | 2281-2286 |

TABLE N4
Alternate exemplary chicken anemia virus (CAV) nucleic acid sequence

| | |
|---|---|
| Name | CAV isolate Cuxhaven 1 |
| Genus/Clade | Gyrovirus |
| Accession Number | M55918 |
| Full Sequence: 2319 bp | |

| | |
|---|---|
| GAATTCCGAG TGGTTACTAT TCCATCACCA TTCTAGCCTG TACACAGAAA GTCAAGATGG | 60 |
| ACGAATCGCT CGACTTCGCT CGCGATTCGT CGAAGGCGGG GGGCCGGAGG CCCCCCGGTG | 120 |
| GCCCCCCTCC AACGAGTGGA GCACGTACAG GGGGGTACGT CATCCGTACA GGGGGGTACG | 180 |
| TCATCCGTAC AGGGGGGTAC GTCACAAAGA GGCGTTCCCG TACAGGGGGG TACGTCACGC | 240 |
| GTACAGGGGG GTACGTCACA GCCAATCAAA AGCTGCCACG TTGCGAAAGT GACGTTTCGA | 300 |
| AAATGGGCGG CGCAAGCCTC TCTATATATT GAGCGCACAT ACCGGTCGGC AGTAGGTATA | 360 |
| CGCAAGGCGG TCCGGGTGGA TGCACGGGAA CGGCGGACAA CCGGCCGCTG GGGGCAGTGA | 420 |
| ATCGGCGCTT AGCCGAGAGG GGCAACCTGG GCCCAGCGGA GCCGCGCAGG GGCAAGTAAT | 480 |
| TTCAAATGAA CGCTCTCCAA GAAGATACTC CACCCGGACC ATCAACGGTG TTCAGGCCAC | 540 |
| CAACAAGTTC ACGGCCGTTG GAAACCCCTC ACTGCAGAGA GATCCGGATT GGTATCGCTG | 600 |
| GAATTACAAT CACTCTATCG CTGTGTGGCT GCGCGAATGC TCGCGCTCCC ACGCTAAGAT | 660 |
| CTGCAACTGC GGACAATTCA GAAAGCACTG GTTTCAAGAA TGTGCCGGAC TTGAGGACCG | 720 |
| ATCAACCCAA GCCTCCCTCG AAGAAGCGAT CCTGCGACCC CTCCGAGTAC AGGGTAAGCG | 780 |
| AGCTAAAAGA AAGCTTGATT ACCACTACTC CCAGCCGACC CCGAACCGCA AAAAGGCGTA | 840 |
| TAAGACTGTA AGATGGCAAG ACGAGCTCGC AGACCGAGAG GCCGATTTTA CTCCTTCAGA | 900 |
| AGAGGACGGT GGCACCACCT CAAGCGACTT CGACGAAGAT ATAAATTTCG ACATCGGAGG | 960 |
| AGACAGCGGT ATCGTAGACG AGCTTTTAGG AAGGCCTTTC ACAACCCCCG CCCCGGTACG | 1020 |
| TATAGTGTGA GGCTGCCGAA CCCCCAATCT ACTATGACTA TCCGCTTCCA AGGGGTCATC | 1080 |
| TTTCTCACGG AAGGACTCAT TCTGCCTAAA AACAGCACAG CGGGGGGCTA TGCAGACCAC | 1140 |
| ATGTACGGGG CGAGAGTCGC CAAGATCTCT GTGAACCTGA AAGAGTTCCT GCTAGCCTCA | 1200 |
| ATGAACCTGA CATACGTGAG CAAAATCGGA GGCCCCATCG CCGGTGAGTT GATTGCGGAC | 1260 |
| GGGTCTAAAT CACAAGCCGC GGACAATTGG CCTAATTGCT GGCTGCCGCT AGATAATAAC | 1320 |
| GTGCCCTCCG CTACACCATC GGCATGGTGG AGATGGGCCT TAATGATGAT GCAGCCCACG | 1380 |
| GACTCTTGCC GGTTCTTTAA TCACCCAAAG CAGATGACCC TGCAAGACAT GGGTCGCATG | 1440 |
| TTTGGGGGCT GGCACCTGTT CCGACACATT GAAACCCGCT TTCAGCTCCT TGCCACTAAG | 1500 |
| AATGAGGGAT CCTTCAGCCC CGTGGCGAGT CTTCTCTCCC AGGGAGAGTA CCTCACGCGT | 1560 |
| CGGGACGATG TTAAGTACAG CAGCGATCAC CAGAACCGGT GGCAAAAAGG CGGACAACCG | 1620 |
| ATGACGGGGG GCATTGCTTA TGCGACCGGG AAAATGAGAC CCGACGAGCA ACAGTACCCT | 1680 |
| GCTATGCCCC CAGACCCCCC GATCATCACC GCTACTACAG CGCAAGGCAC GCAAGTCCGC | 1740 |
| TGCATGAATA GCACGCAAGC TTGGTGGTCA TGGGACACAT ATATGAGCTT TGCAACACTC | 1800 |
| ACAGCACTCG GTGCACAATG GTCTTTTCCT CCAGGGCAAC GTTCAGTTTC TAGACGGTCC | 1860 |
| TTCAACCACC ACAAGGCGAG AGGAGCCGGG GACCCCAAGG GCCAGAGATG GCACACGCTG | 1920 |
| GTGCCGCTCG GCACGGAGAC CATCACCGAC AGCTACATGT CAGCACCCGC ATCAGAGCTG | 1980 |
| GACACTAATT TCTTTACGCT TTACGTAGCG CAAGGCACAA ATAAGTCGCA ACAGTACAAG | 2040 |
| TTCGGCACAG CTACATACGC GCTAAAGGAG CCGGTAATGA AGAGCGATGC ATGGGCAGTG | 2100 |
| GTACGCGTCC AGTCGGTCTG GCAGCTGGGT AACAGGCAGA GGCCATACCC ATGGGACGTC | 2160 |
| AACTGGGCGA ACAGCACCAT GTACTGGGGG ACGCAGCCCT GAAAAGGGGG GGGGGCTAAA | 2220 |
| GCCCCCCCCC CTTAAACCCC CCCCTGGGGG GGATTCCCCC CCAGACCCCC CCTTTATATA | 2280 |
| GCACTCAATA AACGCAGAAA ATAGATTTAT CGCACTATC | 2319 |

(SEQ ID NO: 100)
Annotations:

| Putative Domain | Base range |
|---|---|
| 5′ UTR | 1-379 |
| VP2 | 380-1030 |
| VP3 (Apoptin) | 485-851 |
| VP1 | 853-2202 |
| 3′ UTR | 2203-2319 |

TABLE A3
Exemplary CAV amino acid sequences
CAV

| | |
|---|---|
| VP1 | MARRARRPRGRFYSFRRGRWHHLKRLRRRYKFRHRRRQRYRRRAFRKAFHNPRP |
| | GTYSVRLPNPQSTMTIRFQGVIFLTEGLILPKNSTAGGYADHMYGARVAKISVN |
| | LKEFLLASMNLTYVSKIGGPIAGELIADGSKSQAADNWPNCWLPLDNNVPSATP |
| | SAWWRWALMMMQPTDSCRFFNHPKQMTLQDMGRMFGGWHLFRHIETRFQLLATK |
| | NEGSFSPVASLLSQGEYLTRRDDVKYSSDHQNRWQKGGQPMTGGIAYATGKMRP |
| | DEQQYPAMPPDPPIITATTAQGTQVRCMNSTQAWWSWDTYMSFATLTALGAQWS |
| | FPPGQRSVSRRSFNHHKARGAGDPKGQRWHTLVPLGTETITDSYMSAPASELDT |
| | NFFTLYVAQGTNKSQQYKFGTATYALKEPVMKSDAWAVVRVQSVWQLGNRQRPY |
| | PWDVNWANSTMYWGTQP |
| VP2 | MHGNGGQPAAGGSESALSREGQPGPSGAAQGQVISNERSPRRYSTRTINGVQAT |
| | NKFTAVGNPSLQRDPDWYRWNYNHSIAVWLRECSRSHAKICNCGQFRKHWFQEC |
| | AGLEDRSTQASLEEAILRPLRVQGKRAKRKLDYHYSQPTPNRKKAYKTVRWQDE |
| | LADREADFTPSEEDGGTTSSDFDEDINFDIGGDSGIVDELLGRPFTTPAPVRIV |
| | * |
| VP3 | MNALQEDTPPGPSTVFRPPTSSRPLETPHCREIRIGIAGITITLSLCGCANARA |
| (Apoptin) | PTLRSATADNSESTGFKNVPDLRTDQPKPPSKKRSCDPSEYRVSELKESLITTT |
| | PSRPRTAKRRIRL* |

In some embodiments, an Anelloviridae family vector (e.g. anellovector) as described herein is a chimeric Anelloviridae family vector (e.g. chimeric anellovector). In some embodiments, a chimeric Anelloviridae family vector further comprises one or more elements, polypeptides, or nucleic acids from a virus other than an Anelloviridae family virus.

In some embodiments, the chimeric Anelloviridae family vector comprises a plurality of polypeptides (e.g., ORF1, ORF1/1, ORF1/2, ORF2, ORF2/2, ORF2/3, ORF2t/3, VP1, VP2, and/or VP3) comprising sequences from a plurality of different Anelloviridae family viruses (e.g., as described herein).

In some embodiments, the Anelloviridae family vector comprises a chimeric polypeptide (e.g., ORF1, ORF1/1, ORF1/2, ORF2, ORF2/2, ORF2/3, ORF2t/3, VP1, VP2, and/or VP3), e.g., comprising at least one portion from an Anelloviridae family virus (e.g., as described herein) and at least one portion from a different virus (e.g., as described herein).

In some embodiments, the Anelloviridae family vector comprises a chimeric polypeptide (e.g., ORF1, ORF1/1, ORF1/2, ORF2, ORF2/2, ORF2/3, ORF2t/3, VP1, VP2, and/or VP3), e.g., comprising at least one portion from one Anelloviridae family virus (e.g., as described herein) and at least one portion from a different Anelloviridae family virus (e.g., as described herein). In some embodiments, the Anelloviridae family vector comprises a chimeric ORF1 or VP1 molecule comprising at least one portion of an ORF1 or VP1 molecule from one Anelloviridae family virus (e.g., as described herein), or an ORF1 or VP1 molecule having at least 75%, 80%, 85%, 90%, 95%, 96%, 97%, 98%, or 99% amino acid sequence identity thereto, and at least one portion of an ORF1 or VP1 molecule from a different Anelloviridae family virus (e.g., as described herein), or an ORF1 or VP1 molecule having at least 75%, 80%, 85%, 90%, 95%, 96%, 97%, 98%, or 99% amino acid sequence identity thereto. In some embodiments, the chimeric ORF1 or VP1 molecule comprises an ORF1 or VP1 jelly-roll domain from one Anelloviridae family virus, or a sequence having at least 75%, 80%, 85%, 90%, 95%, 96%, 97%, 98%, or 99% sequence identity thereto, and an ORF1 or VP1 amino acid subsequence (e.g., as described herein) from a different Anelloviridae family virus, or a sequence having at least 75%, 80%, 85%, 90%, 95%, 96%, 97%, 98%, or 99% sequence identity thereto. In some embodiments, the chimeric ORF1 or VP1 molecule comprises an ORF1 or VP1 arginine-rich region from one Anelloviridae family virus, or a sequence having at least 75%, 80%, 85%, 90%, 95%, 96%, 97%, 98%, or 99% sequence identity thereto, and an ORF1 or VP1 amino acid subsequence (e.g., as described herein) from a different Anelloviridae family virus, or a sequence having at least 75%, 80%, 85%, 90%, 95%, 96%, 97%, 98%, or 99% sequence identity thereto. In some embodiments, the chimeric ORF1 or VP1 molecule comprises an ORF1 or VP1 hypervariable domain from one Anelloviridae family virus, or a sequence having at least 75%, 80%, 85%, 90%, 95%, 96%, 97%, 98%, or 99% sequence identity thereto, and an ORF1 or VP1 amino acid subsequence (e.g., as described herein) from a different Anelloviridae family virus, or a sequence having at least 75%, 80%, 85%, 90%, 95%, 96%, 97%, 98%, or 99% sequence identity thereto. In some embodiments, the chimeric ORF1 molecule comprises an ORF1 N22 domain from one Anelloviridae family virus, or a sequence having at least 75%, 80%, 85%, 90%, 95%, 96%, 97%, 98%, or 99% sequence identity thereto, and an ORF1 amino acid subsequence (e.g., as described herein) from a different Anelloviridae family virus, or a sequence having at least 75%, 80%, 85%, 90%, 95%, 96%, 97%, 98%, or 99% sequence identity thereto. In some embodiments, the chimeric ORF1 or VP1 molecule comprises an ORF1 or VP1 C-terminal domain from one Anellovirdae family virus, or a sequence having at least 75%, 80%, 85%, 90%, 95%, 96%, 97%, 98%, or 99% sequence identity thereto, and an ORF1 or VP1 amino acid subsequence (e.g., as described herein) from a different Anelloviridae family virus, or a sequence having at least 75%, 80%, 85%, 90%, 95%, 96%, 97%, 98%, or 99% sequence identity thereto.

In some embodiments, the Anelloviridae family vector comprises a chimeric ORF1/1 molecule comprising at least one portion of an ORF1/1 molecule from one Anelloviridae family virus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anelloviridae family virus (e.g., as described herein), or an ORF1/1 molecule having at least 75%, 80%, 85%, 90%, 95%, 96%, 97%, 98%, or 99% amino acid sequence identity thereto. In some embodiments, the Anelloviridae family vector comprises a chimeric ORF1/2 molecule comprising at least one portion of an ORF1/2 molecule from one Anelloviridae family virus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anelloviridae family virus (e.g., as described herein), or an ORF1/2 molecule having at least 75%, 80%, 85%, 90%, 95%, 96%, 97%, 98%, or 99% amino acid sequence identity thereto. In some embodiments, the Anelloviridae family vector comprises a chimeric ORF2 or VP2 molecule comprising at least one portion of an ORF2 or VP2 molecule from one Anelloviridae family virus (e.g., as described herein), or an ORF2 or VP2 molecule having at least 75%, 80%, 85%, 90%, 95%, 96%, 97%, 98%, or 99% amino acid sequence identity thereto, and at least one portion of an ORF2 or VP2 molecule from a different Anelloviridae family virus (e.g., as described herein), or an ORF2 or VP2 molecule having at least 75%, 80%, 85%, 90%, 95%, 96%, 97%, 98%, or 99% amino acid sequence identity thereto. In some embodiments, the Anelloviridae family vector comprises a chimeric ORF2/2 molecule comprising at least one portion of an ORF2/2 molecule from one Anelloviridae family virus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anelloviridae family virus (e.g., as described herein), or an ORF2/2 molecule having at least 75%, 80%, 85%, 90%, 95%, 96%, 97%, 98%, or 99% amino acid sequence identity thereto. In some embodiments, the Anelloviridae family vector comprises a chimeric ORF2/3 molecule comprising at least one portion of an ORF2/3 molecule from one Anelloviridae family virus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anelloviridae family virus (e.g., as described herein), or an ORF2/3 molecule having at least 75%, 80%, 85%, 90%, 95%, 96%, 97%, 98%, or 99% amino acid sequence identity thereto. In some embodiments, the Anelloviridae family vector comprises a chimeric ORF2T/3 molecule comprising at least one portion of an ORF2T/3 molecule from one Anelloviridae family virus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anelloviridae family virus (e.g., as described herein), or an ORF2T/3 molecule having at least 75%, 80%, 85%, 90%, 95%, 96%, 97%, 98%, or 99% amino acid sequence identity thereto.

In some embodiments, an Anelloviridae family vector comprises a nucleic acid comprising a sequence listed in PCT Application No. PCT/US2018/037379, incorporated herein by reference in its entirety. In some embodiments, an Anelloviridae family vector comprises a polypeptide comprising a sequence listed in PCT Application No. PCT/US2018/037379, incorporated herein by reference in its entirety.

In some embodiments, an Anelloviridae family vector comprises an Anelloviridae family virus genome, e.g., as identified according to the method described in Example 9. In some embodiments, an Anelloviridae family vector comprises an Anelloviridae family virus sequence, or a portion thereof, as described in Example 13.

In some embodiments, an anellovector comprises a genetic element comprising a consensus Anellovirus motif, e.g., as shown in Table 19. In some embodiments, an anellovector comprises a genetic element comprising a consensus Anellovirus ORF1 motif, e.g., as shown in Table 19. In some embodiments, an anellovector comprises a genetic element comprising a consensus Anellovirus ORF1/1 motif, e.g., as shown in Table 19. In some embodiments, an anellovector comprises a genetic element comprising a consensus Anellovirus ORF1/2 motif, e.g., as shown in Table 19. In some embodiments, an anellovector comprises a genetic element comprising a consensus Anellovirus ORF2/2 motif, e.g., as shown in Table 19. In some embodiments, an anellovector comprises a genetic element comprising a consensus Anellovirus ORF2/3 motif, e.g., as shown in Table 19. In some embodiments, an anellovector comprises a genetic element comprising a consensus Anellovirus ORF2t/3 motif, e.g., as shown in Table 19. In some embodiments, X, as shown in Table 19, indicates any amino acid. In some embodiments, Z, as shown in Table 19, indicates glutamic acid or glutamine. In some embodiments, B, as shown in Table 19, indicates aspartic acid or asparagine. In some embodiments, J, as shown in Table 19, indicates leucine or isoleucine.

TABLE 19
Consensus motifs in open reading frames (ORFs) of Anelloviruses

| | Open | | | |
|---|---|---|---|---|
| Consensus | Reading | | | SEQ ID |
| Threshold | Frame | Position | Motif | NO: |
| 50 | ORF1 | 79 | LIJRQWQPXXIRRCXIXGYXPLIXC | 68 |
| 50 | ORF1 | 111 | NYXXHXD | 69 |
| 50 | ORF1 | 135 | FSLXXLYDZ | 70 |
| 50 | ORF1 | 149 | NXWTXSNXDLDLCRYXGC | 71 |
| 50 | ORF1 | 194 | TXPSXHPGXMXLXKHK | 72 |
| 50 | ORF1 | 212 | IPSLXTRPXG | 73 |
| 50 | ORF1 | 228 | RIXPPXLFXDKWYFQXDL | 74 |
| 50 | ORF1 | 250 | LLXIXATA | 75 |
| 50 | ORF1 | 260 | LXXPFXSPXTD | 76 |
| 50 | ORF1 | 448 | YNPXXDKGXGNXIW | 77 |
| 50 | ORF1 | 519 | CPYTZPXL | 78 |
| 50 | ORF1 | 542 | XFGXGXMP | 79 |
| 50 | ORF1 | 569 | HQXEVXEX | 80 |
| 50 | ORF1 | 600 | KYXFXFXWGGNP | 81 |
| 50 | ORF1 | 653 | HSWDXRRG | 82 |
| 50 | ORF1 | 666 | AIKRXQQ | 83 |
| 50 | ORF1 | 750 | XQZQXXLR | 84 |
| 50 | ORF1/1 | 73 | PRXJQXXDP | 85 |
| 50 | ORF1/1 | 91 | HSWDXRRG | 86 |
| 50 | ORF1/1 | 105 | AIKRXQQ | 87 |
| 50 | ORF1/1 | 187 | QZQXXLR | 88 |
| 50 | ORF1/2 | 97 | KXKRRRR | 89 |
| 50 | ORF2/2 | 158 | PIXSLXXYKXXTR | 90 |
| 50 | ORF2/2 | 189 | LAXQLLKECXKN | 91 |
| 50 | ORF2/3 | 39 | HLNXLA | 92 |
| 50 | ORF2/3 | 272 | DRPPR | 93 |
| 50 | ORF2/3 | 281 | DXPFYPWXP | 94 |
| 50 | ORF2/3 | 300 | VXFKLXF | 95 |
| 50 | ORF2t/3 | 4 | WXPPVHBVXGIERXW | 96 |
| 50 | ORF2t/3 | 37 | AKRKLX | 97 |
| 50 | ORF2t/3 | 140 | PSSXDWXXEY | 98 |
| 50 | ORF2t/3 | 156 | DRPPR | 99 |
| 50 | ORF2t/3 | 167 | PFYPW | 100 |
| 50 | ORF2t/3 | 183 | NVXFKLXF | 101 |
| 50 | ORF1 | 84 | JXXXXWQPXXXXXCXIXGXXXJWQP | 102 |
| 50 | ORF1 | 149 | NXWXXXNXXXXLXRY | 103 |
| 50 | ORF1 | 448 | YNPXXDXG | 104 |

### Capsid Proteins (e.g., ORF1 Molecules and VPJ Molecules)

In some embodiments, the anellovector comprises an ORF1 molecule or VP1 molecule and/or a nucleic acid encoding an ORF1 molecule or VP1 molecule.

Generally, an ORF1 molecule comprises a polypeptide having the structural features and/or activity of an Anellovirus ORF1 protein (e.g., an Anellovirus ORF1 protein as described herein, e.g., as listed in Table A1 or A2), or a functional fragment thereof. In some embodiments, the ORF1 molecule comprises a truncation relative to an Anellovirus ORF1 protein (e.g., an Anellovirus ORF1 protein as described herein, e.g., as listed in Table A1 or A2). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 70%, 75%, 80%, 85%, 90%, 95%, 96%, 97%, 98%, 99%, or 100% sequence identity to an Anellovirus ORF1 protein sequence as shown in Table A1 or A2. In some embodiments, an ORF1 molecule comprises an amino acid sequence having at least 75%, 80%, 85%, 90%, 95%, 96%, 97%, 98%, or 99% sequence identity to an Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell. In some embodiments, an ORF1 molecule is encoded by an ORF1 nucleic acid. In some embodiments, the ORF1 nucleic acid comprises an antisense strand, which can be directly transcribed to produce mRNA encoding the ORF1 molecule. In some embodiments, the ORF1 nucleic acid comprises a sense strand.

Generally, a VP1 molecule comprises a polypeptide having the structural features and/or activity of a CAV VP1 protein (e.g., a CAV VP1 protein as described herein, e.g., as listed in Table A3), or a functional fragment thereof. In some embodiments, the VP1 molecule comprises a truncation relative to a CAV VP1 protein (e.g., a CAV VP1 protein as described herein, e.g., as listed in Table A3). In some embodiments, the VP1 molecule is truncated by at least 10, 20, 30, 40, 50, 60, 70, 80, 90, 100, 150, 200, 250, 300, 350, 400, 450, 500, 550, 600, 650, or 700 amino acids of the CAV VP1 protein. In some embodiments, a VP1 molecule comprises an amino acid sequence having at least 70%, 75%, 80%, 85%, 90%, 95%, 96%, 97%, 98%, 99%, or 100% sequence identity to a CAV VP1 protein sequence as shown in Table A3. A VP1 molecule can generally bind to a nucleic acid molecule, such as DNA (e.g., a genetic element, e.g., as described herein). In some embodiments, a VP1 molecule localizes to the nucleus of a cell. In certain embodiments, a VP1 molecule localizes to the nucleolus of a cell. In some embodiments, an VP1 molecule is encoded by an VP1 nucleic acid. In some embodiments, the VP1 nucleic acid comprises an antisense strand, which can be directly transcribed to produce mRNA encoding the VP1 molecule. In some embodiments, the VP1 nucleic acid comprises a sense strand.

In some embodiments, an ORF1 molecule as described herein comprises an amino acid sequence (e.g., an ORF1 sequence, or an arginine-rich region, jelly-roll domain, HVR, N22, or C-terminal domain sequence) as listed in any of Tables A2, A4, A6, A8, A10, A12, C1-C5, 2, 4, 6, 8, 10, 12, 14, 16, 18, 20-37, or D1-D10 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

Without wishing to be bound by theory, an ORF1 or VP1 molecule may be capable of binding to other ORF1 or VP1 molecules, e.g., to form a proteinaceous exterior (e.g., as described herein). Such an ORF1 or VP1 molecule may be described as having the capacity to form a capsid. In some embodiments, the proteinaceous exterior may encapsidate a nucleic acid molecule (e.g., a genetic element as described herein). In some embodiments, a plurality of ORF1 or VP1 molecules may form a multimer, e.g., to produce a proteinaceous exterior. In some embodiments, the multimer may be a homomultimer. In other embodiments, the multimer may be a heteromultimer (e.g., comprising a plurality of distinct ORF1 or VP1 molecules). It is also contemplated that an ORF1 or VP1 molecule may have replicase activity.

An ORF1 or VP1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In some embodiments, a VP1 molecule may, in some embodiments, comprise one or more of: an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a jelly-roll domain.

### Arginine-Rich Region

An arginine rich region (e.g., comprised an ORF1 molecule or VP1 molecule as described herein) has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

### Jelly Roll Domain

A jelly-roll domain or region (e.g., comprised an ORF1 molecule or VP1 molecule as described herein) comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more 0-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) β-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first 1-sheet in antiparallel orientation to a second β-sheet. In certain embodiments, the first 1-sheet comprises about four (e.g., 3, 4, 5, or 6) β-strands. In certain embodiments, the second β-sheet comprises about four (e.g., 3, 4, 5, or 6) β-strands. In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

An ORF1 molecule may also include a third region comprising the structure or activity of an Anellovirus N22 domain (e.g., as described herein, e.g., an N22 domain from an Anellovirus ORF1 protein as described herein), and/or a fourth region comprising the structure or activity of an Anellovirus C-terminal domain (CTD) (e.g., as described herein, e.g., a CTD from an Anellovirus ORF1 protein as described herein). In some embodiments, the ORF1 molecule comprises, in N-terminal to C-terminal order, the first, second, third, and fourth regions.

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization signal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

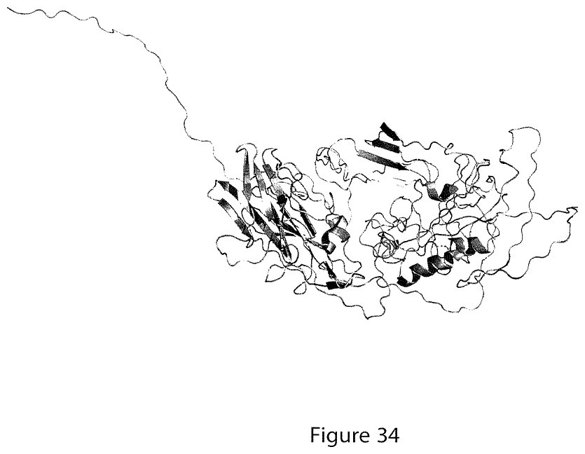

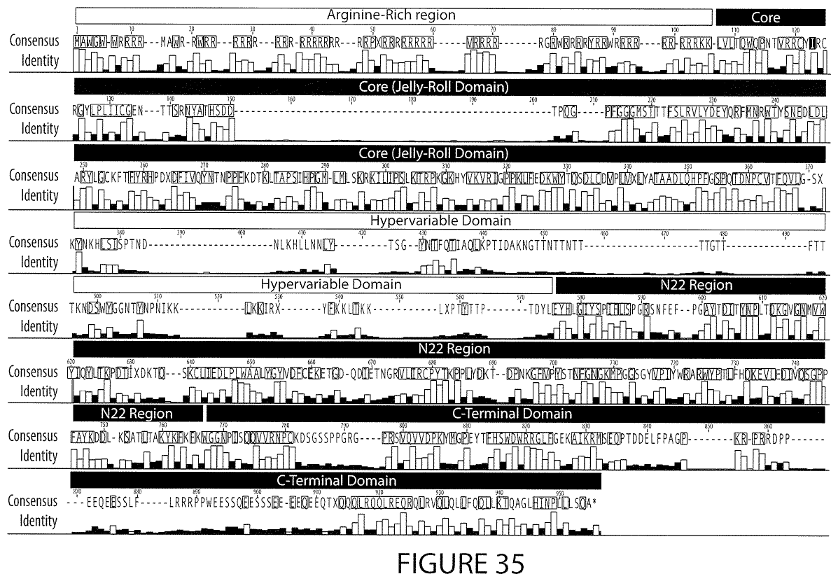

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34). In some embodiments, the conserved motifs may show 60, 70, 80, 85, 90, 95, or 100% sequence identity to an ORF1 protein of one or more wild-type Anellovirus clades (e.g., Betatorquevirus). In some embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule comprises at least one difference (e.g., a mutation, chemical modification, or epigenetic alteration) relative to a wild-type ORF1 protein, e.g., as described herein (e.g., as shown in Table A1 or A2).

### Conserved ORF1 Motif in N22 Domain

In some embodiments, a polypeptide (e.g., an ORF1 molecule) described herein comprises the amino acid sequence YNPX²DXGX²N (SEQ ID NO: 829), wherein Xⁿ is a contiguous sequence of any n amino acids. For example, X² indicates a contiguous sequence of any two amino acids. In some embodiments, the YNPX²DXGX²N (SEQ ID NO: 829) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein Xⁿ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

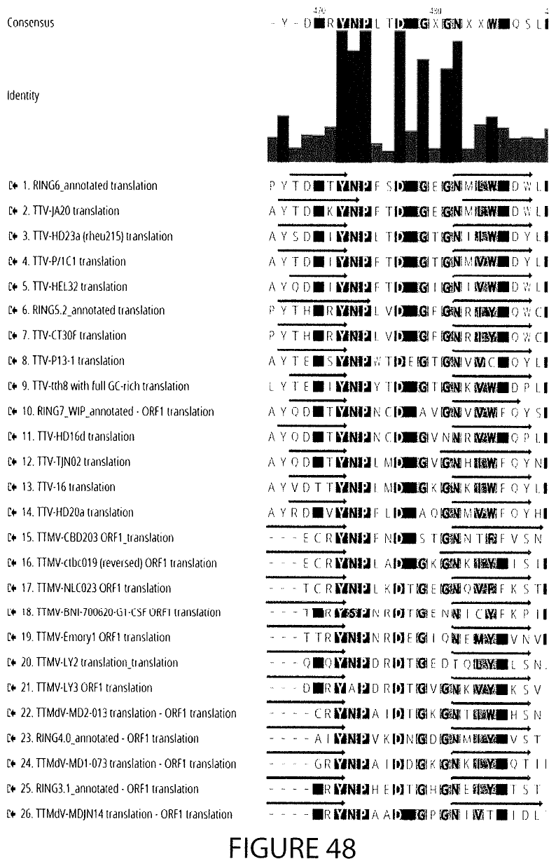

Exemplary YNPX²DXGX²N (SEQ ID NO: 829) motif-flanking secondary structures are described in Example 47 and FIG. 48. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48, flanking a YNPX²DXGX²N (SEQ ID NO: 829) motif (e.g., as described herein).

Conserved Secondary Structural Motif in ORF7 Jelly-Roll Domain In some embodiments, a polypeptide (e.g., an ORF1 molecule) described herein comprises one or more secondary structural elements comprised by an Anellovirus ORF1 protein (e.g., as described herein). In some embodiments, an ORF1 molecule comprises one or more secondary structural elements comprised by the jelly-roll domain of an Anellovirus ORF1 protein (e.g., as described herein). Generally, an ORF1 jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2, 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

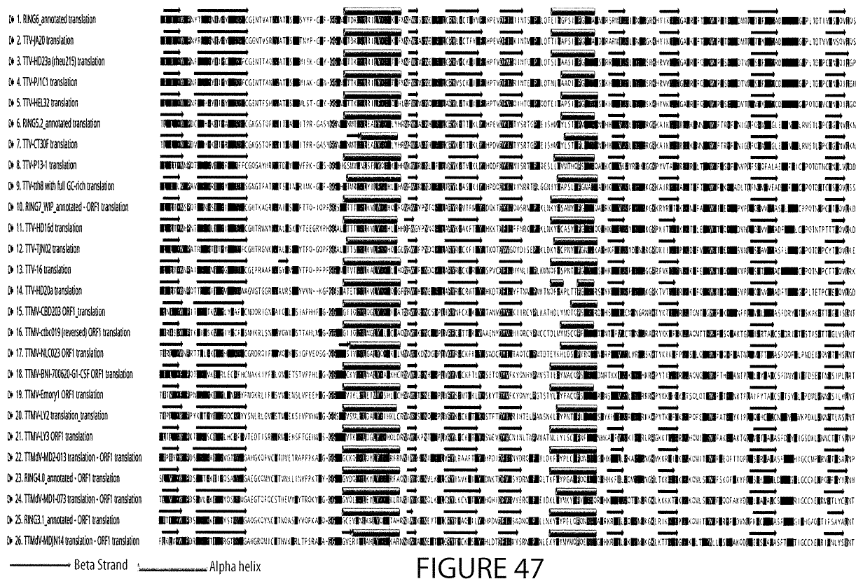

Exemplary jelly-roll domain secondary structures are described in Example 47 and FIG. 47. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the jelly-roll domain secondary structures shown in FIG. 47.

### Exemplary ORF and VP1 Sequences

In some embodiments, a polypeptide (e.g., an ORF1 or VP1 molecule) described herein comprises an amino acid sequence having at least about 70%, 75%, 80%, 85%, 90%, 95%, 96%, 97%, 98%, 99%, or 100% sequence identity to one or more Anellovirus ORF1 or CAV VP1 subsequences, e.g., as described herein). In some embodiments, an Anelloviridae family vector (e.g., anellovector) described herein comprises an ORF1 or VP1 molecule comprising an amino acid sequence having at least about 70%, 75%, 80%, 85%, 90%, 95%, 96%, 97%, 98%, 99%, or 100% sequence identity to one or more Anellovirus ORF1 or CAV VP1 subsequences, e.g., as described herein. In some embodiments, an anellovector described herein comprises a nucleic acid molecule (e.g., a genetic element) encoding an ORF1 or VP1 molecule comprising an amino acid sequence having at least about 70%, 75%, 80%, 85%, 90%, 95%, 96%, 97%, 98%, 99%, or 100% sequence identity to one or more Anellovirus ORF1 or CAV VP1 subsequences, e.g., as described herein.

In some embodiments, the one or more Anellovirus ORF1 or CAV VP1 subsequences comprises one or more of an arginine (Arg)-rich domain, a jelly-roll domain, a hypervariable region (HVR), an N22 domain, or a C-terminal domain (CTD) (e.g., as listed herein), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses. In some embodiments, the ORF1 or VP1 molecule comprises one or more of an Arg-rich domain, a jelly-roll domain, an N22 domain, and a CTD from one Anelloviridae family virus (e.g., Anellovirus), and an HVR from another. In some embodiments, the ORF1 or VP1 molecule comprises one or more of a jelly-roll domain, an HVR, an N22 domain, and a CTD from one Anelloviridae family virus (e.g., Anellovirus), and an Arg-rich domain from another. In some embodiments, the ORF1 or VP1 molecule comprises one or more of an Arg-rich domain, an HVR, an N22 domain, and a CTD from one Anelloviridae family virus (e.g., Anellovirus), and a jelly-roll domain from another. In some embodiments, the ORF1 or VP1 molecule comprises one or more of an Arg-rich domain, a jelly-roll domain, an HVR, and a CTD from one Anelloviridae family virus (e.g., Anellovirus), and an N22 domain from another. In some embodiments, the ORF1 or VP1 molecule comprises one or more of an Arg-rich domain, a jelly-roll domain, an HVR, and an N22 domain from one Anelloviridae family virus (e.g., Anellovirus), and a CTD from another.

Exemplary Anellovirus ORF1 amino acid sequences, and the sequences of exemplary ORF1 domains, are provided in the tables below. In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables P-Q). In some embodiments, an anellovector described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables P-Q. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables P-Q.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed in any of Tables P-Q), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses (e.g., any combination of ORF1 subsequences selected from the Alphatorquevirus Clade 1-7 subsequences listed in Tables P-Q). In embodiments, the ORF1 molecule comprises one or more of an Arg-rich domain, a jelly-roll domain, an N22 domain, and a CTD from one Anellovirus, and an HVR from another. In embodiments, the ORF1 molecule comprises one or more of a jelly-roll domain, an HVR, an N22 domain, and a CTD from one Anellovirus, and an Arg-rich domain from another. In embodiments, the ORF1 molecule comprises one or more of an Arg-rich domain, an HVR, an N22 domain, and a CTD from one Anellovirus, and a jelly-roll domain from another. In embodiments, the ORF1 molecule comprises one or more of an Arg-rich domain, a jelly-roll domain, an HVR, and a CTD from one Anellovirus, and an N22 domain from another. In embodiments, the ORF1 molecule comprises one or more of an Arg-rich domain, a jelly-roll domain, an HVR, and an N22 domain from one Anellovirus, and a CTD from another.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) as described in PCT Publication No. WO2020/123816 (incorporated herein by reference in entirety). In some embodiments, the one or more CAV VP1 subsequences comprises one or more of an arginine (Arg)-rich domain or a jelly-roll domain as described in PCT Application No. PCT/US2021/057292 (incorporated herein by reference in entirety).

TABLE P
Exemplary Anellovirus ORF1 amino acid subsequence (Betatorquevirus)

| | |
|---|---|
| Name | Ring2 |
| Genus/Clade | Betatorquevirus |
| Accession Number | JX134045.1 |
| Protein Accession Number | AGG91484.1 |

```
Full Sequence: 666 AA
1 10 20 30 40 50
  | | | | | |
  MPYYYRRRRYNYRRPRWYGRGWIRRPFRRRFRRKRRVRPTYTTIPLKQWQ
  PPYKRTCYIKGQDCLIYYSNLRLGMNSTMYEKSIVPVHWPGGGSFSVSML
  TLDALYDIHKLCRNWWTSTNQDLPLVRYKGCKITFYQSTFTDYIVRIHTE
  LPANSNKLTYPNTHPLMMMMSKYKHIIPSRQTRRKKKPYTKIFVKPPPQF
  ENKWYFATDLYKIPLLQIHCTACNLQNPFVKPDKLSNNVTLWSLNTISIQ
  NRNMSVDQGQSWPFKILGTQSFYFYFYTGANLPGDTTQIPVADLLPLTNP
  RINRPGQSLNEAKITDHITFTEYKNKFTNYWGNPFNKHIQEHLDMILYSL
  KSPEAIKNEWTTENMKWNQLNNAGTMALTPFNEPIFTQIQYNPDRDTGED
  TQLYLLSNATGTGWDPPGIPELILEGFPLWLIYWGFADFQKNLKKVTNID
  TNYMLVAKTKFTQKPGTFYLVILNDTFVEGNSPYEKQPLPEDNIKWYPQV
  QYQLEAQNKLLQTGPFTPNIQGQLSDNISMFYKFYFKWGGSPPKAINVEN
  PAHQIQYPIPRNEHETTSLQSPGEAPESILYSFDYRHGNYTTTALSRISQ
  DWALKDTVSKITEPDRQQLLKQALECLQISEETQEKKEKEVQQLISNLRQ
  QQQLYRERIISLLKDQ (SEQ ID NO: 215)
Annotations:
```

| Putative Domain | AA range |
|---|---|
| Arg-Rich Region | 1-38 |
| Jelly-roll domain | 39-246 |
| Hypervariable Region | 247-374 |
| N22 | 375-537 |
| C-terminal Domain | 538666 |

TABLE Q
Exemplary Anellovirus ORF1 amino acid subsequence (Betatorquevirus)
Ring2 ORF1 (Betatorquevirus)

| | |
|---|---|
| Arg-Rich | MPYYYRRRRYNYRRPRWYGRGWIRRPFRRRFRRKRRVR (SEQ ID NO: |
| Region | 216) |
| Jelly-roll | PTYTTIPLKQWQPPYKRTCYIKGQDCLIYYSNLRLGMNSTMYEKSIVPV |
| Domain | HWPGGGSFSVSMLTLDALYDIHKLCRNWWTSTNQDLPLVRYKGCKIT |
| | FYQSTFTDYIVRIHTELPANSNKLTYPNTHPLMMMMSKYKHIIPSRQTR |
| | RKKKPYTKIFVKPPPQFENKWYFATDLYKIPLLQIHCTACNLQNPFVKP |
| | DKLSNNVTLWSLNT (SEQ ID NO: 217) |
| Hypervariable | ISIQNRNMSVDQGQSWPFKILGTQSFYFYFYTGANLPGDTTQIPVADLL |
| domain | PLTNPRINRPGQSLNEAKITDHITFTEYKNKFTNYWGNPFNKHIQEHLD |
| | MILYSLKSPEAIKNEWTTENMKWNQLNNAG (SEQ ID NO: 218) |
| N22 | TMALTPFNEPIFTQIQYNPDRDTGEDTQLYLLSNATGTGWDPPGIPELIL |
| | EGFPLWLIYWGFADFQKNLKKVTNIDTNYMLVAKTKFTQKPGTFYLVI |
| | LNDTFVEGNSPYEKQPLPEDNIKWYPQVQYQLEAQNKLLQTGPFTPNI |
| | QGQLSDNISMFYKFYFK (SEQ ID NO: 219) |
| C-terminal | WGGSPPKAINVENPAHQIQYPIPRNEHETTSLOSPGEAPESILYSFDYRH |
| domain | GNYTTTALSRISQDWALKDTVSKITEPDRQQLLKQALECLQISEETQEK |
| | KEKEVQQLISNLRQQQQLYRERIISLLKDQ (SEQ ID NO: 220) |

### Consensus ORF1 Domain Sequences

In some embodiments, an ORF1 molecule, e.g., as described herein, comprises one or more of a jelly-roll domain, N22 domain, and/or C-terminal domain (CTD). In some embodiments, the jelly-roll domain comprises an amino acid sequence having a jelly-roll domain consensus sequence as described herein (e.g., as listed in any of Tables 37A-37C). In some embodiments, the N22 domain comprises an amino acid sequence having a N22 domain consensus sequence as described herein (e.g., as listed in any of Tables 37A-37C). In some embodiments, the CTD domain comprises an amino acid sequence having a CTD domain consensus sequence as described herein (e.g., as listed in any of Tables 37A-37C). In some embodiments, the amino acids listed in any of Tables 37A-37C in the format “(Xₐ-b)” comprise a contiguous series of amino acids, in which the series comprises at least a, and at most b, amino acids. In certain embodiments, all of the amino acids in the series are identical. In other embodiments, the series comprises at least two (e.g., at least 2, 3, 4, 5, 6, 7, 8, 9, 10, 11, 12, 13, 14, 15, 16, 17, 18, 19, 20, or 21) different amino acids.

TABLE 37A
Alphatorquevius ORF1 domain consensus sequences

| Domain | Sequence | SEQ ID NO: |
|---|---|---|
| Jelly-Roll | LVLTQWQPNTVRRCYIRGYLPLIICGEN(X₀₋₃)TTSRNYA | 227 |
| | THSDDTIQKGPFGGGMSTTTFSLRVLYDEYQRFMNRW | |
| | TYSNEDLDLARYLGCKFTFYRHPDXDFIVQYNTNPPFK | |
| | DTKLTAPSIHP(X₁₋₅)GMLMLSKRKILIPSLKTRPKGKHY | |
| | VKVRIGPPKLFEDKWYTQSDLCDVPLVXLYATAADLQ | |
| | HPFGSPQTDNPCVTFQVLGSXYNKHLSISP; | |
| | wherein X = any amino acid. | |
| N22 | SNFEFPGAYTDITYNPLTDKGVGNMVWIQYLTKPDTIX | 228 |
| | DKTQS(X₀₋₃)KCLIEDLPLWAALYGYVDFCEKETGDSAII | |
| | XNXGRVLIRCPYTKPPLYDKT(X₀₋₄)NKGFVPYSTNFGN | |
| | GKMPGGSGYVPIYWRARWYPTLFHQKEVLEDIVQSGP | |
| | FAYKDEKPSTQLVMKYCFNFN; | |
| | wherein X = any amino acid. | |
| CTD | WGGNPISQQVVRNPCKDSG(X₀₋₃)SGXGRQPRSVQVVD | 229 |
| | PKYMGPEYTFHSWDWRRGLFGEKAIKRMSEQPTDDEI | |
| | FTGGXPKRPRRDPPTXQXPEE(X₁₋₄)QKESSSFR(X₂₋₁₄)PW | |
| | ESSSQEXESESQEEEE(X₀₋₃₀)EQTVQQQLRQQLREQRRL | |
| | RVQLQLLFQQLLKT(X₀₋₄)QAGLHINPLLLSQA(X₀₋₄₀)*; | |
| | wherein X = any amino acid. | |

TABLE 37B
Betatorquevius ORF1 domain consensus sequences

| Domain | Sequence | SEQ ID NO: |
|---|---|---|
| Jelly-Roll | LKQWQPSTIRKCKIKGYLPLFQCGKGRISNNYTQYKESI | 230 |
| | VPHHEPGGGGWSIQQFTLGALYEEHLKLRNWWTKSN | |
| | DGLPLVRYLGCTIKLYRSEDTDYIVTYQRCYPMTATKL | |
| | TYLSTQPSRMLMNKHKIIVPSKXT(X₁₋₄)NKKKKPYKKIF | |
| | IKPPSQMQNKWYFQQDIANTPLLQLTXTACSLDRMYL | |
| | SSDSISNNITFTSLNTNFFQNPNFQ; | |
| | wherein X = any amino acid. | |
| N22 | (X₄₋₁₀)TPLYFECRYNPFKDKGTGNKVYLVSNN(X₁₋₈)TG | 231 |
| | WDPPTDPDLIIEGFPLWLLLWGWLDWQKKLGKIQNID | |
| | TDYILVIQSXYYIPP(X₁₋₃)KLPYYVPLDXD(X₀₋₂₎FLHGRS | |
| | PY(X₃₋₁₆)PSDKQHWHPKVRFQXETINNIALTGPGTPKLP | |
| | NQKSIQAHMKYKFYFK; | |
| | wherein X = any amino acid. | |
| CTD | WGGCPAPMETITDPCKQPKYPIPNNLLQTTSLQXPTTPI | 232 |
| | ETYLYKFDERRGLLTKKAAKRIKKDXTTETTLFTDTGX | |
| | XTSTTLPTXXQTETTQEEXTSEEE(X₀₋₅)ETLLQQLQQLR | |
| | RKQKQLRXRILQLLQLLXLL(_{X0-26}) *; | |
| | wherein X = any amino acid. | |

TABLE 37C
Gammatorquevius ORF1 domain consensus sequences

| Domain | Sequence | SEQ ID NO: |
|---|---|---|
| Jelly-Roll | TIPLKQWQPESIRKCKIKGYGTLVLGAEGRQFYCYTNE | 233 |
| | KDEYTPPKAPGGGGFGVELFSLEYLYEQWKARNNIWT | |
| | KSNXYKDLCRYTGCKITFYRHPTTDFIVXYSRQPPFEID | |
| | KXTYMXXHPQXLLLRKHKKIILSKATNPKGKLKKKIKI | |
| | KPPKQMLNKWFFQKQFAXYGLVQLQAAACBLRYPRL | |
| | GCCNENRLITLYYLN; | |
| | wherein X = any amino acid. | |
| N22 | LPIVVARYNPAXDTGKGNKXWLXSTLNGSXWAPPTTD | 234 |
| | KDLIIEGLPLWLALYGYWSYJKKVKKDKGILQSHMFV | |
| | VKSPAIQPLXTATTQXTFYPXIDNSFIQGKXPYDEPJTX | |
| | NQKKLWYPTLEHQQETINAIVESGPYVPKLDNQKNST | |
| | WELXYXYTFYFK; | |
| | wherein X = any amino acid. | |
| CTD | WGGPQIPDQPVEDPKXQGTYPVPDTXQQTIQIXNPLKQ | 235 |
| | KPETMFHDWDYRRGIITSTALKRMQENLETDSSFXSDS | |
| | EETP(X₀₋₂)KKKKRLTXELPXPQEETEEIQSCLLSLCEEST | |
| | CQEE(X₁₋₆)ENLQQLIHQQQQQQQQLKHNILKLLSDLKZ | |
| | KQRLLQLQTGILE(X₁₋₁₀)*; | |
| | wherein X = any amino acid. | |

In some embodiments, the jelly-roll domain comprises a jelly-roll domain amino acid sequence as listed in any of Tables 37A-37C, or an amino acid sequence having at least 70%, 75%, 80%, 8%, 90%, 95%, 96%, 97%, 98%, 99%, or 100% sequence identity thereto. In some embodiments, the N22 domain comprises a N22 domain amino acid sequence as listed in any of Tables 37A-37C, or an amino acid sequence having at least 70%, 75%, 80%, 8%, 90%, 95%, 96%, 97%, 98%, 99%, or 100% sequence identity thereto. In some embodiments, the CTD domain comprises a CTD domain amino acid sequence as listed in any of Tables 37A-37C, or an amino acid sequence having at least 70%, 75%, 80%, 8%, 90%, 95%, 96%, 97%, 98%, 99%, or 100% sequence identity thereto.

### Identification of ORF1 or VP1 Protein Sequences

In some embodiments, an ORF1 or VP1 protein sequence, or a nucleic acid sequence encoding an ORF1 or VP1 protein, can be identified from the genome of an Anelloviridae family virus, e.g., an Anellovirus (e.g., a putative Anelloviridae family virus genome identified, for example, by nucleic acid sequencing techniques, e.g., deep sequencing techniques). In some embodiments, an ORF1 or VP1 protein sequence is identified by one or more (e.g., 1, 2, or all 3) of the following selection criteria:

(i) Length Selection: Protein sequences (e.g., putative ORF1 or VP1 sequences passing the criteria described in (ii) or (iii) below) may be size-selected for those greater than about 600 amino acid residues to identify putative ORF1 or VP1 proteins. In some embodiments, an ORF1 or VP1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an ORF1 or VP1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.

(ii) Presence of ORF7 motif Protein sequences (e.g., putative ORF1 or VP1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN. In some embodiments, a putative Anellovirus ORF1 sequence comprises the sequence Y[NCS]PXXDX[GASKR]XX[NTSVAK].

(iii) Presence of arginine-rich region: Protein sequences (e.g., putative ORF1 or VP1 sequences passing the criteria described in (i) and/or (ii) above) may be filtered for those that include an arginine-rich region (e.g., as described herein). In some embodiments, a putative ORF1 or VP1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues.

In some embodiments, a putative ORF1 or VP1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative ORF1 or VP1 protein. In some embodiments, the arginine-rich region is positioned at least about 50 amino acids downstream of the start codon of the putative ORF1 or VP1 protein.

In some embodiments, an ORF1 protein is identified in an Anellovirus genome sequence as described in Example 36 of PCT Publication No. WO2020/123816 (incorporated herein by reference in its entirety).

### ORF2 or VP2 Molecules

In some embodiments, the anellovector comprises an ORF2 or VP2 molecule and/or a nucleic acid encoding an ORF2 or VP2 molecule. Generally, an ORF2 or VP2 molecule comprises a polypeptide having the structural features and/or activity of an Anellovirus ORF2 protein (e.g., an Anellovirus ORF2 protein as described herein, e.g., as listed in Table A1 or A2) or a CAV VP2 protein (e.g. a CAV VP2 protein as described herein, e.g., as listed in Table A3), or a functional fragment thereof. In some embodiments, an ORF2 or VP2 molecule comprises an amino acid sequence having at least 70%, 75%, 80%, 85%, 90%, 95%, 96%, 97%, 98%, 99%, or 100% sequence identity to an Anellovirus ORF2 protein or a CAV protein sequence as shown in Table A1-A3. In some embodiments, an ORF2 molecule is encoded by an ORF2 nucleic acid. In some embodiments, the ORF2 nucleic acid comprises an antisense strand, which can be directly transcribed to produce mRNA encoding the ORF2 molecule. In some embodiments, the ORF2 nucleic acid comprises a sense strand.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 or VP2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 or VP2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 or VP2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 or VP2 molecule has protein phosphatase activity. In some embodiments, an ORF2 or VP2 molecule comprises at least one difference (e.g., a mutation, chemical modification, or epigenetic alteration) relative to a wild-type ORF2 or CAV protein, e.g., as described herein (e.g., as shown in Table A1-A3).

### Conserved ORF2 Motif

In some embodiments, a polypeptide (e.g., an ORF2 molecule) described herein comprises the amino acid sequence [W/F]X⁷HX³CX¹CX⁵H (SEQ ID NO: 949), wherein Xⁿ is a contiguous sequence of any n amino acids. In embodiments, X⁷ indicates a contiguous sequence of any seven amino acids. In some embodiments, X³ indicates a contiguous sequence of any three amino acids. In some embodiments, X¹ indicates any single amino acid. In some embodiments, X⁵ indicates a contiguous sequence of any five amino acids. In some embodiments, the [W/F] can be either tryptophan or phenylalanine. In some embodiments, the [W/F]X⁷HX³CX¹CX⁵H (SEQ ID NO: 949) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein Xⁿ is a contiguous sequence of any n amino acids.

### Genetic Elements

In some embodiments, the Anelloviridae family vector (e.g., anellovector) comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus or CAV nucleic acid sequence, e.g., has less than 70%, 60%, 50%, 40%, 30%, 20%, 10%, 5% sequence identity to an Anellovirus or CAV nucleic acid sequence, e.g., as described herein. In some embodiments, outside of the packaging or capsid-binding sequence, the genetic element has less than 500, 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus or CAV nucleic acid sequence. In certain embodiments, the genetic element is a circular, single stranded DNA that comprises a promoter sequence, a sequence encoding a therapeutic effector, and a capsid binding protein.

In some embodiments, the genetic element has at least about 70%, 75%, 80%, 8%, 90%, 95%, 96%, 97%, 98%, 99%, or 100% sequence identity to an Anellovirus or CAV nucleic acid sequence, e.g., as described herein (e.g., as described in any of Tables N1-N4), or a fragment thereof, or encodes an amino acid sequence having at least about 70%, 75%, 80%, 8%, 90%, 95%, 96%, 97%, 98%, 99%, or 100% sequence identity to an Anellovirus or CAV amino acid sequence (e.g., as described in any of Tables A1-A3), or a fragment thereof. In some embodiments, the genetic element comprises a sequence encoding an effector (e.g., an endogenous effector or an exogenous effector, e.g., a payload), e.g., a polypeptide effector (e.g., a protein) or nucleic acid effector (e.g., a non-coding RNA, e.g., a miRNA, siRNA, mRNA, lncRNA, RNA, DNA, an antisense RNA, gRNA).

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences described herein, an Anellovirus or CAV amino acid sequence, e.g., as listed in any of Tables A1-A3.

In some embodiments, the genetic element was produced from a double-stranded circular DNA (e.g., produced by in vitro circularization). In some embodiments, the genetic element was produced by rolling circle replication from the double-stranded circular DNA. In some embodiments, the rolling circle replication occurs in a cell (e.g., a host cell, e.g., a mammalian cell, e.g., a human cell, e.g., a HEK293T cell, an A549 cell, or a Jurkat cell). In some embodiments, the genetic element can be amplified exponentially by rolling circle replication in the cell. In some embodiments, the genetic element can be amplified linearly by rolling circle replication in the cell. In some embodiments, the double-stranded circular DNA or genetic element is capable of yielding at least 2, 4, 8, 16, 32, 64, 128, 256, 518, 1024 or more times the original quantity by rolling circle replication in the cell. In some embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus or CAV sequence, e.g., as described herein).

In some embodiments, a genetic element as described herein comprises a sequence (e.g., a TATA box, cap site, transcriptional start site, 5′ UTR, open reading frame (ORF), poly(A) signal, or GC-rich region sequence) as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, a genetic element comprises a sequence encoding an effector (e.g., an exogenous effector). In some embodiments, the effector-encoding sequence is inserted into an Anellovirus or CAV genome sequence (e.g., as described herein). In some embodiments, the effector-encoding sequence replaces a contiguous sequence (e.g., of at least 5, 10, 15, 20, 25, 30, 40, 50, 60, 70, 80, 90, 100, 200, 300, 400, 500, 600, 700, 800, 900, 1000, or more nucleotides) from the Anellovirus or CAV genome sequence. In some embodiments, the effector-encoding sequence replaces a TATA box, cap site, transcriptional start site, 5′ UTR, open reading frame (ORF), poly(A) signal, or GC-rich region sequence, or a portion thereof (e.g., a portion consisting of at least 5, 10, 15, 20, 25, 30, 40, 50, 60, 70, 80, 90, 100, 200, 300, 400, 500, 600, 700, 800, 900, 1000, or more nucleotides) e.g., as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, the sequence of a first nucleic acid element comprised in a genetic element (e.g., a TATA box, cap site, transcriptional start site, 5′ UTR, open reading frame (ORF), poly(A) signal, or GC-rich region) overlaps with the sequence of a second nucleic acid element (e.g., a TATA box, cap site, transcriptional start site, 5′ UTR, open reading frame (ORF), poly(A) signal, or GC-rich region), e.g., by at least 5, 10, 15, 20, 25, 30, 40, 50, 60, 70, 80, 90, 100, 150, 200, 250, 300, 400, or 500 nucleotides. In some embodiments, the sequence of a first nucleic acid element comprised in a genetic element (e.g., a TATA box, cap site, transcriptional start site, 5′ UTR, open reading frame (ORF), poly(A) signal, or GC-rich region) does not overlap with the sequence of a second nucleic acid element (e.g., a TATA box, cap site, transcriptional start site, 5′ UTR, open reading frame (ORF), poly(A) signal, or GC-rich region).

### Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8. In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus or CAV sequence (e.g., as shown in any of Tables N1-N4).

In embodiments, the protein binding sequence has at least about 70%, 75%, 80%, 85%, 90%, 95%, 96%, 97%, 98%, 99%, or 100% sequence identity to the Anellovirus or CAV 5′ UTR conserved domain nucleotide sequence of any of Tables N1-N4.

### 5′ UTR Regions

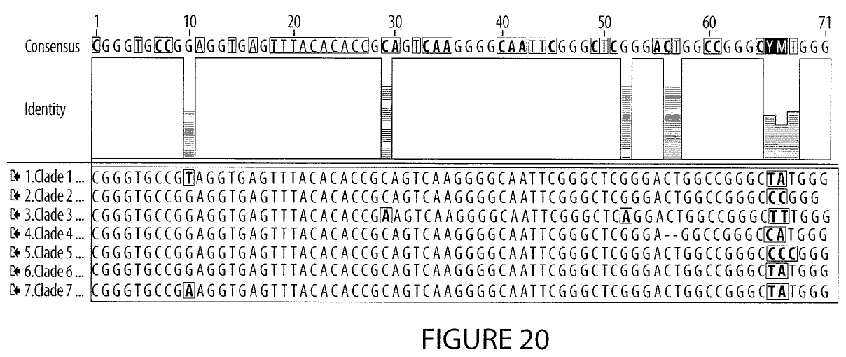

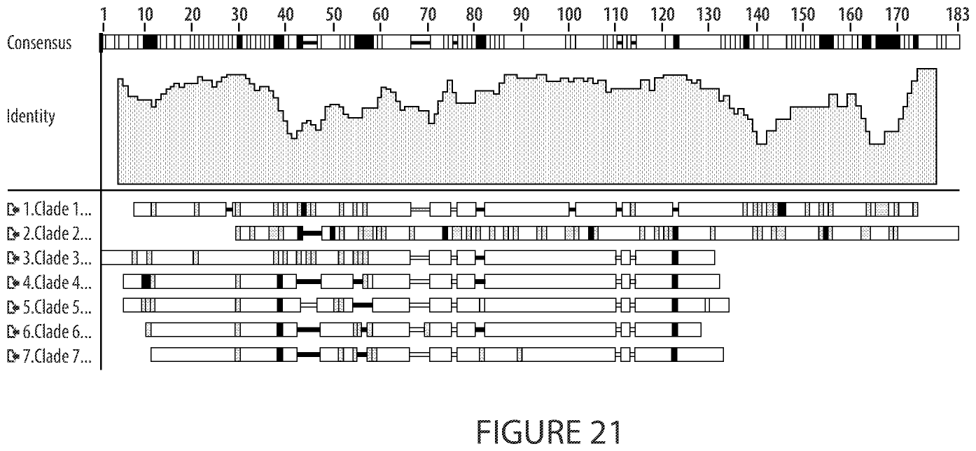

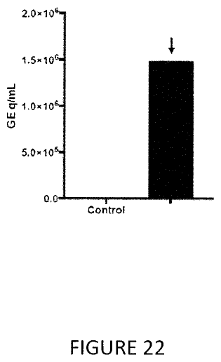

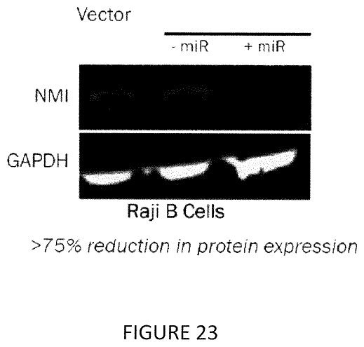

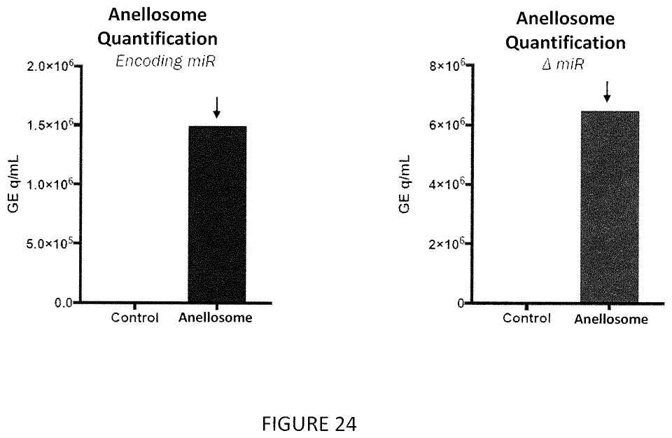

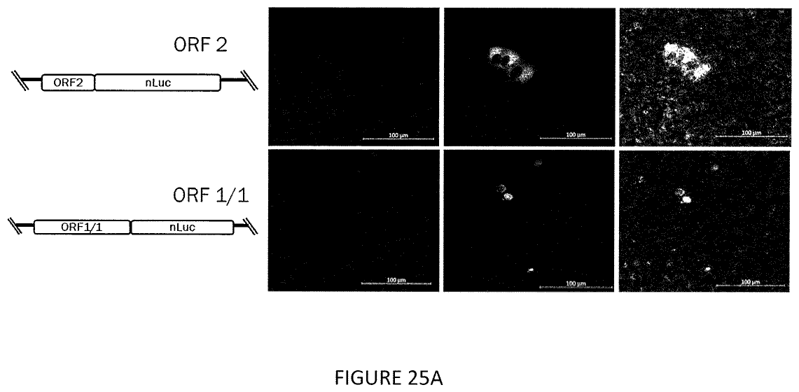

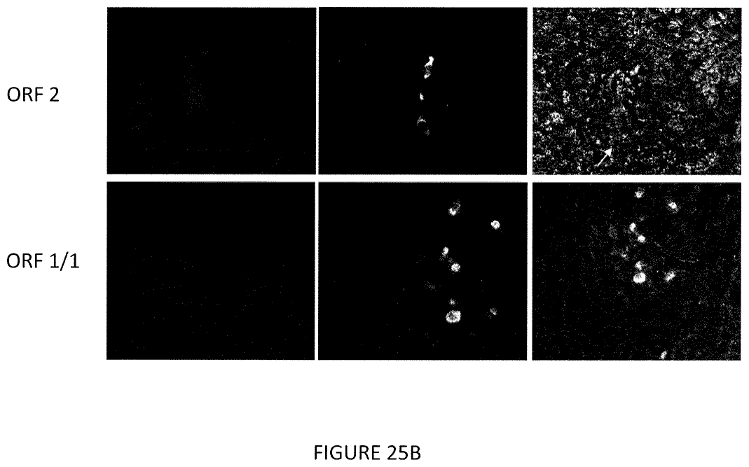

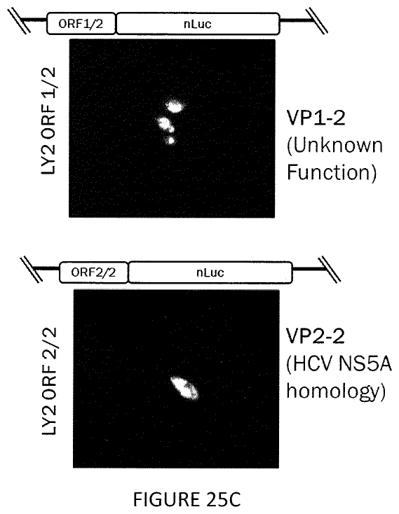

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 38 and/or FIG. 20. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and X₅ are each independently any nucleotide, e.g., wherein X₁=G or T, X₂=C or A, X₃=G or A, X₄=T or C, and X₅=A, C, or T). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJN02 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

In some embodiments, the genetic element comprises a nucleic acid sequence having at least about 70%, 75%, 80%, 85%, 90%, 95%, 96%, 97%, 98%, 99%, or 100% o sequence identity to the Anellovirus or CAV 5′ UTR conserved domain nucleotide sequence of any of Tables N1-N4.

TABLE 38
Exemplary 5′ UTR sequences from Anelloviruses

| Source | Sequence | SEQ ID NO: |
|---|---|---|
| Consensus | CGGGTGCCGX₁AGGTGAGTTTACACACCGX₂AGT | 105 |
| | CAAGGGGCAATTCGGGCTCX₃GGACTGGCCGGG | |
| | CX₄X₅TGGG | |
| | X₁ = G or T | |
| | X₂ = C or A | |
| | X₃ = Gor A | |
| | X₄ = T or C | |
| | X₅ = A, C, or T | |
| Exemplary TTV | CGGGTGCCGGAGGTGAGTTTACACACCGCAGTC | 106 |
| Sequence | AAGGGGCAATTCGGGCTCGGGACTGGCCGGGCT | |
| | WTGGG | |
| TTV-CT30F | CGGGTGCCGTAGGTGAGTTTACACACCGCAGTC | 107 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 108 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 109 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 110 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | 111 |
| | AAGGGGCAATTCGGGCTCAGGACTGGCCGGGCT | |
| | TTGGG | |
| Alphatorquevirus | CGGGTGCCGGAGGTGAGTTTACACACCGCAGTC | 112 |
| Consensus 5′ UTR | AAGGGGCAATTCGGGCTCGGGACTGGCCGGGC | |
| | X₁X₂TGGG; wherein X₁ comprises T or C, and | |
| | wherein X₂ comprises A, C, or T. | |
| Alphatorquevirus | CGGGTGCCGTAGGTGAGTTTACACACCGCAGTC | 113 |
| Clade 1 5′ UTR (e.g., | AAGGGGCAATTCGGGCTCGGGACTGGCCGGGCT | |
| TTV-CT30F) | ATGGG | |
| Alphatorquevirus | CGGGTGCCGGAGGTGAGTTTACACACCGCAGTC | 114 |
| Clade 2 5′ UTR (e.g., | AAGGGGCAATTCGGGCTCGGGACTGGCCGGGCC | |
| TTV-P13-1) | CGGG | |
| Alphatorquevirus | CGGGTGCCGGAGGTGAGTTTACACACCGAAGTC | 115 |
| Clade 3 5′ UTR (e.g., | AAGGGGCAATTCGGGCTCAGGACTGGCCGGGCT | |
| TTV-tth8) | TTGGG | |
| Alphatorquevirus | CGGGTGCCGGAGGTGAGTTTACACACCGCAGTC | 116 |
| Clade 4 5′ UTR (e.g., | AAGGGGCAATTCGGGCTCGGGAGGCCGGGCCAT | |
| TTV-HD20a) | GGG | |
| Alphatorquevirus | CGGGTGCCGGAGGTGAGTTTACACACCGCAGTC | 117 |
| Clade 5 5′ UTR (e.g., | AAGGGGCAATTCGGGCTCGGGACTGGCCGGGCC | |
| TTV-16) | CCGGG | |
| Alphatorquevirus | CGGGTGCCGGAGGTGAGTTTACACACCGCAGTC | 118 |
| Clade 6 5′ UTR (e.g., | AAGGGGCAATTCGGGCTCGGGACTGGCCGGGCT | |
| TTV-TJN02) | ATGGG | |
| Alphatorquevirus | CGGGTGCCGAAGGTGAGTTTACACACCGCAGTC | 119 |
| Clade 7 5′ UTR (e.g., | AAGGGGCAATTCGGGCTCGGGACTGGCCGGGCT | |
| TTV-HD16d) | ATGGG | |

### Identification of 5′ UTR Sequences

In some embodiments, an Anelloviridae family virus (e.g., Anellovirus or CAV) 5′ UTR sequence can be identified within the genome of an Anelloviridae family virus (e.g., Anellovirus or CAV) (e.g., a putative Anelloviridae family virus genome identified, for example, by nucleic acid sequencing techniques, e.g., deep sequencing techniques). In some embodiments, an Anelloviridae family virus (e.g., Anellovirus or CAV) 5′ UTR sequence is identified by one or both of the following steps:

(i) Identification of circularization junction point: In some embodiments, a 5′ UTR will be positioned near a circularization junction point of a full-length, circularized Anelloviridae family virus (e.g., Anellovirus or CAV) genome. A circularization junction point can be identified, for example, by identifying overlapping regions of the sequence. In some embodiments, an overlapping region of the sequence can be trimmed from the sequence to produce a full-length Anelloviridae family virus (e.g., Anellovirus or CAV) genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anelloviridae family virus (e.g., Anellovirus or CAV) genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anelloviridae family virus (e.g., Anellovirus or CAV), e.g., as described herein).

(ii) Identification of 5′ UTR sequence: Once a putative Anelloviridae family virus (e.g., Anellovirus or CAV) genome sequence has been obtained, the sequence (or portions thereof, e.g., having a length between about 40-50, 50-60, 60-70, 70-80, 80-90, or 90-100 nucleotides) can be compared to one or more Anelloviridae family virus (e.g., Anellovirus or CAV) 5′ UTR sequences (e.g., as described herein) to identify sequences having substantial homology thereto. In some embodiments, a putative Anelloviridae family virus (e.g., Anellovirus or CAV) 5′ UTR region has at least 50%, 60%, 70%, 75%, 80%, 85%, 90%, 95%, 96%, 97%, 98%, 99%, or 100% sequence identity to an Anelloviridae family virus (e.g., Anellovirus or CAV) 5′ UTR sequence as described herein.

### GC-Rich Regions

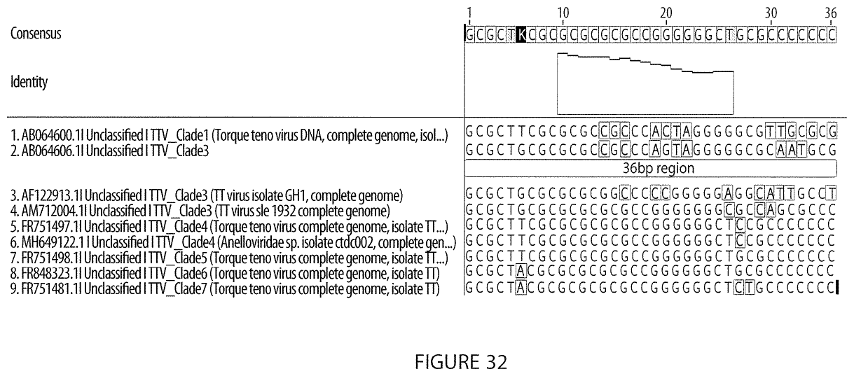

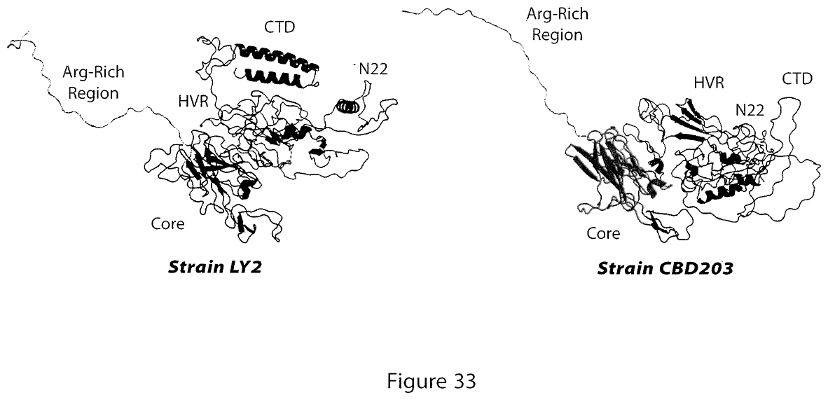

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in any of Table 39 and/or FIGS. 20 and 32. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a GC-rich sequence shown in Table 39.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In some embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39. In some embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 105, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJN02, or TTV-HD16d, e.g., as listed in Table 39.

In some embodiments, the 36-nucleotide GC-rich sequence is selected from:

(i)
(SEQ ID NO: 160)
CGCGCTGCGCGCGCCGCCCAGTAGGGGGAGCCATGC,
(ii)
(SEQ ID NO: 164)
GCGCTX1CGCGCGCGCGCCGGGGGGCTGCGCCCCCCC,

wherein X₁ is selected from T, G, or A;

(iii)
(SEQ ID NO: 165)
GCGCTTCGCGCGCCGCCCACTAGGGGGCGTTGCGCG;
(iv)
(SEQ ID NO: 166)
GCGCTGCGCGCGCCGCCCAGTAGGGGGCGCAATGCG;
(v)
(SEQ ID NO: 167)
GCGCTGCGCGCGCGGCCCCCGGGGGAGGCATTGCCT;
(vi)
(SEQ ID NO: 168)
GCGCTGCGCGCGCGCGCCGGGGGGGCGCCAGCGCCC;
(vii)
(SEQ ID NO: 169)
GCGCTTCGCGCGCGCGCCGGGGGGCTCCGCCCCCCC;
(viii)
(SEQ ID NO: 170)
GCGCTTCGCGCGCGCGCCGGGGGGCTGCGCCCCCCC;
(ix)
(SEQ ID NO: 171)
GCGCTACGCGCGCGCGCCGGGGGGCTGCGCCCCCCC;
or
(x)
(SEQ ID NO: 172)
GCGCTACGCGCGCGCGCCGGGGGGCTCTGCCCCCCC.

In some embodiments, the genetic element (e.g., protein-binding sequence of the genetic element) comprises the nucleic acid sequence CGCGCTGCGCGCGCCGCCCAGTAGGGGGAGCCATGC (SEQ ID NO: 160).

In some embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₈, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJN02 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

TABLE 39
Exemplary GC-rich sequences from Anelloviruses

| | | | SEQ ID |
|---|---|---|---|
| Source | | Sequence | NO: |
| Consensus | | CGGCGGX₁GGX₂GX₃X₄X₅CGCGCTX₆CGCGC | 120 |
| | | GCX₇X₈X₉X₁₀CX₁₁X₁₂X₁₃X₁₄GGGGX₁₅X₁₆X₁₇X₁₈ | |
| | | X₁₉X₂₀X₂₁GCX₂₂X₂₃X₂₄X₂₅CCCCCCCX₂₆CGCGC | |
| | | ATX₂₇X₂₈GCX₂₉CGGGX₃₀CCCCCCCCCX₃₁X₃₂X | |
| | | ₃₃GGGGGGCTCCGX₃₄CCCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G or A | |
| | | X₃₀ = G or T | |
| | | X₃₁ = C, T, or absent | |
| | | X₃₂ = G, C, A, or absent | |
| | | X₃₃ = G or C | |
| | | X₃₄ = C or absent | |
| Exemplary TTV | Full sequence | GCCGCCGCGGCGGCGGSGGNGNSGCGCGCT | 121 |
| Sequence | | DCGCGCGCSNNNCRCCRGGGGGNNNNCWG | |
| | | CSNCNCCCCCCCCCGCGCATGCGCGGGKCC | |
| | | CCCCCCCNNCGGGGGGCTCCGCCCCCCGGC | |
| | | CCCCCCCCGTGCTAAACCCACCGCGCATGC | |
| | | GCGACCACGCCCCCGCCGCC | |
| | Fragment 1 | GCCGCCGCGGCGGCGGSGGNGNSGCGCGCT | 122 |
| | | DCGCGCGCSNNNCRCCRGGGGGNNNNCWG | |
| | | CSNCNCCCCCCCCCGCGCAT | |
| | Fragment 2 | GCGCGGGKCCCCCCCCCNNCGGGGGGCTC | 123 |
| | | CG | |
| | Fragment 3 | CCCCCCGGCCCCCCCCCGTGCTAAACCCAC | 124 |
| | | CGCGCATGCGCGACCACGCCCCCGCCGCC | |
| TTV-CT30F | Full sequence | GCGGCGG-GGGGGCG-GCCGCG- | 125 |
| | | TTCGCGCGCCGCCCACCAGGGGGTG-- | |
| | | CTGCG-CGCCCCCCCCCGCGCAT | |
| | | GCGCGGGGCCCCCCCCC- | |
| | | GGGGGGGCTCCGCCCCCCCGGCCCCCCCCC | |
| | | GTGCTAAACCCACCGCGCATGCGCGACCAC | |
| | | GCCCCCGCCGCC | |
| | Fragment 1 | GCGGCGG | 126 |
| | Fragment 2 | GGGGGCG | 127 |
| | Fragment 3 | GCCGCG | 128 |
| | Fragment 4 | TTCGCGCGCCGCCCACCAGGGGGTG | 129 |
| | Fragment 5 | CTGCG | 130 |
| | Fragment 6 | CGCCCCCCCCCGCGCAT | 131 |
| | Fragment 7 | GCGCGGGGCCCCCCCCC | 132 |
| | Fragment 8 | GGGGGGGCTCCGCCCCCCCGGCCCCCCCCC | 133 |
| | | GTGCTAAACCCACCGCGCATGCGCGACCAC | |
| | | GCCCCCGCCGCC | |
| TTV-HD23a | Full sequence | CGGCGGCGGCGGCG- | 134 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGCTCC | |
| | | G CCCCCCGGCCCCCCCCC | |
| | Fragment 1 | CGGCGGCGGCGGCG | 135 |
| | Fragment 2 | CGCGCGCTGCGCGCGCG | 136 |
| | Fragment 3 | CGCCGGGGGGGGCCAGCG | 137 |
| | Fragment 4 | CCCCCCCCCCCGCGCAT | 138 |
| | Fragment 5 | GCACGGGTCCCCCCCCCCACGGGGGGCTCC | 139 |
| | | G | |
| | Fragment 6 | CCCCCCGGCCCCCCCCC | 140 |
| TTV-JA20 | Full sequence | CCGTCGGGGGGGGGCCGCGCGCTGCGCG | 141 |
| | | CGCGGCCC- | |
| | | CCGGGGGAGGCACAGCCTCCCCCCCCCGCG | |
| | | CGCATGCGCGCGGGTCCCCCCCCCTCCGGG | |
| | | GGGCTCCGCCCCCCGGCCCCCCCC | |
| | Fragment 1 | CCGTCGGCGGGGGGGCCGCGCGCTGCGCG | 142 |
| | | CGCGGCCC | |
| | Fragment 2 | CCGGGGGAGGCACAGCCTCCCCCCCCCGCG | 143 |
| | | CGCATGCGCGCGGGTCCCCCCCCCTCCGGG | |
| | | GGGCTCCGCCCCCCGGCCCCCCCC | |
| TTV-TJN02 | Full sequence | CGGCGGCGGCG-CGCGCGCTACGCGCGCG-- | 144 |
| | | -CGCCGGGGGG----CTGCCGC- | |
| | | CCCCCCCCCGCGCAT | |
| | | GCGCGGGGCCCCCCCCC- | |
| | | GCGGGGGGCTCCG CCCCCCGGCCCCCC | |
| | Fragment 1 | CGGCGGCGGCG | 145 |
| | Fragment 2 | CGCGCGCTACGCGCGCG | 146 |
| | Fragment 3 | CGCCGGGGGG | 147 |
| | Fragment 4 | CTGCCGC | 148 |
| | Fragment 5 | CCCCCCCCCGCGCAT | 149 |
| | Fragment 6 | GCGCGGGGCCCCCCCCC | 150 |
| | Fragment 7 | GCGGGGGGCTCCG | 151 |
| | Fragment 8 | CCCCCCGGCCCCCC | 152 |
| TTV-tth8 | Full sequence | GCCGCCGCGGCGGCGGGGG- | 153 |
| | | GCGGCGCGCTGCGCGCGCCGCCCAGTAGG | |
| | | GGGAGCCATGCG--CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 154 |
| | Fragment 2 | GCGGCGCGCTGCGCGCGCCGCCCAGTAGG | 155 |
| | | GGGAGCCATGCG | |
| | Fragment 3 | CCCCCCCCCGCGCAT | 156 |
| | Fragment 4 | GCGCGGGGCCCCCCCCC | 157 |
| | Fragment 5 | GCGGGGGGCTCCG | 158 |
| | Fragment 6 | CCCCCCGGCCCCCCCCG | 159 |
| | Fragment 7 | CGCGCTGCGCGCGCCGCCCAGTAGGGGGA | 160 |
| | | GCCATGC | |
| | Fragment 8 | CCGCCATCTTAAGTAGTTGAGGCGGACGGT | 161 |
| | | GGCGTGAGTTCAAAGGTCACCATCAGCCAC | |
| | | ACCTACTCAAAATGGTGG | |
| | Fragment 9 | CTTAAGTAGTTGAGGCGGACGGTGGCGTGA | 162 |
| | | GTTCAAAGGTCACCATCAGCCACACCTACT | |
| | | CAAAATGGTGGACAATTTCTTCCGGGTCAA | |
| | | AGGTTACAGCCGCCATGTTAAAACACGTGA | |
| | | CGTATGACGTCACGGCCGCCATTTTGTGAC | |
| | | ACAAGATGGCCGACTTCCTTCC | |
| Additional | 36-nucleotide | CGCGCTGCGCGCGCCGCCCAGTAGGGGGA | 163 |
| GC-rich | consensus GC- | | |
| Sequences (as | rich region | GCCATGC | |
| shown | sequence 1 | | |
| in FIG. 32) | 36-nucleotide | GCGCTX₁CGCGCGCGCGCCGGGGGGCTGCG | 164 |
| | region | CCCCCCC, wherein X₁ is selected | |
| | consensus | from T, G, or A | |
| | sequence 2 | | |
| | TTV Clade 1 | GCGCTTCGCGCGCCGCCCACTAGGGGGCGT | 165 |
| | 36-nucleotide | TGCGCG | |
| | region | | |
| | TTV Clade 3 | GCGCTGCGCGCGCCGCCCAGTAGGGGGCG | 166 |
| | 36-nucleotide | CAATGCG | |
| | region | | |
| | TTV Clade 3 | GCGCTGCGCGCGCGGCCCCCGGGGGAGGC | 167 |
| | isolate GH1 36- | ATTGCCT | |
| | nucleotide | | |
| | region | | |
| | TTV Clade 3 | GCGCTGCGCGCGCGCGCCGGGGGGGCGCC | 168 |
| | sle 1932 36- | AGCGCCC | |
| | nucleotide | | |
| | region | | |
| | TTV Clade 4 | GCGCTTCGCGCGCGCGCCGGGGGGCTCCGC | 169 |
| | ctdc002 36- | CCCCCC | |
| | nucleotide | | |
| | region | | |
| | TTV Clade 5 | GCGCTTCGCGCGCGCGCCGGGGGGCTGCGC | 170 |
| | 36-nucleotide | CCCCCC | |
| | region | | |
| | TTV Clade 6 | GCGCTACGCGCGCGCGCCGGGGGGCTGCG | 171 |
| | 36-nucleotide | CCCCCCC | |
| | region | | |
| | TTV Clade 7 | GCGCTACGCGCGCGCGCCGGGGGGCTCTGC | 172 |
| | 36-nucleotide | CCCCCC | |
| | region | | |
| Additional | TTV-CT30F | GCGGCGGGGGGGCGGCCGCGTTCGCGCGC | 801 |
| Alphatorquevirus | | CGCCCACCAGGGGGTGCTGCGCGCCCCCCC | |
| GC-rich region | | CCGCGCATGCGCGGGGCCCCCCCCCGGGG | |
| sequences | | GGGCTCCGCCCCCCCGGCCCCCCCCCGTGC | |
| | | TAAACCCACCGCGCATGCGCGACCACGCCC | |
| | | CCGCCGCC | |
| | TTV-P13-1 | CCGAGCGTTAGCGAGGAGTGCGACCCTACC | 802 |
| | | CCCTGGGCCCACTTCTTCGGAGCCGCGCGC | |
| | | TACGCCTTCGGCTGCGCGCGGCACCTCAGA | |
| | | CCCCCGCTCGTGCTGACACGCTTGCGCGTG | |
| | | TCAGACCACTTCGGGCTCGCGGGGGTCGGG | |
| | TTV-tth8 | GCCGCCGCGGCGGCGGGGGGCGGCGCGCT | 803 |
| | | GCGCGCGCCGCCCAGTAGGGGGAGCCATG | |
| | | CGCCCCCCCCCGCGCATGCGCGGGGCCCCC | |
| | | CCCCGCGGGGGGCTCCGCCCCCCGGCCCCC | |
| | | CCCG | |
| | TTV-HD20a | CGGCCCAGCGGCGGCGCGCGCGCTTCGCGC | 804 |
| | | GCGCGCCGGGGGGCTCCGCCCCCCCCCGCG | |
| | | CATGCGCGGGGCCCCCCCCCGCGGGGGGCT | |
| | | CCGCCCCCCGGTCCCCCCCCG | |
| | TTV-16 | CGGCCGTGCGGCGGCGCGCGCGCTTCGCGC | 805 |
| | | GCGCGCCGGGGGCTGCCGCCCCCCCCCGCG | |
| | | CATGCGCGCGGGGCCCCCCCCCGCGGGGG | |
| | | GCTCCGCCCCCCGGCCCCCCCCCCCG | |
| | TTV-TJN02 | CGGCGGCGGCGCGCGCGCTACGCGCGCGC | 806 |
| | | GCCGGGGGGCTGCCGCCCCCCCCCCGCGCA | |
| | | TGCGCGGGGCCCCCCCCCGCGGGGGGCTCC | |
| | | GCCCCCCGGCCCCCC | |
| | TTV-HD16d | GGCGGCGGCGCGCGCGCTACGCGCGCGCG | 807 |
| | | CCGGGGAGCTCTGCCCCCCCCCGCGCATGC | |
| | | GCGCGGGTCCCCCCCCCGCGGGGGGCTCCG | |
| | | CCCCCCGGTCCCCCCCCCG | |

In some embodiments, the genetic element comprises a nucleic acid sequence having at least about 70%, 75%, 80%, 85%, 90%, 95%, 96%, 97%, 98%, 99%, or 100% sequence identity to the Anelloviridae family virus (e.g., Anellovirus or CAV) GC-rich nucleotide sequence of any of Tables N1-N4.

### Effector

In some embodiments, the genetic element may include one or more sequences that encode a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described in Example 10, 12, or 22. In some embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the sequence encoding an effector is inserted into the genetic element at about nucleotide 3588 of a TTV-tth8 plasmid, e.g., as described herein or at about nucleotide 2843 of a TTMV-LY2 plasmid, e.g., as described herein. In some embodiments, the sequence encoding an effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an effector replaces part or all of an open reading frame (e.g., an ORF or VP1 as described herein, e.g., an ORF1, ORF1/1, ORF1/2, ORF2, ORF2/2, ORF2/3, and/or ORF2t/3 as shown in Table A1-A3 or N1-N4).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload, e.g., as described in Example 11.

### Regulatory Nucleic Acid

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In some embodiments, the regulatory nucleic acid encodes an miRNA.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the regulatory nucleic acid is at least one miRNA, e.g., 2, 3, 4, 5, 6, or more. In some embodiments, the genetic element comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a sequence described herein.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl: S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the Anelloviridae family vector (e.g., anellovector) may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

### Exemplary Intracellular Polypeptide Effectors

In some embodiments, the effector comprises a cytosolic polypeptide or cytosolic peptide. In some embodiments, the effector comprises cytosolic peptide is a DPP-4 inhibitor, an activator of GLP-1 signaling, or an inhibitor of neutrophil elastase. In some embodiments, the effector increases the level or activity of a growth factor or receptor thereof (e.g., an FGF receptor, e.g., FGFR3). In some embodiments, the effector comprises an inhibitor of n-myc interacting protein activity (e.g., an n-myc interacting protein inhibitor); an inhibitor of EGFR activity (e.g., an EGFR inhibitor); an inhibitor of IDH1 and/or IDH2 activity (e.g., an IDH1 inhibitor and/or an IDH2 inhibitor); an inhibitor of LRP5 and/or DKK2 activity (e.g., an LRP5 and/or DKK2 inhibitor); an inhibitor of KRAS activity; an activator of HTT activity; or inhibitor of DPP-4 activity (e.g., a DPP-4 inhibitor).

In some embodiments, the effector comprises a regulatory intracellular polyeptpide. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

In some embodiments, the effector is an anti-apoptotic agent. In some embodiments, the effector reduces apoptosis of a cell with which the Anelloviridae family vector is contacted, e.g., a cancer cell, e.g., by reducing caspase-3 activity, e.g., by at least about 10%, 20%, 30%, 40%, 50%, 60%, 70%, 80%, 90%, 95%, 99%, or more. In some embodiments, the effector reduces apoptosis of a cell with which the Anelloviridae family vector is contacted, e.g., a cancer cell, e.g., by reducing caspase-3 activity, e.g., by at least about 10%, 20%, 30%, 40%, 50%, 60%, 70%, 80%, 90%, 95%, 99%, or more. In certain embodiments, the effector is an miRNA, e.g., miR-625.

### Exemplary Secreted Polypeptide Effectors

Exemplary secreted therapeutics are described herein, e.g., in the tables below.

TABLE 50
Exemplary cytokines and cytokine receptors

| Cytokine | Cytokine receptor(s) | Entrez Gene ID | UniProt ID |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| IL-17E | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and IL-22R1/ | 50604 | Q9NYY1 |
| | IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and IL- | 11009 | Q13007 |
| | 22R1/IL-20R2 | | |
| IL-25 | IL-17RA and IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBI3, or | IL-12Rβ2/gp130; IL- | 10148 | Q14213 |
| a heterodimer thereof) | 12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

In some embodiments, an effector described herein comprises a cytokine of Table 50, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 50 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 50 or a functional variant or fragment thereof) and a second, heterologous region. In some embodiments, the first region is a first cytokine polypeptide of Table 50. In some embodiments, the second region is a second cytokine polypeptide of Table 50, wherein the first and second cytokine polypeptides form a cytokine heterodimer with each other in a wild-type cell. In some embodiments, the polypeptide of Table 50 or functional variant thereof comprises a signal sequence, e.g., a signal sequence that is endogenous to the effector, or a heterologous signal sequence. In some embodiments, an Anelloviridae family vector (e.g., anellovector) encoding a cytokine of Table 50, or a functional variant thereof, is used for the treatment of a disease or disorder described herein.

In some embodiments, an effector described herein comprises an antibody molecule (e.g., an scFv) that binds a cytokine of Table 50. In some embodiments, an effector described herein comprises an antibody molecule (e.g., an scFv) that binds a cytokine receptor of Table 50. In some embodiments, the antibody molecule comprises a signal sequence.

Exemplary cytokines and cytokine receptors are described, e.g., in Akdis et al., “Interleukins (from IL-1 to IL-38), interferons, transforming growth factor f3, and TNF-α: Receptors, functions, and roles in diseases” October 2016 Volume 138, Issue 4, Pages 984-1010, which is herein incorporated by reference in its entirety, including Table I therein.

TABLE 51
Exemplary polypeptide hormones and receptors

| Hormone | Receptor | Entrez Gene ID | UniProt ID |
|---|---|---|---|
| Natriuretic Peptide, e.g., Atrial | NPRA, NPRB, NPRC | 4878 | P01160 |
| Natriuretic Peptide (ANP) | | | |
| Brain Natriuretic Peptide (BNP) | NPRA, NPRB | 4879 | P16860 |
| C-type natriuretic peptide | NPRB | 4880 | P23582 |
| (CNP) | | | |
| Growth hormone (GH) | GHR | 2690 | P10912 |
| Human growth hormone (hGH) | hGH receptor (human | 2690 | P10912 |
| | GHR) | | |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Antidiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |

In some embodiments, an effector described herein comprises a hormone of Table 51, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 51 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 51 or functional variant thereof comprises a signal sequence, e.g., a signal sequence that is endogenous to the effector, or a heterologous signal sequence. In some embodiments, an Anelloviridae family vector (e.g., anellovector) encoding a hormone of Table 51, or a functional variant thereof, is used for the treatment of a disease or disorder described herein.

In some embodiments, an effector described herein comprises an antibody molecule (e.g., an scFv) that binds a hormone of Table 51. In some embodiments, an effector described herein comprises an antibody molecule (e.g., an scFv) that binds a hormone receptor of Table 51. In some embodiments, the antibody molecule comprises a signal sequence.

TABLE 52
Exemplary growth factors

| Growth Factor | | Entrez Gene ID | UniProt ID |
|---|---|---|---|
| PDGF family | | | |
| PDGF (e.g., PDGF-1, | PDGF receptor, e.g., | 5156 | P16234 |
| PDGF-2, or a | PDGFRα, PDGFRβ | | |
| heterodimer thereof) | | | |
| CSF-1 | CSF1R | 1435 | P09603 |
| SCF | CD117 | 3815 | P10721 |
| VEGF family | | | |
| VEGF (e.g., isoforms | VEGFR-1, VEGFR- | 2321 | P17948 |
| VEGF 121, VEGF 165, | 2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR -3 | | |
| PIGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, | P05230, P09038, |
| FGF-4, FGF-5, FGF-6, | FGFR3, and FGFR4 | 2249, 2250, 2251, | P11487, P08620, |
| FGF-7, FGF-8, FGF-9 | | 2252, 2253, 2254 | P12034, P10767, |
| | | | P21781, P55075, P31371 |
| Insulin family | | | |
| Insulin | IR | 3630 | P01308 |
| IGF-I | IGF-I receptor, IGF- | 3479 | P05019 |
| | II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |

In some embodiments, an effector described herein comprises a growth factor of Table 52, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 52 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 52 or functional variant thereof comprises a signal sequence, e.g., a signal sequence that is endogenous to the effector, or a heterologous signal sequence. In some embodiments, an Anelloviridae family vector (e.g., anellovector) encoding a growth factor of Table 52, or a functional variant thereof, is used for the treatment of a disease or disorder described herein.

In some embodiments, an effector described herein comprises an antibody molecule (e.g., an scFv) that binds a growth factor of Table 52. In some embodiments, an effector described herein comprises an antibody molecule (e.g., an scFv) that binds a growth factor receptor of Table 52. In some embodiments, the antibody molecule comprises a signal sequence.

In some embodiments, an effector described herein comprises a polypeptide that specifically binds to a VEGF (e.g., VEGF 121, VEGF 165, VEGF 189, and/or VEGF 206). In some embodiments, an effector described herein comprises an anti-VEGF antibody molecule, e.g., an antibody molecule that binds specifically to one or more (e.g., 1, 2, 3, or all 4) of VEGF 121, VEGF 165, VEGF 189, and VEGF 206, or a functional fragment, variant, or derivative thereof. In some embodiments, an effector described herein comprises bevacizumab, or a functional fragment, variant, or derivative thereof, or a polypeptide comprising an amino acid sequence having at least 75%, 80%, 85%, 90%, 95%, 96%, 97%, 98%, or 99% sequence identity thereto. In some embodiments, an effector described herein comprises ranibizumab, or a functional fragment, variant, or derivative thereof, or a polypeptide comprising an amino acid sequence having at least 75%, 80%, 85%, 90%, 95%, 96%, 97%, 98%, or 99% sequence identity thereto. In some embodiments, an effector described herein comprises faricimab-svoa, or a functional fragment, variant, or derivative thereof, or a polypeptide comprising an amino acid sequence having at least 75%, 80%, 85%, 90%, 95%, 96%, 97%, 98%, or 99% sequence identity thereto (e.g., for treating a macular degeneration, e.g., wet AMD; and/or diabetic macular edema). In some embodiments, an effector described herein comprises aflibercept, or a functional fragment, variant, or derivative thereof, or a polypeptide comprising an amino acid sequence having at least 75%, 80%, 85%, 90%, 95%, 96%, 97%, 98%, or 99% sequence identity thereto.

In some embodiments, an effector described herein comprises an anti-VEGF receptor antibody molecule. In certain embodiments, an effector described herein comprises an anti-VEGFR1 antibody molecule. In certain embodiments, an effector described herein comprises an anti-VEGFR2 antibody molecule. In certain embodiments, an effector described herein comprises an anti-VEGFR3 antibody molecule.

Exemplary growth factors and growth factor receptors are described, e.g., in Bafico et al., “Classification of Growth Factors and Their Receptors” Holland-Frei Cancer Medicine. 6th edition, which is herein incorporated by reference in its entirety. In some embodiments, an effector as described herein comprises an anti-C4 antibody molecule, or a functional fragment, variant, or derivative thereof, or a polypeptide comprising an amino acid sequence having at least 75%, 80%, 85%, 90%, 95%, 96%, 97%, 98%, or 99% sequence identity thereto. In some embodiments, an effector as described herein comprises an anti-C5 antibody molecule, or a functional fragment, variant, or derivative thereof, or a polypeptide comprising an amino acid sequence having at least 75%, 80%, 85%, 90%, 95%, 96%, 97%, 98%, or 99% sequence identity thereto.

In some embodiments, an effector as described herein comprises an ABCA4 protein (e.g., a human ABCA4 protein), or a polypeptide comprising an amino acid sequence having at least 75%, 80%, 85%, 90%, 95%, 96%, 97%, 98%, or 99% sequence identity thereto. In certain embodiments, the effector is used for treating Stargardt disease.

In some embodiments, an effector as described herein comprises a RPGR protein (e.g., a human RPGR protein), or a polypeptide comprising an amino acid sequence having at least 75%, 80%, 85%, 90%, 95%, 96%, 97%, 98%, or 99% sequence identity thereto. In certain embodiments, the effector is used for treating X-linked retinitis pigmentosa (XLRP).

TABLE 53
Clotting-associated factors

| Effector | Indication | Entrez Gene ID | UniProt ID |
|---|---|---|---|
| Factor I | Afibrinogenomia | 2243, 2266, | P02671, P02679, |
| (fibrinogen) | | 2244 | P02675 |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing | 2162, 2165 | P00488, P05160 |
| | factor deficiency | | |
| vWF | von Willebrand | 7450 | P04275 |
| | disease | | |

In some embodiments, an effector described herein comprises a polypeptide of Table 53, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 53 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, the polypeptide of Table 53 or functional variant thereof comprises a signal sequence, e.g., a signal sequence that is endogenous to the effector, or a heterologous signal sequence. In some embodiments, an Anelloviridae family vector (e.g., anellovector) encoding a polypeptide of Table 53, or a functional variant thereof is used for the treatment of a disease or disorder of Table 53.

### Exemplary Protein Replacement Therapeutics

Exemplary protein replacement therapeutics are described herein, e.g., in the tables below.

TABLE 54
Exemplary enzymatic effectors and corresponding indications

| Effector | Deficiency | Entrez Gene ID | UniProt ID |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenia Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, | Q8TDN7, Q5QJU3, |
| | (lipogranulomatosis) | 55331 | Q9NUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc:Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man:Man5GlcNAc2- | glycosylation CDG Id | | |
| PP-dolichol | | | |
| mannosyltransferase | | | |
| Dolichyl-P-glucose:Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose:Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, | Q12794, Q12891, |
| | IX (hyaluronidase deficiency) | 8372, 23553 | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransferase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl-1- | Mucolipidosis ML III | 79158 | Q3T906 |
| phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl-1- | Mucolipidosis ML III | 84572 | Q9UJJ9 |
| phosphotransferase, | (pseudo-Hurler's | | |
| substrate-recognition | polydystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry's disease | 2717 | P06280 |
| galactosidase | | | |
| tyrosinase, e.g., OCA1 | albinism, e.g., ocular albinism | 7299 | P14679 |
| UDP-GlcNAc:dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-1-Iduronidase | Mucopolysaccharidosis MPS | 2517 | P04066 |
| | I H/S (Hurler-Scheie | | |
| | syndrome) | | |
| α-1-Iduronidase | Mucopolysaccharidosis MPS | 3425 | P35475 |
| | I-H (Hurler's syndrome) | | |
| α-1-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoff's disease | 3073 | P06865 |

In some embodiments, an effector described herein comprises an enzyme of Table 54, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 54 by reference to its UniProt ID. In some embodiments, the functional variant catalyzes the same reaction as the corresponding wild-type protein, e.g., at a rate no less than 10%, 20%, 30%, 40%, or 50% lower than the wild-type protein. In some embodiments, an Anelloviridae family vector (e.g., anellovector) encoding an enzyme of Table 54, or a functional variant thereof is used for the treatment of a disease or disorder of Table 54. In some embodiments, an Anelloviridae family vector (e.g., anellovector) is used to deliver uridine diphosphate glucuronyl-transferase or a functional variant thereof to a target cell, e.g., a liver cell. In some embodiments, an Anelloviridae family vector (e.g., anellovector) is used to deliver OCA1 or a functional variant thereof to a target cell, e.g., a retinal cell.

TABLE 55
Exemplary non-enzymatic effectors and corresponding indications

| Effector | Indication | Entrez Gene ID | UniProt ID |
|---|---|---|---|
| Survival motor neuron | spinal muscular atrophy | 6606 | Q16637 |
| protein (SMN) | | | |
| Dystrophin or micro- | muscular dystrophy | 1756 | P11532 |
| dystrophin | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

In some embodiments, an effector described herein comprises an erythropoietin (EPO), e.g., a human erythropoietin (hEPO), or a functional variant thereof. In some embodiments, an Anelloviridae family vector (e.g., anellovector) encoding an erythropoietin, or a functional variant thereof is used for stimulating erythropoiesis. In some embodiments, an Anelloviridae family vector (e.g., anellovector) encoding an erythropoietin, or a functional variant thereof is used for the treatment of a disease or disorder, e.g., anemia. In some embodiments, an Anelloviridae family vector (e.g., anellovector) is used to deliver EPO or a functional variant thereof to a target cell, e.g., a red blood cell.

In some embodiments, an effector described herein comprises a polypeptide of Table 55, or a functional variant thereof, e.g., a homolog (e.g., ortholog or paralog) or fragment thereof. In some embodiments, an effector described herein comprises a protein having at least 80%, 85%, 90%, 95%, 967%, 98%, 99% sequence identity to an amino acid sequence listed in Table 55 by reference to its UniProt ID. In some embodiments, an Anelloviridae family vector (e.g., anellovector) encoding a polypeptide of Table 55, or a functional variant thereof is used for the treatment of a disease or disorder of Table 55. In some embodiments, an Anelloviridae family vector (e.g., anellovector) is used to deliver SMN or a functional variant thereof to a target cell, e.g., a cell of the spinal cord and/or a motor neuron. In some embodiments, an Anelloviridae family vector (e.g., anellovector) is used to deliver a micro-dystrophin to a target cell, e.g., a myocyte.

Exemplary micro-dystrophins are described in Duan, “Systemic AAV Micro-dystrophin Gene Therapy for Duchenne Muscular Dystrophy.” Mol Ther. 2018 Oct. 3; 26(10):2337-2356. doi: 10.1016/j.ymthe.2018.07.011. Epub 2018 Jul. 17.

In some embodiments, an effector described herein comprises a clotting factor, e.g., a clotting factor listed in Table 54 or Table 55 herein. In some embodiments, an effector described herein comprises a protein that, when mutated, causes a lysosomal storage disorder, e.g., a protein listed in Table 54 or Table 55 herein. In some embodiments, an effector described herein comprises a transporter protein, e.g., a transporter protein listed in Table 55 herein.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polyeptpide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

### Regeneration, Repair, and Fibrosis Factors

Therapeutic polypeptides described herein also include growth factors, e.g., as disclosed in Table 56, or functional variants thereof, e.g., a protein having at least 80%, 85%, 90%, 95%, 967%, 98%, 99% identity to a protein sequence disclosed in Table 56 by reference to its UniProt ID. Also included are antibodies or fragments thereof against such growth factors, or miRNAs that promote regeneration and repair.

TABLE 56
Exemplary regeneration, repair, and fibrosis factors

| Target | Gene accession # | Protein accession # |
|---|---|---|
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR-199-3p | MIMAT0000232 | |
| miR-590-3p | MIMAT0004801 | |
| mi-17-92 | MI0000071 | https://www.ncbi.nlm.nih.gov/pmc/articles/PMC2732113/figure/F1/ |
| miR-222 | MI0000299 | |
| miR-302-367 | MIR302A And | https://www.ncbi.nlm.nih.gov/pmc/articles/PMC4400607/ |
| | MIR367 | |

### Transformation Factors

Therapeutic polypeptides described herein also include transformation factors, e.g., protein factors that transform fibroblasts into differentiated cell e.g., factors disclosed in Table 57 or functional variants thereof, e.g., a protein having at least 80%, 85%, 90%, 95%, 967%, 98%, 99% identity to a protein sequence disclosed in Table 57 by reference to its UniProt ID.

TABLE 57
Exemplary transformation factors

| Target | Indication | Gene accession # | Protein accession # |
|---|---|---|---|
| MESP1 | Organ Repair by | Gene ID: 55897 | EAX02066 |
| | transforming fibroblasts | | |
| ETS2 | Organ Repair by | GeneID: 2114 | NP_005230 |
| | transforming fibroblasts | | |
| HAND2 | Organ Repair by | GeneID: 9464 | NP_068808 |
| | transforming fibroblasts | | |
| MYOCARDIN | Organ Repair by | GeneID: 93649 | NP_001139784 |
| | transforming fibroblasts | | |
| ESRRA | Organ Repair by | Gene ID: 2101 | AAH92470 |
| | transforming fibroblasts | | |
| miR-1 | Organ Repair by | MI0000651 | n/a |
| | transforming fibroblasts | | |
| miR-133 | Organ Repair by | MI0000450 | n/a |
| | transforming fibroblasts | | |
| TGFb | Organ Repair by | GeneID: 7040 | NP_000651.3 |
| | transforming fibroblasts | | |
| WNT | Organ Repair by | Gene ID: 7471 | NP_005421 |
| | transforming fibroblasts | | |
| JAK | Organ Repair by | Gene ID: 3716 | NP_001308784 |
| | transforming fibroblasts | | |
| NOTCH | Organ Repair by | GeneID: 4851 | XP_011517019 |
| | transforming fibroblasts | | |

Proteins that Stimulate Cellular Regeneration

Therapeutic polypeptides described herein also include proteins that stimulate cellular regeneration e.g., proteins disclosed in Table 58 or functional variants thereof, e.g., a protein having at least 80%, 85%, 90%, 95%, 967%, 98%, 99% identity to a protein sequence disclosed in Table 58 by reference to its UniProt ID.

TABLE 58
Exemplary proteins that stimulate cellular regeneration

| Target | Gene accession # | Protein accession # |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

### STING Modulator Effectors

In some embodiments, a secreted effector described herein modulates STING/cGAS signaling. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679, which is incorporated herein by reference in its entirety. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52, each of which is incorporated herein by reference in its entirety.

Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition or Anelloviridae family vector (e.g., anellovector) described herein includes a polypeptide linked to a ligand that is capable of targeting a specific location, tissue, or cell.

### Gene Editing Components

The genetic element of the Anelloviridae family vector (e.g., anellovector) may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124. CRISPR systems are adaptive defense systems originally discovered in bacteria and archaea.

CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (1-111) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the Anelloviridae family vector (e.g., anellovector) includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the Anelloviridae family vector (e.g., anellovector). Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the Anelloviridae family vector (e.g., anellovector) includes a gene encoding a Cas protein, e.g., a Cas9 protein, may be from any of a variety of prokaryotic species. In some embodiments, the Anelloviridae family vector (e.g., anellovector) includes a gene encoding a particular Cas protein, e.g., a particular Cas9 protein, is selected to recognize a particular protospacer-adjacent motif (PAM) sequence. In some embodiments, the Anelloviridae family vector (e.g., anellovector) includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Anelloviridae family vector (e.g., anellovector) includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the Anelloviridae family vector (e.g., anellovector) comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the Anelloviridae family vector (e.g., anellovector) includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the Anelloviridae family vector (e.g., anellovector) includes a gene encoding a dCas9-methylase fusion. In other some embodiments, the Anelloviridae family vector (e.g., anellovector) includes a gene encoding a dCas9-enzyme fusion with a site-specific gRNA to target an endogenous gene.

In other aspects, the Anelloviridae family vector (e.g., anellovector) includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Replication Proteins

In some embodiments, the genetic element of the Anelloviridae family vector (e.g., anellovector), e.g., synthetic Anelloviridae family vector (e.g., anellovector), may include sequences that encode one or more replication proteins. In some embodiments, the Anelloviridae family vector (e.g., anellovector) may replicate by a rolling-circle replication method, e.g., synthesis of the leading strand and the lagging strand is uncoupled. In such embodiments, the Anelloviridae family vector (e.g., anellovector) comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi:10.1186/1743-422X-6-60.

The genetic element may comprise a sequence encoding a polymerase, e.g., RNA polymerase or a DNA polymerase.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the Anelloviridae family vector (e.g., anellovector) in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

### Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

### Viral Sequence

In some embodiments, the genetic element comprises at least one viral sequence. In some embodiments, the sequence has homology or identity to one or more sequence from a single stranded DNA virus, e.g., Anelloviridae family virus (e.g., Anellovirus or CAV), Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Bimavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anelloviridae family virus (e.g., an Anellovirus or CAV). Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. To date Anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element comprises one or more sequences with homology or identity to one or more sequences from one or more non-Anelloviridae family viruses (e.g., non-Anelloviruses), e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the Anelloviridae family vectors (e.g., anellovectors) described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more.

In some embodiments, the genetic element comprises a nucleotide sequence with at least about 75% nucleotide sequence identity, at least about 80%, 85%, 90% 95%, 96%, 97%, 98%, 99% or 100% nucleotide sequence identity to any one of the nucleotide sequences described herein, e.g., as listed in any of Tables N1-N4. Since the genetic code is degenerate, a homologous nucleotide sequence can include any number of silent base changes, i.e., nucleotide substitutions that nonetheless encode the same amino acid.

### Gene Editing Component

The genetic element of the Anelloviridae family vector (e.g., anellovector) may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (1-111) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the Anelloviridae family vector (e.g., anellovector) includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the Anelloviridae family vector (e.g., anellovector). Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the Anelloviridae family vector (e.g., anellovector) includes a gene encoding a Cas protein, e.g., a Cas9 protein, may be from any of a variety of prokaryotic species. In some embodiments, the Anelloviridae family vector (e.g., anellovector) includes a gene encoding a particular Cas protein, e.g., a particular Cas9 protein, is selected to recognize a particular protospacer-adjacent motif (PAM) sequence. In some embodiments, the Anelloviridae family vector (e.g., anellovector) includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Anelloviridae family vector (e.g., anellovector) includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas9 generates only a single-strand break; a catalytically inactive Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the Anelloviridae family vector (e.g., anellovector) comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA.

The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

As used herein, a “biologically active portion of an effector domain” is a portion that maintains the function (e.g. completely, partially, or minimally) of an effector domain (e.g., a “minimal” or “core” domain). In some embodiments, the Anelloviridae family vector (e.g., anellovector) includes a gene encoding a fusion of a dCas9 with all or a portion of one or more effector domains to create a chimeric protein useful in the methods described herein. Accordingly, in some embodiments, the Anelloviridae family vector (e.g., anellovector) includes a gene encoding a dCas9-methylase fusion. In other some embodiments, the Anelloviridae family vector (e.g., anellovector) includes a gene encoding a dCas9-enzyme fusion with a site-specific gRNA to target an endogenous gene.

In other aspects, the Anelloviridae family vector (e.g., anellovector) includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Proteinaceous Exterior

In some embodiments, the Anelloviridae family vector (e.g. anellovector, e.g., synthetic anellovector), comprises a proteinaceous exterior that encloses the genetic element. The proteinaceous exterior can comprise a substantially non-pathogenic exterior protein that fails to elicit an unwanted immune response in a mammal. The proteinaceous exterior of the Anelloviridae family vectors (e.g., anellovectors) typically comprises a substantially non-pathogenic protein that may self-assemble into an icosahedral formation that makes up the proteinaceous exterior.

In some embodiments, the proteinaceous exterior protein is encoded by a sequence of the genetic element of the Anelloviridae family vector (e.g., anellovector) (e.g., is in cis with the genetic element). In other embodiments, the proteinaceous exterior protein is encoded by a nucleic acid separate from the genetic element of the Anelloviridae family vector (e.g., anellovector) (e.g., is in trans with the genetic element).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 sequence or CAV VP1 sequence or a capsid protein sequence as listed in any of Tables A1-A3. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to any one of the nucleotide sequences described herein, e.g., an Anelloviridae family virus capsid sequence or a capsid protein sequence as listed in any of Tables A1-A3. In some embodiments, the protein comprises a capsid protein or a functional fragment of a capsid protein that is encoded by a capsid nucleotide sequence or a sequence having at least about 60%, 65%, 70%, 75%, 80%, 85%, 90% 95%, 96%, 97%, 98%, 99%, or 100% nucleotide sequence identity to any one of the nucleotide sequences described herein, e.g., an Anelloviridae family virus capsid sequence or a capsid protein sequence as listed in any of Tables N1-N4.

In some embodiments, the Anelloviridae family vector (e.g., anellovector) comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to any one of the amino acid sequences described herein, e.g., an Anellovirus capsid sequence or a capsid protein sequence in any of Tables A1-A3. In some embodiments, the Anelloviridae family vector (e.g., anellovector) comprises a nucleotide sequence encoding a capsid protein or a functional fragment of a capsid protein or a sequence having at least about 60%, 65%, 70%, 75%, 80%, 85%, 90% 95%, 96%, 97%, 98%, 99%, or 100% sequence identity to any one of the amino acid sequences described herein, e.g., an Anellovirus capsid sequence or a capsid protein sequence in any of Tables A1-A3.

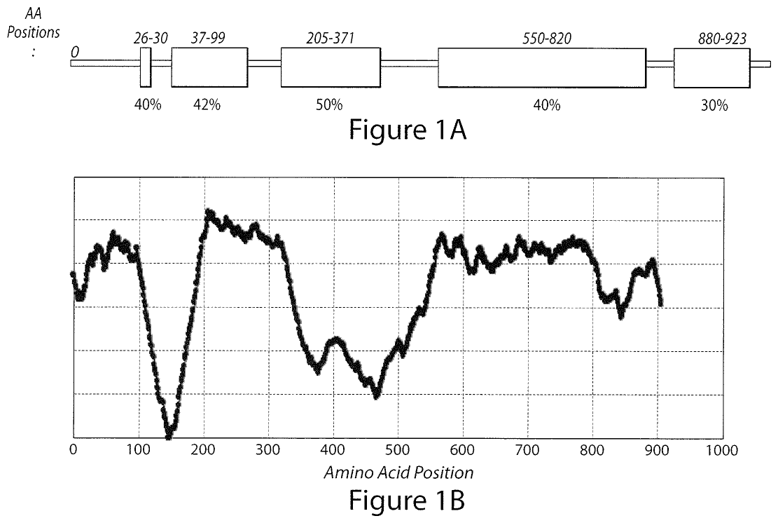

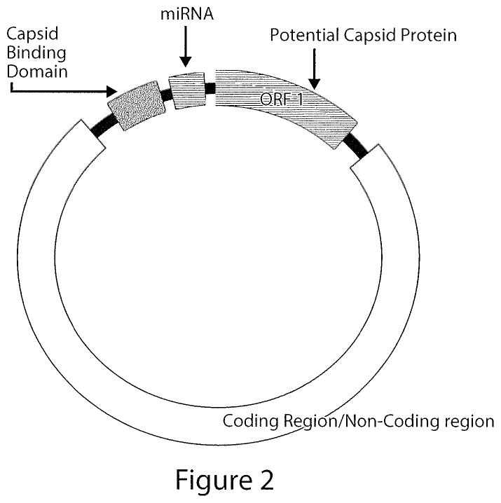

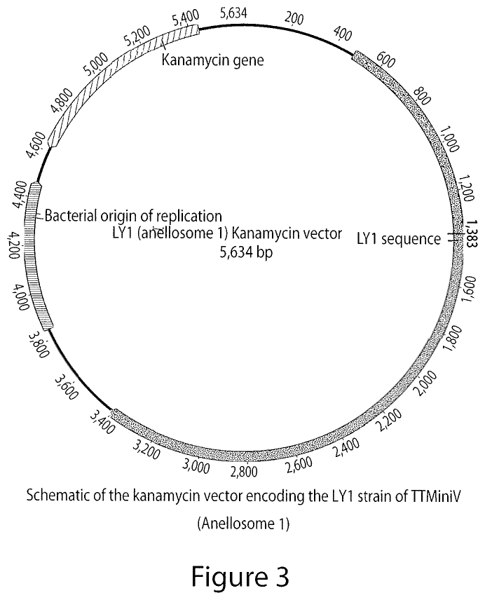

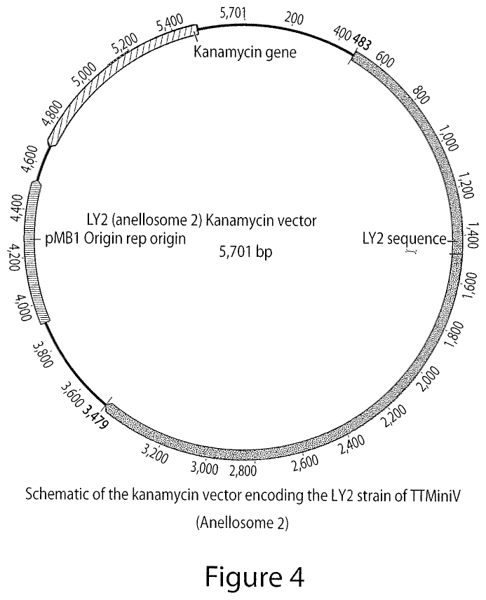

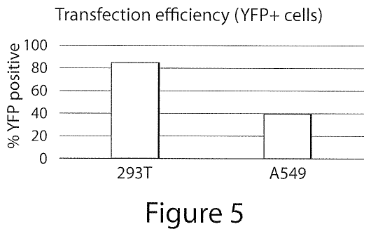

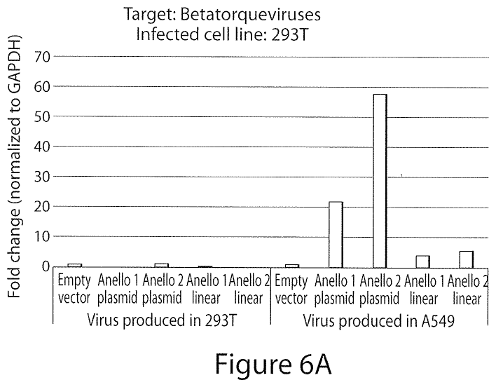

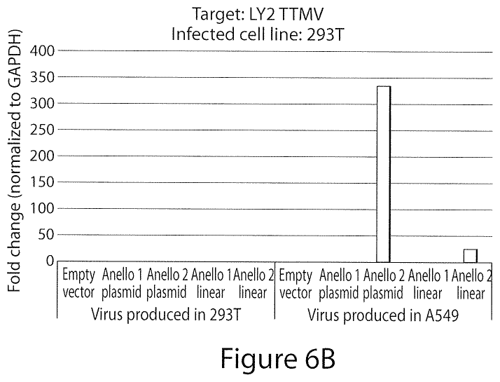

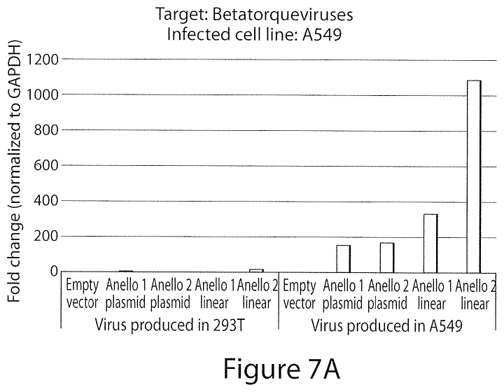

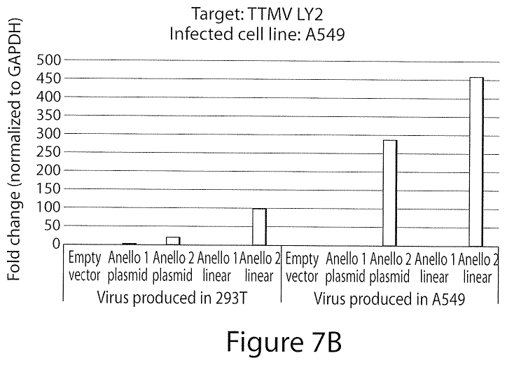

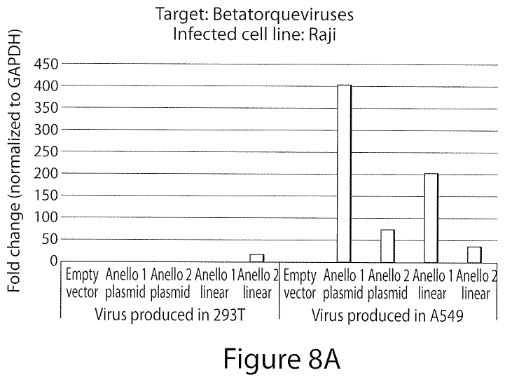

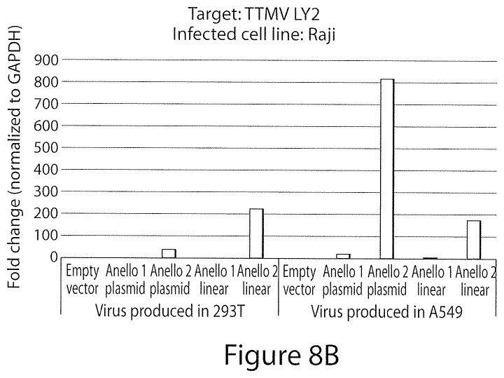

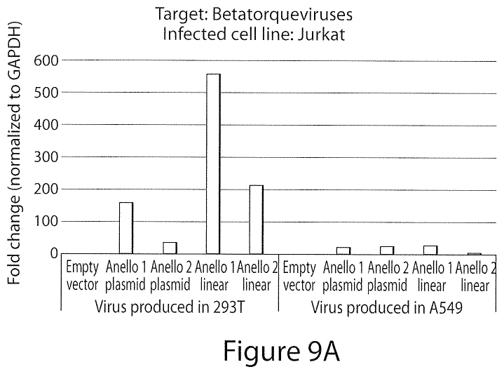

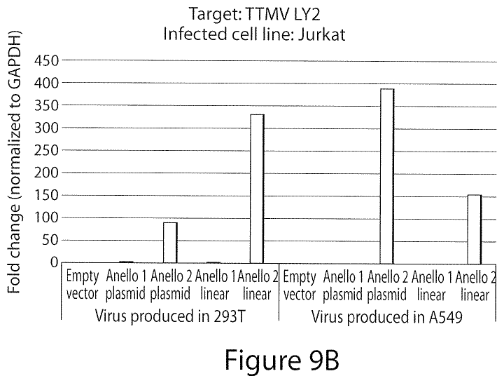

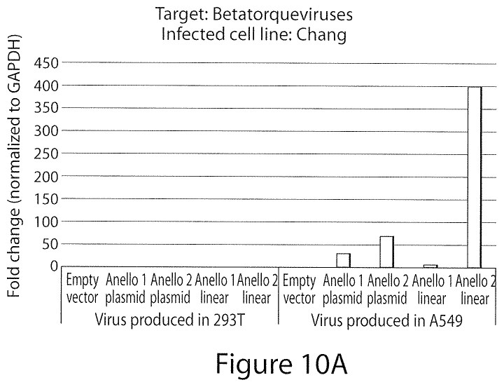

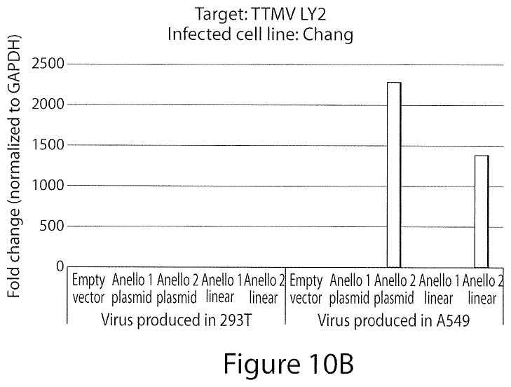

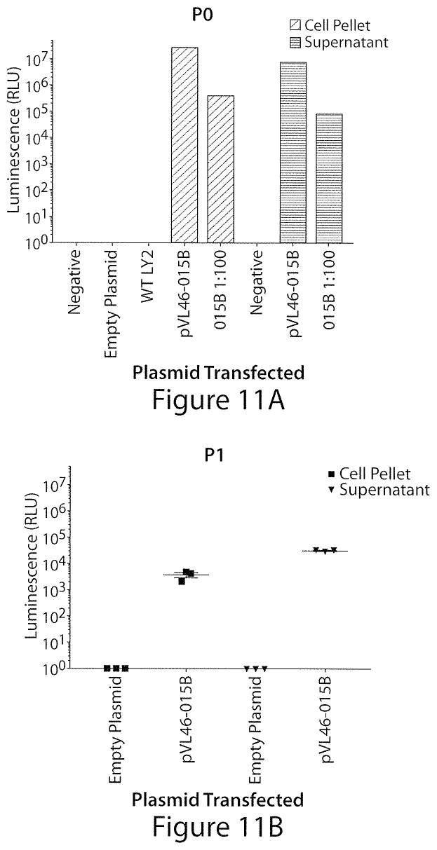

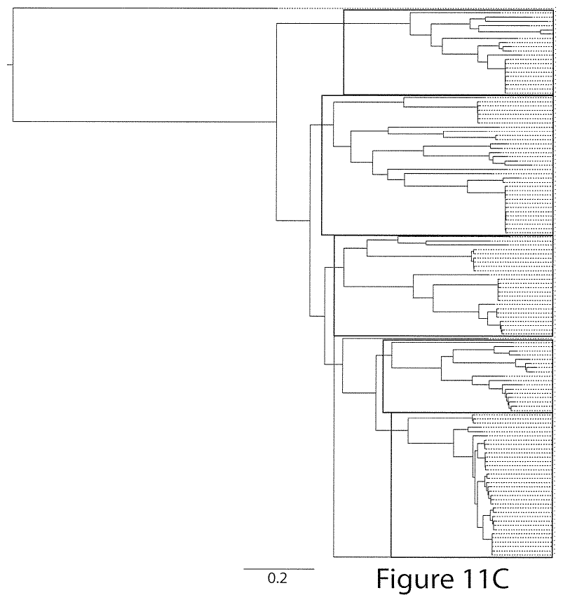

In some embodiments, the Anelloviridae family vector (e.g., anellovector) comprises a nucleotide sequence encoding an amino acid sequence having about position 1 to about position 150 (e.g., or any subset of amino acids within each range, e.g., about position 20 to about position 35, about position 25 to about position 30, about position 26 to about 30), about position 150 to about position 390 (e.g., or any subset of amino acids within each range, e.g., about position 200 to about position 380, about position 205 to about position 375, about position 205 to about 371), about 390 to about position 525, about position 525 to about position 850 (e.g., or any subset of amino acids within each range, e.g., about position 530 to about position 840, about position 545 to about position 830, about position 550 to about 820), about 850 to about position 950 (e.g., or any subset of amino acids within each range, e.g., about position 860 to about position 940, about position 870 to about position 930, about position 880 to about 923) of the amino acid sequences described herein, an Anelloviridae family virus (e.g., an Anellovirus or CAV) amino acid sequence, e.g., as listed in Tables A1-A3, or shown in FIG. 1, or a functional fragment thereof. In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to about position 1 to about position 150 (e.g., or any subset of amino acids within each range as described herein), about position 150 to about position 390, about position 390 to about position 525, about position 525 to about position 850, about position 850 to about position 950 of the amino acid sequences described herein, an Anelloviridae family virus (e.g., an Anellovirus or CAV) amino acid sequence, e.g., as listed in Tables A1-A3, or as shown in FIG. 1.

In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences or ranges of amino acids described herein, an Anelloviridae family virus (e.g., Anellovirus or CAV) amino acid sequence, e.g., as listed in Tables A1-A3, or shown in FIG. 1. In some embodiments, the ranges of amino acids with less sequence identity may provide one or more of the properties described herein and differences in cell/tissue/species specificity (e.g. tropism).

In some embodiments, the Anelloviridae family vector (e.g., anellovector) lacks lipids in the proteinaceous exterior. In some embodiments, the Anelloviridae family vector (e.g., anellovector) lacks a lipid bilayer, e.g., a viral envelope. In some embodiments, the interior of the Anelloviridae family vector (e.g., anellovector) is entirely covered (e.g., 100% coverage) by a proteinaceous exterior. In some embodiments, the interior of the Anelloviridae family vector (e.g., anellovector) is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, so long as the genetic element is retained in the Anelloviridae family vector (e.g., anellovector).

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 or CAV VP1 gene described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### II. Compositions and Methods for Making Anelloviridae Family Vectors

The present disclosure provides, in some aspects, Anelloviridae family vectors (e.g., anellovectors) and methods thereof for delivering effectors. In some embodiments, the Anelloviridae family vectors (e.g., anellovectors) or components thereof can be made as described below. In some embodiments, the compositions and methods described herein can be used to produce a genetic element or a genetic element construct. In some embodiments, the compositions and methods described herein can be used to produce one or more Anelloviridae family virus capsid proteins (e.g., Anellovirus ORF or CAV VP1) molecules (e.g., an ORF1, ORF2, ORF2/2, ORF2/3, ORF1/1, ORF1/2, or VP1 molecule, or a functional fragment or splice variant thereof). In some embodiments, the compositions and methods described herein can be used to produce a proteinaceous exterior or a component thereof (e.g., an ORF1 or VP1 molecule), e.g., in a host cell. In some embodiments, the Anelloviridae family vector (e.g., anellovector) or components thereof can be made using a tandem construct, e.g., as described in PCT Publication No. WO 2021252955, which is incorporated herein by reference in its entirety. In some embodiments, the Anelloviridae family vector (e.g., anellovector) or components thereof can be made using a bacmid/insect cell system, e.g., as described as described in PCT Publication No. WO 2021/252943, which is incorporated herein by reference in its entirety.

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region. The Rep protein may then proceed through the genetic element region, resulting in the synthesis of the genetic element. The genetic element may then be circularized and then enclosed within a proteinaceous exterior to form an Anelloviridae family vector (e.g., anellovector).

### Components and Assembly of Anelloviridae Family Vectors

The compositions and methods herein can be used to produce Anelloviridae family vectors (e.g., anellovectors). As described herein, an Anelloviridae family vector (e.g., anellovector) generally comprises a genetic element (e.g., a single-stranded, circular DNA molecule, e.g., comprising a 5′ UTR region as described herein) enclosed within a proteinaceous exterior (e.g., comprising a polypeptide encoded by an Anelloviridae family virus capsid protein (e.g., an Anellovirus ORF1 or CAV VP1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2) or CAV VP1s. In some embodiments, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described in PCT/US2018/037379 or PCT/US19/65995 (each of which is incorporated by reference herein in their entirety). In embodiments, the genetic element comprises a sequence encoding an Anellovirus ORF1 or CAV VP1, or a splice variant or functional fragment thereof (e.g., a jelly-roll region, e.g., as described herein). In some embodiments, the proteinaceous exterior comprises a polypeptide encoded by an Anellovirus ORF1 or CAV VP1 nucleic acid (e.g., an Anellovirus ORF1 or CAV VP1 molecule or a splice variant or functional fragment thereof).

In some embodiments, an anellovector is assembled by enclosing a genetic element (e.g., as described herein) within a proteinaceous exterior (e.g., as described herein). In some embodiments, the genetic element is enclosed within the proteinaceous exterior in a host cell (e.g., as described herein). In some embodiments, the host cell expresses one or more polypeptides comprised in the proteinaceous exterior (e.g., a polypeptide encoded by an Anellovirus ORF1 or CAV VP1 nucleic acid, e.g., an ORF1 molecule or VP1 molecule). For example, in some embodiments, the host cell comprises a nucleic acid sequence encoding an Anellovirus ORF1 or CAV VP1 molecule, e.g., a splice variant or a functional fragment of an Anellovirus ORF1 or CAV VP1 polypeptide (e.g., a wild-type Anellovirus ORF1 or CAV VP1 protein or a polypeptide encoded by a wild-type Anellovirus ORF1 or CAV VP1 nucleic acid, e.g., as described herein). In embodiments, the nucleic acid sequence encoding the Anellovirus ORF1 or CAV VP1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 or CAV VP1 molecule is integrated into the genome of the host cell.

In some embodiments, the host cell comprises the genetic element and/or a nucleic acid construct comprising the sequence of the genetic element. In some embodiments, the nucleic acid construct is selected from a plasmid, viral nucleic acid, minicircle, bacmid, or artificial chromosome. In some embodiments, the genetic element is excised from the nucleic acid construct and, optionally, converted from a double-stranded form to a single-stranded form (e.g., by denaturation). In some embodiments, the genetic element is generated by a polymerase based on a template sequence in the nucleic acid construct. In some embodiments, the polymerase produces a single-stranded copy of the genetic element sequence, which can optionally be circularized to form a genetic element as described herein. In other embodiments, the nucleic acid construct is a double-stranded minicircle produced by circularizing the nucleic acid sequence of the genetic element in vitro. In embodiments, the in vitro-circularized (IVC) minicircle is introduced into the host cell, where it is converted to a single-stranded genetic element suitable for enclosure in a proteinaceous exterior, as described herein.

### ORF1 or VP1 Molecules, e.g., for Assembly of Anelloviridae Family Vectors (e.g., Anellovectors)

An Anelloviridae family vector (e.g., anellovector) can be made, for example, by enclosing a genetic element within a proteinaceous exterior. The proteinaceous exterior of an Anelloviridae family vector (e.g., anellovector) generally comprises a polypeptide encoded by an Anelloviridae family virus (e.g., Anellovirus ORF1 or CAV VP1) nucleic acid (e.g., an Anellovirus ORF1 or CAV VP1 molecule or a splice variant or functional fragment thereof, e.g., as described herein). An ORF1 molecule or VP1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 or CAV VP1 arginine-rich region, jelly-roll region, N22 domain, hypervariable region, and/or C-terminal domain. In some embodiments, the proteinaceous exterior comprises an Anellovirus ORF1 or CAV VP1 jelly-roll region (e.g., as described herein). In some embodiments, the proteinaceous exterior comprises an Anellovirus ORF1 or CAV VP1 arginine-rich region (e.g., as described herein). In some embodiments, the proteinaceous exterior comprises an Anellovirus ORF1 N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 C-terminal domain (e.g., as described herein).

In some embodiment s, the Anelloviridae family vector (e.g., anellovector) comprises an ORF1 molecule and/or a nucleic acid encoding an ORF1 molecule; or a VP1 molecule and/or a nucleic acid encoding a VP1 molecule. Generally, an ORF1 or VP1 molecule comprises a polypeptide having the structural features and/or activity of an Anellovirus ORF1 or CAV VP1 protein (e.g., an Anellovirus ORF1 or CAV VP1 protein as described herein), or a functional fragment thereof. In some embodiments, the ORF1 or VP1 molecule comprises a truncation relative to an Anellovirus ORF1 or CAV VP1 protein (e.g., an Anellovirus ORF1 or CAV VP1 protein as described herein). In some embodiments, the ORF1 or VP1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 or CAV VP1 protein. In some embodiments, an ORF1 molecule comprises an amino acid sequence having at least 75%, 80%, 85%, 90%, 95%, 96%, 97%, 98%, or 99% sequence identity to a Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule or VP1 molecule may be capable of binding to other ORF1 molecules or VP1 molecule, e.g., to form a proteinaceous exterior (e.g., as described herein). Such an ORF1 molecule or VP1 molecule may be described as having the capacity to form a capsid. In some embodiments, the proteinaceous exterior may enclose a nucleic acid molecule (e.g., a genetic element as described herein, e.g., produced using a composition or construct as described herein). In some embodiments, a plurality of ORF1 molecules or VP1 molecules may form a multimer, e.g., to produce a proteinaceous exterior. In some embodiments, the multimer may be a homomultimer. In other embodiments, the multimer may be a heteromultimer.

In some embodiments, a first plurality of Anelloviridae family vectors (e.g., anellovector) comprising an ORF1 or VP1 molecule as described herein is administered to a subject. In some embodiments, a second plurality of Anelloviridae family vectors (e.g., anellovector) comprising an ORF1 or VP1 molecule described herein, is subsequently administered to the subject following administration of the first plurality. In some embodiments the second plurality of Anelloviridae family vectors (e.g., anellovectors) comprises an ORF1 or VP1 molecule having the same amino acid sequence as the ORF1 or VP1 molecule comprised by the anellovectors of the first plurality. In some embodiments the second plurality of Anelloviridae family vectors (e.g., anellovector) comprises an ORF1 or VP1 molecule having at least 70%, 75%, 80%, 85%, 90%, 95%, 96%, 97%, 98%, 99%, or 100% amino acid sequence identity to the ORF1 or VP1 molecule comprised by the anellovectors of the first plurality.

ORF2 or VP2 Molecules, e.g., for Assembly of Anelloviridae Family Vectors (e.g. Anellovectors)

Producing an Anelloviridae family vector (e.g. anellovector) using the compositions or methods described herein may involve expression of an Anellovirus ORF2 or VP2 molecule (e.g., as described herein), or a splice variant or functional fragment thereof. In some embodiments, the Anelloviridae family ovector comprises an ORF2 or VP2 molecule, or a splice variant or functional fragment thereof, and/or a nucleic acid encoding an ORF2 or VP2 molecule, or a splice variant or functional fragment thereof. In some embodiments, the anellovector does not comprise an ORF2 or VP2 molecule, or a splice variant or functional fragment thereof, and/or a nucleic acid encoding an ORF2 or VP2 molecule, or a splice variant or functional fragment thereof. In some embodiments, producing the anellovector comprises expression of an ORF2 or VP2 molecule, or a splice variant or functional fragment thereof, but the ORF2 or VP2 molecule is not incorporated into the Anelloviridae family vector.

### Production of Protein Components

Protein components of an Anelloviridae family vector (e.g., anellovector), e.g., ORF1 or VP1 molecules, can be produced in a variety of ways, e.g., as described herein. In some embodiments, the protein components of an Anelloviridae family vector (e.g., anellovector), including, e.g., the proteinaceous exterior, are produced in the same host cell that packages the genetic elements into the proteinaceous exteriors, thereby producing the Anelloviridae family vectors (e.g., anellovectors). In some embodiments, the protein components of an Anelloviridae family vector (e.g., anellovector), including, e.g., the proteinaceous exterior, are produced in a cell that does not comprise a genetic element and/or a genetic element construct (e.g., as described herein).

### Baculovirus Expression Systems

A viral expression system, e.g., a baculovirus expression system, may be used to express proteins (e.g., for production of Anelloviridae family vector (e.g., anellovector)), e.g., as described herein. Baculoviruses are rod-shaped viruses with a circular, supercoiled double-stranded DNA genome. Genera of baculoviruses include: Alphabaculovirus (nucleopolyhedroviruses (NPVs) isolated from Lepidoptera), Betabaculoviruses (granuloviruses (GV) isolated from Lepidoptera), Gammabaculoviruses (NPVs isolated from Hymenoptera) and Deltabaculoviruses (NPVs isolated from Diptera). While GVs typically contain only one nucleocapsid per envelope, NPVs typically contain either single (SNPV) or multiple (MNPV) nucleocapsids per envelope. The enveloped virions are further occluded in granulin matrix in GVs and polyhedrin in NPVs. Baculoviruses typically have both lytic and occluded life cycles. In some embodiments, the lytic and occluded life cycles manifest independently throughout the three phases of virus replication: early, late, and very late phase. In some embodiments, during the early phase, viral DNA replication takes place following viral entry into the host cell, early viral gene expression and shut-off of the host gene expression machinery. In some embodiments, in the late phase late genes that code for viral DNA replication are expressed, viral particles are assembled, and extracellular virus (EV) is produced by the host cell. In some embodiments, in the very late phase the polyhedrin and p10 genes are expressed, occluded viruses (OV) are produced by the host cell, and the host cell is lysed. Since baculoviruses infect insect species, they can be used as biological agents to produce exogenous proteins in baculoviruses-permissive insect cells or larvae. Different isolates of baculovirus, such as Autographa californica multiple nuclear polyhedrosis virus (AcMNPV) and Bombyx mori (silkworm) nuclear polyhedrosis virus (BmNPV) may be used in exogenous protein expression. Various baculoviral expression systems are commercially available, e.g., from ThermoFisher.

In some embodiments, the proteins described herein (e.g., an Anellovirus ORF or CAV VP1 molecule, e.g., ORF1, ORF2, ORF2/2, ORF2/3, ORF1/1, ORF1/2, or VP1, or a functional fragment or splice variant thereof) may be expressed using a baculovirus expression vector (e.g., a bacmid) that comprises one or more components described herein. For example, a baculovirus expression vector may include one or more of (e.g., all of) a selectable marker (e.g., kanR), an origin of replication (e.g., one or both of a bacterial origin of replication and an insect cell origin of replication), a recombinase recognition site (e.g., an att site), and a promoter. In some embodiments, a baculovirus expression vector (e.g., a bacmid as described herein) can be produced by replacing the naturally occurring wild-type polyhedrin gene, which encodes for baculovirus occlusion bodies, with genes encoding the proteins described herein. In some embodiments, the genes encoding the proteins described herein are cloned into a baculovirus expression vector (e.g., a bacmid as described herein) containing a baculovirus promoter. In some embodiments, the baculovirual vector comprises one or more non-baculoviral promoters, e.g., a mammalian promoter or an Anelloviridae family virus (e.g., Anellovirus or CAV) promoter. In some embodiments, the genes encoding the proteins described herein are cloned into a donor vector (e.g., as described herein), which is then contacted with an empty baculovirus expression vector (e.g., an empty bacmid) such that the genes encoding the proteins described herein are transferred (e.g., by homologous recombination or transposase activity) from the donor vector into the baculovirus expression vector (e.g., bacmid). In some embodiments, the baculovirus promoter is flanked by baculovirus DNA from the nonessential polyhedrin gene locus. In some embodiments, a protein described herein is under the transcriptional control of the AcNPV polyhedrin promoter in the very late phase of viral replication. In some embodiments, a strong promoter suitable for use in baculoviral expression in insect cells include, but are not limited to, baculovirus p10 promoters, polyhedrin (polh) promoters, p6.9 promoters and capsid protein promoters. Weak promoters suitable for use in baculoviral expression in insect cells include ie1, ie2, ie0, et1, 39K (aka pp31) and gp64 promoters of baculoviruses.

In some embodiments, a recombinant baculovirus is produced by homologous recombination between a baculoviral genome (e.g., a wild-type or mutant baculoviral genome), and a transfer vector. In some embodiments, one or more genes encoding a protein described herein are cloned into the transfer vector. In some embodiments, the transfer vector further contains a baculovirus promoter flanked by DNA from a nonessential gene locus, e.g., polyhedrin gene. In some embodiments, one or more genes encoding a protein described herein are inserted into the baculoviral genome by homologous recombination between the baculoviral genome and the transfer vector. In some embodiments, the baculoviral genome is linearized at one or more unique sites. In some embodiments, the linearized sites are located near the target site for insertion of genes encoding the proteins described herein into the baculoviral genome. In some embodiments, a linearized baculoviral genome missing a fragment of the baculoviral genome downstream from a gene, e.g., polyhedrin gene, can be used for homologous recombination. In some embodiments, the baculoviral genome and transfer vector are co-transfected into insect cells. In some embodiments, the method of producing the recombinant baculovirus comprises the steps of preparing the baculoviral genome for performing homologous recombination with a transfer vector containing the genes encoding one or more protein described herein and co-transfecting the transfer vector and the baculoviral genome DNA into insect cells. In some embodiments, the baculoviral genome comprises a region homologous to a region of the transfer vector. These homologous regions may enhance the probability of recombination between the baculoviral genome and the transfer vector. In some embodiments, the homology region in the transfer vector is located upstream or downstream of the promoter. In some embodiments, to induce homologous recombination, the baculoviral genome, and transfer vector are mixed at a weight ratio of about 1:1 to 10:1.

In some embodiments, a recombinant baculovirus is generated by a method comprising site-specific transposition with Tn7, e.g., whereby the genes encoding the proteins described herein are inserted into bacmid DNA, e.g., propagated in bacteria, e.g., E. coli (e.g., DH 10Bac cells). In some embodiments, the genes encoding the proteins described herein are cloned into a pFASTBAC® vector and transformed into competent cells, e.g., DH10BAC® competent cells, containing the bacmid DNA with a mini-attTn7 target site. In some embodiments, the baculovirus expression vector, e.g., pFASTBAC® vector, may have a promoter, e.g., a dual promoter (e.g., polyhedrin promoter, p10 promoter). Commercially available pFASTBAC® donor plasmids include: pFASTBAC 1, pFASTBAC HT, and pFASTBAC DUAL. In some embodiments, recombinant bacmid DNA containing-colonies are identified and bacmid DNA is isolated to transfect insect cells.

In some embodiments, a baculoviral vector is introduced into an insect cell together with a helper nucleic acid. The introduction may be concurrent or sequential. In some embodiments, the helper nucleic acid provides one or more baculoviral proteins, e.g., to promote packaging of the baculoviral vector. In some embodiments, recombinant baculovirus produced in insect cells (e.g., by homologous recombination) is expanded and used to infect insect cells (e.g., in the mid-logarithmic growth phase) for recombinant protein expression. In some embodiments, recombinant bacmid DNA produced by site-specific transposition in bacteria, e.g., E. coli, is used to transfect insect cells with a transfection agent, e.g., Cellfectin® II. Additional information on baculovirus expression systems is discussed in U.S. patent application Ser. Nos. 14/447,341, 14/277,892, and 12/278,916, which are hereby incorporated by reference.

### Insect Cell Systems

The proteins described herein may be expressed in insect cells infected or transfected with recombinant baculovirus or bacmid DNA, e.g., as described above. In some embodiments, insect cells include: the Sf9 and Sf21 cells derived from Spodoptera frugiperda and the Tn-368 and High Five™ BTI-TN-5B1-4 cells (also referred to as Hi5 cells) derived from Trichoplusia ni. In some embodiments, insect cell lines Sf21 and Sf9, derived from the ovaries of the pupal fall army worm Spodoptera frugiperda, can be used for the expression of recombinant proteins using the baculovirus expression system. In some embodiments, Sf21 and Sf9 insect cells may be cultured in commercially available serum-supplemented or serum-free media. Suitable media for culturing insect cells include: Grace's Supplemented (TNM-FH), IPL-41, TC-100, Schneider's Drosophila, SF-900 II SFM, and EXPRESS-FIVE™ SFM. In some embodiments, some serum-free media formulations utilize a phosphate buffer system to maintain a culture pH in the range of 6.0-6.4 (Licari et al. Insect cell hosts for baculovirus expression vectors contain endogenous exoglycosidase activity. Biotechnology Progress 9: 146-152 (1993) and Drugmand et al. Insect cells as factories for biomanufacturing. Biotechnology Advances 30:1140-1157 (2012)) for both cultivation and recombinant protein production. In some embodiments, a pH of 6.0-6.8 for cultivating various insect cell lines may be used. In some embodiments, insect cells are cultivated in suspension or as a monolayer at a temperature between 25° to 30° C. with aeration. Additional information on insect cells is discussed, for example, in U.S. patent application Ser. Nos. 14/564,512 and 14/775,154, each of which is hereby incorporated by reference.

### Mammalian Cell Systems

In some embodiments, the proteins described herein may be expressed in vitro in animal cell lines infected or transfected with a vector encoding the protein, e.g., as described herein. Animal cell lines envisaged in the context of the present disclosure include porcine cell lines, e.g., immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines suitable for expressing the proteins described herein include, but are not limited to cell lines of human or primate origin, such as human or primate kidney carcinoma cell lines.

### Genetic Element Constructs, e.g., for Assembly of Anelloviridae Family Vectors

The genetic element of an Anelloviridae family vector (e.g., anellovector) as described herein may be produced from a genetic element construct that comprises a genetic element region and optionally other sequence such as vector backbone. Generally, the genetic element construct comprises an Anelloviridae family virus (e.g., Anellovirus) 5′ UTR (e.g., as described herein). A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, or a minicircle, e.g., as described herein). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some aspects, the present disclosure provides a method for replication and propagation of the Anelloviridae family vector (e.g., anellovector) as described herein (e.g., in a cell culture system), which may comprise one or more of the following steps: (a) introducing (e.g., transfecting) a genetic element (e.g., linearized) into a cell line sensitive to Anelloviridae family vector (e.g., anellovector) infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

### Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone).

### Small Circular Nucleic Acid Constructs

In some embodiments, the genetic element construct is a circular nucleic acid construct, e.g., lacking a backbone (e.g., lacking a bacterial origin of replication and/or selectable marker). In embodiments, the genetic element is a double-stranded circular nucleic acid construct. In embodiments, the double-stranded circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

### In Vitro Circularization

In some instances, the genetic element to be packaged into a proteinaceous exterior is a single stranded circular DNA. The genetic element may, in some instances, be introduced into a host cell via a genetic element construct having a form other than a single stranded circular DNA. For example, the genetic element construct may be a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or TraI, e.g., as described in Wawrzyniak et al. 2017, Front. Microbiol. 8: 2353; incorporated herein by reference with respect to the listed enzymes). In some embodiments, the double-stranded circular DNA is produced by in vitro circularization (IVC), e.g., as described in Example 15.

Generally, in vitro circularized DNA constructs can be produced by digesting a genetic element construct (e.g., a plasmid comprising the sequence of a genetic element) to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an Anelloviridae family vector (e.g., anellovector), e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Tandem Constructs

In some embodiments, a genetic element construct comprises a first copy of a genetic element sequence (e.g., the nucleic acid sequence of a genetic element, e.g., as described herein) and at least a portion of a second copy of a genetic element sequence (e.g., the nucleic acid sequence of the same genetic element, or the nucleic acid sequence of a different genetic element), arranged in tandem. Genetic element constructs having such a structure are generally referred to herein as tandem constructs. Such tandem constructs are used for producing an Anelloviridae family vector (e.g., anellovector) genetic element. The first copy of the genetic element sequence and the second copy of the genetic element sequence may, in some instances, be immediately adjacent to each other on the genetic acid construct. In other instances, the first copy of the genetic element sequence and the second copy of the genetic element sequence may be separated, e.g., by a spacer sequence. In some embodiments, the second copy of the genetic element sequence, or the portion thereof, comprises an upstream replication-facilitating sequence (uRFS), e.g., as described herein. In some embodiments, the second copy of the genetic element sequence, or the portion thereof, comprises a downstream replication-facilitating sequence (dRFS), e.g., as described herein. In some embodiments, the uRFS and/or dRFS comprises an origin of replication (e.g., a mammalian origin of replication, an insect origin of replication, or a viral origin of replication, e.g., a non-Anelloviridae family virus (e.g., Anellovirus) origin of replication, e.g., as described herein) or portion thereof. In some embodiments, the uRFS and/or dRFS does not comprise an origin of replication. In some embodiments, the uRFS and/or dRFS comprises a hairpin loop (e.g., in the 5′ UTR). In some embodiments, a tandem construct produces higher levels of a genetic element than an otherwise similar construct lacking the second copy of the genetic element or portion thereof. Without being bound by theory, a tandem construct described herein may, in some embodiments, replicate by rolling circle replication. In some embodiments, a tandem construct is a plasmid. In some embodiments, a tandem construct is circular. In some embodiments, a tandem construct is linear. In some embodiments, a tandem construct is single-stranded. In some embodiments, a tandem construct is double-stranded. In some embodiments, a tandem construct is DNA.

A tandem construct may, in some instances, include a first copy of the sequence of the genetic element and a second copy of the sequence of the genetic element, or a portion thereof. It is understood that the second copy can be an identical copy of the first copy or a portion thereof, or can comprise one or more sequence differences, e.g., substitutions, additions, or deletions. In some instances, the second copy of the genetic element sequence or portion thereof is positioned 5′ relative to the first copy of the genetic element sequence. In some instances, the second copy of the genetic element sequence or portion thereof is positioned 3′ relative to the first copy of the genetic element sequence. In some instances, the second copy of the genetic element sequence or portion thereof and the first copy of the genetic element sequence are adjacent to each other in the tandem construct. In some instances, the second copy of the genetic element sequence or portion thereof and the first copy of the genetic element sequence are separated, e.g., by a spacer sequence.

In some embodiments, the tandem constructs described herein can be used to produce the genetic element of a vector (e.g., Anelloviridae family vector as described herein), vehicle, or particle (e.g., viral particle) comprising a capsid (e.g., a capsid comprising an Anellovirus ORF, e.g., an ORF1 molecule, e.g., as described herein; or a capsid comprising a CAV VP1, e.g., a VP1 molecule, e.g., as described herein) encapsulating a genetic element comprising a protein binding sequence that binds to the capsid and a heterologous (e.g., relative to the Anellovirus from which the ORF1 molecule was derived or the CAV from which the VP1 molecule was derived) sequence encoding a therapeutic effector. In embodiments, the vector is capable of delivering the genetic element into a mammalian, e.g., human, cell. In some embodiments, the genetic element has less than about 50% (e.g., less than 50%, 40%, 30%, 25%, 20%, 15%, 10%, 9%, 8%, 7%, 6%, 5.5%, 5%, 4.5%, 4%, 3.5%, 3%, 2.5%, 2%, 1.5%, or less) identity to a wild type Anelloviridae family virus (e.g., Anellovirus or CAV) genome sequence. In some embodiments, the genetic element has no more than 1.5%, 2%, 2.5%, 3%, 3.5%, 4%, 4.5%, 5%, 5.5%, 6%, 7%, 8%, 9%, 10%, 15%, 20%, 25%, 30%, 40%, 50%, 60%, 70%, 75%, or 80% identity to a wild type Anelloviridae family virus (e.g., Anellovirus or CAV) genome sequence. In some embodiments, the genetic element has greater than about 2000, 3000, 4000, 4500, or 5000 contiguous nucleotides of non-Anelloviridae family virus (e.g., Anellovirus or CAV) genome sequence. In some embodiments, the genetic element has greater than about 2000 to 5000, 2500 to 4500, 3000 to 4500, 2500 to 4500, 3500, or 4000, 4500 (e.g., between about 3000 to 4500) nucleotides nucleotides of non-Anelloviridae family virus (e.g., Anellovirus or CAV) genome sequence.

In some embodiments of the systems and methods herein, a vector (e.g., an Anelloviridae family vector, e.g., as described herein) is made by introducing into a cell a first nucleic acid molecule that is a genetic element or genetic element construct, e.g., a tandem construct, and a second nucleic acid molecule encoding one or more additional proteins (e.g., a Rep molecule and/or a capsid protein), e.g., as described herein. In some embodiments, the first nucleic acid molecule and the second nucleic acid molecule are attached to each other (e.g., in a genetic element construct described herein, e.g., in cis). In some embodiments, the first nucleic acid molecule and the second nucleic acid molecule are separate (e.g, in trans). In some embodiments, the first nucleic acid molecule is a plasmid, cosmid, bacmid, minicircle, or artificial chromosome. In some embodiments, the second nucleic acid molecule is a plasmid, cosmid, bacmid, minicircle, or artificial chromosome. In some embodiments, the second nucleic acid molecule is integrated into the genome of the host cell.

In some embodiments, the method further includes introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is or comprises or is part of a helper construct, helper virus or other helper vector, e.g., as described herein.

Additional descriptions of tandem constructs that can be used with the invention are described, for example, PCT Publication No. WO 2021252955, incorporated herein by reference in its entirety.

### Cis Trans Constructs

In some embodiments, a genetic element construct as described herein comprises one or more sequences encoding one or more Anelloviridae family virus ORFs, e.g., proteinaceous exterior components (e.g., polypeptides encoded by an Anellovirus ORF1 or CAV VP1 nucleic acid, e.g., as described herein). For example, the genetic element construct may comprise a nucleic acid sequence encoding an Anellovirus ORF1 or CAV VP1 molecule. Such genetic element constructs can be suitable for introducing the genetic element and the Anelloviridae family virus ORF(s) into a host cell in cis. In other embodiments, a genetic element construct as described herein does not comprise sequences encoding one or more Anelloviridae family virus ORFs, e.g., proteinaceous exterior components (e.g., polypeptides encoded by an Anellovirus ORF1 or CAV VP1 nucleic acid, e.g., as described herein). For example, the genetic element construct may not comprise a nucleic acid sequence encoding an Anellovirus ORF1 molecule or CAV VP1 molecule. Such genetic element constructs can be suitable for introducing the genetic element into a host cell, with the one or more Anelloviridae family virus ORFs to be provided in trans (e.g., via introduction of a second nucleic acid construct encoding one or more of the Anelloviridae family virus ORFs, or via an Anelloviridae family virus ORF cassette integrated into the genome of the host cell). In some embodiments, an ORF1 molecule is provided in trans, e.g., as described herein. In some embodiments, an ORF2 molecule is provided in trans, e.g., as described herein. In some embodiments, an ORF1 molecule and an ORF2 molecule are both provided in trans, e.g., as described herein. In some embodiments, a VP1 molecule is provided in trans, e.g., as described herein.

In some embodiments, the genetic element construct comprises a sequence encoding an Anellovirus ORF1 or CAV VP1 molecule, or a splice variant or functional fragment thereof (e.g., a jelly-roll region, e.g., as described herein). In embodiments, the portion of the genetic element that does not comprise the sequence of the genetic element comprises the sequence encoding the Anellovirus ORF1 or CAV VP1 molecule, or splice variant or functional fragment thereof (e.g., in a cassette comprising a promoter and the sequence encoding the Anellovirus ORF1 or CAV VP1 molecule, or splice variant or functional fragment thereof). In further embodiments, the portion of the construct comprising the sequence of the genetic element comprises a sequence encoding an Anellovirus ORF1 or CAV VP1 molecule, or a splice variant or functional fragment thereof (e.g., a jelly-roll region, e.g., as described herein). In embodiments, enclosure of such a genetic element in a proteinaceous exterior (e.g., as described herein) produces a replication-component Anelloviridae family vector (e.g., anellovector) (e.g., an Anelloviridae family vector that upon infecting a cell, enables the cell to produce additional copies of the anellovector without introducing further nucleic acid constructs, e.g., encoding one or more Anelloviridae family virus ORFs as described herein, into the cell).

In other embodiments, the genetic element does not comprise a sequence encoding an Anellovirus ORF1 molecule or CAV VP1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent Anelloviridae family vector (e.g., anellovector) (e.g., an Anelloviridae family vector that, upon infecting a cell, does not enable the infected cell to produce additional Anelloviridae family vector, e.g., in the absence of one or more additional constructs, e.g., encoding one or more Anellovirus or CAV ORFs as described herein).

### Expression Cassettes

In some embodiments, a genetic element construct comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anelloviridae family virus (e.g., Anellovirus or CAV) protein (e.g., an Anellovirus ORF1, ORF2, ORF2/2, ORF2/3, ORF1/1, or ORF1/2, or a CAV VP1, or a functional fragment thereof). The expression cassettes may, in some embodiments, be located within the genetic element sequence. In embodiments, an expression cassette for an effector is located within the genetic element sequence. In embodiments, an expression cassette for an Anelloviridae family virus protein is located within the genetic element sequence. In other embodiments, the expression cassettes are located at a position within the genetic element construct outside of the sequence of the genetic element (e.g., in the backbone). In embodiments, an expression cassette for an Anelloviridae family virus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anelloviridae family virus protein (e.g., a sequence encoding an Anellovirus ORF1, ORF2, ORF2/2, ORF2/3, ORF1/1, or ORF1/2, or a CAV VP1, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

### Design and Production of a Genetic Element Construct

Various methods are available for synthesizing a genetic element construct. For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, or a CAV VP1, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of an Anellovirus ORF1, ORF1/1, ORF1/2, ORF2, ORF2/2, ORF2/3, or ORF2t/3, or a CAV VP1, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

In some embodiments, the genetic element construct may be designed using computer-aided design tools.

General methods of making constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

### Effectors

The compositions and methods described herein can be used to produce a genetic element of an Anelloviridae family vector (e.g., anellovector) comprising a sequence encoding an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding an Anellovirus ORF1, ORF1/1, ORF1/2, ORF2, ORF2/2, ORF2/3, and/or ORF2t/3, or a CAV VP1, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

### Host Cells

The Anelloviridae family vector (e.g., anellovector) described herein can be produced, for example, in a host cell. Generally, a host cell is provided that comprises an Anelloviridae family vector (e.g., anellovector) genetic element and the components of an Anelloviridae family vector (e.g., anellovector) proteinaceous exterior (e.g., a polypeptide encoded by an Anellovirus ORF1 nucleic acid or CAV VP1 nucleic acid, or an Anellovirus ORF1 or CAV VP1 molecule). The host cell is then incubated under conditions suitable for enclosure of the genetic element within the proteinaceous exterior (e.g., culture conditions as described herein). In some embodiments, the host cell is further incubated under conditions suitable for release of the Anelloviridae family vector (e.g., anellovector) from the host cell, e.g., into the surrounding supernatant. In some embodiments, the host cell is lysed for harvest of Anelloviridae family vector (e.g., anellovector) from the cell lysate. In some embodiments, an Anelloviridae family vector (e.g., anellovector) may be introduced to a host cell line grown to a high cell density. In some embodiments, a host cell is an Expi-293 cell.

Introduction of Genetic Elements into Host Cells

The genetic element, or a nucleic acid construct comprising the sequence of a genetic element, may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an Anelloviridae family vector (e.g., anellovector) comprising the genetic element. In some embodiments, cells are suspended in 2S Chica buffers (e.g., as described herein, e.g., in Example 20).

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the Anelloviridae family vector (e.g., anellovector). To this end, cell lines that express an Anelloviridae family vector (e.g., anellovector) polymerase protein may be utilized as appropriate host cells. Host cells may be similarly engineered to provide other viral functions or additional functions.

To prepare the Anelloviridae family vector (e.g., anellovector) disclosed herein, a genetic element construct may be used to transfect cells that provide Anelloviridae family vector (e.g., anellovector) proteins and functions required for replication and production. Alternatively, cells may be transfected with a second construct (e.g., a virus) providing Anelloviridae family vector (e.g., anellovector) proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the Anelloviridae family vector (e.g., anellovector). In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into Anelloviridae family vectors (e.g., anellovectors) using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

### Methods for Providing Protein(s) in Cis or Trans

In some embodiments (e.g., cis embodiments described herein), the genetic element construct further comprises one or more expression cassettes comprising a coding sequence for an Anelloviridae family virus ORF (e.g., an Anellovirus ORF1, ORF2, ORF2/2, ORF2/3, ORF1/1, or ORF1/2, or a CAV VP1, or a functional fragment thereof). In embodiments, the genetic element construct comprises an expression cassette comprising a coding sequence for an Anellovirus ORF1 or CAV VP1, or a splice variant or functional fragment thereof. Such genetic element constructs, which comprise expression cassettes for the effector as well as the one or more Anelloviridae family virus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis Anelloviridae family vectors (e.g., anellovectors) production methods in host cells, e.g., as described herein.

In some embodiments (e.g., trans embodiments described herein), the genetic element does not comprise an expression cassette comprising a coding sequence for one or more Anelloviridae family virus ORFs (e.g., an Anellovirus ORF1, ORF2, ORF2/2, ORF2/3, ORF1/1, or ORF1/2, or CAV VP1, or a functional fragment thereof). In embodiments, the genetic element construct does not comprise an expression cassette comprising a coding sequence for an Anellovirus ORF1 or CAV VP1, or a splice variant or functional fragment thereof. Such genetic element constructs, which comprise expression cassettes for the effector but lack expression cassettes for one or more Anelloviridae family virus ORFs (e.g., Anellovirus ORF1, CAV VP1, or a splice variant or functional fragment thereof), may be introduced into host cells. Host cells comprising such genetic element constructs may, in some instances, require additional nucleic acid constructs or integration of expression cassettes into the host cell genome for production of one or more components of the anellovector (e.g., the proteinaceous exterior proteins). In some embodiments, host cells comprising such genetic element constructs are incapable of enclosure of the genetic elements within proteinaceous exteriors in the absence of an additional nucleic construct encoding an Anellovirus ORF1 or CAV VP1 molecule. In other words, such genetic element constructs may be used for trans anellovector production methods in host cells, e.g., as described herein.

In some embodiments (e.g., cis embodiments described herein), the genetic element construct further comprises one or more expression cassettes comprising a coding sequence for one or more non-Anelloviridae family virus ORF (e.g., a non-Anellovirus or non-CAV Rep molecule, e.g., an AAV Rep molecule, e.g., an AAV Rep protein, e.g., an AAV Rep2 protein). Such genetic element constructs, which comprise expression cassettes for the effector as well as the one or more non-Anelloviridae family virus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis Anelloviridae family vector (e.g., anellovector) production methods in host cells, e.g., as described herein.

In some embodiments (e.g., trans embodiments described herein), the genetic element does not comprise an expression cassette comprising a coding sequence for one or more non-Anelloviridae family virus ORFs (e.g., a non-Anellovirus or non-CAV Rep molecule, e.g., an AAV Rep molecule, e.g., an AAV Rep protein, e.g., an AAV Rep2 protein). Such genetic element constructs, which comprise expression cassettes for the effector but lack expression cassettes for one or more non-Anelloviridae family virus ORFs (e.g., a non-Anellovirus or non-CAV Rep molecule, e.g., an AAV Rep molecule, e.g., an AAV Rep protein, e.g., an AAV Rep2 protein), may be introduced into host cells. Host cells comprising such genetic element constructs may, in some instances, require additional nucleic acid constructs or integration of expression cassettes into the host cell genome for production of one or more components of the Anelloviridae family vector (e.g., anellovector) (e.g., for replication of the genetic element). In some embodiments, host cells comprising such genetic element constructs are incapable of replicating the genetic elements in the absence of an additional nucleic construct, e.g., encoding a non-Anellovirus or non-CAV Rep molecule, e.g., an AAV Rep molecule, e.g., an AAV Rep protein, e.g., an AAV Rep2 protein. In other words, such genetic element constructs may be used for trans Anelloviridae family vector (e.g., anellovector) production methods in host cells, e.g., as described herein.

### Exemplary Cell Types

Exemplary host cells suitable for production of Anelloviridae family vector (e.g., anellovector) include, without limitation, mammalian cells, e.g., human cells and insect cells. In some embodiments, the host cell is a human cell or cell line. In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, an eye cell (e.g., a photoreceptor cell, a retinal cell, a cell of the posterior eye cup (PEC), retinal ganglion cell, a cell of the optic nerve, a cell of the optic nerve head, or a retinal pigmented epithelium (RPE) cell), a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, or hamster cell, or insect cell).

In some embodiments, the host cell is a lymphoid cell. In some embodiments, the host cell is a T cell or an immortalized T cell. In embodiments, the host cell is a Jurkat cell. In embodiments, the host cell is a MOLT cell (e.g., a MOLT-4 or a MOLT-3 cell). In embodiments, the host cell is a MOLT-4 cell. In embodiments, the host cell is a MOLT-3 cell. In some embodiments, the host cell is an acute lymphoblastic leukemia (ALL) cell, e.g., a MOLT cell, e.g., a MOLT-4 or MOLT-3 cell. In some embodiments, the host cell is a B cell or an immortalized B cell. In some embodiments, the host cell comprises a genetic element construct (e.g., as described herein).

In some embodiments, the host cell is a MOLT cell (e.g., a MOLT-4 or a MOLT-3 cell).

In some embodiments, the host cell is an acute lymphoblastic leukemia (ALL) cell, e.g., a MOLT cell, e.g., a MOLT-4 or MOLT-3 cell.

In some embodiments, the host cell is an Expi-293 cell. In some embodiments, the host cell is an Expi-293F cell.

In an aspect, the present disclosure provides a method of manufacturing an Anelloviridae family vector (e.g., anellovector) comprising a genetic element enclosed in a proteinaceous exterior, the method comprising providing a MOLT-4 cell comprising an Anelloviridae family vector (e.g., anellovector) genetic element, and incubating the MOLT-4 cell under conditions that allow the Anelloviridae family vector (e.g., anellovector) genetic element to become enclosed in a proteinaceous exterior in the MOLT-4 cell. In some embodiments, the MOLT-4 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the MOLT-4 cell, e.g., from a genetic element construct (e.g., as described herein). In some embodiments, the method further comprises introducing the Anelloviridae family vector (e.g., anellovector) genetic element construct into the MOLT-4 cell.

In an aspect, the present disclosure provides a method of manufacturing an Anelloviridae family vector (e.g., anellovector) comprising a genetic element enclosed in a proteinaceous exterior, the method comprising providing a MOLT-3 cell comprising an Anelloviridae family vector (e.g., anellovector) genetic element, and incubating the MOLT-3 cell under conditions that allow the Anelloviridae family vector (e.g., anellovector) genetic element to become enclosed in a proteinaceous exterior in the MOLT-3 cell. In some embodiments, the MOLT-3 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the MOLT-3 cell, e.g., from a genetic element construct (e.g., as described herein). In some embodiments, the method further comprises introducing the Anelloviridae family vector (e.g., anellovector) genetic element construct into the MOLT-3 cell.

In some embodiments, the host cell is a human cell. In embodiments, the host cell is a HEK293T cell, HEK293F cell, A549 cell, Jurkat cell, Raji cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the Anelloviridae family vector (e.g., anellovector) is cultivated in continuous animal cell line (e.g., immortalized cell lines that can be serially propagated). According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST.

### Culture Conditions

Host cells comprising a genetic element and components of a proteinaceous exterior can be incubated under conditions suitable for enclosure of the genetic element within the proteinaceous exterior, thereby producing an Anelloviridae family vector (e.g., anellovector). Suitable culture conditions include those described, e.g., in any of Examples 4, 5, 7, 8, 9, 10, 11, or 15. In some embodiments, the host cells are incubated in liquid media (e.g., Grace's Supplemented (TNM-FH), IPL-41, TC-100, Schneider's Drosophila, SF-900 II SFM, or and EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release Anelloviridae family vectors (e.g., anellovectors) produced therein into the surrounding supernatant.

The production of Anelloviridae family vector (e.g., anellovector)-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of Anelloviridae family vectors (e.g., anellovectors).

### Harvest

Anelloviridae family vectors (e.g., anellovectors) produced by host cells can be harvested, e.g., according to methods known in the art. For example, Anelloviridae family vectors (e.g., anellovectors) released into the surrounding supernatant by host cells in culture can be harvested from the supernatant (e.g., as described in Example 4). In some embodiments, the supernatant is separated from the host cells to obtain the Anelloviridae family vectors (e.g., anellovectors). In some embodiments, the host cells are lysed before or during harvest. In some embodiments, the Anelloviridae family vectors (e.g., anellovectors) are harvested from the host cell lysates (e.g., as described in Example 10). In some embodiments, the Anelloviridae family vectors (e.g., anellovectors) are harvested from both the host cell lysates and the supernatant. In some embodiments, the purification and isolation of Anelloviridae family vectors (e.g., anellovectors) is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the Anelloviridae family vector (e.g., anellovector) may be harvested and/or purified by separation of solutes based on biophysical properties, e.g., ion exchange chromatography or tangential flow filtration, prior to formulation with a pharmaceutical excipient.

### In Vitro Assembly Methods

An Anelloviridae family vector (e.g., anellovector) may be produced, e.g., by in vitro assembly, e.g., in a cell-free suspension or in a supernatant. In some embodiments, the genetic element is contacted to an ORF1 molecule in vitro, e.g., under conditions that allow for assembly.

In some embodiments, baculovirus constructs are used to produce Anelloviridae family virus (e.g., Anellovirus or CAV) proteins. These proteins may then be used, e.g., for in vitro assembly to encapsidate a genetic element, e.g., a genetic element comprising RNA. In some embodiments, a polynucleotide encoding one or more Anelloviridae family virus (e.g., Anellovirus or CAV) protein is fused to a promoter for expression in a host cell, e.g., an insect or animal cell. In some embodiments, the polynucleotide is cloned into a baculovirus expression system. In some embodiments, a host cell, e.g., an insect cell is infected with the baculovirus expression system and incubated for a period of time. In some embodiments, an infected cell is incubated for about 1, 2, 3, 4, 5, 10, 15, or 20 days. In some embodiments, an infected cell is lysed to recover the Anelloviridae family virus (e.g., Anellovirus or CAV) protein.

In some embodiments, an isolated Anelloviridae family virus (e.g., Anellovirus or CAV) protein is purified. In some embodiments, an Anellovirus protein is purified using purification techniques including but not limited to chelating purification, heparin purification, gradient sedimentation purification, and/or SEC purification. In some embodiments, a purified Anelloviridae family virus (e.g., Anellovirus or CAV) protein is mixed with a genetic element to encapsidate the genetic element, e.g., a genetic element comprising RNA. In some embodiments, a genetic element is encapisdated using an ORF1 protein, ORF2 protein, or modified version thereof. In some embodiments two nucleic acids are encapsidated. For instance, the first nucleic acid may be an mRNA e.g., chemically modified mRNA, and the second nucleic acid may be DNA.

In some embodiments, DNA encoding Anellovirus (AV) ORF1 (e.g., wildtype ORF1 protein, ORF1 proteins harboring mutations, e.g., to improve assembly efficiency, yield or stability, chimeric ORF1 protein, or fragments thereof) or CAV VP1 are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding AV ORF1 or CAV VP1 may be untagged. In some embodiments, DNA encoding AV ORF1 or CAV VP1 may contain tags fused N-terminally and/or C-terminally. In some embodiments, DNA encoding AV ORF1 or CAV VP1 may harbor mutations, insertions or deletions within the ORF1 or VP1 protein to introduce a tag, e.g., to aid in purification and/or identity determination, e.g., through immunostaining assays (including but not limited to ELISA or Western Blot). In some embodiments, DNA encoding AV ORF1 or CAV VP1 may be expressed alone or in combination with any number of helper proteins. In some embodiments, DNA encoding AV ORF1 is expressed in combination with AV ORF2 and/or ORF3 proteins.

In some embodiments, ORF1 or VP1 proteins harboring mutations to improve assembly efficiency may include, but are not limited to, ORF1 or VP1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 or VP1 proteins harboring mutations that improve stability may include mutations to an interprotomer contacting beta strands F and G of the canonical jellyroll beta-barrel to alter hydrophobic state of the protomer surface and improve thermodynamic favorability of capsid formation.

In some embodiments, chimeric ORF1 or VP1 proteins may include, but are not limited to, ORF1 or VP1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 or VP1 proteins may also include ORF1 or VP1 proteins which have a portion or portions of their sequence replaced with comparable portions of another AV ORF1 or CAV VP1 protein (e.g., jellyroll fragments or the C-terminal portion of Ring 2 ORF1 replaced with comparable portions of Ring 9 ORF1).

In some embodiments, the present disclosure describes a method of making an anellovector, the method comprising: (a) providing a mixture comprising: (i) a genetic element comprising RNA, and (ii) an ORF1 molecule or VP1 molecule; and (b) incubating the mixture under conditions suitable for enclosing the genetic element within a proteinaceous exterior comprising the ORF1 molecule or VP1 molecule, thereby making an anellovector; optionally wherein the mixture is not comprised in a cell. In some embodiments, the method further comprises, prior to the providing of (a), expressing the ORF1 molecule or VP1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule or VP1 molecule under conditions suitable for producing the ORF1 molecule or VP1 molecule. In some embodiments, the method further comprises, prior to the providing of (a), purifying the ORF1 molecule or VP1 molecule expressed by the host cell. In some embodiments, the method is performed in a cell-free system. In some embodiments, the present disclosure describes a method of manufacturing an anellovector composition, comprising: (a) providing a plurality of anellovectors or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anellovectors, e.g., as a pharmaceutical composition suitable for administration to a subject, e.g., if one or more of the parameters of (b) meet a specified threshold.

### Enrichment and Purification

Harvested Anelloviridae family vectors can be purified and/or enriched, e.g., to produce an anellovector preparation. In some embodiments, the harvested anellovectors are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested Anelloviridae family vectors are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### III. Vectors

The genetic element described herein may be included in a vector. Suitable vectors as well as methods for their manufacture and their use are well known in the prior art.

In one aspect, the invention includes a vector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid.

The genetic element or any of the sequences within the genetic element can be obtained using any suitable method. Various recombinant methods are known in the art, such as, for example screening libraries from cells harboring viral sequences, deriving the sequences from a vector known to include the same, or isolating directly from cells and tissues containing the same, using standard techniques. Alternatively or in combination, part or all of the genetic element can be produced synthetically, rather than cloned.

In some embodiments, the vector includes regulatory elements, nucleic acid sequences homologous to target genes, and various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector is substantially non-pathogenic and/or substantially non-integrating in a host cell or is substantially non-immunogenic in a host.

In some embodiments, the vector is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

The Anelloviridae family vector, anellovector, or other vector described herein may also be included in pharmaceutical compositions with a pharmaceutical excipient, e.g., as described herein. In some embodiments, the pharmaceutical composition comprises at least 10⁵, 10⁶, 10⁷, 10¹, 10¹, 10¹⁰, 10¹¹, 10¹², 10¹³, 10¹⁴, or 10¹⁵ Anelloviridae family vectors. In some embodiments, the pharmaceutical composition comprises about 10⁵-10¹⁵, 10⁵-10¹⁰, or 10¹⁰-10⁵ Anelloviridae family vectors. In some embodiments, the pharmaceutical composition comprises about 10′ (e.g., about 10⁵, 10⁶, 10⁷, 10⁸, 10⁹, or 10¹⁰) genomic equivalents/mL of the Anelloviridae family vector. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹³, 10⁵-10¹⁴, 10⁵-10¹⁵, or 10¹⁰-10¹⁵ genomic equivalents/mL of the Anelloviridae family vector, e.g., as determined according to the method of Example 18. In some embodiments, the pharmaceutical composition comprises sufficient Anelloviridae family vectors to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the Anelloviridae family vectors per cell to a population of the eukaryotic cells. In some embodiments, the pharmaceutical composition comprises sufficient Anelloviridae family vectors to deliver at least about 1×10⁴, 1×10⁵, 1×10⁶, 1×10⁵ or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the Anelloviridae family vectors per cell to a population of the eukaryotic cells. It is understood that applicable embodiments described herein with respect to anellovectors may also be applied to Anelloviridae family vectors (e.g., a vector based on or derived from a chicken anemia virus (CAV), e.g., as described herein).

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:
a) an Anelloviridae family vector (e.g., anellovector) comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and
b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., an Anelloviridae family vector (e.g., anellovector), Anellovirus, CAV, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et al. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

### Membrane Penetrating Polypeptides

In some embodiments, the composition further comprises a membrane penetrating polypeptide (MPP) to carry the components into cells or across a membrane, e.g., cell or nuclear membrane. Membrane penetrating polypeptides that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

### Combinations

In one aspect, the Anelloviridae family vector (e.g., anellovector) or composition comprising an Anelloviridae family vector (e.g., anellovector) described herein may also include one or more heterologous moiety. In one aspect, the anellovector or composition comprising an Anelloviridae family vector (e.g., anellovector) described herein may also include one or more heterologous moiety in a fusion. In some embodiments, a heterologous moiety may be linked with the genetic element. In some embodiments, a heterologous moiety may be enclosed in the proteinaceous exterior as part of the Anelloviridae family vector (e.g., anellovector). In some embodiments, a heterologous moiety may be administered with the Anelloviridae family vector (e.g., anellovector).

In one aspect, the invention includes a cell or tissue comprising any one of the Anelloviridae family vectors (e.g., anellovectors) and heterologous moieties described herein.

In another aspect, the invention includes a pharmaceutical composition comprising an Anelloviridae family vector (e.g., anellovector) and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

### Viruses

In some embodiments, the composition may further comprise a virus as a heterologous moiety, e.g., a single stranded DNA virus, e.g., Anelloviridae family virus (e.g., Anellovirus), Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricomavirus, Rubivirus, Birnavirus, Cystovirus, Partitivirus, and Reovirus. In some embodiments, the Anelloviridae family vector (e.g., anellovector) is administered with a virus as a heterologous moiety.

In some embodiments, the heterologous moiety may comprise a non-pathogenic, e.g., symbiotic, commensal, native, virus. In some embodiments, the non-pathogenic virus is one or more Anelloviridae family vectors (e.g., anellovectors), e.g., Alphatorquevirus (TT), Betatorquevirus (TTM), and Gammatorquevirus (TTMD). In some embodiments, the Anelloviridae family vector (e.g., anellovector) may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein, e.g., as listed in Table N1-N4.

In some embodiments, the heterologous moiety may comprise one or more viruses that are identified as lacking in the subject. For example, a subject identified as having dyvirosis may be administered a composition comprising an Anelloviridae family vector (e.g., anellovector) and one or more viral components or viruses that are imbalanced in the subject or having a ratio that differs from a reference value, e.g., a healthy subject.

In some embodiments, the heterologous moiety may comprise one or more non-Anelloviridae family viruses (e.g., non-anelloviruses), e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the Anelloviridae family vector (e.g., anellovector) or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective Anelloviridae family vector (e.g., anellovector) or virus under the control of regulatory sequences within the LTR. Suitable cell lines for replicating the Anelloviridae family vectors (e.g., anellovectors) described herein include cell lines known in the art, e.g., A549 cells, which can be modified as described herein.

### Targeting Moiety

In some embodiments, the composition or Anelloviridae family vector (e.g., anellovector) described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the Anelloviridae family vector (e.g., anellovector) or genetic element. For example, a targeting moiety may include a therapeutic that interacts with a specific molecule of interest to increase, decrease or otherwise modulate its function.

### Tagging or Monitoring Moiety

In some embodiments, the composition or Anelloviridae family vector (e.g., anellovector) described herein may further comprise a tag to label or monitor the Anelloviridae family vector (e.g., anellovector) or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

### Nanoparticles

In some embodiments, the composition or Anelloviridae family vector (e.g., anellovector) described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

### Small Molecules

In some embodiments, the composition or Anelloviridae family vector (e.g., anellovector) described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organomettallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.e.g, PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007; 80:813-26) can be used. In one embodiment, the invention includes a composition comprising an antibiotic, anti-inflammatory drug, angiogenic or vasoactive agent, growth factor or chemotherapeutic agent.

### Peptides or Proteins

In some embodiments, the composition or Anelloviridae family vector (e.g., anellovector) described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such small antigen binding peptides may bind a cytosolic antigen, a nuclear antigen, an intra-organellar antigen.

In some embodiments, the composition or Anelloviridae family vector (e.g., anellovector) described herein includes a polypeptide linked to a ligand that is capable of targeting a specific location, tissue, or cell.

### Oligonucleotide Aptamers

In some embodiments, the composition or Anelloviridae family vector (e.g., anellovector) described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR), (see en.wikipedia.org/wiki/Aptamer-cite_note-10), hemin, interferon γ, vascular endothelial growth factor (VEGF), prostate specific antigen (PSA), dopamine, and the non-classical oncogene, heat shock factor 1 (HSF1).

### Peptide Aptamers

In some embodiments, the composition or Anelloviridae family vector (e.g., anellovector) described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### Additional Therapeutics

In some embodiments, the composition or Anelloviridae family vector (e.g., anellovector) described herein may be administered in combination with other methods or therapeutic regiments, including, for example, in combination with anti-angiogenic drugs, photodynamic therapy (e.g., for wet AMD), laser photocoagulation (e.g., for diabetic retinopathy and wet AMD), and intraocular pressure reducing drugs (e.g., for glaucoma).

In some embodiments, an Anelloviridae family vector as described herein is administered with (e.g., prior to, concurrently with, or after) a second therapeutic agent. In some embodiments, the second therapeutic agent comprises an anti-VEGF antibody molecule (e.g., bevacizumab, ranibizumab, or faricimab-svoa), or a functional fragment, variant, or derivative thereof, e.g., for treating a disease, disorder, or condition as described herein. In some embodiments, the second therapeutic agent comprises aflibercept, or a functional fragment, variant, or derivative thereof. In some embodiments, the second therapeutic agent comprises an anti-C4 antibody molecule, anti-C5 antibody molecule, ABCA4 protein, or RPGR protein, e.g., for treating a disease, disorder, or condition as described herein.

### V. Host Cells

The invention is further directed to a host or host cell comprising an Anelloviridae family vector (e.g., anellovector) described herein. In some embodiments, the host or host cell is a plant, insect, bacteria, fungus, vertebrate, mammal (e.g., human), or other organism or cell. In certain embodiments, as confirmed herein, provided Anelloviridae family vectors (e.g., anellovectors) infect a range of different host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the Anelloviridae family vector (e.g., anellovector) is substantially non-immunogenic in the host. The Anelloviridae family vector (e.g., anellovector) or genetic element fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) an Anelloviridae family vector (e.g., anellovector). In some embodiments, the host is a mammal, such as a human. The amount of the Anelloviridae family vector (e.g., anellovector) in the host can be measured at any time after administration. In certain embodiments, a time course of Anelloviridae family vector (e.g., anellovector) growth in a culture is determined.

In some embodiments, the Anelloviridae family vector (e.g., anellovector), e.g., an Anelloviridae family vector (e.g., anellovector) as described herein, is heritable. In some embodiments, the Anelloviridae family vector (e.g., anellovector) is transmitted linearly in fluids and/or cells from mother to child. In some embodiments, daughter cells from an original host cell comprise the Anelloviridae family vector (e.g., anellovector). In some embodiments, a mother transmits the Anelloviridae family vector (e.g., anellovector) to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the Anelloviridae family vector (e.g., anellovector) in a host cell has a transmission efficiency during meiosis of at 25%, 50%, 60%, 70%, 80%, 85%, 90%, 95%, or 99%. In some embodiments, the Anelloviridae family vector (e.g., anellovector) in a host cell has a transmission efficiency during mitosis of at least 25%, 50%, 60%, 70%, 80%, 85%, 90%, 95%, or 99%. In some embodiments, the Anelloviridae family vector (e.g., anellovector) in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the Anelloviridae family vector (e.g., anellovector), e.g., Anelloviridae family vector (e.g., anellovector) replicates within the host cell. In one embodiment, the Anelloviridae family vector (e.g., anellovector) is capable of replicating in a mammalian cell, e.g., human cell. In other embodiments, the Anelloviridae family vector (e.g., anellovector) is replication deficient or replication incompetent.

While in some embodiments the Anelloviridae family vector (e.g., anellovector) replicates in the host cell, the Anelloviridae family vector (e.g., anellovector) does not integrate into the genome of the host, e.g., with the host's chromosomes. In some embodiments, the Anelloviridae family vector (e.g., anellovector) has a negligible recombination frequency, e.g., with the host's chromosomes. In some embodiments, the Anelloviridae family vector (e.g., anellovector) has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The Anelloviridae family vectors, e.g., anellovectors, and compositions comprising Anelloviridate family vectors, e.g., anellovectors, described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. In some embodiments, an Anelloviridae family vector, e.g., anellovector, or pharmaceutical composition as described herein is administered subretinally. In some embodiments, an Anelloviridae family vector, e.g., anellovector, or pharmaceutical composition as described herein is administered intravitreally. In some embodiments, an Anelloviridae family vector, e.g., anellovector, or pharmaceutical composition as described herein is administered suprachoroidally. The anellovectors may be administered alone or formulated as a pharmaceutical composition.

It is understood that applicable embodiments described herein with respect to anellovectors may also be applied to Anelloviridae family vectors (e.g., a vector based on or derived from a chicken anemia virus (CAV), e.g., as described herein).

The Anelloviridae family vector (e.g., anellovector) may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of an Anelloviridae family vector (e.g., anellovector) or composition comprising same, e.g., as described herein, may result in delivery of a genetic element comprised by the Anelloviridae family vector (e.g., anellovector) to a target cell, e.g., in a subject.

An Anelloviridae family vector (e.g., anellovector) or composition thereof described herein, e.g., comprising an effector (e.g., an endogenous or exogenous effector), may be used to deliver the effector to a cell, tissue, or subject. In some embodiments, the Anelloviridae family vector (e.g., anellovector) or composition thereof is used to deliver the effector to the eye of a subject, e.g., a mammalian subject, e.g., a human subject. In some embodiments, the Anelloviridae family vector (e.g., anellovector) or composition thereof is used to deliver the effector to a cell of the eye of a subject, e.g., a mammalian subject, e.g., a human subject. In certain embodiments, the cell of the eye is a photoreceptor cell, a retinal cell, a cell of the posterior eye cup (PEC), retinal ganglion cell, a cell of the optic nerve, a cell of the optic nerve head, or a retinal pigmented epithelium (RPE) cell. In some embodiments, the Anelloviridae family vector (e.g., anellovector) or composition thereof is used to deliver the effector to bone marrow, blood, heart, GI or skin. Delivery of an effector by administration of an Anelloviridae family vector (e.g., anellovector) composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the Anelloviridae family vector (e.g., anellovector), or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, an Anelloviridae family vector (e.g., anellovector) or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the Anelloviridae family vector (e.g., anellovector) or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

In some embodiments, a diseases, disorder, or condition that can be treated with the Anelloviridae family vector (e.g., anellovector) described herein, or a composition comprising the Anelloviridae family vector (e.g., anellovector), is a disease of the eye.

In some embodiments, the disease of the eye is selected from the group consisting of: neovascular age-related macular degeneration (nAMD) (also known as wet AMD or WAMD), dry AMD, retinal vein occlusion (RVO), diabetic macular edema (DME), or diabetic retinopathy (DR) (in particular, wet AMD), comprising delivering to the retina or to the posterior eye cup (PEC) of said human subject a therapeutically effective amount of anti-hVEGF antigen-binding fragment. In a specific aspect, described herein are methods of treating a human subject diagnosed with nAMD, dry AMD, retinal vein occlusion (RVO), diabetic macular edema (DME), or diabetic retinopathy (DR) (in particular, wet AMD), comprising delivering to the retina or posterior eye cup (PEC) of said human subject a therapeutically effective amount of anti-hVEGF antigen-binding fragment, by administering to the intravitreal space, suprachoroidal space, subretinal space, or outer surface of the sclera in the eye of said human subject (e.g., by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with nAMD, dry AMD, retinal vein occlusion (RVO), diabetic macular edema (DME), or diabetic retinopathy (DR) (in particular, wet AMD), comprising delivering to the retina or posterior eye cup (PEC) of said human subject a therapeutically effective amount of anti-hVEGF antigen-binding fragment, by the use of a suprachoroidal drug delivery device such as a microinjector. In a specific aspect, described herein are methods of treating a human subject diagnosed with neovascular age-related macular degeneration (nAMD), dry AMD, retinal vein occlusion (RVO), diabetic macular edema (DME), or diabetic retinopathy (DR) (in particular, wet AMD), comprising delivering to the retina or posterior eye cup (PEC) of said human subject a therapeutically effective amount of anti-hVEGF antigen-binding fragment, wherein the human subject has a Best-Corrected Visual Acuity (BCVA) that is ≤20/20 and ≥20/400.

In some embodiments, a disease, disorder, or condition that can be treated with the Anelloviridae family vector (e.g., anellovector) described herein, or a composition comprising such an Anelloviridae family vector, is a monogenic disease.

In some embodiments, a disease, disorder, or condition that can be treated with the Anelloviridae family vector (e.g., anellovector) described herein, or a composition comprising such an Anelloviridae family vector, is a polygenic disease (e.g., glaucoma).

In some embodiments, a disease, disorder, or condition that can be treated with the Anelloviridae family vector (e.g., anellovector) described herein, or a composition comprising such an Anelloviridae family vector, is a macular degeneration (e.g., age-related macular degeneration (AMD), Stargardt disease, or myopic macular degeneration). In certain embodiments, the macular degeneration is wet AMD. In certain embodiments, the macular degeneration is dry AMD (e.g., AMD with geographic atrophy).

In some embodiments, a disease, disorder, or condition that can be treated with the Anelloviridae family vector (e.g., anellovector) described herein, or a composition comprising such an Anelloviridae family vector, is a retinal disease. In certain embodiments, the retinal disease is an inherited retinal disease (IRD), e.g., as described in Stone et al. (2017, Ophthalmology; incorporated herein by reference with respect to diseases and disorders described therein). In certain embodiments, the retinal disease is retinitis pigmentosa (e.g., X-linked retinitis pigmentosa (XLRP).

In some embodiments, a disease, disorder, or condition that can be treated with the Anelloviridae family vector (e.g., anellovector) described herein, or a composition comprising such an Anelloviridae family vector, is a VEGF-associated disorder (e.g., a cancer, e.g., as described herein; a macular edema; or a proliferative retinopathy).

In some embodiments, the disease, disorder, or condition is selected from the group consisting of: retinal leakage, Leber congenital amaurosis (LCA) (e.g., wherein the genetic element comprises a human RPE65 sequence, e.g., a sequence encoding a human RPE65 protein, or an amino acid sequence having at least 75%, 80%, 85%, 90%, 95%, 96%, 97%, 98%, or 99% sequence identity thereto), amaurosis congenita, cone rod dystrophy, choroideremia, vitelliform macular dystrophy, hyperferritinemia-cataract syndrome, optic atrophy, XLR retinoschisis, cytomegalovirus retinitis, achromatopsia, Leber hereditary optical neuropathy, keratitis, uveitis, Grave's opthalmolopathy, diabetic retinopathy, or diabetic macular edema.

In some embodiments, a disease, disorder, or condition (e.g., as described herein) is treated by intravitreal administration of an Anelloviridae family vector as described herein. In some embodiments, a disease, disorder, or condition (e.g., as described herein) is treated by subretinal administration of an Anelloviridae family vector as described herein. In some embodiments, a disease, disorder, or condition (e.g., as described herein) is treated by suprachoroidal administration of an Anelloviridae family vector as described herein.

Examples of diseases, disorders, and conditions that can be treated with the Anelloviridae family vector (e.g., anellovector) described herein, or a composition comprising the Anelloviridae family vector (e.g., anellovector), include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the Anelloviridae family vector (e.g., anellovector) modulates (e.g., increases or decreases) an activity or function in a cell with which the Anelloviridae family vector (e.g., anellovector) is contacted. In some embodiments, the Anelloviridae family vector (e.g., anellovector) modulates (e.g., increases or decreases) the level or activity of a molecule (e.g., a nucleic acid or a protein) in a cell with which the Anelloviridae family vector (e.g., anellovector) is contacted. In some embodiments, the Anelloviridae family vector (e.g., anellovector) decreases viability of a cell, e.g., a cancer cell, with which the Anelloviridae family vector (e.g., anellovector) is contacted, e.g., by at least about 10%, 20%, 30%, 40%, 50%, 60%, 70%, 80%, 90%, 95%, 99%, or more. In some embodiments, the Anelloviridae family vector (e.g., anellovector) comprises an effector, e.g., an miRNA, e.g., miR-625, that decreases viability of a cell, e.g., a cancer cell, with which the Anelloviridae family vector (e.g., anellovector) is contacted, e.g., by at least about 10%, 20%, 30%, 40%, 50%, 60%, 70%, 80%, 90%, 95%, 99%, or more.

In some embodiments, the Anelloviridae family vector (e.g., anellovector) increases apoptosis of a cell, e.g., a cancer cell, e.g., by increasing caspase-3 activity, with which the Anelloviridae family vector (e.g., anellovector) is contacted, e.g., by at least about 10%, 20%, 30%, 40%, 50%, 60%, 70%, 80%, 90%, 95%, 99%, or more. In some embodiments, the Anelloviridae family vector (e.g., anellovector) comprises an effector, e.g., an miRNA, e.g., miR-625, that increases apoptosis of a cell, e.g., a cancer cell, e.g., by increasing caspase-3 activity, with which the Anelloviridae family vector (e.g., anellovector) is contacted, e.g., by at least about 10%, 20%, 30%, 40%, 50%, 60%, 70%, 80%, 90%, 95%, 99%, or more.

In some embodiments, the Anelloviridae family vector (e.g., anellovector) reduces apoptosis of a cell with which the Anelloviridae family vector (e.g., anellovector) is contacted, e.g., a cancer cell, e.g., by reducing caspase-3 activity, e.g., by at least about 10%, 20%, 30%, 40%, 50%, 60%, 70%, 80%, 90%, 95%, 99%, or more. In some embodiments, the Anelloviridae family vector (e.g., anellovector) comprises an effector, e.g., an miRNA, e.g., miR-625, that reduces apoptosis of a cell with which the Anelloviridae family vector (e.g., anellovector) is contacted, e.g., a cancer cell, e.g., by reducing caspase-3 activity, e.g., by at least about 10%, 20%, 30%, 40%, 50%, 60%, 70%, 80%, 90%, 95%, 99%, or more.

### VII. Methods of Production

### Producing the Genetic Element

Methods of making the genetic element of the Anelloviridae family vector (e.g., anellovector) are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

In some embodiments, the genetic element may be designed using computer-aided design tools. The Anelloviridae family vector (e.g., anellovector) may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the Anelloviridae family vector (e.g., anellovector). The segments or ORFs may be assembled into the Anelloviridae family vector (e.g., anellovector), e.g., in vitro recombination or unique restriction sites at 5′ and 3′ ends to enable ligation.

The genetic element can alternatively be synthesized with a design algorithm that parses the Anelloviridae family vector (e.g., anellovector) into oligo-length fragments, creating optimal design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element or segment of the genetic element may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. AnyDot.chips and methods for using them are described in part in International Publication Application Nos. WO 02088382, WO 03020968, WO 0303 1947, WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012301.

Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. The sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In some embodiments, shotgun sequencing is performed. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence.

In some embodiments, factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, or a CAV VP1, e.g., as described herein. In some embodiments, replication and/or packaging signals can be incorporated into a genetic element, for example, to induce amplification and/or encapsulation. In some embodiments, this is done both in context of larger regions of the Anelloviridae family vector (e.g., anellovector) genome (e.g., inserting effectors into a specific site in the genome, or replacing viral ORFs with effectors).

In another example, when supplied in trans, the genetic element may lack genes encoding one or more of an Anellovirus ORF1, ORF1/1, ORF1/2, ORF2, ORF2/2, ORF2/3, or ORF2t/3, or a CAV VP1, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

### In Vitro Circularization

In some instances, the genetic element to be packaged into a proteinaceous exterior is a single stranded circular DNA. The genetic element may, in some instances, be introduced into a host cell in a form other than a single stranded circular DNA. For example, the genetic element may be introduced into the host cell as a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or TraI, e.g., as described in Wawrzyniak et al. 2017, Front. Microbiol. 8: 2353; incorporated herein by reference with respect to the listed enzymes). In some embodiments, the double-stranded circular DNA is produced by in vitro circularization, e.g., as described in Example 35. Generally, in vitro circularized DNA constructs can be produced by digesting a plasmid comprising the sequence of a genetic element to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an Anelloviridae family vector (e.g., anellovector), e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Producing the Anelloviridae Family Vector (e.g., Anellovector)

The genetic elements and vectors comprising the genetic elements prepared as described herein can be used in a variety of ways to express the Anelloviridae family vector (e.g., anellovector) in appropriate host cells. In some embodiments, the genetic element and vectors comprising the genetic element are transfected in appropriate host cells and the resulting RNA may direct the expression of the Anelloviridae family vector (e.g., anellovector) gene products, e.g., non-pathogenic protein and protein binding sequence, at high levels. Host cell systems which provide for high levels of expression include continuous cell lines that supply viral functions, such as cell lines superinfected with APV or MPV, respectively, cell lines engineered to complement APV or MPV functions, etc.

In some embodiments, the Anelloviridae family vector (e.g., anellovector) is produced as described in any of Examples 1, 2, 5, 6, or 15-17.

In some embodiments, the Anelloviridae family vector (e.g., anellovector) is cultivated in continuous animal cell lines in vitro. According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines susceptible to infection with Anelloviridae family vector (e.g., anellovector) include, but are not limited to cell lines of human or primate origin, such as human or primate kidney carcinoma cell lines.

In some embodiments, the genetic elements and vectors comprising the genetic elements are transfected into cell lines that express a viral polymerase protein in order to achieve expression of the Anelloviridae family vector (e.g., anellovector). To this end, transformed cell lines that express an Anelloviridae family vector (e.g., anellovector) polymerase protein may be utilized as appropriate host cells. Host cells may be similarly engineered to provide other viral functions or additional functions.

To prepare the Anelloviridae family vector (e.g., anellovector) disclosed herein, a genetic element or vector comprising the genetic element disclosed herein may be used to transfect cells which provide Anelloviridae family vector (e.g., anellovector) proteins and functions required for replication and production. Alternatively, cells may be transfected with helper virus before, during, or after transfection by the genetic element or vector comprising the genetic element disclosed herein. In some embodiments, a helper virus may be useful to complement production of an incomplete viral particle. The helper virus may have a conditional growth defect, such as host range restriction or temperature sensitivity, which allows the subsequent selection of transfectant viruses. In some embodiments, a helper virus may provide one or more replication proteins utilized by the host cells to achieve expression of the Anelloviridae family vector (e.g., anellovector). In some embodiments, the host cells may be transfected with vectors encoding viral proteins such as the one or more replication proteins. In some embodiments, a helper virus comprises an antiviral sensitivity.

The genetic element or vector comprising the genetic element disclosed herein can be replicated and produced into Anelloviridae family vector (e.g., anellovector) particles by any number of techniques known in the art, as described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

The production of Anelloviridae family vector (e.g., anellovector)-containing cell cultures according to the present invention can be carried out in different scales, such as in flasks, roller bottles or bioreactors. The media used for the cultivation of the cells to be infected are known to the skilled person and can generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of Anelloviridae family vector (e.g., anellovector).

The purification and isolation of Anelloviridae family vector (e.g., anellovector) can be performed according to methods known by the skilled person in virus production and is described for example by Rinaldi, et al., DNA Vaccines: Methods and Protocols (Methods in Molecular Biology), 3rd ed. 2014, Humana Press.

In one aspect, the present invention includes a method for the in vitro replication and propagation of the Anelloviridae family vector (e.g., anellovector) as described herein, which may comprise the following steps: (a) transfecting a linearized genetic element into a cell line sensitive to Anelloviridae family vector (e.g., anellovector) infection; (b) harvesting the cells and isolating cells showing the presence of the genetic element; (c) culturing the cells obtained in step (b) for at least three days, such as at least one week or longer, depending on experimental conditions and gene expression; and (d) harvesting the cells of step (c).

In some embodiments, an Anelloviridae family vector (e.g., anellovector) may be introduced to a host cell line grown to a high cell density. In some embodiments, the Anelloviridae family vector (e.g., anellovector) may be harvested and/or purified by separation of solutes based on biophysical properties, e.g., ion exchange chromatography or tangential flow filtration, prior to formulation with a pharmaceutical excipient.

### VIII. Administration/Delivery

The composition (e.g., a pharmaceutical composition comprising an Anelloviridae family vector (e.g., anellovector) as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an Anelloviridae family vector (e.g., anellovector) to a subject, e.g., to an eye of a subject (e.g., to a photoreceptor, retina, posterior eye cup (PEC), retinal ganglion, optic nerve, optic nerve head, subretinal space, intravitreal space, or retinal pigmented epithelium (RPE) of the subject). The method includes administering a pharmaceutical composition comprising an Anelloviridae family vector (e.g., anellovector) as described herein to the subject, e.g., to an eye of a subject (e.g., to a photoreceptor, retina, posterior eye cup (PEC), retinal ganglion, optic nerve, optic nerve head, subretinal space, intravitreal space, or retinal pigmented epithelium (RPE) of the subject). In some embodiments, the administered Anelloviridae family vector (e.g., anellovector) replicates in the subject (e.g., becomes a part of the virome of the subject).

In some embodiments, the method of delivering an Anelloviridae family vector (e.g., anellovector) to a subject comprises contacting the Anelloviridae family vector (e.g., anellovector) to any suitable ocular cell. Ocular cells associated with age-related macular degeneration include, but are not limited to, cells of neural origin, cells of all layers of the retina, especially retinal pigment epithelial cells, glial cells, and pericytes. Other ocular cells that can be contacted as a result of the inventive method include, for example, endothelial cells, iris epithelial cells, corneal cells, ciliary epithelial cells, Mueller cells, astrocytes, muscle cells surrounding and attached to the eye (e.g., cells of the lateral rectus muscle), fibroblasts (e.g., fibroblasts associated with the episclera), orbital fat cells, cells of the sclera and episclera, connective tissue cells, muscle cells, and cells of the trabecular meshwork. Other cells linked to various ocular-related diseases include, for example, fibroblasts and vascular endothelial cells.

Generally, the vector can be delivered in the form of a suspension injected intraocularly (subretinally) under direct observation using an operating microscope. This procedure may involve vitrectomy followed by injection of vector suspension using a fine cannula through one or more small retinotomies into the subretinal space.

Briefly, an infusion cannula can be sutured in place to maintain a normal globe volume by infusion (of e.g. saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of vector, and (3) its controlled removal reduces the possibility of retinal tears and unplanned retinal detachment.

In some embodiments, the vector is directly injected into the subretinal space outside the central retina, by utilizing a cannula of the appropriate bore size (e.g. 27-45 gauge), thus creating a bleb in the subretinal space. In other embodiments, the subretinal injection of vector suspension is preceded by subretinal injection of a small volume (e.g. about 0.1 to about 0.5 ml) of an appropriate fluid (such as saline or Ringer's solution) into the subretinal space outside the central retina. This initial injection into the subretinal space establishes an initial fluid bleb within the subretinal space, causing localized retinal detachment at the location of the initial bleb. This initial fluid bleb can facilitate targeted delivery of vector suspension to the subretinal space (by defining the plane of injection prior to vector delivery), and minimize possible vector administration into the choroid and the possibility of vector injection or reflux into the vitreous cavity. In some embodiments, this initial fluid bleb can be further injected with fluids comprising one or more vector suspensions and/or one or more additional therapeutic agents by administration of these fluids directly to the initial fluid bleb with either the same or additional fine bore cannulas.

Intraocular administration of the vector suspension and/or the initial small volume of fluid can be performed using a fine bore cannula (e.g. 21-A5 gauge) attached to a syringe. In some embodiments, the plunger of this syringe may be driven by a mechanised device, such as by depression of a foot pedal. The fine bore cannula is advanced through the sclerotomy, across the vitreous cavity and into the retina at a site pre-determined in each subject according to the area of retina to be targeted (but outside the central retina). Under direct visualisation the vector suspension is injected mechanically under the neurosensory retina causing a localised retinal detachment with a self-sealing non-expanding retinotomy. As noted above, the vector can be either directly injected into the subretinal space creating a bleb outside the central retina or the vector can be injected into an initial bleb outside the central retina, causing it to expand (and expanding the area of retinal detachment). In some embodiments, the injection of vector suspension is followed by injection of another fluid into the bleb.

Without wishing to be bound by theory, the rate and location of the subretinal injection(s) can result in localized shear forces that can damage the macula, fovea and/or underlying RPE cells. The subretinal injections may be performed at a rate that minimizes or avoids shear forces. In some embodiments, the vector is injected over about 15-17 minutes. In some embodiments, the vector is injected over about 17-20 minutes. In some embodiments, the vector is injected over about 20-22 minutes. In some embodiments, the vector is injected at a rate of about 35 to about 65 μl/ml. In some embodiments, the vector is injected at a rate of about 35 μl/ml. In some embodiments, the vector is injected at a rate of about 40 μl/ml. In some embodiments, the vector is injected at a rate of about 45 μl/ml. In some embodiments, the vector is injected at a rate of about 50 W/ml. In some embodiments, the vector is injected at a rate of about 55 μl/ml. In some embodiments, the vector is injected at a rate of about 60 μl/ml. In some embodiments, the vector is injected at a rate of about 65 μl/ml. One of ordinary skill in the art would recognize that the rate and time of injection of the bleb may be directed by, for example, the volume of the vector or size of the bleb necessary to create sufficient retinal detachment to access the cells of central retina, the size of the cannula used to deliver the vector, and the ability to safely maintain the position of the canula of the invention.

One or multiple (e.g. 2, 3, or more) blebs can be created. Generally, the total volume of bleb or blebs created by the methods and systems of the invention can not exceed the fluid volume of the eye, for example about 4 ml in a typical human subject. The total volume of each individual bleb is preferably at least about 0.3 ml, and more preferably at least about 0.5 ml in order to facilitate a retinal detachment of sufficient size to expose the cell types of the central retina and create a bleb of sufficient dependency for optimal manipulation. One of ordinary skill in the art will appreciate that in creating the bleb according to the methods and systems of the invention that the appropriate intraocular pressure must be maintained in order to avoid damage to the ocular structures. The size of each individual bleb may be, for example, about 0.5 to about 1.2 ml, about 0.8 to about 1.2 ml, about 0.9 to about 1.2 ml, about 0.9 to about 1.0 ml, about 1.0 to about 2.0 ml, about 1.0 to about 3.0 ml. Thus, in one example, to inject a total of 3 ml of vector suspension, 3 blebs of about 1 ml each can be established. The total volume of all blebs in combination may be, for example, about 0.5 to about 3.0 ml, about 0.8 to about 3.0 ml, about 0.9 to about 3.0 ml, about 1.0 to about 3.0 ml, about 0.5 to about 1.5 ml, about 0.5 to about 1.2 ml, about 0.9 to about 3.0 ml, about 0.9 to about 2.0 ml, about 0.9 to about 1.0 ml.

In order to safely and efficiently transduce areas of target retina (e.g. the central retina) outside the edge of the original location of the bleb, the bleb may be manipulated to reposition the bleb to the target area for transduction. Manipulation of the bleb can occur by the dependency of the bleb that is created by the volume of the bleb, repositioning of the eye containing the bleb, repositioning of the head of the human with an eye or eyes containing one or more blebs, and/or by means of a fluid-air exchange. This is particularly relevant to the central retina since this area typically resists detachment by subretinal injection. In some embodiments fluid-air exchange is utilized to reposition the bleb; fluid from the infusion cannula is temporarily replaced by air, e.g. from blowing air onto the surface of the retina. As the volume of the air displaces vitreous cavity fluid from the surface of the retina, the fluid in the vitreous cavity may flow out of a cannula. The temporary lack of pressure from the vitreous cavity fluid causes the bleb to move and gravitate to a dependent part of the eye. By positioning the eye globe appropriately, the bleb of subretinal vector is manipulated to involve adjacent areas (e.g. the macula and/or fovea). In some cases, the mass of the bleb is sufficient to cause it to gravitate, even without use of the fluid-air exchange. Movement of the bleb to the desired location may further be facilitated by altering the position of the subject's head, so as to allow the bleb to gravitate to the desired location in the eye. Once the desired configuration of the bleb is achieved, fluid is returned to the vitreous cavity. The fluid is an appropriate fluid, e.g., fresh saline. Generally, the subretinal vector may be left in situ without retinopexy to the retinotomy and without intraocular tamponade, and the retina will spontaneously reattach within about 48 hours.

The composition is administered directly to the eye of a mammal, such as, for example, a mouse, a rat, a non-human primate, or a human. Any administration route is appropriate so long as the composition contacts an appropriate ocular cell. The composition can be appropriately formulated and administered in the form of an injection, eye lotion, ointment, implant, and the like. The composition can be administered, for example, topically, intracamerally, subconjunctivally, intraocularly, retrobulbarly, periocularly (e.g., subtenon delivery), subretinally, or suprachoroidally. Topical formulations are well known in the art. Patches, corneal shields (see, e.g., U.S. Pat. No. 5,185,152), ophthalmic solutions (see, e.g., U.S. Pat. No. 5,710,182), and ointments also are known in the art and can be used in the context of the inventive method. The composition also can be administered non-invasively using a needleless injection device, such as the Biojector 2000 Needle-Free Injection Management System™ available from Bioject Medical Technologies Inc. (Tigard, Oreg.).

Alternatively, the composition can be administered using invasive procedures, such as, for instance, intravitreal injection or subretinal injection, optionally preceded by a vitrectomy, or periocular (e.g., subtenon) delivery. The composition can be injected into different compartments of the eye, e.g., the vitreal cavity or anterior chamber. Preferably, the composition is administered intravitreally, most preferably by intravitreal injection.

In some embodiments, the composition may be administered using an ocular delivery system comprising the use of a microneedle (U.S. Pat. No. 8,808,225, incorporated herein in its entirety).

The pharmaceutical composition may include wild-type or native viral elements and/or modified viral elements. The Anelloviridae family vector (e.g., anellovector) may include one or more of the sequences (e.g., nucleic acid sequences or nucleic acid sequences encoding amino acid sequences thereof) in any of Tables N1-N4 or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any of Tables N1-N4. The Anelloviridae family vector (e.g., anellovector) may comprise a nucleic acid molecule comprising a nucleic acid sequence with at least about 60%, 65%, 70%, 75%, 80%, 85%, 90% 95%, 96%, 97%, 98% and 99% sequence identity to one or more of the sequences in any of Tables N1-N4. The Anelloviridae family vector (e.g., anellovector) may comprise a nucleic acid molecule encoding an amino acid sequence with at least about 60%, 65%, 70%, 75%, 80%, 85%, 90% 95%, 96%, 97%, 98% and 99% sequence identity to any one of the amino acid sequences in Table A1 or A2. The Anelloviridae family vector (e.g., anellovector) may comprise a polypeptide comprising an amino acid sequence with at least about 60%, 65%, 70%, 75%, 80%, 85%, 90% 95%, 96%, 97%, 98% and 99% sequence identity to any one of the amino acid sequences in Table A1 or A2. The Anelloviridae family vector (e.g., anellovector) may include one or more of the sequences in Table A1 or A2 or N1-N4, or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any of Tables N1-N4.

In some embodiments, the Anelloviridae family vector (e.g., anellovector) is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the Anelloviridae family vector (e.g., anellovector) is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the Anelloviridae family vector (e.g., anellovector) inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising an Anelloviridae family vector (e.g., anellovector) described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the Anelloviridae family vector (e.g., anellovector) is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the Anelloviridae family vector (e.g., anellovector) is sufficient to compete with chronic or acute viral infection. In certain embodiments, the Anelloviridae family vector (e.g., anellovector) may be administered prophylactically to protect from viral infections (e.g. a provirotic). In some embodiments, the Anelloviridae family vector (e.g., anellovector) is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

Pharmaceutical compositions suitable for internal use include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants such as polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HITAB), polyoxyl 10 lauryl ether, Brij 721 r, bile salts (sodium deoxycholate, sodium cholate), pluronic acids (F-68, F-127), polyoxyl castor oil (Cremophor™) nonylphenol ethoxylate (Tergitol™), cyclodextrins and, ethylbenzethonium chloride (Hyarnine™) Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof

In one aspect, active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated by reference herein.

### Ocular Delivery Systems

In some embodiments, a composition (e.g., an Anelloviridae family vector (e.g., anellovector) or pharmaceutical composition) or method described herein involves an ocular delivery system, such as the Orbit Subretinal Delivery System. Briefly, such a delivery system may comprise a cannula to be inserted into the eye for delivering the Anelloviridae family vector (e.g., anellovector) into the eye, a device body for delivering saline solution or Anelloviridae family vector (e.g., anellovector) to the cannula, a first line for delivering the saline solution to the device body, and a second line for delivering the Anelloviridae family vector (e.g., anellovector) to the device body. More particularly, in some embodiments, the delivery system is provided as three “sets”. The first set is a subretinal injection device set comes with a subretinal injection device, which comprises a cannula tip/needle, a needle advancement knob, a subretinal injection device body (with a magnet), a dose line luer, and a BSS line luer. The system can also comprise with a magnetic pad and an ophthalmic marker. The magnet provides stabilization during injection. The second set (which can be referred to as the tubing set) includes tubing assembly, a BSS syringe, two syringe snap collars, and a CPC adapter. The third set (which can be referred to as the dosing set) comprises a dose syringe and a tubing clamp.

For the subretinal injection device, the internal needle is connected to the needle advancement knob, which is connected to the subretinal injection device body. This has two lines, each attaching to either the BSS line luer or the dose line luer.

Accordingly, in some embodiments, a composition (e.g., an Anelloviridae family vector (e.g., anellovector) or pharmaceutical composition) described herein is situated in an ocular delivery system.

The ocular delivery system may comprise, for example:
a) a cannula comprising a first end and a second end, wherein the first end of the cannula optionally comprises a needle,
b) a device body which optionally comprises a magnet, wherein the second end of the cannula is operably connected to the device body,
c) needle advancement knob situated on the device body, wherein the needle advancement knob can be adjusted by a user to advance the needle to extend beyond the first end of the cannula, e.g., extend into the subretinal space;
d) a first line operably attached to the connected to device body (e.g., attached with a luer), wherein the first line may be used to deliver a wash solution, e.g., a saline solution, e.g., BSS, wherein optionally the first line is operably connected to a first syringe (e.g., connected using a syringe snap collar);
e) a second line operably attached to the connected to device body (e.g., attached with a luer), wherein the second line may be used to deliver a composition (e.g., an Anelloviridae family vector (e.g., anellovector) or pharmaceutical composition) described herein, wherein optionally the second line is operably connected to a second syringe (e.g., connected using a syringe snap collar).

In some embodiments, the ocular delivery system is part of a kit. The kit may further comprise one or both of a magnetic pad and an ophthalmic marker.

In some embodiments, a method described herein comprises administering a composition described herein (e.g., an Anelloviridae family vector (e.g., anellovector) or pharmaceutical composition) using an ocular delivery system, e.g., the ocular delivery system described above. In some embodiments, the method comprises surgically preparing the eye for administration of the composition, e.g., by exposing the sclera (e.g., by conjunctival peritomy), optionally transferring ink to the sclera to create a suturing template, creating a suture loop, and creating a sclerotomy. The cannula may be inserted into the sclerotomy. The ocular delivery system may be placed. For instance, the magnetic pad may be placed on the subject's forehead, and the device body may be placed on the magnetic pad, e.g., in the same meridian as the sclerotomy. The first end of the cannula may be positioned directly above the opposite edge of the cornea. Cannulation may be performed. For instance, the suture loops may be lifted and the cannula may be passed through the suture loops. The device body may be slid toward the eye. The cannula may be inserted into the sclerotomy. The needle may be advanced into the subretinal space using the needle advancement knob. A saline solution (e.g., BSS) may be administered, e.g., using the syringe connected to the first line. The saline solution may form a visible bleb. The Anelloviridae family vector (e.g., anellovector) or pharmaceutical composition may be administered, e.g., using the syringe connected to the second line. The Anelloviridae family vector (e.g., anellovector) or pharmaceutical composition may be released into the bleb. The needle may then be retracted. The ocular delivery system may be removed from the eye.

The delivery method may also comprise one or more of the following steps.

The BSS syringe of the tubing set is attached to the delivery system via the BSS line of the subretinal injection device. A plunger is inserted into the dose syringe, followed by attachment of a sterile needle to the dose syringe.

A plunger is also inserted into the dose syringe, and a sterile needle is attached to the dose syringe. This needle is then inserted into the vial and is used to aspirate the subretinal infusate into the syringe. The needle is then removed.

The tab is then rotated into the latched position in order to prime the dose line. The dose syringe is attached to the dose line of the Subretinal injection device. The dose syringe plunger is then advanced slowly until it reaches a hard stop and a tactile click is reached. This primes the dose line and the dose syringe assures the correct subretinal dose volume is ready for injection into the subretinal area.

If using pneumatic injection, the plunger is to be rotated counterclockwise to remove the threaded rod from the BSS syringe and leave the seal in the BSS syringe. The tubing is to be inserted in the open barrel of the BSS syringe and secured in place by sliding the syringe snap collar over both components. The tubing assembly is then attached to the pneumatic source of choice (e.g., a vitrectomy machine) using the CPC adaptor, if necessary. The viscous fluid control injection pressure is to be set to 36 psi.

The provided tubing claims supplied with the subretinal delivery system are only to be used with an alternate dose syringe (not supplied in the set) that is validated for use with the Orbit SDS. The alternate syringe's labeling must indicate that it is validated for use with the Orbit SDS and include instructions for use with the Orbit SDS. If using an alternate dose syringe, the tubing clamp is placed on the dose line following priming to prevent potential backflow into the alternate dose syringe during BSS syringe use. Immediately before injecting the infusate, the tubing clamp is to be removed.

### Exemplary Surgical Steps:

The site it prepared by inserting the lid speculum, and inserting a valved port for the chandelier. The eye is rotated inferonasally to expose the superotemporal quadrant, and a conjunctival peritomy is performed to expose the sclera. A cannulation path that does not interfere with identified vortex veins or long posterior ciliary neurovascular bundles is selected. Ink is applied to the tips of the ophthalmic marker with the limbus, and gently press it against the sclera to transfer the ink. After drying the scleral surface, the marker is aligned with the limbus and is gently pressed against the sclera to transfer ink, and thereby creating the suturing template (about 10 ink dots). The suture loop is created. A sclerotomy is performed.

### Exemplary Device Placement:

The adhesive backing is removed from the magnetic pad, and the pad is placed over the sterile fenestrated drape, on top of the patient's forehead. The primed subretinal injection device body on top of the magnetic pad is placed in the same meridian as the sclerotomy. The distal tip of the subretinal injection cannula is positioned directly above the opposite edge of the cornea to ensure sufficient slack for advancement. The needle is advanced and the flow of BSS or BSS PLUS is checked. The needle is fully retracted.

### Exemplary Cannulation:

Using smooth forceps, the flexible cannula is grasped, approximately 10 mm from the distal tip. Using toothed forceps on the eye to help with insertion, the suture loops are lifted and then the posterior lip of the sclerotomy is grasped. The cannula is passed through the suture loops. Prior to insertion, the subretinal injection device body is slid toward the eye to provide additional slack and maintain a tangential path to the eye's curvature. While grapsing the center of the posterior lip of the sclerotomy and pulling away from the eye, the flexible cannula is inserted into the sclerotomy. The eye is rotated back to the neutral axis.

### Exemplary Method for Using Clearside Biomedical SCS Microinjector:

In some embodiments, a composition (e.g., an Anelloviridae family vector (e.g., anellovector) or pharmaceutical composition) or method described herein involves an ocular delivery system, such as an SCS microinjector. Briefly, such a delivery system may comprise a needle sized appropriately to deliver an Anelloviridae family vector (e.g., anellovector) to the suprachoroidal space, a chamber to contain the Anelloviridae family vector (e.g., anellovector), and a plunger to administer the Anelloviridae family vector (e.g., anellovector). In some embodiments, the microinjector is comprised of a needle of various lengths (needle length is printed on the needle—either 900 um or 1100 um). The needle is a 30 gauge needle. The needle is connected to a conjunctiva compressing hub, which is connected to a chamber (e.g., a barrel), which has a 100 uL capacity and has indicators in increments of 25 uL. The barrel is connected to a plunger and plunger handle to inject the drug. The microinjector also comes with a needle safety cap with integrated fixed length calipers of 4.5 mm.

The Clearside SCS microinjector is designed for suprachoroidal drug delivery.

Accordingly, in some embodiments, a composition (e.g., an Anelloviridae family vector (e.g., anellovector) or pharmaceutical composition) described herein is situated in an ocular delivery system. The ocular delivery system may comprise:
a) a needle, e.g., a 30 gauge needle, wherein optionally the needle is 800-1200 um (e.g., about 900 um or 1100 um in length);
b) optionally, a conjunctiva compressing hub connected to the needle;
c) a chamber connected to one or both of the conjunctiva compressing hub and the needle; and
d) optionally, a plunger connected to the chamber.

In some embodiments, the ocular delivery system is part of a kit. The kit may further comprise, one or both of a needle safety cap and calipers.

In some embodiments, a method described herein comprises administering a composition described herein (e.g., an Anelloviridae family vector (e.g., anellovector) or pharmaceutical composition) using an ocular delivery system, e.g., the ocular delivery system described above. In some embodiments, the method comprises inserting the needle into the suprachoroidal space and administering the Anelloviridae family vector (e.g., anellovector) or pharmaceutical composition into the suprachoroidal space.

### Dosing

A wide variety of assays may be utilized in order to determine appropriate dosages for administration, or to assess the ability of a gene delivery vector to treat or prevent a particular disease. Certain of these assays are discussed in more detail below.

Therapeutically effective doses of the composition or Anelloviridae family vector (e.g., anellovector) as described herein may be administered subretinally and/or intraretinally (e.g., by subretinal injection via the transvitreal approach (a surgical procedure), or subretinal administration via the suprachoroidal space) in a volume ranging from 0.1 mL to 0.5 mL, preferably in 0.1 to 030 ml (100-300 μl), and most preferably, in a volume of 025 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera (e.g., by a posterior juxtascleral depot procedure) in a volume of 500 μl or less, for example, in a volume of 10-20 μl, 20-50 μl, 50-100 μl, 100-200 μl, 200-300 μl, 300-400 μl, or 400-500 μl. Subretinal injection is a surgical procedure performed by trained retinal surgeons that involves a vitrectomy with the subject under local anesthesia, and subretinal injection of the gene therapy into the retina (see, e.g., Campochiaro et al., 2017, Hum Gen Ther 28(1):99-111, which is incorporated by reference herein in its entirety). In a specific embodiment, the subretinal administration is performed via the suprachoroidal space using a suprachoroidal catheter which injects drug into the subretinal space, such as a subretinal drug delivery device that comprises a catheter which can be inserted and tunneled through the suprachoroidal spece to the posterior pole, where a small needle injects into the subretinal space (see, e.g., Baldassarre et al. 2017, Subretinal Delivery of Cells via the Suprachoroidal Space: Janssen Trial, In: Schwartz et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. The subretinal drug delivery devices that can be used to deposit the expression vector in the subretinal space via the suprachoroidal space according to the invention described herein include, but are not limited to, subretinal drug delivery devices manufactured by Janssen Pharmaceuticals, Inc. (see, for example, International Patent Application Publication No, WO 2016/040635 A1). In a specific embodiment, administration to the outer surface of the sclera is performed by a juxtascleral drug delivery device comprising a cannula whose tip can be inserted and kept in direct apposition to the scleral surface. See Section 5.3.2 for more details of the different modes of administration. Suprachoroidal, subretinal, juxtascleral and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. Doses that maintain a concentration of the transgene product at a Cumin of at least 0.330 μg/mL in the Vitreous humour, or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 220 μg/ml should be maintained. However, because the transgene product is continuously produced, maintenance of lower concentrations can be effective. The concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

Anelloviridae family vectors can be delivered to the eye by intraocular injection into the vitreous. In this application, the injection volume of the gene delivery vector could be substantially larger, as the volume is not constrained by the anatomy of the subretinal space. Acceptable dosages in this instance can range from 25 ul to 1000 ul. In this application, the target cells to be transduced include the retinal ganglion cells, which are the retinal cells primarily affected in glaucoma.

In certain embodiments, the composition or Anelloviridae family vector encoding a transgene is administered at a dose ranging from 3×10⁷, 3×10⁸, 3×10⁹, or 3×10¹⁰ genome copies to 2.5×10¹¹, 2.5×10¹², or 2.5×10¹³ genome copies. In certain embodiments, the composition or Anelloviridae family vector encoding a transgene is administered at a dose ranging from 3×10⁹ genome copies to 25×10¹⁰ genome copies. In certain embodiments, the composition or Anelloviridae family vector encoding a transgene is administered at a dose ranging from 3×10⁹ genome copies to 2.5×10¹¹ genome copies. In certain embodiments, the composition or Anelloviridae family vector encoding a transgene is administered at a dose ranging from 3×10⁹ genome copies to 2.5×10¹² genome copies. In some embodiments, the composition or Anelloviridae family vector encoding a transgene is administered at a dose ranging from 3×10⁹ genome copies to 2.5×10¹³ genome copies.

In certain embodiments, the composition or Anelloviridae family vector encoding a transgene is administered at a dose of about 3×10⁷, 4×10⁷, 5×10⁷, 6×10⁷, 7×10⁷, 8×10⁷, or 9×10⁷ genome copies. In certain embodiments, the composition or Anelloviridae family vector encoding a transgene is administered at a dose of about 1×10⁸, 2×10⁸, 3×10⁸, 4×10⁸, 5×10⁸, 6×10⁸, 7×10⁸, 8×10⁸, or 9×10⁸ genome copies. In certain embodiments, the composition or Anelloviridae family vector encoding a transgene is administered at a dose of about 1×10⁹, 2×10⁹, 3×10⁹, 4×10⁹, 5×10⁹, 6×10⁹, 7×10⁹, 8×10⁹, or 9×10⁹ genome copies. In certain embodiments, the composition or Anelloviridae family vector encoding a transgene is administered at a dose of about 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ genome copies. In certain embodiments, the composition or Anelloviridae family vector encoding a transgene is administered at a dose of about 1×10¹¹, 2×10¹¹, 3×10¹¹, 4×10¹¹, 5×10¹¹, 6×10¹¹, 7×10¹¹, 8×10¹¹, or 9×10¹¹ genome copies. In certain embodiments, the composition or Anelloviridae family vector encoding a transgene is administered at a dose of about 1×10¹², 2×10¹², 3×10¹², 4×10¹², 5×10¹², 6×10¹², 7×10¹², 8×10¹², or 9×10¹² genome copies. In certain embodiments, the composition or Anelloviridae family vector encoding a transgene is administered at a dose of about 1×10¹³, 2×10¹³, 3×10¹³, 4×10¹³, 5×10¹³, 6×10¹³, 7×10⁴, 8×10¹³, or 9×10¹³ genome copies.

### Redosing

The Anelloviridae family vector (e.g., anellovector)s described herein can, in some instances, be used as a delivery vehicle that can be administered in multiple doses (e.g., doses administered separately). While not wishing to be bound by theory, in some embodiments, an Anelloviridae family vector (e.g., anellovector) (e.g., as described herein) induces a relatively low immune response (as measured, for example, as 50% GMT values, e.g., as observed in Example 12), e.g., allowing for repeated dosing of a subject with one or more Anelloviridae family vectors (e.g., anellovectors) (e.g., multiple doses of the same Anelloviridae family vector (e.g., anellovector) or different Anelloviridae family vectors (e.g., anellovectors)). In an aspect, the invention provides a method of delivering an effector, comprising administering to a subject a first plurality of Anelloviridae family vectors (e.g., anellovectors) and then a second plurality of Anelloviridae family vectors (e.g., anellovectors). In some embodiments, the second plurality of Anelloviridae family vectors (e.g., anellovectors) comprise the same proteinaceous exterior as the Anelloviridae family vectors (e.g., anellovectors) of the first plurality. In another aspect, the invention provides a method of selecting a subject (e.g., a human subject) to receive an effector, wherein the subject previously received, or was identified as having received, a first plurality of Anelloviridae family vectors (e.g., anellovectors) comprising a genetic element encoding an effector, in which the method involves selecting the subject to receive a second plurality of Anelloviridae family vectors (e.g., anellovectors) comprising a genetic element encoding an effector (e.g., the same effector as that encoded by the genetic element of the first plurality of Anelloviridae family vectors (e.g., anellovectors), or a different effector as that encoded by the genetic element of the first plurality of Anelloviridae family vectors (e.g., anellovectors)). In another aspect, the invention provides a method of identifying a subject (e.g., a human subject) as suitable to receive a second plurality of Anelloviridae family vectors (e.g., anellovectors), the method comprising identifying the subject has having previously received a first plurality of Anelloviridae family vectors (e.g., anellovectors) comprising a genetic element encoding an effector, wherein the subject being identified as having received the first plurality of Anelloviridae family vectors (e.g., anellovectors) is indicative that the subject is suitable to receive the second plurality of Anelloviridae family vectors (e.g., anellovectors).

In some embodiments, the second plurality of Anelloviridae family vectors (e.g., anellovectors) comprises a proteinaceous exterior with at least one surface epitope in common with the Anelloviridae family vectors (e.g., anellovectors) of the first plurality of Anelloviridae family vectors (e.g., anellovectors). In some embodiments, the Anelloviridae family vectors (e.g., anellovectors) of the first plurality and the Anelloviridae family vectors (e.g., anellovectors) of the second plurality carry genetic elements encoding the same effector. In some embodiments, the Anelloviridae family vectors (e.g., anellovectors) of the first plurality and the Anelloviridae family vectors (e.g., anellovectors) of the second plurality carry genetic elements encoding different effectors.

In some embodiments, the second plurality comprises about the same quantity and/or concentration of Anelloviridae family vectors (e.g., anellovectors) as the first plurality (e.g., when normalized to the body mass of the subject at the time of administration), e.g., the second plurality comprises 90-110%, e.g., 95-105% of the number of Anelloviridae family vectors (e.g., anellovectors) in the first plurality when normalized to body mass of the subject at the time of administration. In some embodiments, wherein the first plurality comprises a greater dosage of Anelloviridae family vectors (e.g., anellovectors) than the second plurality, e.g., wherein the first plurality comprises a greater quantity and/or concentration of Anelloviridae family vectors (e.g., anellovectors) relative to the second plurality. In some embodiments, wherein the first plurality comprises a lower dosage of Anelloviridae family vectors (e.g., anellovectors) than the second plurality, e.g., wherein the first plurality comprises a lower quantity and/or concentration of Anelloviridae family vectors (e.g., anellovectors) relative to the second plurality.

In some embodiments, the subject is evaluated between the administration of the first and second pluralities of Anelloviridae family vectors (e.g., anellovectors), e.g., for the presence (e.g., persistence) of Anelloviridae family vectors (e.g., anellovectors) from the first plurality, or progeny thereof. In some embodiments, the subject is administered the second plurality of Anelloviridae family vectors (e.g., anellovectors) if the presence of Anelloviridae family vectors (e.g., anellovectors) from the first plurality, or the progeny thereof, are not detected.

In some embodiments, the second plurality is administered to the subject at least 1, 2, 3, or 4 weeks, or 1, 2, 3, 4, 5, 6, 7, 8, 9, 10, 11, or 12 months, or 1, 2, 3, 4, 5, 10, or 20 years after the administration of the first plurality to the subject. In some embodiments, the second plurality is administered to the subject between 1-2 weeks, 2-3 weeks, 3-4 weeks, 1-2 months, 3-4 months, 4-5 months, 5-6 months, 6-7 months, 7-8 months, 8-9 months, 9-10 months, 10-11 months, 11-12 months, 1-2 years, 2-3 years, 3-4 years, 4-5 years, 5-10 years, or 10-20 years after the administration of the first plurality to the subject. In some embodiments, the method comprises administering a repeated dose of Anelloviridae family vectors (e.g., anellovectors) over the course of at least 1, 2, 3, 4, or 5 years.

In some embodiments, the method further comprises assessing, after administration of the first plurality and before administration of the second plurality, one or more of:
a) the level or activity of the effector in the subject (e.g., by detecting a protein effector, e.g., by ELISA; by detecting a nucleic acid effector, e.g., by RT-PCR, or by detecting a downstream effect of the effector, e.g., level of an endogenous gene affected by the effector);
b) the level or activity of the Anelloviridae family vector (e.g., anellovector) of the first plurality in the subject (e.g., by detecting the level of the VP1 of the Anelloviridae family vector (e.g., anellovector));
c) the presence, severity, progression, or a sign or symptom of a disease in the subject that the Anelloviridae family vector (e.g., anellovector) was administered to treat; and/or
d) the presence or level of an immune response, e.g., neutralizing antibodies, against an Anelloviridae family vector (e.g., anellovector).

In some embodiments, the method further comprises administering to the subject a third, fourth, fifth, and/or further plurality of Anelloviridae family vectors (e.g., anellovectors), e.g., as described herein.

In some embodiments, the first plurality and the second plurality are administered via the same route of administration, e.g., intravenous administration. In some embodiments, the first plurality and the second plurality are administered via different routes of administration. In some embodiments, the first and the second pluralities are administered by the same entity (e.g., the same health care provider). In some embodiments, the first and the second pluralities are administered by different entities (e.g., different health care providers). In some embodiments, one or both of the first and second pluralities are administered subretinally, intravitreally, or suprachoroidally.

### Method of Treatment

In one aspect, the present disclosure provides a method for treating disease, disorder, or condition (e.g., a disease of the eye), the method comprising administering a pharmaceutically effective amount of an Anelloviridae family vector or a pharmaceutical composition comprising an Anelloviridae family vector provided herein to a subject (e.g., a human subject) in need of such treatment. In some aspects, the disease is selected from the group of ocular neovascular diseases consisting of: age-related macular degeneration (AMD), wet-AMD, dry-AMD, retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemic retinopathy and diabetic retinal edema.

In some embodiments, a disease, disorder, or condition that can be treated with the Anelloviridae family vector (e.g., anellovector) described herein, or a composition comprising such an Anelloviridae family vector, is a monogenic disease.

In some embodiments, a disease, disorder, or condition that can be treated with the Anelloviridae family vector (e.g., anellovector) described herein, or a composition comprising such an Anelloviridae family vector, is a polygenic disease (e.g., glaucoma).

In some embodiments, a disease, disorder, or condition that can be treated with the Anelloviridae family vector (e.g., anellovector) described herein, or a composition comprising such an Anelloviridae family vector, is a macular degeneration (e.g., age-related macular degeneration (AMD), Stargardt disease, or myopic macular degeneration). In certain embodiments, the macular degeneration is wet AMD. In certain embodiments, the macular degeneration is dry AMD (e.g., AMD with geographic atrophy).

In some embodiments, a disease, disorder, or condition that can be treated with the Anelloviridae family vector (e.g., anellovector) described herein, or a composition comprising such an Anelloviridae family vector, is a retinal disease. In certain embodiments, the retinal disease is an inherited retinal disease (IRD), e.g., as described in Stone et al. (2017, Ophthalmology; incorporated herein by reference with respect to diseases and disorders described therein). In certain embodiments, the retinal disease is retinitis pigmentosa (e.g., X-linked retinitis pigmentosa (XLRP).

In some embodiments, a disease, disorder, or condition that can be treated with the Anelloviridae family vector (e.g., anellovector) described herein, or a composition comprising such an Anelloviridae family vector, is a VEGF-associated disorder (e.g., a cancer, e.g., as described herein; a macular edema; or a proliferative retinopathy).

In some embodiments, the disease, disorder, or condition is selected from the group consisting of: retinal leakage, Leber congenital amaurosis (LCA) (e.g., wherein the genetic element comprises a human RPE65 sequence, e.g., a sequence encoding a human RPE65 protein, or an amino acid sequence having at least 75%, 80%, 85%, 90%, 95%, 96%, 97%, 98%, or 99% sequence identity thereto), amaurosis congenita, cone rod dystrophy, choroideremia, vitelliform macular dystrophy, hyperferritinemia-cataract syndrome, optic atrophy, XLR retinoschisis, cytomegalovirus retinitis, achromatopsia, Leber hereditary optical neuropathy, keratitis, uveitis, Grave's opthalmolopathy, diabetic retinopathy, or diabetic macular edema.

In some cases, dry AMD may be treated. In some cases, dry AMD may be referred to as central geographic atrophy, characterized by atrophy of the retinal pigment epithelial later below the retina and subsequent loss of photoreceptors in the central part of the eye. The composition and methods of this disclosure provide for the treatment of any and all forms of AMD.

In another aspect, the present disclosure provides a method for prophylactic treatment of AMD or ocular neovascular diseases as described herein, comprising administering a pharmaceutically effective amount of the pharmaceutical compositions provided herein to a human subject in need of such treatment. The present disclosure may be used to treat patients at risk of developing AMD, or presenting early symptoms of the disease. This may include treatment of eyes either simultaneously or sequentially. Simultaneous treatment may mean that the treatment is administered to each eye at the same time or that both eyes are treated during the same visit to a treating physician or other healthcare provider. It has been documented that patients have a higher risk of developing AMD in a healthy fellow eye of an eye that presents symptoms of AMD, or in patients who have a genetic predisposition toward developing AMD. The present disclosure can be used as a prophylactic treatment in prevention of AMD in the fellow eye. While the mechanism underlying the increased risk for the progression of ocular neovascular disease in a fellow eye is unknown, there are multiple studies in the art detailing this elevated risk. For example, in one such large scale study, of 110 fellow eyes observed that progressed to advanced AMD, choroidal neovascularization (CNV) developed in 98 eyes and foveal geographic atrophy (GA) in 15 eyes. Ophthalmologica 2011; 226(3):110-8. doi: 10.1159/000329473. Curr Opin Ophthalmol. 1998 June; 9(3):38-46. No non-ocular characteristic (age, gender, history of hypertension or smoking) or ocular feature of the study eye at baseline (lesion composition, lesion size, or visual acuity) was predictive of progression to advanced AMD in this cohort. However, statistical analysis indicates that AMD symptoms of the first eye, including drusen size, focal hyperpigmentation, and nonfoveal geographic atrophy had significant independent relationships in assessing risk of developing of AMD in the fellow eye. Recent studies have indicated that of ocular characteristics, genetic factors and certain environmental factors may play a role in the increased risk of developing AMD in the fellow eye. JAMA Ophthalmol. 2013 Apr. 1; 131(4):448-55. doi: 10.1001/jamaophthalmol.2013.2578. Given the well characterized elevated risk of AMD development in untreated fellow eyes, there is need in the art of methods for preventing onset and subsequent vision loss due to the disease.

In some aspects, no vector is detected in the human subject's tear, blood, saliva or urine samples 7, 14, 21 or 30 days after administering said pharmaceutical composition. In some aspects, the presence of the viral vector is detected by qPCR or ELISA as known in the art.

In some aspects, the human subject shows no clinically significant retinal toxicity as assessed by serial ophthalmic examinations over at least about a 2, 3, 4, 5, 6, 7, 8, 9, 10, 11 or 12 month months period. In some aspects, the human subject shows no clinically significant retinal toxicity as assessed by serial ophthalmic examinations over at most about a 2, 3, 4, 5, 6, 7, 8, 9, 10, 11 or 12 month months period.

In some aspects, no superficial, anterior segment or vitreous inflammatory signs are present in the human subject over at least a two months period. In some cases, no superficial, anterior segment or vitreous inflammatory signs are present in the human subject at 1 week or at 3, 6, 9 or 12 months after administration of the pharmaceutical composition.

In some aspects, there is no evidence of visual acuity loss, TOP elevation, retinal detachment, or any intraocular or systemic immune response in said human subject at least 120 days post administration.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents

Example 1: Immunologic effects of Anellovectors (Anellovector A): in vivo effector function, e.g., expression of the miRNA, of the anellovector after administration
Example 2: Identification and use of protein binding sequences: putative protein-binding sites in the Anellovirus genome
Example 3: Replication-deficient anellovectors and helper viruses
Example 4: Manufacturing process for replication-competent anellovectors
Example 5: Manufacturing process of replication-deficient anellovectors: recovery and scaling up of production of replication-deficient anellovectors
Example 6: Production of anellovectors using suspension cells: production of anellovectors in cells in suspension.
Example 7: Quantification of anellovector genome equivalents by qPCR: development of a hydrolysis probe-based quantitative PCR assay to quantify anellovectors
Example 8: Functional effects of an anellovector expressing an exogenous microRNA sequence: use of an anellovector to express a functional nucleic acid effector
Example 9: Characterization of regions required for anellovector development
Example 10: Anellovector delivery of exogenous proteins in vivo: This example demonstrates in vivo effector function (e.g. expression of proteins) of anellovectors after administration
Example 11: Tandem copies of the Anellovirus genome
Example 12: In vitro circularized Anellovirus genomes: constructs comprising circular, double stranded Anelloviral genome DNA with minimal non-viral DNA
Example 13: Production of anellovectors containing chimeric ORF1 with hypervariable domains from different Torque Teno Virus strains
Example 14: Design of an anellovector harboring a DNA payload
Example 15: Transduction of Anellovector-encoding antibody transgene
Example 16: Anellovectors based on tth8 and LY2 each successfully transduced the EPO gene into lung cancer cells
Example 17: Anellovectors with therapeutic transgenes can be detected in vivo after intravenous (i.v.) administration
Example 18: In vitro circularized genome as input material for producing anellovectors in vitro
Example 19: Rescue of recombinant Ring 19 human Anelloviruses
Example 20: Production and purification of Anelloviruses using MOLT-4 cells
Example 21: Ring19 particles demonstrated infectivity in vivo
Example 22. Ring 2 infectivity in retina and PEC following subretinal and intravitreal injection
Example 23. CAV infectivity and transduction in retina and PEC following subretinal and intravitral injection

### Example 1: Immunologic Effects of Anellovectors (Anellovector A)

This example describes in vivo effector function, e.g., expression of the miRNA, of the anellovector after administration.

Purified anellovectors prepared as described herein are intravenously administered to healthy pigs at various doses using hundred-fold dilutions starting from 10¹⁴ genome equivalents per kilogram down to 0 genome equivalents per kilogram. In order to evaluate the effects on immune tolerance, pigs are injected daily for 3 days with anellovectors or vehicle control PBS and sacrificed after 3 days.

Spleen, bone marrow and lymph nodes are harvested. Single cell suspensions are prepared from each of the tissues and stained with extracellular markers for MHC-II, CD11c, and intracellular IFN. MHC+, CD11c+, IFN+ antigen presenting cells are analyzed via flow cytometry from each tissue, e.g., wherein a cell that is positive for a given one of the above-mentioned markers is a cell that exhibits higher fluorescence than 99% of cells in a negative control population that lack expression of the marker but is otherwise similar to the assay population of cells, under the same conditions.

In an embodiment, a decreased number of IFN+ cells in the anellovector treatment group compared to the control will indicate that the anellovectors decrease IFN production in cells after administration.

### Example 2: Identification and Use of Protein Binding Sequences

This example describes putative protein-binding sites in the Anellovirus genome, which can be used for amplifying and packaging effectors, e.g., in an anellovector as described herein. In some instances, the protein-binding sites may be capable of binding to an exterior protein, such as a capsid protein.

Two conserved domains within the Anellovirus genome are putative origins of replication: the 5′ UTR conserved domain (5CD) and the GC-rich domain (GCR) (de Villiers et al., Journal of Virology 2011; Okamoto et al., Virology 1999). In one example, in order to confirm whether these sequences act as DNA replication sites or as capsid packaging signals, deletions of each region are made in plasmids harboring an Anellovirus sequence. A539 cells are transfected with the deletion constructs. Transfected cells are incubated for four days, and then virus is isolated from supernatant and cell pellets. A549 cells are infected with virus, and after four days, virus is isolated from the supernatant and infected cell pellets. qPCR is performed to quantify viral genomes from the samples. Disruption of an origin of replication prevents viral replicase from amplifying viral DNA and results in reduced viral genomes isolated from transfected cell pellets compared to wild-type virus. A small amount of virus is still packaged and can be found in the transfected supernatant and infected cell pellets. In some embodiments, disruption of a packaging signal will prevent the viral DNA from being encapsulated by capsid proteins. Therefore, in embodiments, there will still be an amplification of viral genomes in the transfected cells, but no viral genomes are found in the supernatant or infected cell pellets.

In a further example, in order to characterize additional replication or packaging signals in the DNA, a series of deletions across the entire TTMV-LY2 genome is used. Deletions of 100 bp are made stepwise across the length of the sequence. Plasmids harboring Anellovirus genome deletions are transfected into A549 and tested as described above. In some embodiments, deletions that disrupt viral amplification or packaging will contain potential cis-regulatory domains.

Replication and packaging signals can be incorporated into effector-encoding DNA sequences (e.g., in a genetic element in an anellovector) to induce amplification and encapsulation. This is done both in context of larger regions of the anellovector genome (i.e., inserting effectors into a specific site in the genome, or replacing viral ORFs with effectors, etc.), or by incorporating minimal cis signals into the effector DNA. In cases where the anellovector lacks trans replication or packaging factors (e.g., replicase and capsid proteins, etc.), the trans factors are supplied by helper genes. The helper genes express all of the proteins and RNAs sufficient to induce amplification and packaging, but lack their own packaging signals. The anellovector DNA is co-transfected with helper genes, resulting in amplification and packaging of the effector but not of the helper genes.

### Example 3: Replication-Deficient Anellovectors and Helper Viruses

For replication and packaging of an anellovector, some elements (e.g., an ORF1, ORF1/1, ORF1/2, ORF2, ORF2/2, ORF2/3, and/or ORF2t/3 molecule, or a nucleic acid sequence encoding same) can be provided in trans. These include proteins or non-coding RNAs that direct or support DNA replication or packaging. Trans elements can, in some instances, be provided from a source alternative to the anellovector, such as a helper virus, plasmid, or from the cellular genome.

Other elements are typically provided in cis (e.g., a TATA box, cap site, initiator element, transcriptional start site, 5′ UTR conserved domain, three open-reading frame region, poly(A) signal, or GC-rich region). These elements can be, for example, sequences or structures in the anellovector DNA that act as origins of replication (e.g., to allow amplification of anellovector DNA) or packaging signals (e.g., to bind to proteins to load the genome into the capsid). Generally, a replication deficient virus or anellovector will be missing one or more of these elements, such that the DNA is unable to be packaged into an infectious virion or anellovector even if other elements are provided in trans.

Replication deficient viruses can be useful for controlling replication of an anellovector (e.g., a replication-deficient or packaging-deficient anellovector) in the same cell. In some instances, the virus will lack cis replication or packaging elements, but express trans elements such as proteins and non-coding RNAs. Generally, the therapeutic anellovector would lack some or all of these trans elements and would therefore be unable to replicate on its own, but would retain the cis elements. When co-transfected/infected into cells, the replication-deficient virus would drive the amplification and packaging of the anellovector. The packaged particles collected would thus be comprised solely of therapeutic anellovector, without contamination from the replication-deficient virus.

To develop a replication deficient anellovector, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of anellovector DNA amplification within the cell, e.g., as measured by qPCR, but will support some infectious anellovector production, e.g., as monitored by assays on infected cells that can include any or all of qPCR, western blots, fluorescence assays, or luminescence assays. Successful deletion of a packaging element will not disrupt anellovector DNA amplification, so an increase in anellovector DNA will be observed in transfected cells by qPCR. However, the anellovector genomes will not be encapsulated, so no infectious anellovector production will be observed.

### Example 4: Manufacturing Process for Replication-Competent Anellovectors

This example describes a method for recovery and scaling up of production of replication-competent anellovectors. Anellovectors are replication competent when they encode in their genome all the required nucleic acid elements and ORFs necessary to replicate in cells. Since these anellovectors are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins).

In this example, double-stranded DNA encoding the full sequence of a synthetic anellovector either in its linear or circular form is introduced into 5E+05 adherent mammalian cells in a T75 flask by chemical transfection or into 5E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g. DNAse I, Benzonase), is added and incubated at 25-37° C. for 0.5-4 hours. Anellovector suspension is centrifuged at 1000×g for 10 minutes at 4° C. The clarified supernatant is transferred to a new tube and diluted 1:1 with a cryoprotectant buffer (also known as stabilization buffer) and stored at −80° C. if desired. This produces passage 0 of the anellovector (P0). To bring the concentration of detergent below the safe limit to be used on cultured cells, this inoculum is diluted at least 100-fold or more in serum-free media (SFM) depending on the anellovector titer.

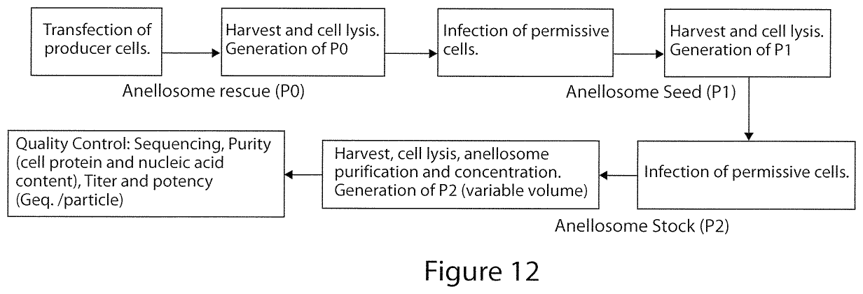

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of anellovector (P1). Depending on the titer of the stock, it is diluted no less than 100-fold in SFM and added to cells grown on multilayer flasks of the required size. Multiplicity of infection (MOI) and time of incubation is optimized at smaller scale to ensure maximal anellovector production. After harvest, anellovectors may then be purified and concentrated as needed. A schematic showing a workflow, e.g., as described in this example, is provided in FIG. 12.

### Example 5: Manufacturing Process of Replication-Deficient Anellovectors

This example describes a method for recovery and scaling up of production of replication-deficient anellovectors.

Anellovectors can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient anellovectors can be grown in a complementing cell line. Such cell line constitutively expresses components that promote anellovector growth but that are missing or nonfunctional in the genome of the anellovector.

In one example, the sequence(s) of any ORF(s) involved in anellovector propagation are cloned into a lentiviral expression system suitable for the generation of stable cell lines that encode a selection marker, and lentiviral vector is generated as described herein. A mammalian cell line capable of supporting anellovector propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered anellovector, and hence to support growth and propagation of such anellovectors, it is expanded and banked in cryogenic storage. During expansion and maintenance of these cells, the selection antibiotic is added to the culture medium to maintain the selective pressure. Once anellovectors are introduced into these cells, the selection antibiotic may be withheld.

Once this cell line is established, growth and production of replication-deficient anellovectors is carried out, e.g., as described in Example 15.

### Example 6: Production of Anellovectors Using Suspension Cells

This example describes the production of anellovectors in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising anellovector sequences, along with any complementing plasmids suitable or required to package the anellovector (e.g., in the case of a replication-deficient anellovector, e.g., as described in Example 16). The complementing plasmids can, in some instances, encode for viral proteins that have been deleted from the anellovector genome (e.g., an anellovector genome based on a viral genome, e.g., an Anellovirus genome, e.g., as described herein) but are useful or required for replication and packaging of the anellovectors. Transfected cells are grown in the WAVE bioreactor bags and the supernatant is harvested at the following time points: 48, 72, and 96 hours post transfection. The supernatant is separated from the cell pellets for each sample using centrifugation. The packaged anellovector particles are then purified from the harvested supernatant and the lysed cell pellets using ion exchange chromatography.

The genome equivalents in the purified prep of the anellovectors can be determined, for example, by using a small aliquot of the purified prep to harvest the anellovector genome using a viral genome extraction kit (Qiagen), followed by qPCR using primers and probes targeted towards the anellovector DNA sequence, e.g., as described in Example 18.

The infectivity of the anellovectors in the purified prep can be quantified by making serial dilutions of the purified prep to infect new A549 cells. These cells are harvested 72 hours post transfection, followed by a qPCR assay on the genomic DNA using primers and probes that are specific to the anellovector DNA sequence.

### Example 7: Quantification of Anellovector Genome Equivalents by qPCR

This example demonstrates the development of a hydrolysis probe-based quantitative PCR assay to quantify anellovectors. Sets of primers and probes are designed based on an Anellovirus genome sequence. using the software Geneious with a final user optimization. Exemplary primer sequences for TTV (Accession No. AJ620231.1) and TTMV (Accession No. JX134045.1) are shown in Table 44 below.

TABLE 44
Sequences of forward and reverse primers and hydrolysis probes
used to quantify TTMV and TTV genome equivalents by
quantitative PCR.

| | | SEQ ID NO: |
|---|---|---|
| TTMV | | |
| Forward Primer | 5′-GAAGCCCACCAAAAGCAATT-3′ | 697 |
| Reverse Primer | 5′-AGTTCCCGTGTCTATAGTCGA-3′ | 698 |
| Probe | 5′-ACTTCGTTACAGAGTCCAGGGG-3′ | 699 |
| TTV | | |
| Forward Primer | 5′-AGCAACAGGTAATGGAGGAC-3′ | 700 |
| Reverse Primer | 5′-TGGAAGCTGGGGTCTTTAAC-3′ | 701 |
| Probe | 5′-TCTACCTTAGGTGCAAAGGGCC-3′ | 702 |

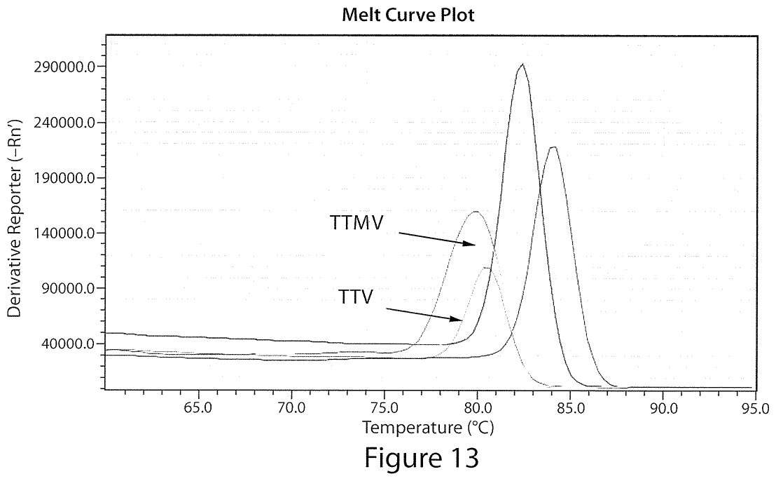

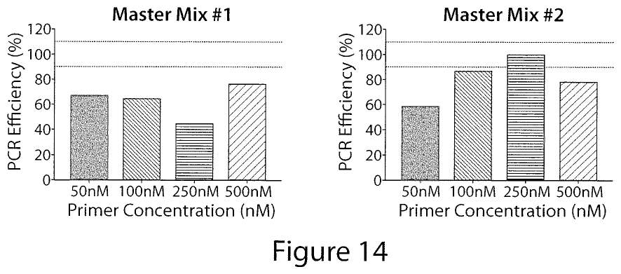

As a first step in the development process, qPCR is run using the Anellovirus primers with SYBR-green chemistry to check for primer specificity. FIG. 13 shows one distinct amplification peak for each primer pair.

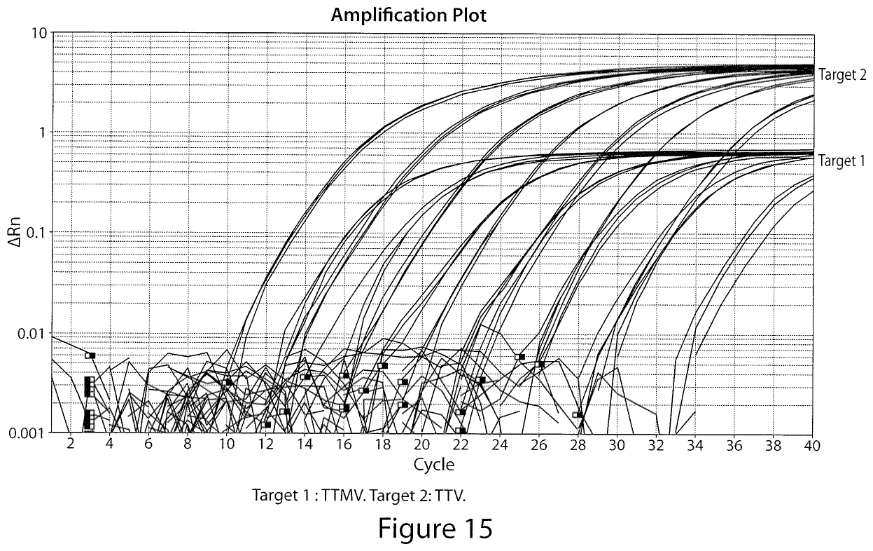

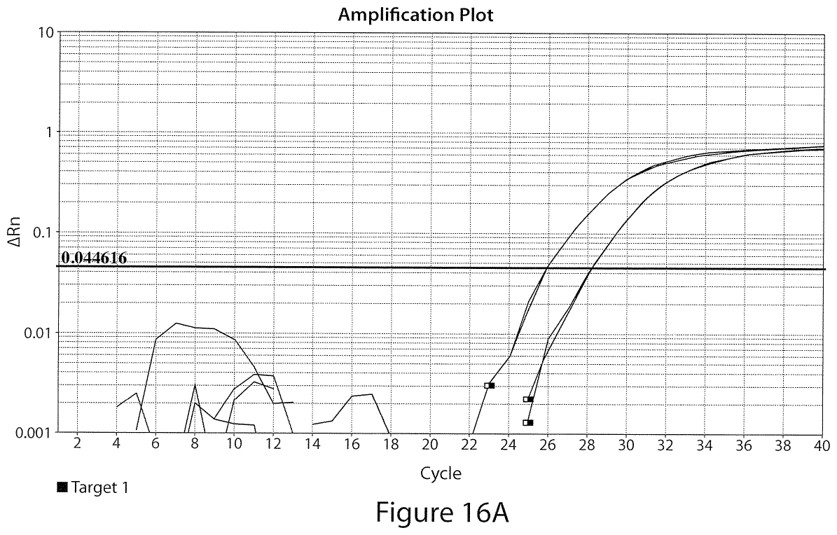

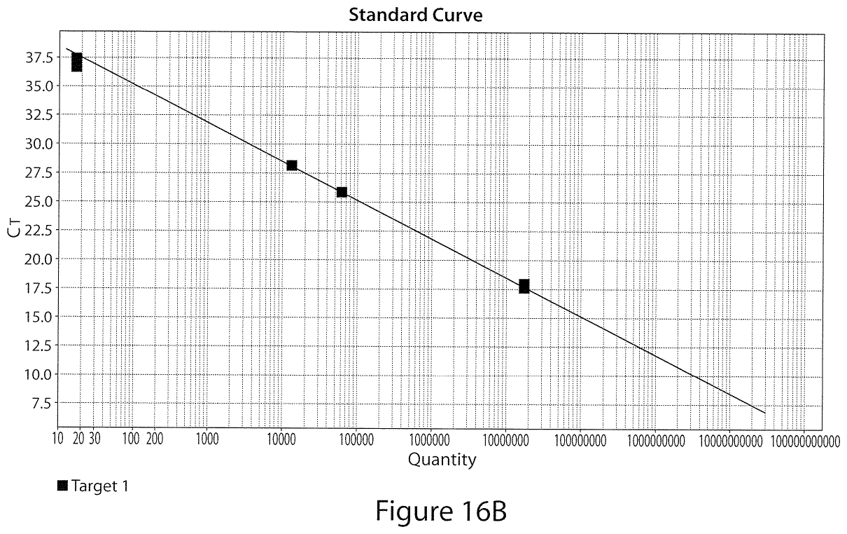

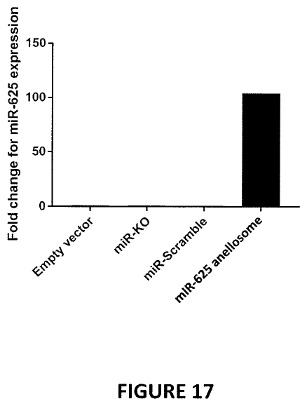

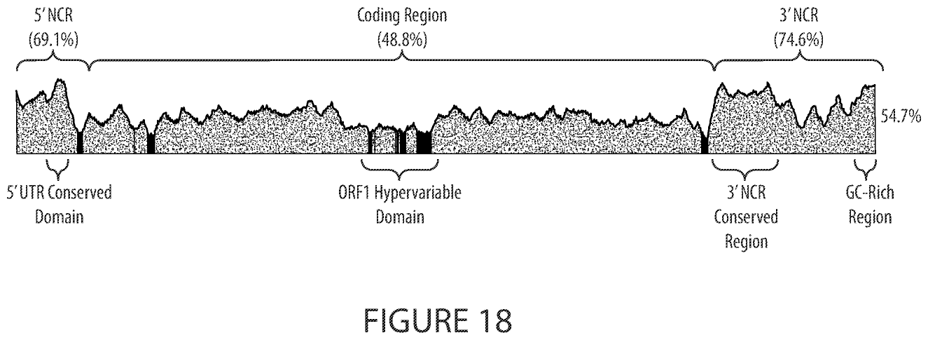

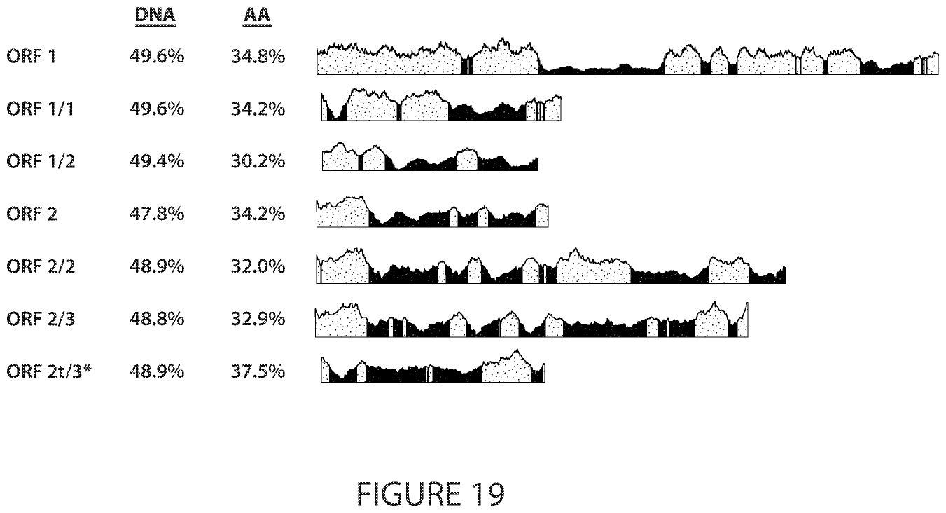

Hydrolysis probes are ordered labeled with the fluorophore 6FAM at the 5′ end and a minor groove binding, non-fluorescent quencher (MGBNFQ) at the 3′ end. The PCR efficiency of the new primers and probes was evaluated using two different commercial master mixes using purified plasmid DNA as component of a standard curve and increasing concentrations of primers. The standard curve is set up by using purified plasmids containing the target sequences for the different sets of primers-probes. Seven tenfold serial dilutions are performed to achieve a linear range over 7 logs and a lower limit of quantification of 15 copies per 20 ul reaction. All primers for qPCR are ordered from a commercial vendor such as IDT. Hydrolysis probes conjugated to the fluorophore 6FAM and a minor groove binding, non-fluorescent quencher (MGBNFQ) as well as all the qPCR master mixes are obtained from Thermo Fisher. An exemplary amplification plot is shown in FIG. 15.

Using these primer-probe sets and reagents, the genome equivalent (GEq)/ml in anellovector stocks is quantified. The linear range is then used to calculate the GEq/ml. Samples with higher concentrations than the linear range can be diluted as needed.

### Example 8: Functional Effects of an Anellovector Expressing an Exogenous MicroRNA Sequence

This example demonstrates the successful expression of an exogenous miRNA (miR-625) from anellovector genome using a native promoter.

500 ng of following plasmid DNAs are transfected into 60% confluent wells of HEK293T cells in a 24 well plate:
i) Empty plasmid backbone
ii) Plasmid containing an Anellovirus genome in which endogenous miRNA is knocked out (KO)
iii) Anellovirus genome in which endogenous miRNA is replaced with a non-targeting scramble miRNA
iv) Anellovirus genome in which endogenous miRNA sequence is replaced with miRNA encoding miR-625

72 hours post transfection, total miRNA is harvested from the transfected cells using the Qiagen miRNeasy kit, followed by reverse transcription using miRNA Script RT II kit. Quantitative PCR is performed on the reverse transcribed DNA using primer that should specifically detect miRNA-625 or RNU6 small RNA. RNU6 small RNA is used as a housekeeping gene and data is plotted as a fold change relative to empty vector.

### Example 9: Characterization of Regions Required for Anellovector Development

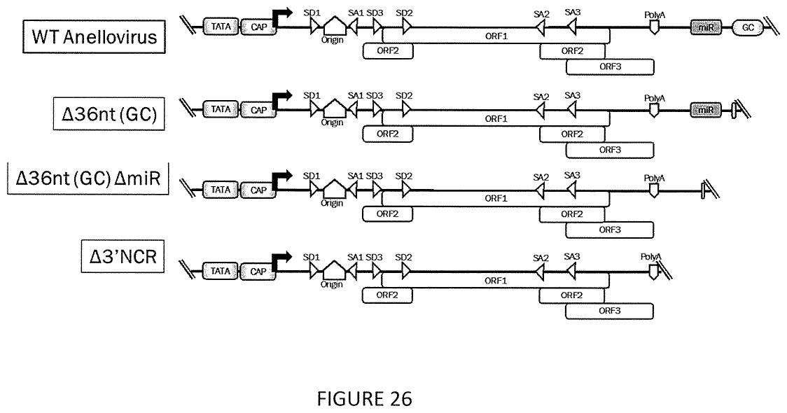

This Example describes deletions in the Anellovirus genome to help characterize the portions of the genome sufficient for replicating virus and anellovector production. A series of deletions were made in the non-coding region (NCR) of TTV-tth8 downstream of the ORFs (nts 3016 to 3753). A 36-nucleotide (nt) sequence (CGCGCTGCGCGCGCCGCCCAGTAGGGGGAGCCATGC (SEQ ID NO: 160)) was deleted from the GC region (labeled Δ36 nt (GC)). Additionally, a 78-nt pre-microRNA sequence (CCGCCATCTTAAGTAGTTGAGGCGGACGGTGGCGTGAGTTCAAAGGTCACCATCAGCCACA CCTACTCAAAATGGTGG (SEQ ID NO: 161)) was deleted from the 3′ NCR (labeled Δ36 nt (GC) ΔmiR). And lastly, an extra 171 nts in the 3′NCR of Δ36 nt (GC) was deleted (CTTAAGTAGTTGAGGCGGACGGTGGCGTGAGTTCAAAGGTCACCATCAGCCACACCTACTC AAAATGGTGGACAATTTCTTCCGGGTCAAAGGTTACAGCCGCCATGTTAAAACACGTGACGT ATGACGTCACGGCCGCCATTTTGTGACACAAGATGGCCGACTTCCTTCC (SEQ ID NO: 162)) and labeled Δ3′NCR (FIG. 26). 2 μg of circular pTTV-tth8 (WT), pTTV-tth8(Δ36 nt (GC)), pTTV-tth8(Δ36 nt (GC) ΔmiR), pTTV-tth8(Δ3′NCR) DNA plasmids harboring the altered 3′NCRs TTV-tth8 respectively described above, were transfected into HEK293 at 60% confluency in a 12-well plate using lipofectamine 2000, in triplicates. 48 hours after transfection, cell pellets were harvested and lysed to isolate mRNA transcripts (RNeasy, Qiagen cat #74104) and converted to cDNA (High-Capacity cDNA Reverse Transcription kit, ThermoFisher, cat #4368814). qPCR was performed on all samples measuring viral transcripts expression with each deletion and normalized to the internal control mRNA of GAPDH.

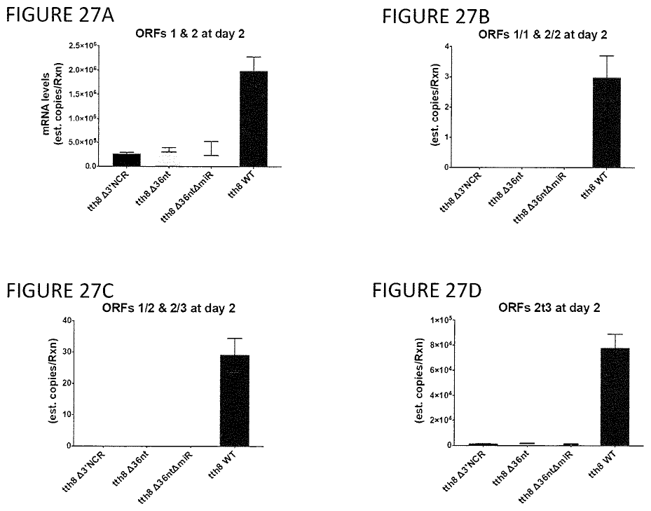

As shown in FIGS. 27A-27D, all three of the deletion mutants significantly inhibited viral transcript expression in vitro. Therefore, the 3′ NCR of TTV-tth8 is necessary for anellovector production for expression of transgene.

The TTV strain tth8, GeneBank Accession No. AJ620231.1, was deposited as a full-genome sequence. In the GC-rich region, however, there is a stretch of 36 nucleotides annotated as generic Ns. This region is highly conserved among TTV strains and therefore might be important for the biology of these viruses. The DNA sequences of several hundred TTV strains were computationally aligned and used to generate a strong consensus sequence for those 36 nucleotides (CGCGCTGCGCGCGCCGCCCAGTAGGGGGAGCCATGC (SEQ ID NO: 160)). The TTV-tth8 genome sequence referred to herein as the “wild-type” sequence accordingly had this consensus sequence inserted in place of the stretch of 36 Ns listed in the publicly available TTV-tth8 sequence.

### Example 10: Anellovector Delivery of Exogenous Proteins In Vivo

This example demonstrates in vivo effector function (e.g. expression of proteins) of anellovectors after administration.

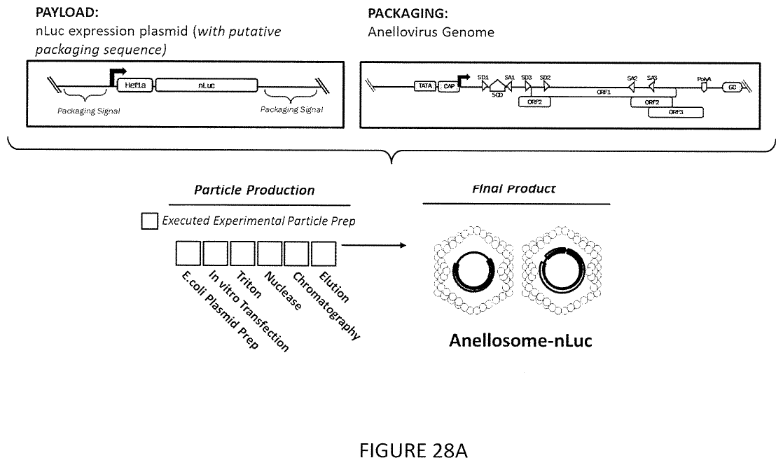

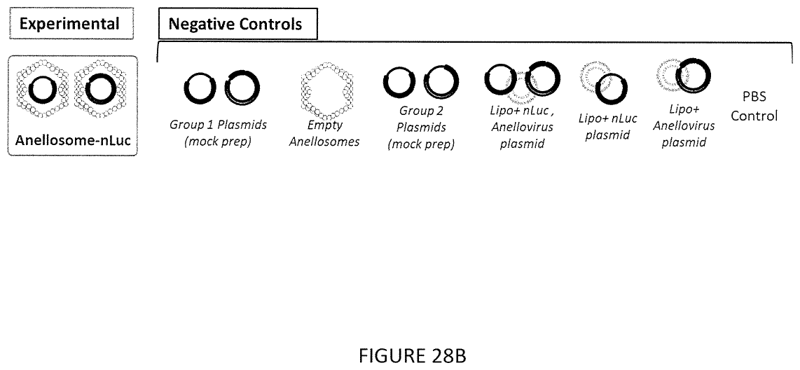

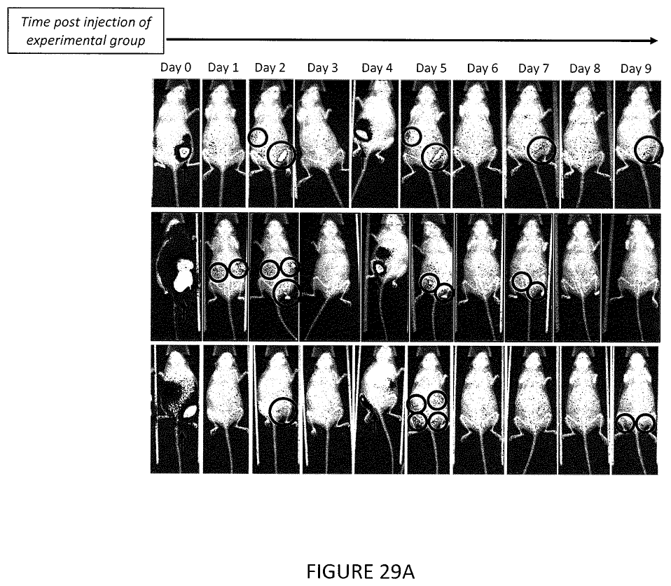

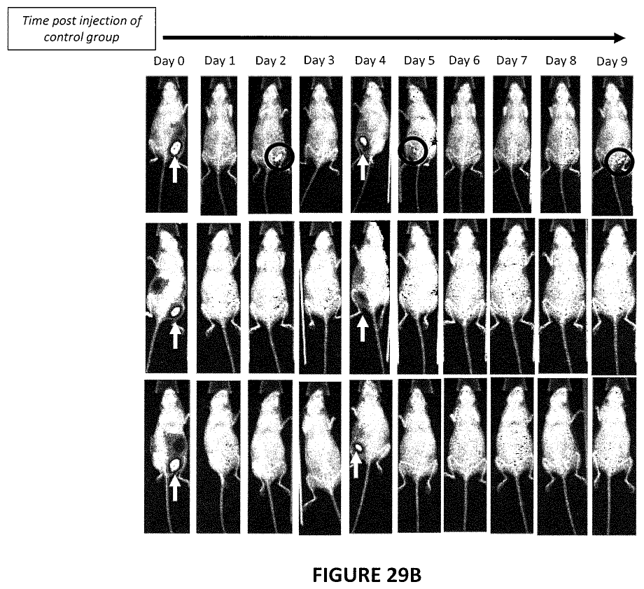

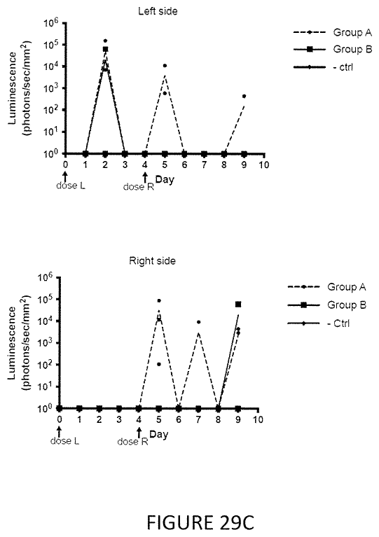

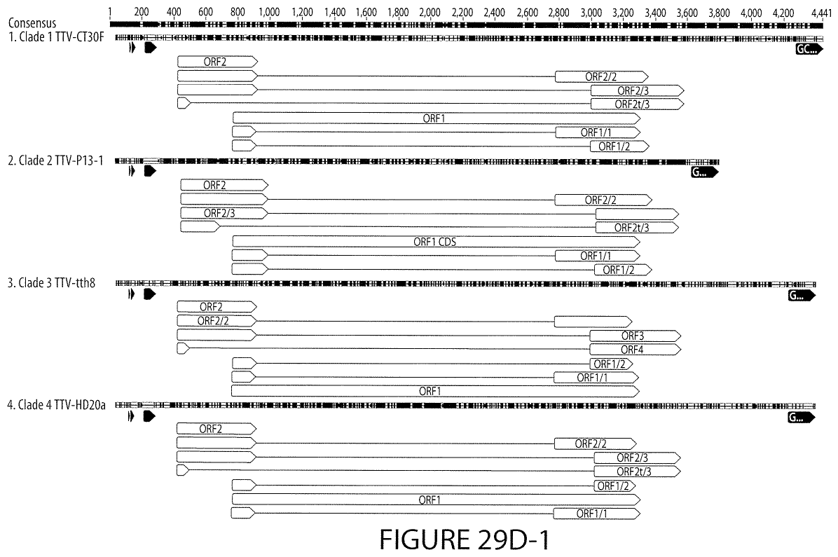

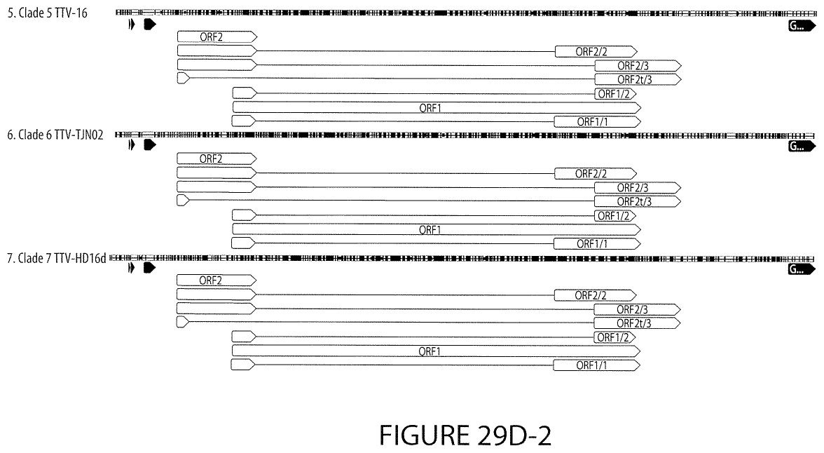

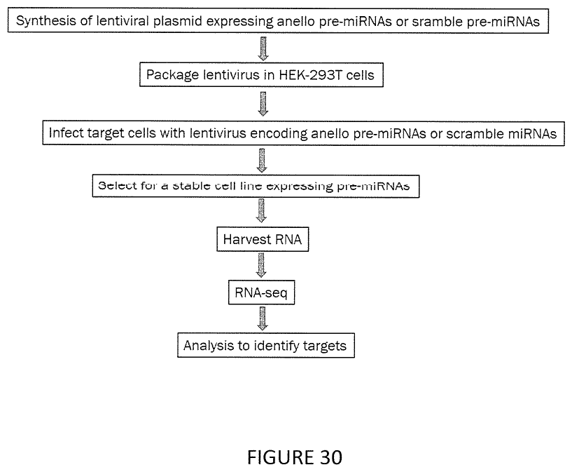

Anellovectors comprising a transgene encoding nano-luciferase (nLuc) (FIGS. 28A-28B) are prepared. Briefly, double-stranded DNA plasmids harboring the Anellovirus non-coding regions and an nLuc expression cassette are transfected into HEK293T cells along with double-stranded DNA plasmids encoding the full Anellovirus genome to act as trans replication and packaging factors. After transfection, cells are incubated to permit anellovector production and anellovector material is harvested and enriched via nuclease treatment, ultrafiltration/diafiltration, and sterile filtration. Additional HEK293T cells are transfected with non-replicating DNA plasmids harboring nLuc expression cassettes and Anellovirus ORF transfection cassettes, but lacking non-coding domains essential for replication and packaging, to act as a “non-viral” negative control. The non-viral samples are prepared following the same protocol as the anellovector material.

Anellovector preparation is administered to a cohort of three healthy mice intramuscularly, and monitored by IVIS Lumina imaging (Bruker) over the course of nine days. As a non-viral control, the non-replicating preparation is administered to three additional mice. Injections of 25 μL of anellovector or non-viral preparations are administered to the left hind leg on Day 0, and re-administered to the right hind leg on Day 4. Observation of more occurences of nLuc luminescent signal in mice injected with the anellovector preparation than the non-viral preparation would be consistent with trans gene expression after in vivo anellovector transduction.

### Example 11: Tandem Copies of the Anellovirus Genome

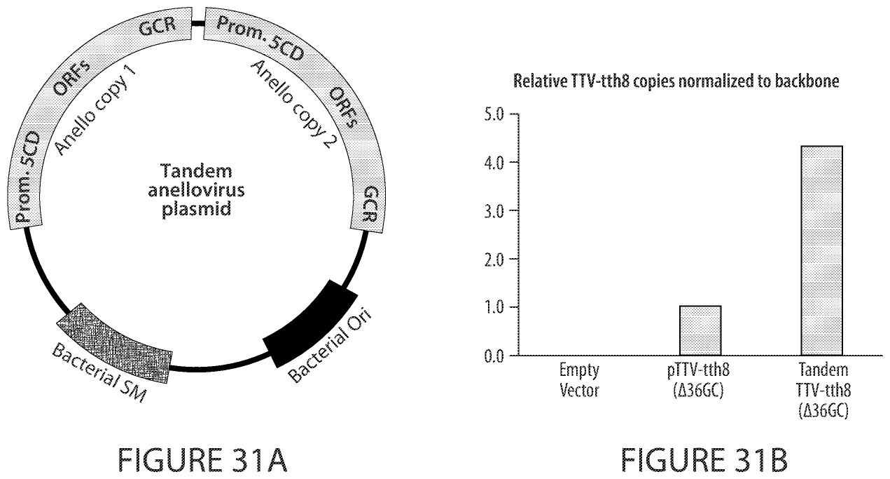

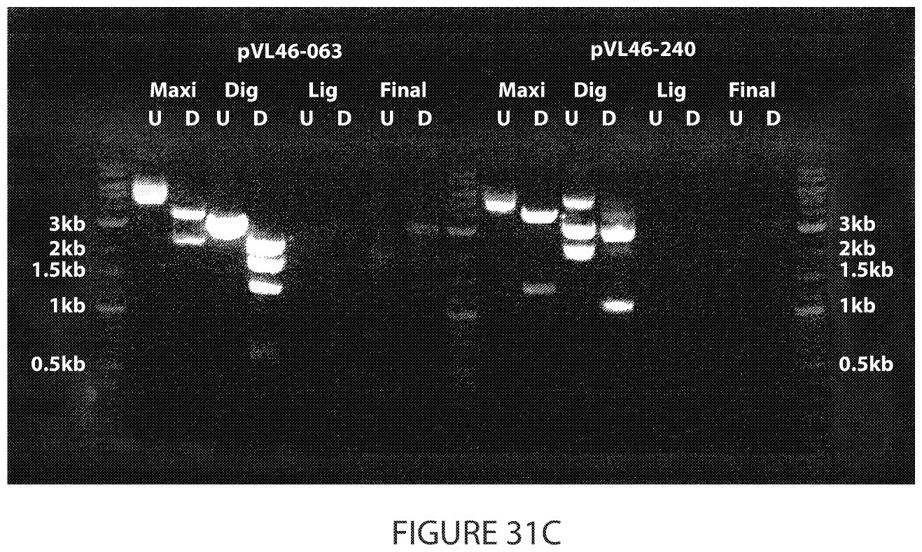

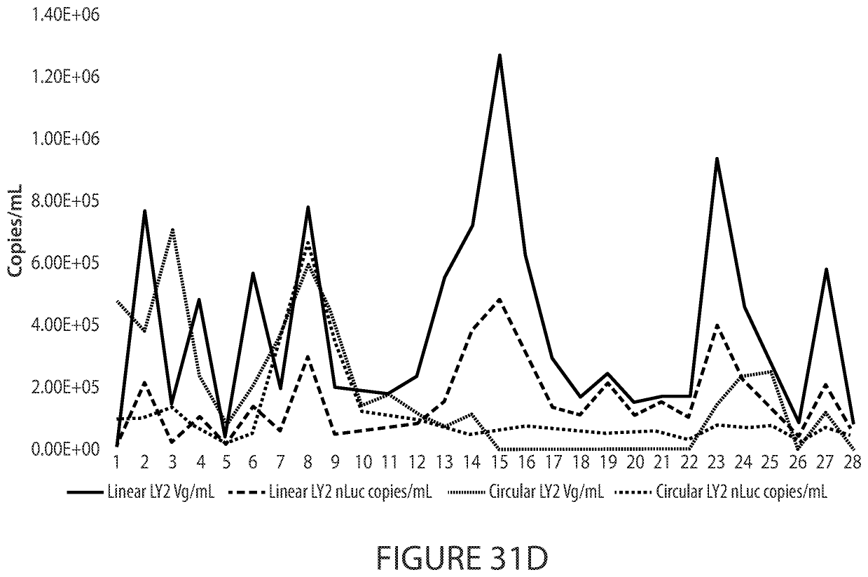

This example describes plasmid-based expression vectors harboring two copies of a single anelloviral genome, arranged in tandem such that the GC-rich region of the upstream genome is near the 5′ region of the downstream genome (FIG. 31A).

Anelloviruses replicate via rolling circle, in which a replicase (Rep) protein binds to the genome at an origin of replication and initiates DNA synthesis around the circle. For anellovirus genomes contained in plasmid backbones, this requires either replication of the full plasmid length, which is longer than the native viral genome, or recombination of the plasmid resulting in a smaller circle comprising the genome with minimal backbone. Therefore, viral replication off of a plasmid can be inefficient. To improve viral genome replication efficiency, plasmids are engineered with tandem copies of TTV-tth8 and TTMV-LY2. These plasmids present every possible circular permutation of the anelloviral genome: regardless of where the Rep protein binds, it will be able to drive replication of the viral genome from the upstream origin of replication to the downstream origin. A similar strategy has been used to produce porcine Anelloviruses (Huang et al., 2012, Journal of Virology 86 (11) 6042-6054).

Tandem anellovector can be assembled, for example, by sequentially cloning copies of the genome into a plasmid backbone, leaving 12 bp of non-viral DNA between the two sequences. Alternatively, tandem anellovector can be assembled via Golden-gate assembly, simultaneously incorporating two copies of the genome into a backbone and leaving no extra nucleotides between the genomes.

Plasmid harboring tandem copies of an anellovector genetic element sequence is transfected into HEK239T cells. Cells are incubated for five days, then lysed using 0.1% Triton X-100 and treated with nucleases to digest DNA not protected by viral capsids. qPCR is then performed using Taqman probes for the TTV-tth8 genome sequence and the plasmid backbone. TTV-tth8 genome copies are normalized to backbone copies.

### Example 12: In Vitro Circularized Anellovirus Genomes

This example describes constructs comprising circular, double stranded Anelloviral genome DNA with minimal non-viral DNA. These circular viral genomes more closely match the double-stranded DNA intermediates found during wild-type Anellovirus replication. When introduced into a cell, such circular, double stranded Anelloviral genome DNA with minimal non-viral DNA can undergo rolling circle replication to produce, for example, a genetic element as described herein.

In one example, plasmids harboring an Anellovirus genome sequence are digested with restriction endonucleases recognizing sites flanking the genomic DNA. The resulting linearized genomes are then ligated to form circular DNA. These ligation reactions are done with varying DNA concentrations to optimize the intramolecular ligations. The ligated circles are either directly transfected into mammalian cells, or further processed to remove non-circular genome DNA by digesting with restriction endonucleases to cleave the plasmid backbone and exonucleases to degrade linear DNA. To demonstrate the improvements in Anellovirus production, circularized Anellovirus genome constructs are transfected into HEK293T cells. After 7 days of incubation, cells are lysed, and qPCR is performed to compare the levels of anellovirus genome between circularized and plasmid-based anelloviral genomes. Increased levels of Anelloviral genomes show that circularization of the viral DNA is a useful strategy for increasing Anellovirus production.

Digested plasmid can be purified on 1% agarose gels prior to electroelution or Qiagen column purification and ligation with T4 DNA Ligase. Circularized DNA is concentrated on a 100 kDa UF/DF membrane before transfection. Circularization is confirmed by gel electrophoresis. T-225 flasks are seeded with HEK293T at 3×10⁴ cells/cm² one day prior to lipofection with Lipofectamine 2000. Nine micrograms of circularized Anellovirus DNA and 50 μg of circularized Anellovirus-nLuc are co-transfected one day post flask seeding. As a comparison, an additional T-225 flask is co-transfected with 50 μg of linearized Anellovirus and 50 μg of linearized Anellovirus-nLuc.

Anellovector production proceeds for eight days prior to cell harvest in Triton X-100 harvest buffer. Generally, anellovectors can be enriched, e.g., by lysis of host cells, clarification of the lysate, filtration, and chromatography. In this example, harvested cells are nuclease treated prior to sodium chloride adjustment and 1.2 μm/0.45 m normal flow filtration. Clarified harvest is concentrated and buffer exchanged into PBS on a 750 kDa MWCO mPES hollow fiber membrane. The TFF retentate is filtered with a 0.45 m filter before loading on a Sephacryl S-500 HR SEC column pre-equilibrated in PBS. Anellovectors are processed across the SEC column at 30 cm/hr. Individual fractions are collected and assayed by qPCR for viral genome copy number and transgene copy number. Viral genomes and transgene copies are observed beginning at the void volume, Fraction 7, of the SEC chromatogram. Agreement between copy number for Anellovirus genomes and Anellovirus-nLuc transgene for Anellovectors produced using circularized input DNA at Fraction 7-Fraction 10 indicates packaged Anellovectors containing nLuc transgene. SEC fractions are pooled and concentrated using a 100 kDa MWCO PVDF membrane and then 0.2 μm filtered prior to in vivo administration.

### Example 13: Production of Anellovectors Containing Chimeric ORF1 with Hypervariable Domains from Different Torque Teno Virus Strains

This example describes domain swapping of hypervariable regions of ORF1 to produce chimeric anellovectors containing the ORF1 arginine-rich region, jelly-roll domain, N22, and C-terminal domain of one TTV strain, and the hypervariable domain from an ORF1 protein of a different TTV strain.

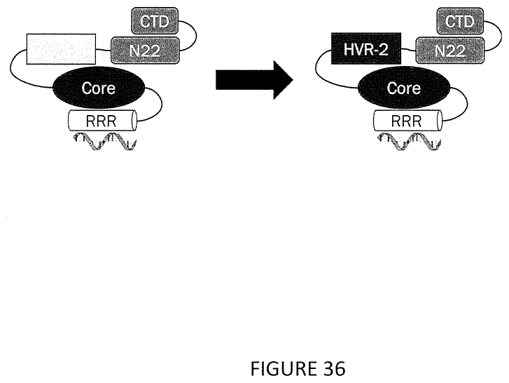

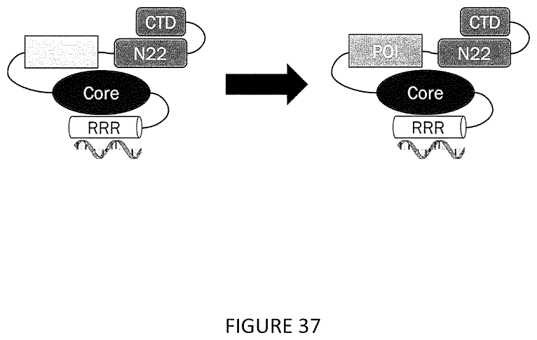

The full-length genome of a first Anellovirus is cloned into expression vectors for expression in mammalian cells. This genome is mutated to remove the hypervariable domain of the ORF1 coding sequence and replace it with the hypervariable domain of the ORF1 coding sequence from a second Anellovirus genome (FIG. 36). The plasmid containing the first Anellovirus genome with the swapped hypervariable domain is then linearized and circularized as described herein. HEK293T cells are transfected with the circularized genome and incubated for 5-7 days to allow anellovector production. After the incubation period anellovectors are purified from the supernatant and cell pellet of transfected cells by gradient ultracentrifugation.

To determine if the chimeric anellovectors are still infectious, the isolated viral particles are added to uninfected cells. The cells are incubated for 5-7 days to allow viral replication. After incubation the ability of the chimeric anellovectors to establish infection will be monitored by immunofluorescence, western blot, and qPCR. The structural integrity of the chimeric viruses is assessed by negative stain and cryo-electron microscopy. Chimeric anellovectors can further be tested for ability to infect cells in vivo. Establishment of the ability to produce functional chimeric anellovectors through hypervariable domain swapping could allow for engineering of viruses to alter tropism and potentially evade immune detection.

### Example 14: Design of an Anellovector Harboring a Payload

This example describes the design of an exemplary anellovector genetic element harboring a trans gene. The genetic element is composed of the essential cis replication and packaging domains from an Anellovirus genome (e.g., as described herein) along with a non-Anellovirus payload, which may include, e.g., protein or non-coding RNA-expressing genes. The anellovector lacks essential trans protein elements for replication and packaging, and requires proteins provided by other sources (e.g., helpers, e.g., replicating viruses, expression plasmids, or genome integrations) for rolling circle replication and encapsidation.

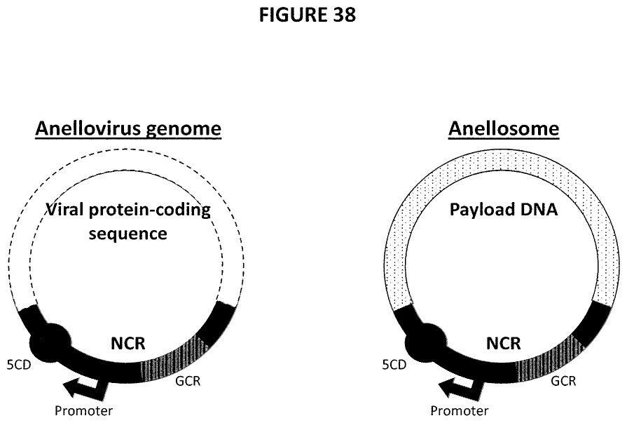

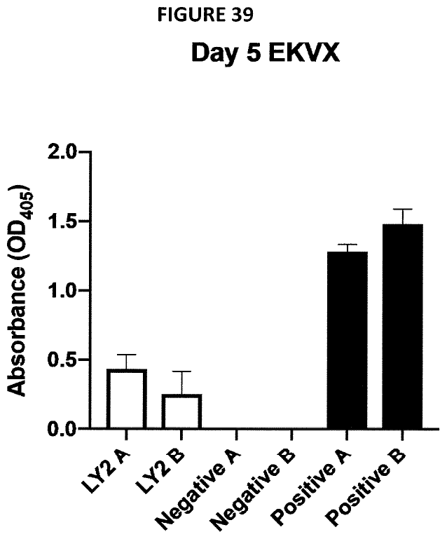

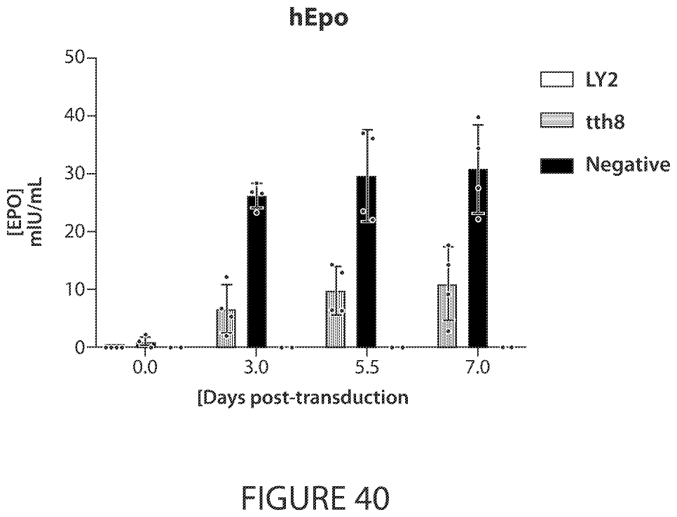

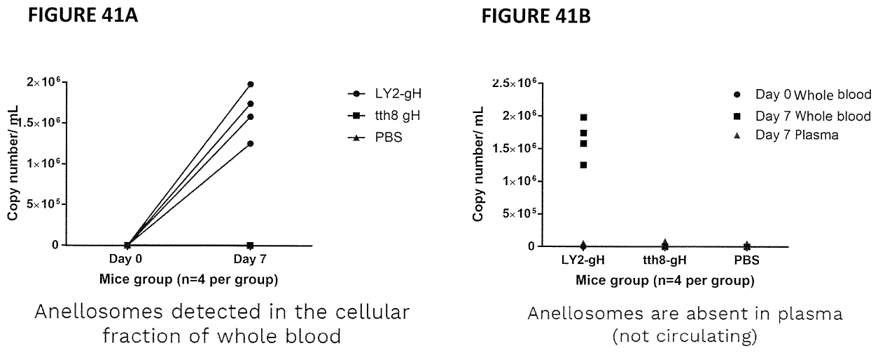

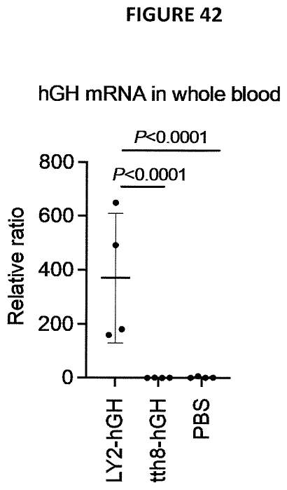

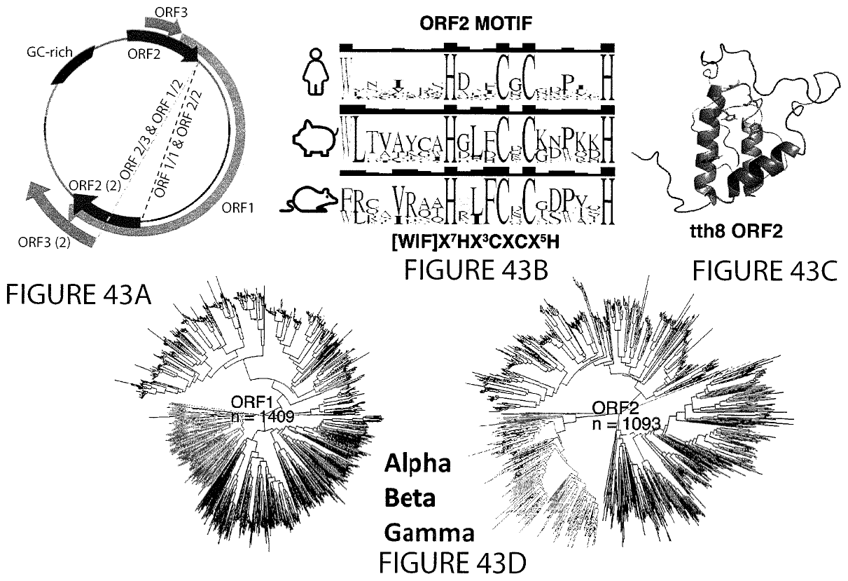

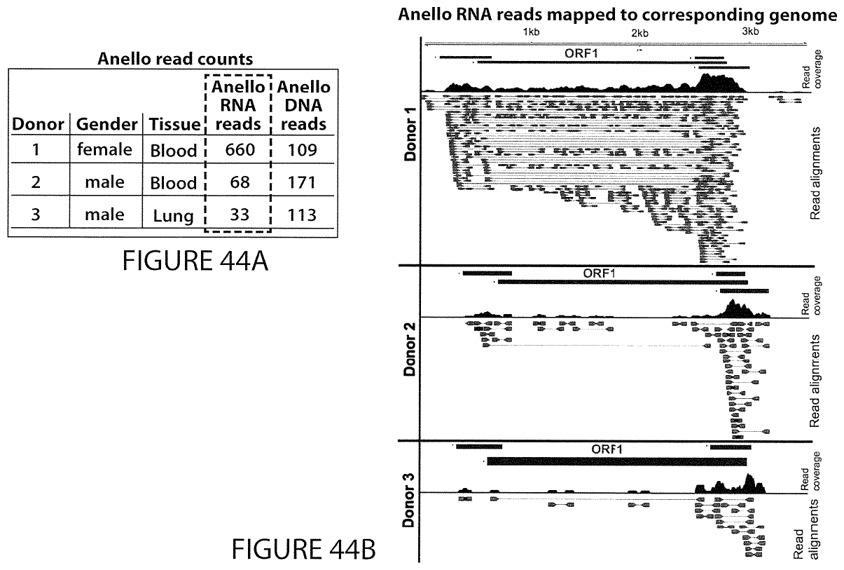

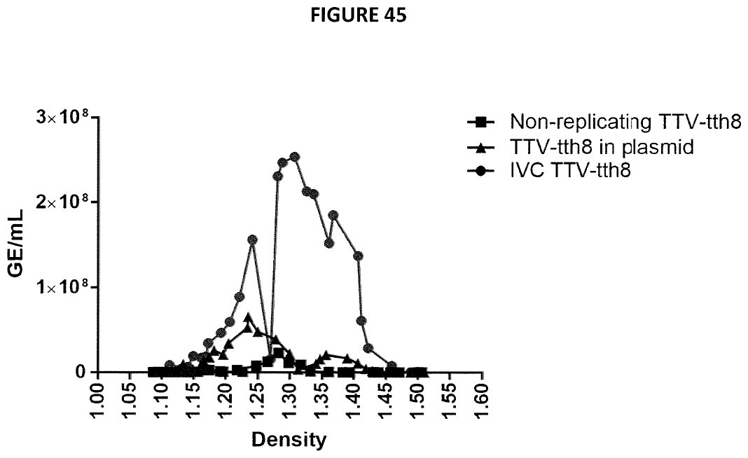

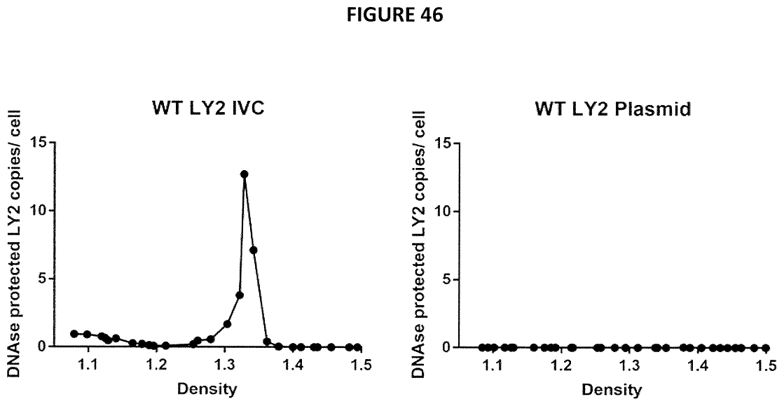

In one set of examples, the entire protein-coding DNA sequence is deleted, from the first start codon to the last stop codon (FIG. 38). The resulting DNA retains the viral non-coding region (NCR), including the viral promoter, the 5′ UTR conserved domain, the 3′ UTR (which encodes miRNAs in some anellovirus strains), and the GC-rich region. The anellovector NCR harbors essential cis domains, including the viral origin of replication and capsid binding domains. However, lacking the anellovirus protein-coding open reading frames, the anellovector is unable to express essential protein factors required for DNA replication and encapsidation, and therefore cannot amplify or package unless these elements are provided in trans.

Payload DNA, including but not limited to protein-encoding sequences, full trans genes (including non-anelloviral promoter sequences), and non-coding RNA genes are incorporated into the anellovector genetic element by insertion into the site of the deleted anelloviral open reading frames (FIG. 38). Expression from protein-coding sequences can be driven, for example, by either the native viral promoter or a synthetic promoter incorporated as a trans gene.

Replication-deficient or incompetent anellovector genetic elements (e.g., as described herein) may lack the protein-coding sequences for viral replication and/or capsid factors. Therefore, packaged anellovectors are produced by co-transfecting cells with the anellovector DNA described in this example and viral-protein-encoding DNA. The viral proteins are expressed off of replication-competent wild-type viral genomes, non-replicating plasmids harboring the viral proteins under control of the viral promoter, or plasmids harboring the viral proteins under control of a strong constitutive promoter.

### Example 15: Transduction of Anellovector Encoding a Transgene

In this example, an Anellovector carrying the payload immunoadhesin (IA) is made using an Anellovirus genome (e.g., as described herein), and then engineered to deliver a human immunoadhesin. A double-stranded circular IA anellovector DNA, which includes the Anellovirus non-coding regions (5′ UTR, GC-rich region) and an IA-encoding cassette, but did not include the Anellovirus ORFs, is designed (e.g., as described herein) and then produced by in vitro circularization, as described herein. The Anellovirus ORFs are provided in trans in a separate in vitro circularized DNA. Both DNAs are co-transfected into HEK293T cells in two biological replicates. Two biological replicates each of a negative control (mock transfection) and a positive control (IA expression cassette in a plasmid) are also tested. Transduction of the anellovector preparation into the lung-derived human cell lines EKVX and A549 is expected to result in detection of secreted immunoadhesin by ELISA. Moreover, immunofluorescence analysis of the IA anellovector-transduced EKVX cells is expected to reveal cells that are positive for expression of the immunoadhesin.

### Example 16: Anellovectors Carrying the EPO Gene into Lung Cancer Cells

In this example, a non-small cell lung cancer line (EKVX) is transduced with anellovectors carrying the erythropoeitin gene (EPO). The anellovectors are generated by in vitro circularization, as described herein. Each of the anellovectors included a genetic element that included the EPO-encoding cassette and non-coding regions of the Anellovirus genome (5′ UTR, GC-rich region), respectively, but did not include the Anellovirus ORFs, e.g., as described herein. Cells are inoculated with purified anellovectors or a positive control (AAV2-EPO at high dose or at the same dose as the anellovectors) and incubated for 7 days. Anellovirus ORFs are provided in trans in a separate in vitro circularized DNA. Culture supernatant is sampled 3, 5.5, and 7 days post-inoculation and assayed using a commercial ELISA kit to detect EPO. Successful transduction of cells is expected to result in significantly higher EPO titers compared to untreated (negative) control cells (P<0.013 at all time points).

### Example 17: Anellovectors with Therapeutic Transgenes can be Detected In Vivo after Intravenous (i.v.) Administration

In this example, anellovectors encoding human growth hormone (hGH) are detected in vivo after intravenous (i.v.) administration. Replication-deficient anellovectors encoding an exogenous hGH are generated by in vitro circularization as described herein. The genetic element of the hGH anellovectors includes Anellovirus non-coding regions (5′ UTR, GC-rich region) and the hGH-encoding cassette, but does not include Anellovirus ORFs. hGH anellovectors are administered to mice intravenously. The Anellovirus ORFs are provided in trans in a separate in vitro circularized DNA. Briefly, anellovectors or PBS are injected intravenously at day 0 (n=4 mice/group). Anellovectors are administered to independent animal groups at 4.66E+07 anellovector genomes per mouse.

In a first example, anellovector viral genome DNA copies are detected. At day 7, blood and plasma are collected and analyzed for the hGH DNA amplicon by qPCR. Presence of hGH anellovectors in the cellular fraction of whole blood after 7 days post infection in vivo and the absence of anellovectors in plasma would demonstrate the inability of such anellovectors to replicate in vivo.

In a second example, hGH mRNA transcripts are detected after in vivo transduction. At day 7, blood is collected and analyzed for the hGH mRNA transcript amplicon by qRT-PCR. GAPDH is used as a control housekeeping gene. hGH mRNA transcripts are measured in the cellular fraction of whole blood.

### Example 18: In Vitro Circularized Genome as Input Material for Producing Anellovectors In Vitro

This example demonstrates whether in vitro circularized (IVC) double stranded anellovirus DNA, as source material for an anellovector genetic element as described herein, is more robust than an anellovirus genome DNA in a plasmid to yield packaged anellovector genomes of the expected density.

1.2E+07 HEK293T cells (human embryonic kidney cell line) in T75 flasks are transfected with 11.25 ug of either (i) in vitro circularized double stranded Anellovirus genome (IVC Anellovirus), (ii) Anellovirus genome in a plasmid backbone, or (iii) plasmid containing just the ORF1 sequence of Anellovirus (non-replicating Anellovirus). Cells are harvested 7 days post transfection, lysed with 0.1% Triton, and treated with 100 units per ml of Benzonase. The lysates are used for cesium chloride density analysis; density is measured and TTV-tth8 copy quantification is performed for each fraction of the cesium chloride linear gradient.

1E+07 Jurkat cells (human T lymphocyte cell line) are nucleofected with either in-vitro circularized Anellovirus genome (IVC Anellovirus) or Anellovirus genome in plasmid. Cells are harvested 4 days post transfection and lysed using a buffer containing 0.5% triton and 300 mM sodium chloride, followed by two rounds of instant freeze-thaw. The lysates are treated with 100 units/ml benzonase, followed by cesium chloride density analysis. Density measurement and LY2 genome quantification is performed on each fraction of the cesium chloride linear gradient.

### Example 19: Rescue of Recombinant Ring 19 Human Anelloviruses

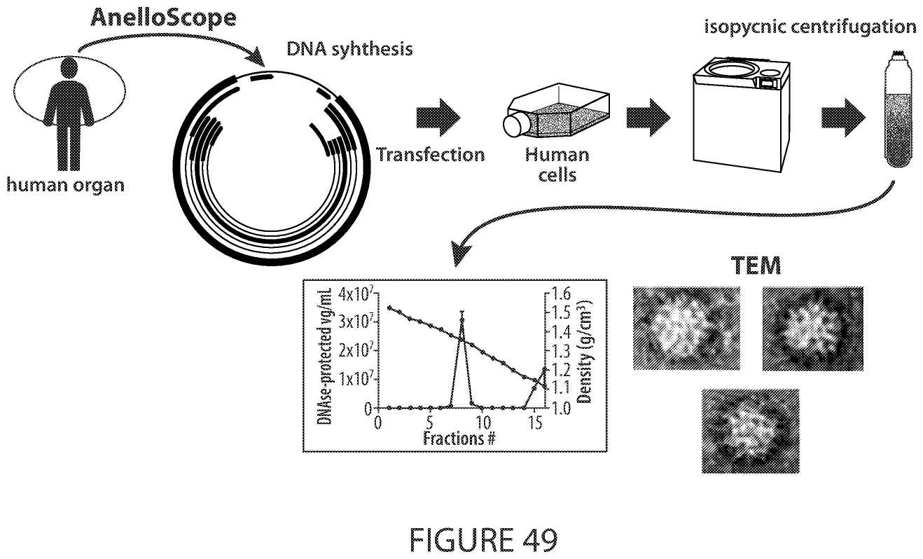

A human Anellovirus was identified from human eye tissue using methods as described herein and designated Ring 19 (FIG. 49). The Ring19 nucleotide and amino acid sequences are provided herein in Tables N1 and A1, respectively. The Ring 19 genome was then synthesized as described herein and transfected into a human cell line. Following incubation, lysis of the cells, isopycnic centrifugation, and qPCR quantitation of the resulting fractions, a peak fraction was identified at a density consistent with viral particles. Transmission electron microscopy (TEM) will be used to characterize fractions from the purification as described herein. Ring 19 particles are contemplated to be approximately 30 nm in diameter.

### Example 20: Production and Purification of Anelloviruses Using MOLT-4 Cells

This example describes the production of Anelloviruses using a human lymphoblastic cell line, MOLT-4.

### Methods and Materials

### Plasmid Construction

Plasmids containing one or two copies of the genomes of two distinct anelloviruses belonging to the Betatorquevirus genus, referred to as RING2 and RING19, were constructed.

To construct a plasmid containing a single copy of Ring2, the sequence of RING2 (GenBank accession number: JX134045.1) was synthesized by Integrated DNA Technologies into pUCIDT-Kan plasmid (pUCIDT-RING2). SapI and Esp3I restriction cut sites were added on each side of the genome in this plasmid to enable subcloning, scarless restriction digest, and for ligating the two ends of the genome to make double stranded circular genomes. The template plasmid was amplified with the following primers: FWD 5′-ACAGCTCTTCAAGGCGTCTCACCTAATAAATATTCAACAGGAAAACCACCTAATTTAAATTG CC-3′ and REV 5′-ACAGCTCTTCAGTGCGTCTCATAGGGGGTGTAAGGGGGCGTAG-3′. PCR reactions (50 μl) contained 1.0 unit Phusion DNA polymerase, 1× Phusion HF buffer, 200 μM dNTPs, 0.5 μM of each primer, 3% DMSO, and 1 ng of template DNA (New England Biolabs). All PCR reactions were run with the following parameters: initial denaturing at 98 C for 30 seconds followed by 40 cycles of denaturing at 98 C for 15 seconds, annealing at 60 C for 30 seconds, extension at 72 C for 3 minutes, and a final extension of 72 C for 10 minutes.

Purified PCR product was cloned into a pcDNA 6.2/V5-PL-DEST (Thermo Fisher Scientific) destination plasmid in a one-pot reaction containing 50 ng destination vector, 30 ng of PCR product, 1×BSA, 1×T4 DNA ligase buffer, 10 units BspQI, and 400 units T4 DNA ligase. Cloning reaction was incubated at 50 C for one hour followed by 15 minutes at 16 C.

To construct the plasmid containing two copies of RING2 in tandem, a plasmid harboring two copies of the RING2 genome arranged in a tandem configuration was assembled using a Golden Gate cloning method. The RING2 genome was subcloned into Level 1 plasmids as genome 1 (G1) and genome 2 (G2) with PCR primers containing different Esp3I overhangs for later assembly. The plasmids were amplified by PCR with forward G1-F 5′-ACAGCTCTTCAAGGCGTCTCAATGGTAATAAATATTCAACAGGAAAACCACCTAATTTAAAT TGCC-3′ and reverse G1-R 5′-ACAGCTCTTCAGTGCGTCTCATAGGGGGTGTAAGGGGGCGTAG-3′ for G1; and forward G2-F 5′-ACAGCTCTTCAAGGCGTCTCACCTAATAAATATTCAACAGGAAAACCACCTAATTTAAATTG CC-3′ and reverse G2-R 5′-ACAGCTCTTCAGTGCGTCTCATTCAGGGGGTGTAAGGGGGCGTAG-3′ for G2. PCR reactions (50 μl) contained 1.0 unit of Phusion DNA polymerase, 1× Phusion HF buffer, 200 μM of dNTPs, 0.5 μM of each primer, 3% DMSO, and 1 ng of template DNA (New England Biolabs). All PCR reactions were run with the following parameters: initial denaturing at 98° C. for 30 seconds followed by 40 cycles of denaturing at 98° C. for 15 seconds, annealing at 60° C. for 30 seconds, extension at 72° C. for 3 minutes, and a final extension at 72° C. for 10 minutes. For assembling the tandem genome plasmid, the destination plasmid, G1 subclone, and G2 subclone were cloned in a one-pot Golden Gate reaction containing 50 ng of the destination plasmid, 30 ng of each genome subclone, 1×BSA, 1×T4 DNA ligase buffer, 10 units of Esp3I, and 400 units of T4 DNA ligase. The cloning reaction was run at 37° C. for 15 minutes, 20 cycles at 37° C. for 2 minutes followed by 15° C. for 5 minutes, at 37° C. for 15 minutes, at 50° C. for 5 minutes, and at 80° C. for 5 minutes.

Another plasmid was constructed that contained two copies of RING19 in tandem. To construct this plasmid, a single copy of the RING19 genome, flanked by BsaI cut sites, was synthesized by GenScript into a pUC57-Kan vector. The RING19 genome was excised and separated from its plasmid backbone using BsaI-HFv2 and PvuI-HF restriction enzymes (New England Biolabs); the excised band was purified and ligated to itself to form an in vitro circularized (IVC) genome. A plasmid containing tandem copies of RING19 was cloned by linearizing both the IVC genome and a plasmid containing a single copy of Ring19 (described above) with NheI-HF restriction enzyme and ligating with T4 DNA ligase (New England Biolabs).

All clones were verified through Sanger sequencing at Genewiz.

### Cell Culture

MOLT-4 cells were obtained from the National Cancer Institute. Cells were scaled-up and maintained in suspension culture in complete growth medium (Gibco's RPMI 1640 with 10% fetal bovine serum [FBS], supplemented with 1 mM sodium pyruvate, Pluronic F-68 [0.1%], and 2 mM L-glutamine) at 37° C. with 5% CO₂. Cells were seeded into shake flasks (2-L, flat-bottomed, Erlenmeyer flask), each with a working volume of 800 mL, at a density of 0.1E+06 viable cells/mL and cultured in an orbital shaker (New Brunswick Innova 2100, 19-mm circular orbit) at 37° C. and 100 rpm with >85% relative humidity (RH) for 4 days.

### Transfection of MOLT-4 Cells

MOLT-4 cells were transfected with the indicated plasmids either by nucleofection or electroporation.

For nucleofection at 25 mL scale, cells were counted using the BioProfile FLEX2 analyzer (Nova Biomedical), and 1E7 cells were pelleted by spinning at 200×g for 10 minutes. Pelleted cells were resuspended in SF Cell Line Nucleofector Solution with added supplement (Lonza). 25 μg of the plasmid to be transfected (Aldevron) was added to the resuspended cells and nucleofected using the CM-150 program on the 4D-Nucleofector X Unit (Lonza). Nucleofected cells were allowed to recover in a 37° C. incubator with 5% CO₂ for 20 minutes, after which they were added to a flask containing pre-warmed complete growth medium.

For electroporation at 25 mL scale, 1E7 pelleted cells were resuspended in homemade 2S Chica buffer (5 mM KCl, 15 mM MgCl₂, 15 mM HEPES buffer solution, 150 mM Na₂HPO₄ pH 7.2, 50 mM sodium succinate). 100 μg of the plasmid to be transfected (Aldevron) was added to the resuspended cells and electroporated using a NEPA21 electroporator (Bulldog Bio). The poring pulse parameters were 2 pulses at 150 V for 5 milliseconds with an interval of 50 milliseconds. The transfer pulse parameters were 5 pulses at 20 V for 50 milliseconds with an interval of 50 milliseconds. Electroporated cells were then transferred to a flask containing pre-warmed complete growth medium.

Transfected cells were allowed to incubate at 37° C. with 5% CO₂ and harvested at the indicated times.

### Western Blotting

Cell pellets were resuspended in lysis buffer containing 50 mM Tris pH 8.0, 0.5% Triton-X100, 100 mM NaCl, and 1× Halt protease inhibitor cocktail (ThermoFisher Scientific), followed by two rounds of freeze-thawing. The cell lysates were clarified by centrifugation at 10,000×g for 30 minutes at 4° C., and the protein concentration was quantified using Pierce BCA Protein Assay Kit (ThermoFisher Scientific) according to the manufacturer's protocol. Equal amounts of the cell lysates were mixed with loading dye and Bolt sample reducing agent (ThermoFisher Scientific), followed by boiling at 95° C. for 5 minutes.

For ORF2 and GAPDH, proteins were separated on Bolt 4-12% Bis-Tris gel in 1× Bolt MOPS SDS running buffer (ThermoFisher Scientific). Separated proteins were electro-transferred to nitrocellulose membrane using Trans-Blot Turbo Transfer System (Bio-Rad). For ORF1, proteins were separated on Bolt 12% Bis-Tris gel and transferred to nitrocellulose membrane at 100 volts for 1.5 hours by a wet transfer method using cold 1× Bolt transfer buffer (ThermoFisher Scientific) supplemented with 20% methanol.

After transfer, membranes were blocked in Odyssey blocking buffer (LI-COR) for 1 hour and then incubated with relevant primary antibodies overnight. Anti-ORF2 antibody was generated by immunizing rabbits with purified full-length ORF2 protein expressed in E. coli. Anti-ORF1 antibody was generated in mice against the jelly roll domain of the ORF1 protein. Anti-ORF2 and -ORF1 antibodies were used at a concentration of 1:500. Anti-GAPDH antibody (Cell Signaling Technologies, catalog #97166) was used at a concentration of 1:1000 to detect GAPDH as a loading control.

Membranes were washed three times by rocking in a mixture of tris-buffered saline (TBS) and Polysorbate 20 for 10 minutes each. Membranes were then incubated in the relevant secondary antibodies conjugated with fluorescent dyes. Secondary antibodies used were goat anti-mouse IgG paraproteins (IRDye 680RD, LI-COR, catalog #926-68070, 1:5000 dilution) and goat anti-rabbit IgG IRDye® 680RD, LI-COR, catalog #926-68071, 1:5000 dilution).

Specific immunoreactive proteins were detected using Odyssey DLx imaging system (LI-COR).

### Reverse Transcriptase Quantitative PCR (RT-qPCR)

Transfected MOLT-4 cells were harvested by centrifugation at 500×g for 5 minutes. Pelleted cells were lysed using 700 μl QIAzol lysis reagent (Qiagen), followed by RNA extraction using miRNeasy Mini Kit (Qiagen, catalog #217004) as per the manufacturer's protocol. Additional DNAse treatment was also performed on the harvested RNA using RQ1 RNase-Free DNase (Promega, catalog #M6101) according to the manufacturer's protocol to remove any carryover of double-stranded or single-stranded DNA. cDNA synthesis was performed from DNAse-treated RNA with oligo(dT) primer using SuperScript III First-Strand Synthesis System (Invitrogen, 18080-051). qPCR was performed in triplicate using gene-specific primers with SYBR Green PCR Master Mix (ThermoFisher Scientific) in QuantStudio 5 Real-Time PCR machine (Applied Biosystems). Relative quantity was calculated using human GAPDH as a loading control.

### Southern Blotting

Isolation of total DNA from a total of 1E7 transfected MOLT-4 cells was done using DNeasy Blood & Tissue Kit (Qiagen). Cells were loaded into two columns, 5E6 cells per column, and the eluted DNA from both columns was pooled. Isolated DNA was digested with either NcoI-HF or NcoI-HF/DpnI restriction enzymes (New England Biolabs) overnight at 37° C. NcoI-HF cuts the RING2 genome once. The digested samples were separated by gel electrophoresis and subsequently transferred overnight onto a Hybond-N+ membrane. The membrane was hybridized overnight in ULTRAhyb hybridization buffer (ThermoFisher Scientific) and probed using in-house-generated, biotin-labeled oligos to detect the RING2 genome. These RING2-specific probes were made by random priming and labeled with biotin using the BioPrime Array CGH Genomic Labeling System (Invitrogen). Membranes were incubated with IRDye800 and imaged using Odyssey DLx imaging system (LI-COR).

### In Vitro Circularization (IVC) of RING2 Genome

### Cesium Chloride (CsCl) Linear Gradients

Four days after transfection, MOLT-4 cells were harvested by centrifugation at 500×g for 10 minutes. Pelleted cells were resuspended in lysis buffer containing 50 mM Tris pH 8.0, 0.5% Triton-X100, 100 mM NaCl, and 1× Halt protease inhibitor cocktail (ThermoFisher Scientific), followed by two rounds of freeze-thawing and addition of equal volumes of buffer containing 50 mM Tris pH 8.0 and 2 mM MgCl2. Cell lysates were subjected to treatment with 100 U/mL of Benzonase endonuclease (Sigma-Aldrich) and nutation at room temperature (RT) for 90 minutes. Benzonase-treated cell lysates were clarified at 10,000×g for 30 minutes at 4° C. to pellet any cellular debris.

CsCl linear gradients were prepared by overlaying 8.5 mL of 1.46 g/cm3 CsCl solution with 8.5 mL of 1.2 g/cm3 CsCl solution in 17 mL Ultra-Clear tubes (Beckman Coulter), which were then spun at a 45-degree angle and a speed of 20 rpm for 13.5 minutes using Gradient Master (BioComp).

2 mL of CsCl solution from the top of the tube was replaced with 2 mL of the processed MOLT 4 cell lysates. The sample-containing tube was spun at 31,000×g for 18 hours using SW 32.1 rotor (Beckman Coulter). 1-mL fractions were collected from the bottom of the tube. The refractive index of each fraction was measured using Refracto handheld refractometer (Mettler Toledo) to calculate density. Each fraction was desalted using a desalting kit (ThermoFisher Scientific) and then subjected to DNAse protected qPCR assay as described below.

### Iodixanol Linear Gradients

MOLT-4 cells were harvested and processed as described above for CsCl linear gradients.

To prepare iodixanol linear gradients, 13 mL of 60% OptiPrep (Sigma-Aldrich) was overlayed with 13 mL of 20% OptiPrep in 26.3-mL polycarbonate tubes, which were then spun at a 46-degree angle and a speed of 20 rpm for 16 minutes using Gradient Master (BioComp).

The sample-containing tube was spun at 347,000×g and 20° C. for 4 hours using Type 70 rotor (Beckman Coulter). 1-mL fractions were collected from the top of the tube. The refractive index of each fraction was measured using Refracto handheld refractometer (Mettler Toledo) to calculate density. Each fraction was then subjected to DNAse protected qPCR assay as described below.

DNase Protected qPCR Assay

5 μl of the sample to be titrated was incubated with 200 U of DNAse I endonuclease (New England Biolabs) in a 20-μl reaction. The reaction was incubated at 37° C. for 2 hours, followed by inactivation of DNAse I at 95° C. for 10 minutes.

4 μl of the 1:10 diluted DNAse reaction was subjected to qPCR analysis in a 20-μl reaction using TaqMan Universal PCR Master Mix (Applied Biosystems) according to the manufacturer's protocol. Primer and probe sequences are listed in Table 1.

### RING2 Scale-Up Production

### Nucleofection

Cells were counted using BioProfile FLEX2 analyzer (Nova Biomedical), and 2.0E+9 viable cells were pelleted using Sorvall BIOS A floor model centrifuge (ThermoFisher Scientific) in 1-L bottles at 500 relative centrifugal force (RCF) for 30 minutes. The supernatant was discarded, the pellets were resuspended in 20 mL of P3 solution with added supplement (Lonza), and 2 mg of the plasmid encoding tandem copies of the RING2 genome (Aldevron) was added. The cells were nucleofected using 4D Nucleofector LV Unit (Lonza) and collected in 5 mL of complete growth medium. The nucleofected cells were then transferred to 600 mL of pre-warmed complete growth medium in a shake flask and incubated in a shaker at 37° C. and 100 rpm with 5% CO₂ and >85% RH for 1 hour.

After incubation, the cells were counted using BioProfile FLEX2 analyzer (Nova Biomedical). They were then diluted to 0.4E+6 viable cells/mL in pre-warmed complete growth medium in shake flasks (800 mL maximum Working volume) and incubated in a shaker at 37° C. and 100 rpm with 5% CO₂ and >85% RH for 4 days.

### Harvest and Cell Lysis

Four days after nucleofection, cells were counted using BioProfile FLEX2 analyzer (Nova Biomedical). Cells were then harvested by pelleting using Sorvall BIOS A floor model centrifuge (ThermoFisher Scientific) at 1000 RCF for 30 minutes, and supernatant was discarded. Cell pellets were resuspended in 30 mL of 20 mM Tris pH 8, 100 mM NaCl, and 2 mM MgCl2 buffer, lysed using LM10 Microfluidizer (Microfluidics) at 10,000 psi, and washed with 30 mL of the same buffer to make a final cell lysate volume of 60 mL. Then the cell lysates were treated with 1× Halt protease inhibitor cocktail (ThermoFisher Scientific) and 100 U/mL Benzonase endonuclease (Sigma-Aldrich) and incubated for 1.5 hours on a stir plate at RT. Next, 0.5% Triton X-100 detergent was added to the cell lysates and returned to incubate at RT on the stir plate for 45 minutes. The treated cell lysates were then centrifuged using 5810 R benchtop centrifuge (Eppendorf) at 10,000 RCF for 30 minutes to pellet any cellular debris. Cellular debris was discarded, and the supernatant (lysate) was purified using density gradients.

CsCl step gradient—A CsCl step gradient was prepared by underlaying 30 mL R2 supernatant with 3 mL 1.2 g/L CsCl solution and 3 mL 1.4 g/L CsCl solution made in 30 mM Tris and 100 mM NaCl (TN) buffer in 38.6 mL Ultra-Clear ultracentrifuge tubes (Beckman Coulter). Next, the tubes were ultracentrifuged using Optima XE (Beckman Coulter) at 31,000 rpm and 10 □C for 3 hours. After the spin, the band at the junction of the 1.2 g/L and 1.4 g/L CsCl was extracted and transferred to 3-12 mL Slide-A-Lyzer dialysis cassettes with a molecular weight cutoff (MWCO) of 10K (ThermoFisher Scientific). The membranes were placed in 1× Dulbecco's phosphate-buffered saline (DPBS) with Mg and Ca salts (Gibco), 0.001% Pluronic F-68 (Gibco), and 100 mM NaCl as a dialysis buffer overnight (0/N) on a stir plate at 4° C.

CsCl linear gradient and concentration—A CsCl linear gradient was prepared by underlaying 15 mL 1.2 g/L CsCl solution and 15 mL 1.4 g/L CsCl solution in a 30 mL OptiSeal ultracentrifuge tube (Backman Coulter) and spinning using Gradient Master 108 (BioComp) at a 45-degree angle and a speed of 20 RPM for 13.5 minutes. Next, the top 3 mL of CsCl solution was replaced by 3 mL of dialyzed R2 lysate. The tubes were then ultracentrifuged at 25,000 rpm and 10 □C for 18 hours. After the O/N spin, 1 mL fractions were collected in 96 mL-deep well plates from the bottoms of the tubes. Refractive index of each fraction was measured using Refracto handheld refractometer (Mettler Toledo) to calculate density. An aliquot of each fraction was desalted using Zeba 96-well spin desalting plates (ThermoFisher Scientific) to remove any CsCl and analyzed for RING2 titer using DNAse qPCR. Fractions of interest were determined based on qPCR titer and density. They were then pooled and transferred to 3-12 mL Slide-A-Lyzer dialysis cassettes with a MWCO of 10K (ThermoFisher Scientific). The membranes were placed in 1×DPBS with Mg and Ca salts (Gibco), 0.001% Pluronic F-68 (Gibco), and 100 mM NaCl as a dialysis buffer O/N on a stir plate at 4D C. The dialyzed sample was concentrated ten-fold using Amicon Ultra centrifugal filter units (Sigma-Aldrich, Catalog #Z648043) with a MWCO of 100 kD.

### Dissection of Human Samples

Human eyes were obtained through the National Disease Research Institute (NDRI) and were dissected within 24-48 hours of procurement. Each individual eye was placed on a dissecting plate and the sclera was incised at a point between the cornea and the optic nerve using a razor blade. From that point, the sclera was cut all the way around. The aqueous humor and vitreous humor were isolated separately. The choroidal layer was then removed and the retina slowly peeled off and processed. Other compartments in the eye that were isolated and analyzed were the sclera, the iris, the cornea, the conjunctiva, and the optic nerve. Many donors had had cataract surgery; however, if the natural lens was available, it was also processed for further analysis.

### DNA Extraction and Processing

Dissected tissue sections were homogenized with DNA lysis buffer (PureLink) using a bead-beating grinder (MP Biomedicals) in homogenization tubes containing 1.4 mm ceramic spheres. The sections were homogenized at 10 m/second at 4 intervals of 15 seconds. The tubes were placed on ice for 5 minutes before being centrifuged at 13,000×g for 3 minutes. The supernatant was transferred to a new tube. 10% sodium deoxcycholate was added and incubated at 37° C. for 1 hour. Benzonase and Benzonase buffer were added to the supernatant and incubated at 37° C. for 1 hour. DNA was extracted with a PureLink viral DNA/RNA kit from Invitrogen. The samples were processed according to the manufacturer's protocol with an increase to 60 minutes for the Proteinase K incubation. Samples were eluted in 50 μL of nuclease-free water. The extracted DNA then underwent rolling circle amplification (RCA) amplification following the procedure outlined by Arze et al. The presence of Anelloviridae in the samples was tested by PCR with pan-anellovirus primers developed by Ninomiya et al. 2 μL of sample was added to 1×PCR Master Mix (Sigma-Aldrich) and the 4 degenerate primers at a final concentration of 1 μM each in a final volume of 25 μL. Positive samples were identified by the presence of the 128-base-pair band in a 2% agarose gel.

### Illumina Library Preparation and Sequencing

Post-RCA DNA was diluted to a volume of 50 μL to reduce viscosity of the samples and then the concentration of DNA was assessed by Qubit. Post-RCA DNA was library-prepped using the Nextera DNA kit (Illumina). The samples were prepared following the manufacturer's protocol for 100-500 ng input. Post-RCA DNA was also library-prepped using SureSelect XT HS2 DNA Reagent Kit (Agilent) with target enrichment probes specifically designed for Anelloviridae. Library quality control was carried out with D5000 ScreenTape on a4200 TapeStation (Agilent). All libraries were then sequenced on either an iSeq 100 or a NextSeq 550 (Illumina).

### Nanopore Library Preparation and Sequencing

Post-RCA DNA was debranched and fragmented to 20 kb-sized fragments following the NanoAmpli-Seq (Calus et al., 2018) protocol. 4.5 μg of RCA material was diluted in 65 μL of nuclease-free water and treated with 2 μL of T7 endonuclease I (New England Biolabs) for 5 minutes at RT. The reaction was then loaded in a g-TUBE (Covaris) and centrifuged at 1800 rpm for 4 minutes. The g-TUBE was then reversed, and the centrifugation process was repeated. An additional round of T7 endonuclease I and g-TUBE was performed before the mixture was then cleaned up with SPRI beads at a ratio of 1.8× with a final elution in 20 μL of nuclease-free water. The concentration of DNA was assessed by Qubit. The fragmented samples were then library-prepped with a SQK-LSK109 kit (Oxford Nanopore Technologies) following the manufacturer's protocol. Additionally, the samples were prepared with the SureSelect XT HS2 DNA Reagent Kit (Agilent) following the manufacturer's protocol with an increased elongation to 6 minutes in amplification steps. The samples were then library-prepped with the SQK-LSK109 kit (Oxford Nanopore Technologies) following the manufacturer's protocol. Libraries were loaded onto a R9.5 (FLO-MIN107) flow cell and placed onto the MinION Mk1B (Oxford Nanopore Technologies) and run for 48 hours. Only flow cells that passed the manufacturer's flow cell check test were used.

### Sequence Quality Control

Both Illumina and Nanopore raw sequencing reads were subjected to quality control utilizing FastQC (Andrews, 2019) on the sequence datasets derived from each instrument. Reports generated by FastQC for each individual sample were then aggregated into a single report using the MultiQC (Ewels et al., 2016) utility. Metrics from these reports influenced parameter selection to downstream quality control steps during analysis.

Illumina sequence data were filtered to remove low quality sequences and common adapters using bbduk (Bushnell, 2014) with the following parameters: ktrim=r, k=23, mink=11, tpe=t, tbo=t, qtrim=rl, trimq=20, minlength=50, maxns=2. The target contaminant file used was assembled by pulling contaminant sequences from NCBI GenBank covering several bacterial, human genetic elements and common lab synthetic sequences to be removed.

Nanopore sequence data were filtered to remove adapter sequences with porechop (Wick, 2018a) using default parameters followed by quality and length filtering using filtlong with parameters—min_length 2000—keep_percent 90 (Wick, 2018b). Reads passing quality control were mapped to anellovirus contig sequences (Li, 2018) with the following parameters: -cx map-ont. The resultant PAF file was both visualized in Alvis (Martin, 2021) and parsed to identify best hits to the reference contig sequences, and these reads were further analyzed with pairwise alignments in Geneious (Biomatters, 2021) with the MAFFT alignment plug-in with the G-INS-i algorithm. These long reads were used to validate the assembled short-reads and to verify that these contigs were not chimeras.

Next, human sequences were removed in two passes with both NextGenMap (Sedlazeck et al., 2013) and BWA (Li, 2013; Li and Durbin, 2009) against the GRCh37/hg19 build of the human reference genome. NextGenMap was run with parameters -affine, -s 0.7, and -p, and BWA was run with default parameters. Mapped reads output in SAM file format were converted to paired-end FASTQ format with both SAMtools (Li et al., 2009) and Picard's (Broad Institute, 2018) SamToFastq utility configured with the parameter VALIDATION_STRINGENCY=“silent”.

rRNA contaminants and common laboratory bacterial contaminants were removed with bbmap (Bushnell, 2014) with the following parameters: minid=0.95, bwr=0.16, bw=12, quickmatch=t, fastt, minhits=2. An accounting of all reference sequences screened against can be found in the provided supplementary data.

Finally, we de-duplicated the short read data passing all QC and decontamination steps to speed up and aid in genome assembly quality by using clumpify (Bushnell, 2014) configured with the parameter dedupe=t.

### Genome Assembly

Short, trimmed, decontaminated and de-duplicated sequencing reads were assembled with metaSPAdes (Nurk et al., 2017), with the error correction module disabled via the use of the—only-assembler parameter. The resulting contigs were filtered with PRINSEQ lite (Schmieder and Edwards, 2011), using the parameters out_format 1, −lc_method dust, and lc_threshold 20. Contigs passing this filtering step were then clustered at 99.5% similarity to remove any duplicate sequences via the VSEARCH software's cluster_fast algorithm (Rognes et al., 2016) using default parameters. Any putative complete, circular genomes were recovered from contigs using ccfind (Nishimura et al., 2017), with all parameters set to defaults.

### Long Read Error Correction

Nanopore reads classified as anellovirus sequences were error corrected using paired short-read data utilizing racon (Vaser R et al., 2017). First, short reads classified as anellovirus were mapped to long anellovirus reads using BWA's (Li, 2013) mem algorithm with default parameters. The Resulting SAM alignment and the short-reads and long reads used to produce the alignment were supplied to racon for error correction. Execution of racon was conducted using default parameters for multiple rounds of error correction until the polished product showed no variation from the previous iteration.

### Anellovirus Contig Identification

Assembled contigs were screened using NCBI's blastn software (citation), with default parameters, to identify putative anellovirus sequence using a custom in-house anellvirus database consisting of 728 curated anellovirus sequences.

### Anellovirus Genome Annotation

ORF sequences were identified and extracted from assembled anellovirus contigs using the OrfM (Woodcroft et al., 2016) software with parameters configured to print stop codons (-p), print ORF's in the same frame as a stop codon (-s) and constrained to ORF sequences longer than 50 amino acids (-m 150).

Predicted ORF sequences were further filtered using seqkit's seq and grep utilities (Shen et al., 2016) to subdivide ORF sequences into bins corresponding to ORF1, ORF2 and ORF3. ORF1 sequences were identified by filtering ORF sequences using seqkit seq for those no shorter than 600 amino acids (-m 600) and using seqkit grep to search through the ORF sequence data (-s), for the conserved motif YNPX²DXGX²N with a regular expression (-r) based pattern (-p “YNP.{2}D.G.{2}”). Similarly, ORF2 sequences were recovered using the conserved motif WX⁷HX³CXCX⁵H previously identified in literature (Takahashi et al., 2000) through seqkit's grep utility (-p “W.{7}H.{3}C.C.{5}H”).

ORF3 sequences were predicted by utilizing the presence and coordinate positions of predicted ORF1 and ORF2 sequences on the same contig. Predicted ORF3's use a STOP codon downstream of those used by ORF1 and its reading frame is different from that of ORF1 and ORF2. Additionally, parsing the ORF3 sequences from internal datasets (median length: 68aa, minimum length: 50aa, maximum length: 159aa) through MEME (Bailey et al. 2009) revealed the presence of two previously unknown and highly conserved motifs located near the 3′ end of ORF3. Both novel motifs were also utilized to identify ORF3 sequences using seqkit's grep command.

Identified ORF sequences required an additional trimming step as OrfM produces ORF calls with peptides upstream of canonical start codons. ORF1 Sequences were timed to the proper start codon via an in-house written python script that used the presence of the arginine rich region to identify the first methionine located upstream of it in the direction of the 5′ end. In some cases, a non-canonical start codon was predicted as the ORF1 start codon by searching for the amino acid's threonine-proline-tryptophan or threonine-alanine-tryptophan just upstream of the arginine-rich region. ORF2 and ORF3 sequences were trimmed to the first start codon identified nearest the 5′ end of the sequence.

### Anellovirus Genera Classification

Anellovirus contig sequences were identified into one of the three known generea by use of the tblastx software (citation) to conduct a homology search against a custom in-house database consisting of 720 curated and classified Anellovirus sequences. The top hits that contained suitable coverage across the majority of the contig sequence were then used in genera classification.

### Primer Walking and Genome Recovery

Primers were designed around regions of inconsistencies between the long read and short read sequencing data. Post-RCA DNA was amplified using these primers with a Q5 Hot Start polymerase (New England Biolabs). The product was run on a 2% gel to confirm specific binding before sending the PCR product to GeneWiz for Sanger sequencing. Sanger sequencing results were analyzed using Geneious bioinformatics software (Biomatters).

### RING19 Scale-Up Production

Nucleofection, cell harvest, and lysis were performed as described for RING2 above except that the transfected plasmid encoded two copies of the RING19 genome in tandem.

### Iodixanol Linear Gradient and Concentration

An iodixanol linear gradient was prepared by overlaying 19 mL of 20% iodixanol solution made in TN buffer with 19 mL of OptiPrep 60% iodixanol solution (Sigma-Aldrich) in 38.6 mL Ultra-Clear ultracentrifuge tubes (Beckman Coulter) and spinning on the Gradient Master (BioComp) at a 45-degree angle and a speed of 20 rpm for 16 minutes. Then the top 5 mL of iodixanol solution was replaced with 5 mL R19 lysate, and the tubes were ultracentrifuged at 32,000 rpm and 20 □C for 18 hours. After the O/N spin, 1-mL fractions were collected in 96 mL-deep well plates from the tops of the tubes. An aliquot of each fraction was used to measure refractive index using Refracto handheld refractometer (Mettler Toledo) as well as RING19 titer, as per the protocol described above for the DNAse protected qPCR. Fractions of interest were determined based on the viral titer and density measurements. They were then pooled and concentrated ten-fold using the Amicon Ultra centrifugal filter units (Sigma-Aldrich, Catalog #Z648043) with a MWCO of 100 kD.

### Size Exclusion Chromatography (SEC):

Prior to SEC, the sample was centrifuged at 12000 rpm for 1 minute. The supernatant was loaded onto a HiPrep 16/60 Sephacryl S-500 HR column (Cytiva) with buffer conditions at 50 mM Tris pH 8.0, 150 mM NaCl, and 0.01% poloxamer. The entire purification was performed at 4° C. with a 1 mL/minute flow rate. The fractions with significant qPCR numbers were pooled and concentrated using Vivaspin 2, 10,000 MWCO PES concentrator (Sartorius, catalog #VS0201) and Nanosep centrifugal devices with Omega membrane at MWCO of 30K (Pall, catalog #OD030C34).

### Electron Microscopy

To visualize virus particles, negative-stained transmission electron microscopy was conducted at Harvard Medical School using Jeol 1200 EX equipped with an AMT 2 k CCD camera. 10 μl of sample was blotted on 400-mesh carbon support film (EMS CF400-Cu) for 30 seconds. After washing with double-distilled water for 30 seconds, the grid was stained by 1% of uranyl acetate for 10 seconds before imaging.

### In Vivo RING19 Infectivity Studies

### Care and Use of Animals

All mouse studies were approved and governed by the Laronde Institutional Animal Care and Use Committee. Female C57Bl/6J mice 8-12 weeks of age were obtained from Jackson Laboratories for use in these ocular studies.

### Subretinal Injections

Pupils were first dilated with one to two drops of 1% tropicamide/2.5% phenylephrine HCl (Tropi-Phen, Pine Pharmaceuticals). The mouse was subsequently anesthetized using an intraperitoneal injection of a ketamine/xylazine cocktail (100/10 mg/kg). One or two drops of 0.5% proparacaine (McKesson Corp.) were applied to the eye. An incision approximately 0.5 mm in length was made with a micro scalpel 1 mm posterior to the nasal limbus. A 33 g blunt-ended needle on a 5 μl Hamilton syringe was inserted through the scleral incision, posterior to the lens, toward the temporal retina until resistance was felt. One microliter of either PBS, virus, or vector containing 0.1% of sodium fluorescein (AK-Fluor 10%, Akorn) was then injected slowly into the subretinal space. The eye was examined and the success of the subretinal injection was confirmed by visualizing the fluorescein-containing bleb through the dilated pupil with a Leica M620 TTS ophthalmic surgical microscope (Leica Microsystems, Inc). Eyes with significant hemorrhage or leakage of vector solution from the subretinal space into the vitreous were excluded from the study. After the procedure, 0.3% tobramycin ophthalmic ointment 0.3% (Tobrex, Alcon) was applied to each treated eye and the mouse was allowed to recover from the anesthesia prior to being returned to its cage in the housing room.

### Intravitreal Injections

Pupils were first dilated with one to two drops of 1% tropicamide/2.5% phenylephrine HCl (Tropi-Phen, Pine Pharmaceuticals). The mouse was subsequently anesthetized using an intraperitoneal injection of a ketamine/xylazine cocktail (100/10 mg/kg). One or two drops of 0.5% proparacaine (McKesson Corp.) were applied to the eye. A 34 g beveled needle on a 5 μl Hamilton syringe was inserted 1 mm posterior to the nasal limbus, taking care not to damage the lens. One microliter of either PBS, virus, or vector containing 0.1% of sodium fluorescein (AK-Fluor 10%, Akorn) was then injected slowly into the subretinal space. The eye was examined, and the success of the intravitreal injection was confirmed by visualizing the fluorescein-containing vitreous through the dilated pupil with a Leica M620 TTS ophthalmic surgical microscope (Leica Microsystems, Inc). Eyes with significant hemorrhage, lens damage, or leakage of vector solution outside of the eye were excluded from the study. After the procedure, 0.3% tobramycin ophthalmic ointment 0.3% (Tobrex, Alcon) was applied to each treated eye and the mouse was allowed to recover from the anesthesia prior to being returned to its cage in the housing room.

### Harvesting and Processing of Tissue Samples for DNA Extraction

Mouse eyes were dissected at indicated time points following SR or IVT injections (n=5 for each time point). After enucleation, the retina and posterior eyecup (PEC) were separated and processed individually. These tissues were collected in tubes containing stainless steel beads and flash-frozen immediately. They were stored at −80 C until ready for homogenization. Frozen tissues were homogenized using Geno/Grinder 2010 (SPEX SamplePrep, LLC) at 1,250 rpm for 30 seconds. Genomic DNA was isolated from homogenized tissues using the DNEasy Blood and Tissue Kit (Qiagen) according to the manufacturer's instructions and quantified on Qubit Fluorometer using the Qubit DNA broad range Assay Kit (Thermo Fisher).

### Quantitative PCR Analysis

Genomic DNA was assayed by qPCR on the QuantStudio 5—Real-Time PCR System (Thermo Fisher) using TaqMan Universal PCR Mastermix (Thermo Fisher). The sequence detection primers and the custom Taqman probe that were used in this study were synthesized by IDT (Table Z1). All the reactions including the DNA samples and different dilutions of a known quantity of the linearized mCherry or Ring19 plasmid standards were run in triplicates on the same plate. Standard curve method was used to calculate the amount of viral/vector DNA and was normalized with the total amount of genomic DNA for each sample (quantified using Qubit as described above).

TABLE Z1
Primer and probes designed to quantify AAV2.mCherry and WT
Ring19

| Target | Label | Sequence (5′ 3′) |
|---|---|---|
| mCherry | Forward Primer | CCGACTACTTGAAGCTGTCC |
| | Reverse Primer | CGCAGCTTCACCTTGTAGAT |
| | TaqMan Probe (FAM) | TGATGAACTTCGAGGACGGC |
| WT Ring19 | Forward Primer | GGATTTTGGGAGGGTCACTC |
| | Reverse Primer | TACAGTTCCTGGACCTGTGT |
| | TaqMan Probe (FAM) | ACACTGGTACCCTAAAAATAGATTTCA |

### Results

### RING2 Promoter is Active in MOLT-4 Cells

Viral load of anelloviruses in human plasma has been reported to be hundred-fold lower than the whole blood [Tyschik et al., 2017], suggestive of cellular component of the blood harboring anellovirus infection. In addition to being infected, lymphocytes have been previously reported to be a major site of anellovirus replication [Mariscal et al, 2001; Maggi et al, 2001; Focosi et al, 2015; Maggi et al, 2001]. Therefore, we examined whether anellovirus genes can be expressed in MOLT-4, a T-cell line derived from a patient with acute lymphoblastic leukemia. We synthesized a plasmid encoding two copies of the genome of LY2 (referred to hereafter as RING2) in a tandem arrangement as described above. RING2 is a human anellovirus belonging to the Betatorquevirus genus that was previously sequenced from the pleural fluids of children hospitalized in France with parapneumonic empyema [Galmès 2013]. MOLT-4 cells electroporated with this plasmid were harvested at Days 1, 2, 3 and 4 post-transfection and analyzed for the detection of RING2 transcripts by reverse transcriptase quantitative PCR (RT-qPCR).

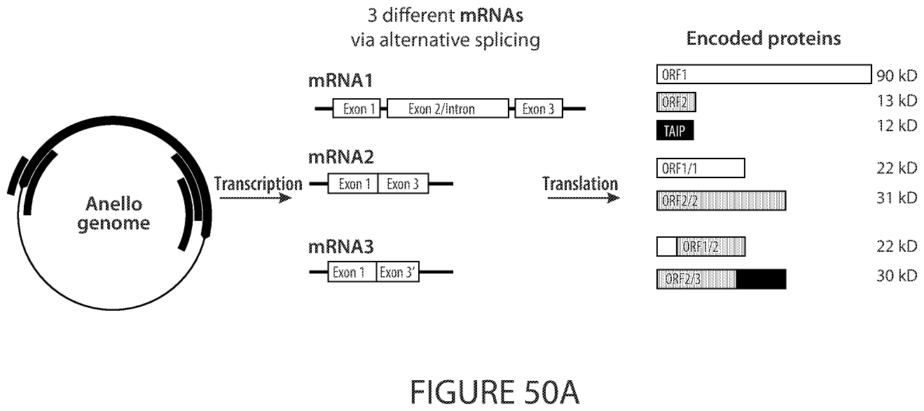

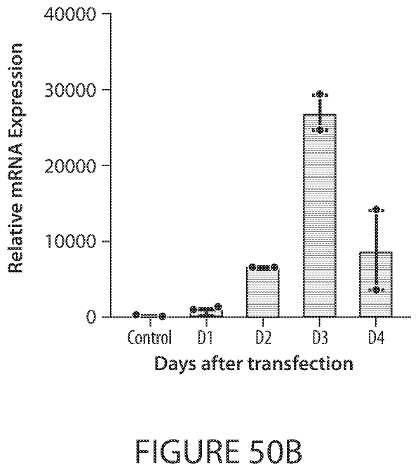

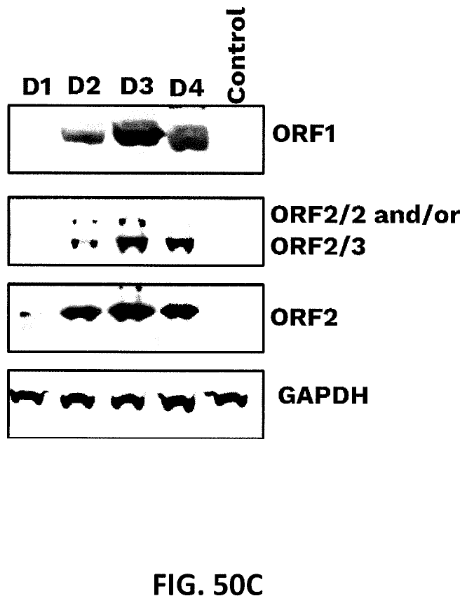

Previous anellovirus gene expression studies have described three major mRNA isoforms that are produced as a result of alternative splicing (FIG. 50A). For our analysis, we used a primer pair that would detect all 3 isoforms of RING2 transcripts. Expression of GAPDH transcript was used for normalization. We were able to detect RING2 transcripts starting at day 1 post electroporation. Expression peaked at day 3 post transfection and was reduced at day 4 (FIG. 1B). As expected, we did not detect any RING2 transcripts in untransfected MOLT-4 cells. (FIG. 50B). Having detected RING2 transcripts, next we performed a similar time-course experiment to determine RING2 protein expression. Antibodies to detect the putative nucleocapsid protein, ORF1, as well as ORF2 and its variants were generated. As shown in FIG. 50C, ORF1 is detectable beginning on Day 2 post-transfection, peaks on Day 3, and plateaus beyond Day 3. As the epitope used to generate the anti-ORF1 antibody is specific to the jelly roll domain of ORF1, it cannot detect isoforms such as ORF1/1 and ORF1/2.

The anti-ORF2 antibody that we generated can detect all three isoforms of ORF2, including ORF2, ORF2/2, and ORF2/3. The predicted molecular weights for ORF2, ORF2/2, and ORF2/3 are 17, 31, and 30 kDa respectively. Since ORF2/2 and ORF2/3 are nearly equal in molecular weight, it is challenging to distinguish them based on migration on SDS-page gel in a denatured state. Like ORF1, the expression of ORF2 and its isoforms also peaked on Day 3 post-transfection and plateaued thereafter (FIG. 50C).

Collectively, these results suggest that the RING2 promoter is active in MOLT-4 cells, enabling transcription and translation of anellovirus genes in this human cell line.

### MOLT-4 is Permissive for the Replication of the RING2 Anellovirus

Having detected RING2 gene expression in MOLT-4 cells, next we tested whether the cell line is permissive for replication of the anellovirus.

Plasmids encoding either a single copy of the RING2 genome or two copies of the RING2 genome in tandem were used to nucleofect the cells. The cells were harvested four days post-nucleofection, followed by DNA extraction. Extracted DNA was either left untreated or treated with a restriction enzyme that digests the plasmid backbone once, a restriction enzyme that digests the RING2 genome once, or DpnI. DNA replicated in bacterial cells contains methylated adenine and therefore is sensitive to digestion with DpnI. On the other hand, DNA that replicates in eukaryotic cells lacks methylated adenine and therefore is resistant to digestion with DpnI. Hence, DpnI digestion can be used to distinguish between the transfected genome and the genome that replicated in MOLT-4 cells. Untreated and treated DNA samples were subjected to Southern blot analysis using probes designed to specifically detect the RING2 genome.

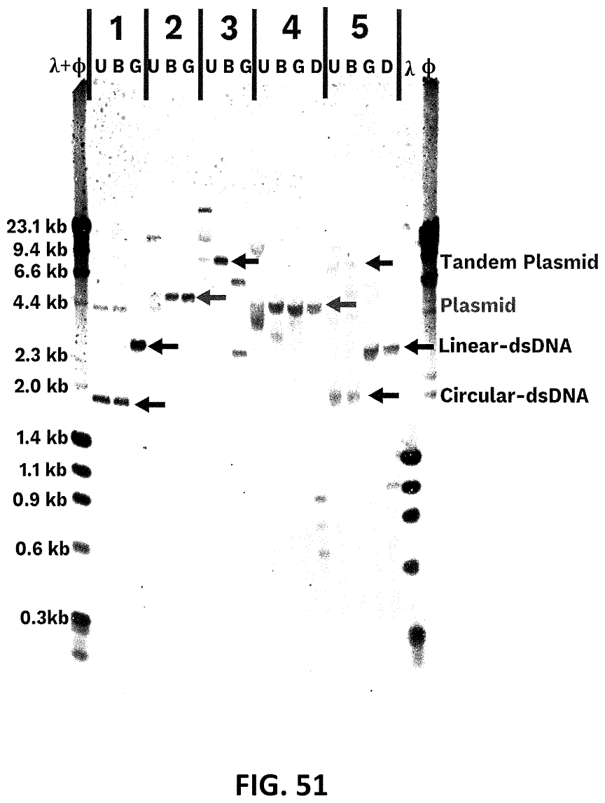

For DNA extracted from the sample transfected with a tandem RING2 genome-containing plasmid (FIG. 51, Sample #5), we detected a band of the same size as would be expected for a unit-length, double-stranded RING2 genome. This band was insensitive to digestion with an enzyme that cuts the plasmid backbone but became linear when treated with an enzyme that cuts the RING2 genome once. These observations suggest that this band represents the RING2 genome and not the plasmid backbone. Furthermore, this band was resistant to treatment with DpnI, indicating that the genome RING2 replicated in the transfected MOLT-4 cells. Overall, based on the patterns of banding observed for samples and controls, we conclude that MOLT-4 cells transfected with a tandem RING2 genome-containing plasmid are permissive for replication of the anellovirus and lead to a unit-length, replicated anellovirus genome.

A DpnI-resistant band was also detected for the sample transfected with a single RING2 genome-containing plasmid (FIG. 51, Sample #4). When this sample was treated with a restriction enzyme that digests either the plasmid backbone once or the RING2 genome once, we detected a band a band of the same size as would be expected for a linear plasmid containing the entire RING2 genome. This result suggests that the single RING2 genome-containing plasmid can also replicate in MOLT4 cells but does not yield a detectable unit-length anellovirus genome.

To our knowledge, this is the first study to demonstrate the replication of a unit-length, circular, human anellovirus genome in a human cell line using recombinant DNA as an input material.

### MOLT-4 Cells are Permissive for RING2 Packaging

Since we demonstrated that MOLT-4 cells are permissive for RING2 replication, we next tested whether RING2 particles can be produced in this human cell line. We nucleofected MOLT-4 cells with either a plasmid containing a qPCR amplicon of RING2 (RING2 non-rep) or an in vitro circularized, double-stranded RING2 genome (RING2 IVC). Four days after nucleofection, cells were harvested, lysed by two rounds of freeze-thawing, treated with Benzonase, clarified, and subjected to isopycnic ultracentrifugation using CsCl linear gradient. 1-mL fractions of CsCl linear gradient were collected from the bottom of the tube. Each fraction was analyzed for its density as well as for titers of RING2 titer using a DNAse protected qPCR assay.

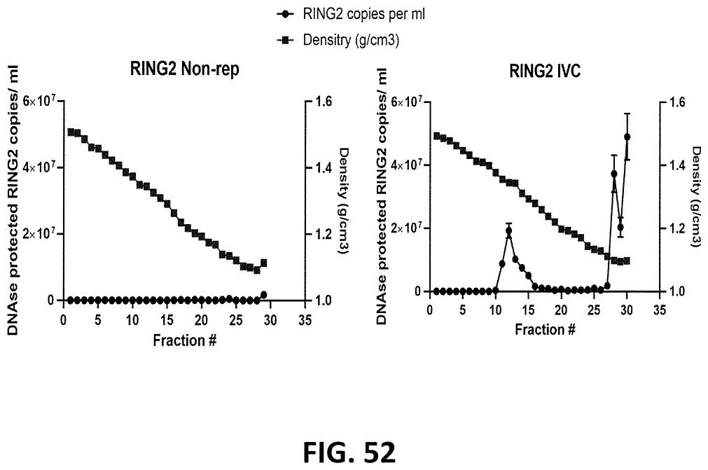

As shown in FIG. 52, the sample transfected with RING2 IVC showed a peak viral titer in fractions with a density of approximately 1.32 g/cm³. This density is consistent with the previously described density of anelloviruses in CsCl. In contrast, the sample transfected with negative control had no detectable viral titer in any fractions. These results demonstrate that the MOLT-4 cell line is permissive not only for RING2 gene expression and replication, but also for production of RING2 particles.

### Production of RING2 in MOLT-4 Cells is Dependent on Viral Protein Expression

To assess whether the production of RING2 particles is dependent on viral protein expression, we created RING2 mutant genomes in which either all 3 ORF1 variants including ORF1, ORF1/1, and ORF1/2 were knocked out (ORF1 KO), or all 3 ORF2 variants including ORF2, ORF2/2, and ORF2/3 were knocked out (ORF2 KO). These mutant genomes were generated by inserting premature stop codons into the open reading frames as specified in the Methods section.

To confirm successful knockout of the target proteins, plasmids encoding a single copy of either the wild-type RING2 genome, the ORF1 KO genome, or the ORF2 KO genome were transfected into MOLT-4 cells. Western blot analysis was performed at 2 days post-transfection. As expected, the ORF1 KO mutant did not express any detectable ORF1 protein. Similarly, ORF2 KO mutant did not express any detectable levels of ORF2 proteins or its isoforms.

To test whether these mutants can produce RING2 virus particles, MOLT-4 cells were transfected with either a plasmid encoding two copies of the wild-type RING2 genome in tandem (WT RING2 tandem), an in vitro circularized ORF1 knockout genome (ORF1 KO IVC), or an in vitro circularized ORF2 knockout genome (ORF2 KO IVC), or were co-transfected with ORF1 KO IVC and ORF2 KO IVC. Samples were assayed for RING2 production using isopycnic CsCl step gradient.

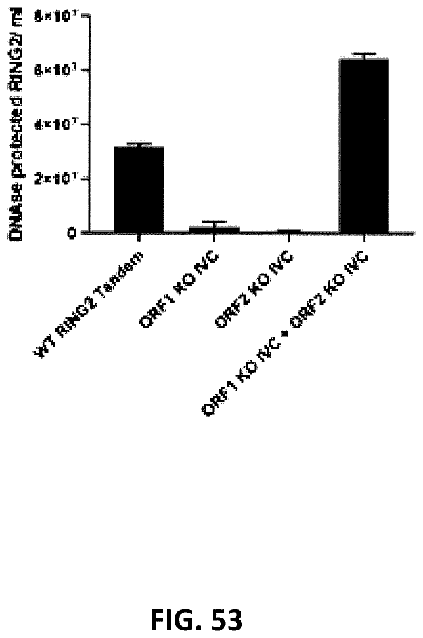

As expected, WT RING2 tandem produced RING2 particles (FIG. 53). Knocking out the expression of ORF1 and its variants or ORF2 and its variants significantly disrupted the ability to produce the virus in MOLT-4 cells. Interestingly, the mutant genomes were able to trans-complement each other, which was expected assuming co-transfection of the same cells, given that ORF1 KO IVC can produce ORF2 and its variants while ORF2 KO IVC can produce ORF1 and its variants. Overall, these findings demonstrate that the production of RING2 particles in transfected MOLT-4 cells is dependent on viral protein expression.

### Transmission Electron Microscopy (TEM) Analysis of RING2 Anellovirus

Visualization of recombinant anellovirus particles has been previously performed only for chicken anemia virus, an avian virus in the Anelloviridae family. To further our understanding of the structural biology of human anelloviruses, we analyzed RING2 by TEM.

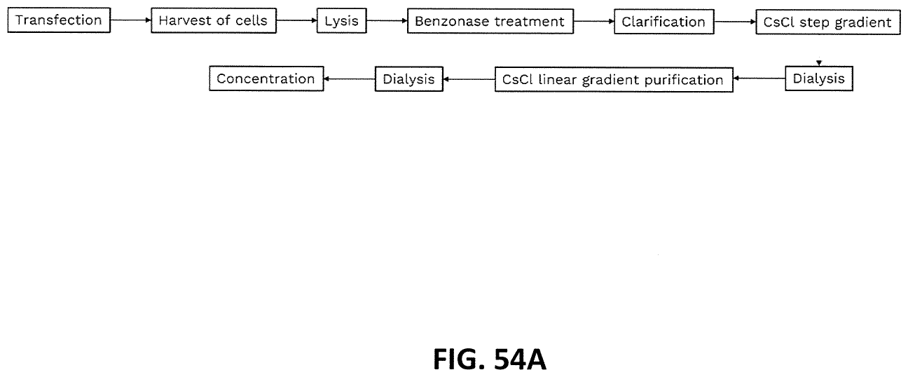

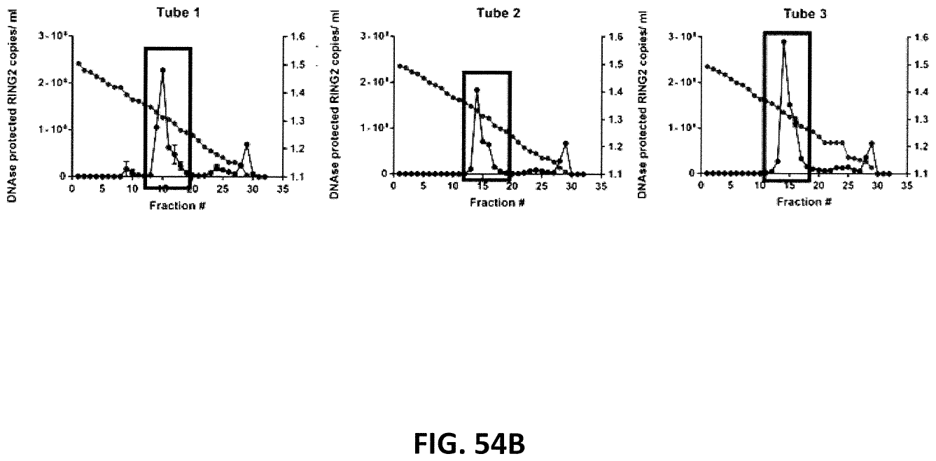

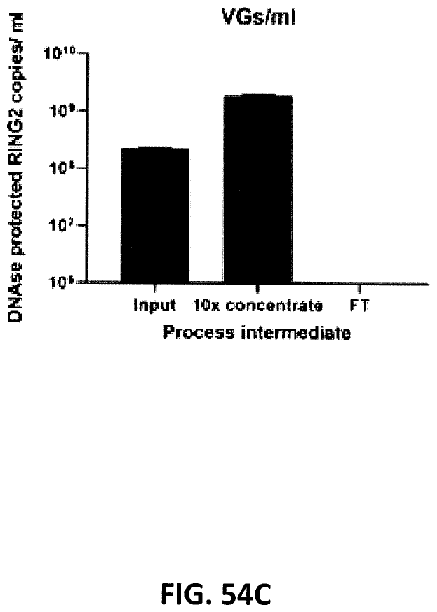

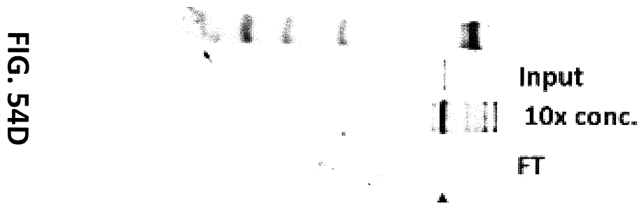

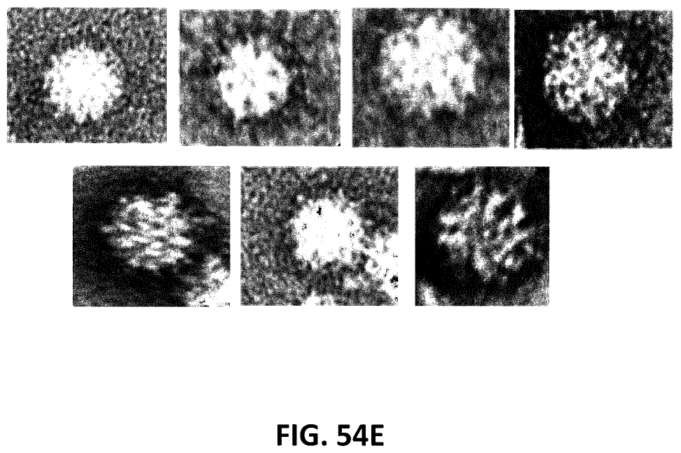

A schematic of the production and purification methodology of RING2 is depicted in FIG. 54A. Briefly, MOLT-4 cells were transfected with a plasmid containing two copies of the RING2 genome in tandem and harvested four days post-transfection. Harvested cells were lysed and treated with Benzonase and detergent. Cell lysates were clarified to remove any cellular debris and subjected to CsCl step gradient to concentrate the virus particles followed by overnight dialysis to remove any CsCl. Dialyzed material was subjected to CsCl linear gradient followed by fractionation. Each fraction was analyzed for density and viral titer.

As expected, all linear gradient tubes had a peak for DNAse protected RING2 titer at the expected density of 1.32 g/cm³. Representative profiles for density against viral titer in each fraction of the linear gradient are shown in FIG. 54B.

Next, we pooled the fractions in the peak for all 12 tubes of linear gradient, dialyzed it overnight to remove any CsCl, and concentrated the volume tenfold using diafiltration. As we used 100 kD cutoff diafiltration units, we were able to concentrate the titer of RING2 particles (FIG. 54C). Concomitantly, an increase in the titer of capsid protein ORF1 as assessed by western blot was observed (FIG. 54D).

Negative staining TEM of this purified virus preparation revealed multiple RING2 particles (FIG. 54E). The structure of the anellovirus particles we detected was consistent with the structure previously described for chicken anemia virus, i.e., an icosahedral capsid with trumpet-shaped capsomeres.

### Discovery of an Anellovirus in Human Retinal Pigment Epithelial Cells

Anelloviruses have been isolated from numerous human non-blood tissues, such as bone marrow, liver, and both the ocular surface and vitreous fluid of the eye. The low abundance of anelloviruses in such tissues has previously made measuring levels of diversity and isolating complete genomes difficult. We investigated the specific anellovirus lineages present in four eye sub-section tissues from the same subject using our AnelloScope platform. We recovered several anellovirus genomes, across all three genera, from half of the investigated eye sub-section tissues and successfully isolated a putative full-length circularized genome designated as RING19.

To explore the diversity in the four eye sub-section tissues (cornea, macula, sclera and retina pigment epithelia) we conducted two deep short-read sequencing (with and without bead baited target enrichment) and one shallow long-read sequencing run to recover an appropriate amount of genomic anellovirus data. An aggregate of 28.71 Gbp of short read sequence data and 1.41 Gbp of long read sequence data were generated across all sequencing runs of which 3.71 Gbp of and 269.3 Mbp of sequence data were classified as anellovirus from short and long read sequencing runs respectively. Strikingly, when examining the two short read sequencing runs, the run utilizing our bead baited target enrichment protocol contributed 99.9% of those reads identified as anellovirus. These findings highlight the difficulty in isolating anellovirus genome data from non-blood tissue samples and the need for targeted approaches that both amplify the amount of anellovirus in samples and reduce the amount of host background being sequenced.

To quantify the number of anellovirus lineages in each eye sub-section tissue we assembled putative anellovirus genomes utilizing each set of short read sequencing data individually. We found eleven putative genomes that produced valid ORF1 capsid proteins that were recovered from macula and RPE tissues. We clustered the ORF1 capsid protein sequences from these eleven genomes to produce eight distinct genomes that ranged in size from 2,762 bp to 3,881 bp. The recovery of putative genomes at lengths that are near to known confirmed complete genomes indicates that our methods can recover candidate genomes that could be used vector construction and synthesis.

We next classified each of these eight representative genomes into one of the three known human anellovirus genera. We observed at least one genome in each of these genera, with the Alphatorquevirus having the most at four, followed by Betatorquevirus with 2 and finally Gammatorquevirus with one. These findings suggest that anellvirus tropism in the eye may not be restricted to a specific genus.

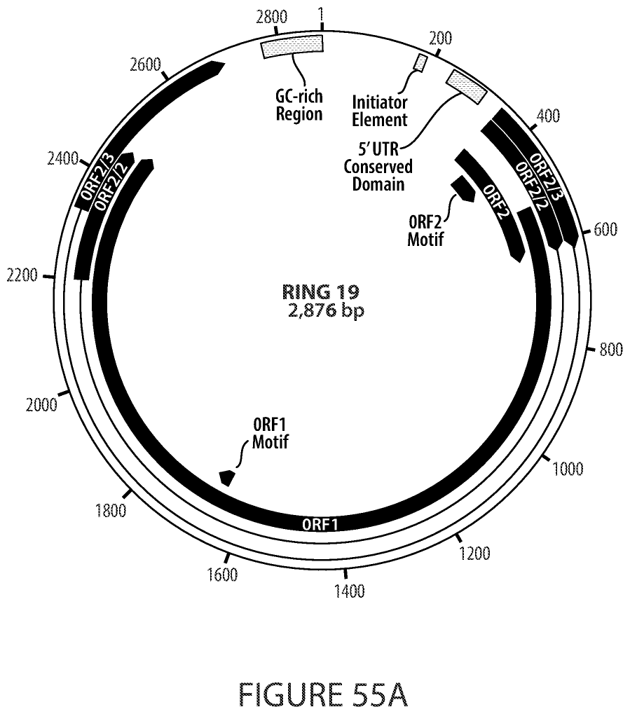

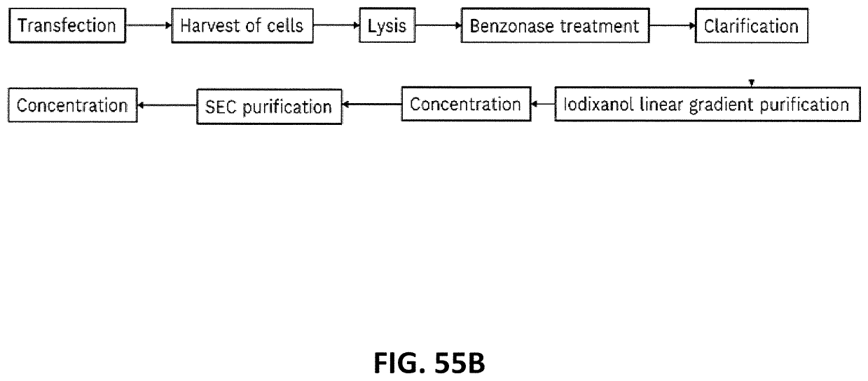

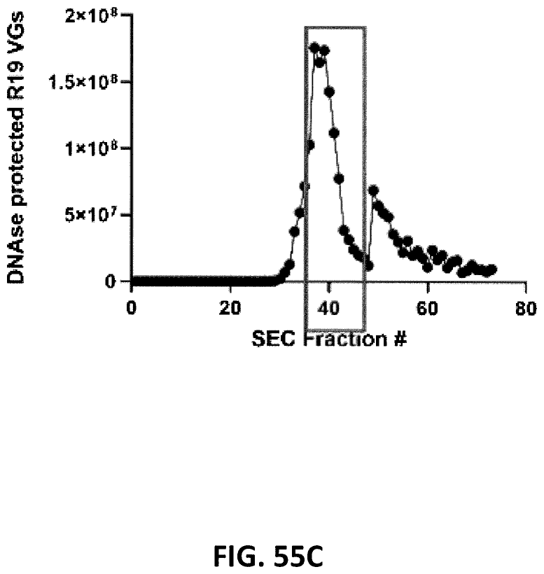

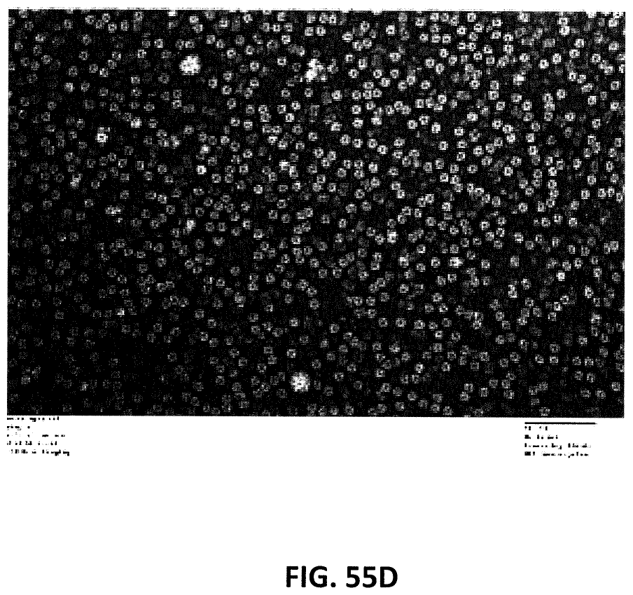

We further evaluated whether we were able to recover full-length, circularize anellovirus genomes by utilizing paired RPE-derived long-read sequencing data to resolve problematic genome regions near the GC-rich and repetitive regions. We first mapped the long read data to these genomes and observed 289,631 hits across all queried genomes. We evaluated the similarity of each of these hits to the short-read derived genomes and chose the long reads with the highest similarity to proceed to error-correction to address the high error rate encountered. We used the short-read sequence data to correct any variable positions using the consensus found at each of these sites. Once polished we attempted to circularize each sequence by looking for overlap at each of the arbitrary ends of these correct long reads. Of these RPE-derived putative genomes only one circularized and was designated RING19 (FIG. 55A).

### Production of RING19 Virus Particles In Vitro

Whereas RING2 had been previously sequenced from human pleural effusion samples and reported in the literature [Galmès 2013], RING19 is an anellovirus that we isolated from human retinal pigment epithelium as described above. As RING2 and RING19 both belong to the Betatorquevirus genus of human anelloviruses, we hypothesized that the MOLT-4 cell line would similarly be permissive for the production of RING19. We generated plasmids containing either a single copy or tandem copies of the RING19 genome. The plasmids were electroporated into MOLT-4 cells, incubated for 4 days, and processed for analysis using iodixanol linear gradient. We detected a clear peak for the RING19 titer in fractions with a density of 1.25 g/cm³ for samples transfected with the tandem RING19 genome-containing plasmid. This peak was significantly higher in these samples compared to samples transfected with a single RING19 genome-containing plasmid, indicating that MOLT-4 cells indeed are permissive for the production of RING19 as well as RING2.

Next, we repeated the production of RING19 in MOLT-4 cells at a higher scale to purify virus particles for visualization by TEM. Briefly, the processed lysates of the transfected cells were subjected to a two-step purification including iscopycnic centrifugation using an iodixanol linear gradient followed by size exclusion chromatography (SEC) (FIG. 55B). A clear peak for the RING19 titer was detected in SEC fractions in which anellovirus particles would be expected to migrate (FIG. 55C). When these fractions were pooled and concentrated for TEM analysis, we detected multiple RING19 particles as shown in FIGS. 55D and 55E. The morphology of these RING19 particles was consistent with the morphology of RING2 particles (FIG. 54E).

### Example 21. Ring19 Particles Demonstrated Infectivity In Vivo

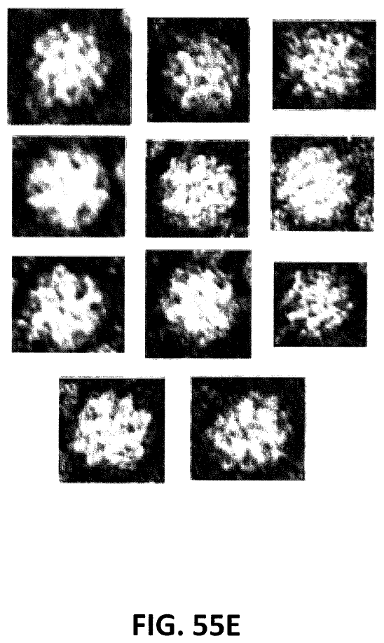

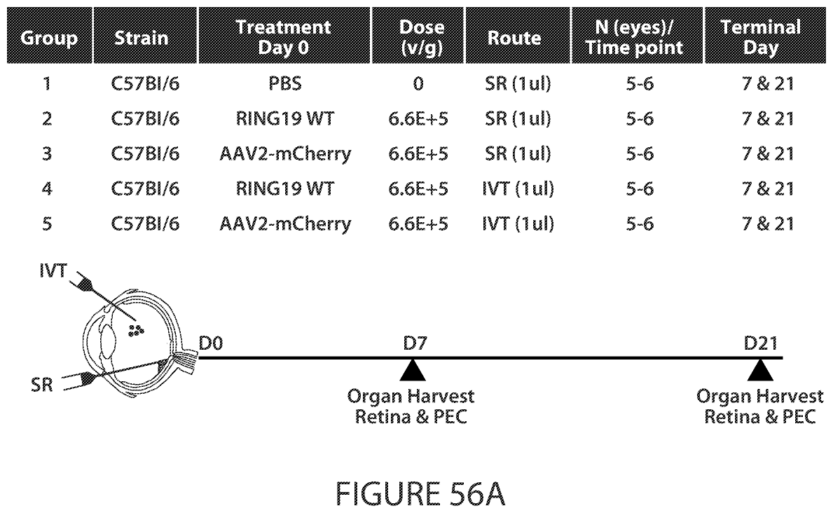

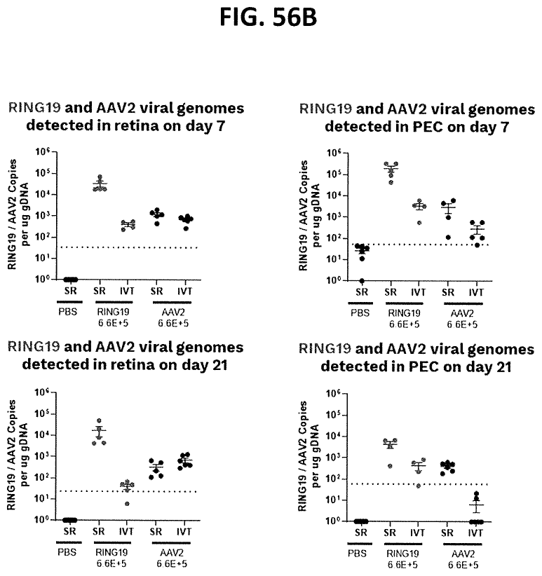

Since RING19 genome was isolated from human retinal epithelium, we examined whether it has tropism for eye tissue by testing RING19 infectivity and tropism in the eye in vivo. Mice were injected either subretinally (SR) or intravitreally (IVT) with PBS, purified WT RING19, or dose matched AAV2.mCherry as shown (FIG. 56A). Eyes were harvested and separated into the neuroretina (which contains the photoreceptors, bipolar, and ganglion cells) and the posterior eye cup (PEC, which contains the retinal pigmented epithelium, choroid, and sclera). DNA was harvested from these tissues followed by qPCR analysis to detect RING19 and AAV2.mCherry genomes, as described in the materials and methods above. RING19 demonstrated infectivity in both the neuroretina and PEC on days 7 and 21 following either subretinal or intravitreal injections (FIG. 56B). RING19 demonstrated superior targeting of the neuroretina and PEC following subretinal injection than AAV2.mCherry. This increase in infectivity was observed on both days 7 and 21. For intravitreal injections, RING19 also demonstrated superior infectivity in the PEC on days 7 and 21 compared to AAV2.mCherry. Additionally, we also detected RING19 infectivity in the neuroretina following intravitreal delivery on days 7 and 21. These data show that RING19, which was isolated from the RPE of a human donor, demonstrates superior targeting of the PEC than AAV2.

### Example 22. Ring 2 Infectivity in Retina and PEC Following Subretinal and Intravitreal Injection

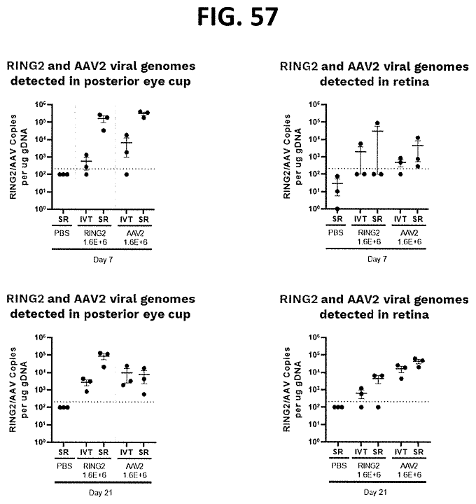

In one example, infectivity and tropism of Ring2 anellovirus for the eye was tested in vivo in mice. Briefly, mice were injected either subretinally or intravitreally with PBS, Ring 2, or dose-matched AAV2.mCherry. Eyes were harvested and separated into the neuroretina (which contains the photoreceptors, bipolar, and ganglion cells) and the posterior eye cup (PEC, which contains the retinal pigmented epithelium, choroid, and sclera). As shown in FIG. 57, Ring 2 demonstrated infectivity in both the neuroretina and PEC on days 7 and 21 following either subretinal or intravitreal injections. Ring 2 demonstrated similar targeting of the neuroretina and PEC following subretinal injection to AAV2.mCherry. These data show that Ring 2, which was isolated from the pleural effusion of a human donor, can target eye tissues at a similar level to AAV2.

### Example 23. CAV Infectivity and Transduction in Retina and PEC Following Subretinal and Intravitral Injection

In an example, the capacity of chicken anemia virus (CAV) for infectivity, tropism, and transduction of the eye were tested in vivo in mice. Briefly, mice were injected subretinally with PBS, CAV carrying nanoluciferase payload (Ring46.nLuc), dose-matched AAV2.nLuc, or a high-dose of AAV2.nLuc. Eyes were harvested and separated into the neuroretina (which contains the photoreceptors, bipolar, and ganglion cells) and the posterior eye cup (PEC, which contains the retinal pigmented epithelium, choroid, and sclera).

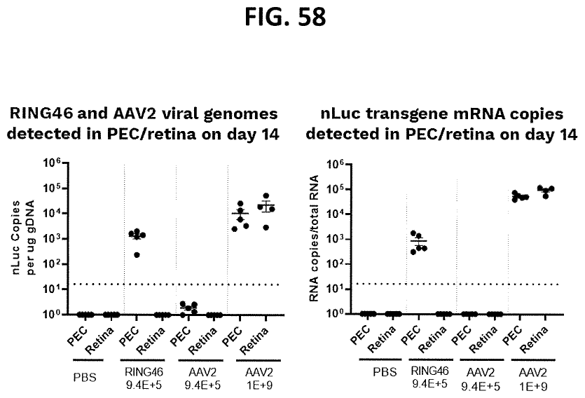

As shown in FIG. 58, CAV demonstrated infectivity in the PEC on day 14 following subretinal injections. CAV vector carrying nLuc payload demonstrated superior targeting of the PEC following subretinal injection than dose-matched AAV2.nLuc. In addition, nLuc mRNA was detected in the PEC following subretinal delivery with CAV vector carrying nLuc payload, whereas nLuc mRNA could only be detected in the high dose group of AAV2.nLuc. These data show that CAV demonstrates greater targeting and transduction of the PEC than AAV2.

### Example 24. Vectorization of Ring19 and Rescue of Ring19 Anellovectors Carrying Exogenous nLuc Payload

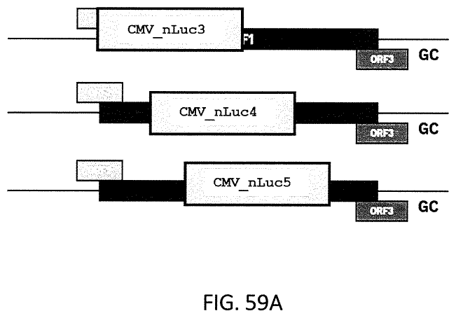

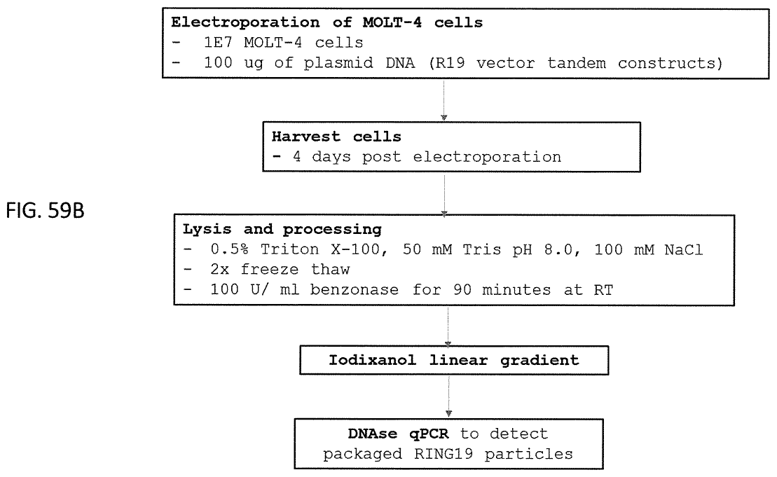

In this example, Ring19 was vectorized and packaged. Ring19 anellovector particles were shown to be produced that carry a genetic element encoding an exogenous payload. In brief, a set of exemplary tandem anellovector genome constructs were generated in which the first copy of the Ring19 genome is wild type and the second copy of the Ring19 genome comprises a CMV_nLuc cassette inserted at various positions of the Ring19 genome. As shown in FIG. 59A, in the second copy of the Ring19 genome of the tandem construct, the nLuc cassette replaced a corresponding portion of the Ring19 genome sequence. In a first exemplary Ring19 anellovector construct (referred to herein as the CMV_nLuc3 construct or pTandem R19_nLuc3), the CMV_nLuc cassette replaces a C-terminal portion of the ORF2 gene as well as an N-terminal portion of the ORF1 gene. In a second exemplary Ring19 anellovector construct (referred to herein as the CMV_nLuc4 construct or pTandem R19_nLuc4), the CMV_nLuc cassette replaces an internal portion of the ORF1 gene. In a third exemplary Ring19 anellovector construct (referred to herein as the CMV_nLuc5 construct or pTandem R19_nLuc5), the CMV_nLuc cassette replaces an internal portion of the ORF1 gene that is more C-terminal relative to the position replaced in the CMV_nLuc4 construct.

In particular, a sliding window approach was used to determine permissive deletion limits within Ring19 genome. The nLuc3 deletion removed from amino acid 56 of ORF2, 2/2, and 2/3 to amino acid 324 of ORF1. The nLuc4 deletion removed from amino acid 96 to amino acid 424 of ORF1. The nLuc5 deletion removed from amino acid 196 to amino acid 524 of ORF1. The Methionine of ORF1 and ORF2, 2/2, 2/3 were changed to encode a Lysine (ATG to AAA) in the second copy of the Ring19 genome with the nano-luciferase cassette insertion to knock out gene expression of these ORFs such that only the first wild type copy of the Ring19 genome is capable of ORF1 and ORF2 gene expression.

The nano-luciferase (nLuc) sequence used herein comprised the amino acid sequence:

(SEQ ID NO: 10)
MVFTLEDFVGDWRQTAGYNLDQVLEQGGVSSLFQNLGVSVTPIQRIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLCERILA

Table D1 provides the sequence for the parent construct comprising two wild-type Ring19 genome sequences in tandem. Tables D2-D4 provide sequences for the Ring19 nLuc anellovector tandem constructs. Selected silent mutations were introduced to remove internal BsaI restriction sites and facilitate cloning.

TABLE D1
Exemplary Ring19 Anellovirus tandem construct sequence

| | |
|---|---|
| Name | RING 19 WT-WT tandem construct |
| Genus/Clade | Betatorquevirus |
| Accession | N/A |

Full Sequence: 5752 bp

| | |
|---|---|
| 1 | cctaataaat attcaacagg aaaaccacct aattagaatt gccgaccaca aaccgtcact |
| 61 | tacttctcct ttttgcactt acttcctctt ttacttatta ttattcatta cattaattaa |
| 121 | taatcactgt aattccgggg aggagctaac aatctatata actaactaca cttccgaatg |
| 181 | gctgagttta tgccgccaga cggagacggg atcacttcag tgactccagg ctgaacttgg |
| 241 | gcgggagccg aaggtgagtg caaccaccgt agtctagggg caattcgggc tagttcagta |
| 301 | tggcggaacg ggcaagaaac ttaaatatta ttattttaca gatgcaaata caaccaccta |
| 361 | ttagaacctt caaacaaaca atttcagatt ggaaaaactt aattgtccac gttcacgaca |
| 421 | acatttgcaa ctgcaataaa ccattagaac acactattga tacctgtatc accaatccag |
| 481 | atgaattaag attaaacaaa tctactaaac aacaactaca aaaatgcctt ggtaccccag |
| 541 | aagaagatac ccaagaagac gttatcgatg gcttcgcaga tggagagcta gacgcccttt |
| 601 | tcgcccaaga tacagaagaa gatactgggt aagaaactat tctcgaaaga gaaaactatt |
| 661 | taaaataaca accaaagaat ggcaaccaaa agttataaga aagactcatg taaagggcac |
| 721 | ctatcctttg tttctttgta caaagcacag aattaacaat aatatgatac aatatttaga |
| 781 | ctctatagct ccagaacact attacggagg aggaggattt tcaataatgc aattttcctt |
| 841 | acaagcctta tatgaagaat ttataaaagc aaaaaactgg tggactaata caaactgctt |
| 901 | tttaccactt gtaagatata tgggttgctc attcaaattt tataaaactg aattttatga |
| 961 | ttatattgta ctaattgaaa gatgttatcc acttgcttgt actgatgaaa tgtacttatc |
| 1021 | tactcaacct agtattatga tgcttacaag aaaatgtatt tttgtaccat gcaaacaaaa |
| 1081 | cagcaaaggt aaaaaacctt acaaaaaagt tagagtaaga ccaccttcac aaatgactac |
| 1141 | aggatggcat ttctcacaag acttagcaaa catgccactt gtagtactaa aaacttcagt |
| 1201 | atgcagcttt gacagatatt acacagacag tacagctaaa tcaaccacaa taggctttaa |
| 1261 | aacacttaac acacaaacat ttagatatca tgactggcag gaaccaccta caacaggata |
| 1321 | caaaccacaa aacctactat ggttttatgg agcagaaaac ggatcaccag tagaccccaa |
| 1381 | caacacaata gtatcaaacc taatatactt aggaggcaca ggaccttatg aaaaaggcac |
| 1441 | accaataaaa acaaacataa gcaattactt ttcagagcct aaactgtggg gaaatatatt |
| 1501 | tcacgatgat tatacatcag gaacatcacc cgtgtttgtt acaaacaaat caccatcaga |
| 1561 | aattaaaacc gcatggaaca ctataaaaga cttaactgtt aaagctagcg gtgtatttac |
| 1621 | attaagaaca attccactat ggctaccttg cagatacaac ccatttgcag acaaagcaac |
| 1681 | caacaacaaa atatggctag tttctataca ttcagaccac acagaatgga aaccaataga |
| 1741 | caatccatta ctacaacgaa cagaccttcc tttatggtta cttgtatggg gttggcaaga |
| 1801 | ttggcagaaa aaaaaccaac aaacttcaca acctgatatt aattatttaa cagtaatatc |
| 1861 | ttcaccatat atatcatgct acccaaaatt agattactat gtgttactag atgaaggatt |
| 1921 | ttgggagggt cactcaacat acatagagtc aattacagac tcagacaaaa aacactggta |
| 1981 | ccctaaaaat agatttcaaa tagaaacact taatctaata gctaacacag gtccaggaac |
| 2041 | tgtaaaacta agagaaaacc aagcagcaga aggtcacatg gtatatcgct ttaattttaa |
| 2101 | gcttggagga tgtcccgcac cgatggaaaa aatatgtgac cctagcaaac aatccaaata |
| 2161 | tcctattccc aataaccagc aacaaacaac ttcgttgcag agtccagaaa acccaattca |
| 2221 | aacctatctc tacgacttcg acgaaaggag gggcctactt acagaaagag ctacaaaaag |
| 2281 | aatcaaacaa gatcacacat ctgaaaaaac tgttttgcca tttacaggag cagcaacaga |
| 2341 | cctccccata ctccaaacaa catcacagga ggaaagctcc tcggaagaag aagaagagca |
| 2401 | acaagcggag aagaaactac tccagctccg aagaaagcag caccgactcc gggagcgaat |
| 2461 | cctccagcta ttagacatac aaaatacata ataaaacaaa gtactgtaaa aattgatatg |
| 2521 | tttggagata ctcatgtacc taaccgtaga atgaccccag aagaatttga acaagaacta |
| 2581 | attgtcgctg gtgtttttcg cagacctcct tgttactata taaaagatag acctacttat |
| 2641 | ccttatgtac caaaacctac tgatgaaaaa tgtatggtaa actttgactt aaactttcct |
| 2701 | taataaacta cgcctgcaaa ctttcactct cggtgtccat ttatataaga taaaacttaa |
| 2761 | ataaacatcc accactctcc caaatacgca ggcgcacaag ggggctccgc ccccttaaac |
| 2821 | ccccaagggg gctccgcccc cttaaacccc caagggggct ccgccccctt acacccccta |
| 2881 | ataaatattc aacaggaaaa ccacctaatt agaattgccg accacaaacc gtcacttact |
| 2941 | tctccttttt gcacttactt cctcttttac ttattattat tcattacatt aattaataat |
| 3001 | cactgtaatt ccggggagga gctaacaatc tatataacta actacacttc cgaatggctg |
| 3061 | agtttatgcc gccagacgga gacgggatca cttcagtgac tccaggctga acttgggcgg |
| 3121 | gagccgaagg tgagtgcaac caccgtagtc taggggcaat tcgggctagt tcagtatggc |
| 3181 | ggaacgggca agaaacttaa atattattat tttacagatg caaatacaac cacctattag |
| 3241 | aaccttcaaa caaacaattt cagattggaa aaacttaatt gtccacgttc acgacaacat |
| 3301 | ttgcaactgc aataaaccat tagaacacac tattgatacc tgtatcacca atccagatga |
| 3361 | attaagatta aacaaatcta ctaaacaaca actacaaaaa tgccttggta ccccagaaga |
| 3421 | agatacccaa gaagacgtta tcgatggctt cgcagatgga gagctagacg cccttttcgc |
| 3481 | ccaagataca gaagaagata ctgggtaaga aactattctc gaaagagaaa actatttaaa |
| 3541 | ataacaacca aagaatggca accaaaagtt ataagaaaga ctcatgtaaa gggcacctat |
| 3601 | cctttgtttc tttgtacaaa gcacagaatt aacaataata tgatacaata tttagactct |
| 3661 | atagctccag aacactatta cggaggagga ggattttcaa taatgcaatt ttccttacaa |
| 3721 | gccttatatg aagaatttat aaaagcaaaa aactggtgga ctaatacaaa ctgcttttta |
| 3781 | ccacttgtaa gatatatggg ttgctcattc aaattttata aaactgaatt ttatgattat |
| 3841 | attgtactaa ttgaaagatg ttatccactt gcttgtactg atgaaatgta cttatctact |
| 3901 | caacctagta ttatgatgct tacaagaaaa tgtatttttg taccatgcaa acaaaacagc |
| 3961 | aaaggtaaaa aaccttacaa aaaagttaga gtaagaccac cttcacaaat gactacagga |
| 4021 | tggcatttct cacaagactt agcaaacatg ccacttgtag tactaaaaac ttcagtatgc |
| 4081 | agctttgaca gatattacac agacagtaca gctaaatcaa ccacaatagg ctttaaaaca |
| 4141 | cttaacacac aaacatttag atatcatgac tggcaggaac cacctacaac aggatacaaa |
| 4201 | ccacaaaacc tactatggtt ttatggagca gaaaacggat caccagtaga ccccaacaac |
| 4261 | acaatagtat caaacctaat atacttagga ggcacaggac cttatgaaaa aggcacacca |
| 4321 | ataaaaacaa acataagcaa ttacttttca gagcctaaac tgtggggaaa tatatttcac |
| 4381 | gatgattata catcaggaac atcacccgtg tttgttacaa acaaatcacc atcagaaatt |
| 4441 | aaaaccgcat ggaacactat aaaagactta actgttaaag ctagcggtgt atttacatta |
| 4501 | agaacaattc cactatggct accttgcaga tacaacccat ttgcagacaa agcaaccaac |
| 4561 | aacaaaatat ggctagtttc tatacattca gaccacacag aatggaaacc aatagacaat |
| 4621 | ccattactac aacgaacaga ccttccttta tggttacttg tatggggttg gcaagattgg |
| 4681 | cagaaaaaaa accaacaaac ttcacaacct gatattaatt atttaacagt aatatcttca |
| 4741 | ccatatatat catgctaccc aaaattagat tactatgtgt tactagatga aggattttgg |
| 4801 | gagggtcact caacatacat agagtcaatt acagactcag acaaaaaaca ctggtaccct |
| 4861 | aaaaatagat ttcaaataga aacacttaat ctaatagcta acacaggtcc aggaactgta |
| 4921 | aaactaagag aaaaccaagc agcagaaggt cacatggtat atcgctttaa ttttaagctt |
| 4981 | ggaggatgtc ccgcaccgat ggaaaaaata tgtgacccta gcaaacaatc caaatatcct |
| 5041 | attcccaata accagcaaca aacaacttcg ttgcagagtc cagaaaaccc aattcaaacc |
| 5101 | tatctctacg acttcgacga aaggaggggc ctacttacag aaagagctac aaaaagaatc |
| 5161 | aaacaagatc acacatctga aaaaactgtt ttgccattta caggagcagc aacagacctc |
| 5221 | cccatactcc aaacaacatc acaggaggaa agctcctcgg aagaagaaga agagcaacaa |
| 5281 | gcggagaaga aactactcca gctccgaaga aagcagcacc gactccggga gcgaatcctc |
| 5341 | cagctattag acatacaaaa tacataataa aacaaagtac tgtaaaaatt gatatgtttg |
| 5401 | gagatactca tgtacctaac cgtagaatga ccccagaaga atttgaacaa gaactaattg |
| 5461 | tcgctggtgt ttttcgcaga cctccttgtt actatataaa agatagacct acttatcctt |
| 5521 | atgtaccaaa acctactgat gaaaaatgta tggtaaactt tgacttaaac tttccttaat |
| 5581 | aaactacgcc tgcaaacttt cactctcggt gtccatttat ataagataaa acttaaataa |
| 5641 | acatccacca ctctcccaaa tacgcaggcg cacaaggggg ctccgccccc ttaaaccccc |
| 5701 | aagggggctc cgccccctta aacccccaag ggggctccgc ccccttacac cc (SEQ ID NO: 6) |

Annotations:

| Putative Domain | Base range |
|---|---|
| ORF1 | 524-2491 |
| ORF2 | 342-632 |
| ORF2/2 | 342-628; 2201-2474 |
| ORF2/3 | 342-628; 2328-2703 |
| GC-rich region, or a portion thereof | 2767-2876 |
| 5′ UTR Conserved Domain, or a portion thereof | 242-312 |
| ORF1 | 3400-5367 |
| ORF2 | 3218-3508 |
| ORF2/2 | 3218-3504; 5077-5350 |
| ORF2/3 | 3218-3504; 5204-5579 |
| ORF3 | 5394-5579 |
| GC-rich region, or a portion thereof | 5643-5752 |
| 5′ UTR Conserved Domain, or a portion thereof | 3118-3188 |

TABLE D2
Exemplary Ring19 CMV_nLuc3 tandem construct sequence

| | |
|---|---|
| Name | RING 19 WT-CMV_nLuc3 tandem construct |
| Genus/Clade | Betatorquevirus |
| Accession | N/A |

Full Sequence: 5743 bp

| | |
|---|---|
| 1 | cctaataaat attcaacagg aaaaccacct aattagaatt gccgaccaca aaccgtcact |
| 61 | tacttctcct ttttgcactt acttcctctt ttacttatta ttattcatta cattaattaa |
| 121 | taatcactgt aattccgggg aggagctaac aatctatata actaactaca cttccgaatg |
| 181 | gctgagttta tgccgccaga cggagacggg atcacttcag tgactccagg ctgaacttgg |
| 241 | gcgggagccg aaggtgagtg caaccaccgt agtctagggg caattcgggc tagttcagta |
| 301 | tggcggaacg ggcaagaaac ttaaatatta ttattttaca gatgcaaata caaccaccta |
| 361 | ttagaacctt caaacaaaca atttcagatt ggaaaaactt aattgtccac gttcacgaca |
| 421 | acatttgcaa ctgcaataaa ccattagaac acactattga tacctgtatc accaatccag |
| 481 | atgaattaag attaaacaaa tctactaaac aacaactaca aaaatgcctt ggtaccccag |
| 541 | aagaagatac ccaagaagac gttatcgatg gcttcgcaga tggagagcta gacgcccttt |
| 601 | tcgcccaaga tacagaagaa gatactgggt aagaaactat tctcgaaaga gaaaactatt |
| 661 | taaaataaca accaaagaat ggcaaccaaa agttataaga aagactcatg taaagggcac |
| 721 | ctatcctttg tttctttgta caaagcacag aattaacaat aatatgatac aatatttaga |
| 781 | ctctatagct ccagaacact attacggagg aggaggattt tcaataatgc aattttcctt |
| 841 | acaagcctta tatgaagaat ttataaaagc aaaaaactgg tggactaata caaactgctt |
| 901 | tttaccactt gtaagatata tgggttgctc attcaaattt tataaaactg aattttatga |
| 961 | ttatattgta ctaattgaaa gatgttatcc acttgcttgt actgatgaaa tgtacttatc |
| 1021 | tactcaacct agtattatga tgcttacaag aaaatgtatt tttgtaccat gcaaacaaaa |
| 1081 | cagcaaaggt aaaaaacctt acaaaaaagt tagagtaaga ccaccttcac aaatgactac |
| 1141 | aggatggcat ttctcacaag acttagcaaa catgccactt gtagtactaa aaacttcagt |
| 1201 | atgcagcttt gacagatatt acacagacag tacagctaaa tcaaccacaa taggctttaa |
| 1261 | aacacttaac acacaaacat ttagatatca tgactggcag gaaccaccta caacaggata |
| 1321 | caaaccacaa aacctactat ggttttatgg agcagaaaac ggatcaccag tagaccccaa |
| 1381 | caacacaata gtatcaaacc taatatactt aggaggcaca ggaccttatg aaaaaggcac |
| 1441 | accaataaaa acaaacataa gcaattactt ttcagagcct aaactgtggg gaaatatatt |
| 1501 | tcacgatgat tatacatcag gaacatcacc cgtgtttgtt acaaacaaat caccatcaga |
| 1561 | aattaaaacc gcatggaaca ctataaaaga cttaactgtt aaagctagcg gtgtatttac |
| 1621 | attaagaaca attccactat ggctaccttg cagatacaac ccatttgcag acaaagcaac |
| 1681 | caacaacaaa atatggctag tttctataca ttcagaccac acagaatgga aaccaataga |
| 1741 | caatccatta ctacaacgaa cagaccttcc tttatggtta cttgtatggg gttggcaaga |
| 1801 | ttggcagaaa aaaaaccaac aaacttcaca acctgatatt aattatttaa cagtaatatc |
| 1861 | ttcaccatat atatcatgct acccaaaatt agattactat gtgttactag atgaaggatt |
| 1921 | ttgggagggt cactcaacat acatagagtc aattacagac tcagacaaaa aacactggta |
| 1981 | ccctaaaaat agatttcaaa tagaaacact taatctaata gctaacacag gtccaggaac |
| 2041 | tgtaaaacta agagaaaacc aagcagcaga aggtcacatg gtatatcgct ttaattttaa |
| 2101 | gcttggagga tgtcccgcac cgatggaaaa aatatgtgac cctagcaaac aatccaaata |
| 2161 | tcctattccc aataaccagc aacaaacaac ttcgttgcag agtccagaaa acccaattca |
| 2221 | aacctatctc tacgacttcg acgaaaggag gggcctactt acagaaagag ctacaaaaag |
| 2281 | aatcaaacaa gatcacacat ctgaaaaaac tgttttgcca tttacaggag cagcaacaga |
| 2341 | cctccccata ctccaaacaa catcacagga ggaaagctcc tcggaagaag aagaagagca |
| 2401 | acaagcggag aagaaactac tccagctccg aagaaagcag caccgactcc gggagcgaat |
| 2461 | cctccagcta ttagacatac aaaatacata ataaaacaaa gtactgtaaa aattgatatg |
| 2521 | tttggagata ctcatgtacc taaccgtaga atgaccccag aagaatttga acaagaacta |
| 2581 | attgtcgctg gtgtttttcg cagacctcct tgttactata taaaagatag acctacttat |
| 2641 | ccttatgtac caaaacctac tgatgaaaaa tgtatggtaa actttgactt aaactttcct |
| 2701 | taataaacta cgcctgcaaa ctttcactct cggtgtccat ttatataaga taaaacttaa |
| 2761 | ataaacatcc accactctcc caaatacgca ggcgcacaag ggggctccgc ccccttaaac |
| 2821 | ccccaagggg gctccgcccc cttaaacccc caagggggct ccgccccctt acacccccta |
| 2881 | ataaatattc aacaggaaaa ccacctaatt agaattgccg accacaaacc gtcacttact |
| 2941 | tctccttttt gcacttactt cctcttttac ttattattat tcattacatt aattaataat |
| 3001 | cactgtaatt ccggggagga gctaacaatc tatataacta actacacttc cgaatggctg |
| 3061 | agtttatgcc gccagacgga gacgggatca cttcagtgac tccaggctga acttgggcgg |
| 3121 | gagccgaagg tgagtgcaac caccgtagtc taggggcaat tcgggctagt tcagtatggc |
| 3181 | ggaacgggca agaaacttaa atattattat tttacagaaa caaatacaac cacctattag |
| 3241 | aacgttcaaa caaacaattt cagattggaa aaacttaatt gtccacgttc acgacaacat |
| 3301 | ttgcaactgc aataaaccat tagaacacac tattgatacc tgtatcacca atccagatga |
| 3361 | attaagatta aacaaatcta ctaagatggt tacgcaggtg aagtacgccc cctattgacg |
| 3421 | tcaatgacgg taaatggccc gcctggcatt atgcccagta catgacctta tgggactttc |
| 3481 | ctacttggca gtacatctac gtattagtca tcgctattac catggtgatg cggttttggc |
| 3541 | agtacatcaa tgggcgtgga tagcggtttg actcacgggg atttccaagt ctccacccca |
| 3601 | ttgacgtcaa tgggagtttg ttttggcacc aaaatcaacg ggactttcca aaatgtcgta |
| 3661 | acaactccgc cccattgacg caaatgggcg gtaggcgtgt acggtgggag gtctatataa |
| 3721 | gcagagctct ctggctaact ggatctacaa aaaagcagat ccaccggtcg ccaccatggt |
| 3781 | gttcacactc gaagatttcg ttggggactg gcgacagaca gccggctaca acctggacca |
| 3841 | agtccttgaa cagggaggtg tgtccagttt gtttcagaat ctcggggtgt ccgtaactcc |
| 3901 | gatccaaagg attgtcctga gcggtgaaaa tgggctgaag atcgacatcc atgtcatcat |
| 3961 | cccgtatgaa ggtctgagcg gcgaccaaat gggccagatc gaaaaaattt ttaaggtggt |
| 4021 | gtaccctgtg gatgatcatc actttaaggt gatcctgcac tatggcacac tggtaatcga |
| 4081 | cggggttacg ccgaacatga tcgactattt cggacggccg tatgaaggca tcgccgtgtt |
| 4141 | cgacggcaaa aagatcactg taacagggac cctgtggaac ggcaacaaaa ttatcgacga |
| 4201 | gcgcctgatc aaccccgacg gctccctgct gttccgagta accatcaacg gagtgaccgg |
| 4261 | ctggcggctg tgcgaacgca ttctggcgta ataactgttc aataaaatat ctttattttc |
| 4321 | attacatctg tgtgttggtt ttttgtgtgc acctgctata tgaaatttca cgatgattat |
| 4381 | acatcaggaa catcacccgt gtttgttaca aacaaatcac catcagaaat taaaaccgca |
| 4441 | tggaacacta taaaagactt aactgttaaa gctagcggtg tatttacatt aagaacaatt |
| 4501 | ccactatggc taccttgcag atacaaccca tttgcagaca aagcaaccaa caacaaaata |
| 4561 | tggctagttt ctatacattc agaccacaca gaatggaaac caatagacaa tccattacta |
| 4621 | caacgaacag accttccttt atggttactt gtatggggtt ggcaagattg gcagaaaaaa |
| 4681 | aaccaacaaa cttcacaacc tgatattaat tatttaacag taatatcttc accatatata |
| 4741 | tcatgctacc caaaattaga ttactatgtg ttactagatg aaggattttg ggagggtcac |
| 4801 | tcaacataca tagagtcaat tacagactca gacaaaaaac actggtaccc taaaaataga |
| 4861 | tttcaaatag aaacacttaa tctaatagct aacacaggtc caggaactgt aaaactaaga |
| 4921 | gaaaaccaag cagcagaagg tcacatggta tatcgcttta attttaagct tggaggatgt |
| 4981 | cccgcaccca tggaaaaaat atgtgaccct agcaaacaat ccaaatatcc tattcccaat |
| 5041 | aaccagcaac aaacaacttc gttgcagagt ccagaaaacc caattcaaac ctatctctac |
| 5101 | gacttcgacg aaaggagggg cctacttaca gaaagagcta caaaaagaat caaacaagat |
| 5161 | cacacatctg aaaaaactgt tttgccattt acaggagcag caacagacct ccccatactc |
| 5221 | caaacaacat cacaggagga aagctcctcg gaagaagaag aagagcaaca agcggagaag |
| 5281 | aaactactcc agctccgaag aaagcagcac cgactccggg agcgaatcct ccagctatta |
| 5341 | gacatacaaa atacataata aaacaaagta ctgtaaaaat tgatatgttt ggagatactc |
| 5401 | atgtacctaa ccgtagaatg accccagaag aatttgaaca agaactaatt gtcgctggtg |
| 5461 | tttttcgcag acctccttgt tactatataa aagatagacc tacttatcct tatgtaccaa |
| 5521 | aacctactga tgaaaaatgt atggtaaact ttgacttaaa ctttccttaa taaactacgc |
| 5581 | ctgcaaactt tcactctcgg tgtccattta tataagataa aacttaaata aacatccacc |
| 5641 | actctcccaa atacgcaggc gcacaagggg gctccgcccc cttaaacccc caagggggct |
| 5701 | ccgccccctt aaacccccaa gggggctccg cccccttaca ccc (SEQ ID NO: 7) |

Annotations:

| Putative Domain | Base range |
|---|---|
| Full length ORF1 | 524-2491 |
| Full length ORF2 | 342-632 |
| ORF2/2 | 342-628; 2201-2474 |
| ORF2/3 | 342-628; 2328-2703 |
| GC-rich region, or a portion thereof | 2767-2876 |
| 5′ UTR Conserved Domain, or a portion thereof | 242-312 |
| Truncated ORF1 | 4367-5358 |
| Truncated ORF2 | 3218-3385 |
| ORF2/2 | 3218-3385; 5068-5341 |
| ORF2/3 | 3218-3385; 5195-5570 |
| ORF3 | 5385-5570 |
| CMV promoter | 3525-3728 |
| Nano-Luciferase (nLuc) | 3776-4291 |
| SV40 poly A | 4301-4349 |
| GC-rich region, or a portion thereof | 5634-5743 |
| 5′ UTR Conserved Domain, or a portion thereof | 3118-3188 |

TABLE D3
Exemplary Ring19 CMV_nLuc4 tandem construct sequence

| | |
|---|---|
| Name | RING 19 WT-CMV_nLuc4 tandem construct |
| Genus/Clade | Betatorquevirus |
| Accession | N/A |

Full Sequence: 5743 bp

| | |
|---|---|
| 1 | cctaataaat attcaacagg aaaaccacct aattagaatt gccgaccaca aaccgtcact |
| 61 | tacttctcct ttttgcactt acttcctctt ttacttatta ttattcatta cattaattaa |
| 121 | taatcactgt aattccgggg aggagctaac aatctatata actaactaca cttccgaatg |
| 181 | gctgagttta tgccgccaga cggagacggg atcacttcag tgactccagg ctgaacttgg |
| 241 | gcgggagccg aaggtgagtg caaccaccgt agtctagggg caattcgggc tagttcagta |
| 301 | tggcggaacg ggcaagaaac ttaaatatta ttattttaca gatgcaaata caaccaccta |
| 361 | ttagaacctt caaacaaaca atttcagatt ggaaaaactt aattgtccac gttcacgaca |
| 421 | acatttgcaa ctgcaataaa ccattagaac acactattga tacctgtatc accaatccag |
| 481 | atgaattaag attaaacaaa tctactaaac aacaactaca aaaatgcctt ggtaccccag |
| 541 | aagaagatac ccaagaagac gttatcgatg gcttcgcaga tggagagcta gacgcccttt |
| 601 | tcgcccaaga tacagaagaa gatactgggt aagaaactat tctcgaaaga gaaaactatt |
| 661 | taaaataaca accaaagaat ggcaaccaaa agttataaga aagactcatg taaagggcac |
| 721 | ctatcctttg tttctttgta caaagcacag aattaacaat aatatgatac aatatttaga |
| 781 | ctctatagct ccagaacact attacggagg aggaggattt tcaataatgc aattttcctt |
| 841 | acaagcctta tatgaagaat ttataaaagc aaaaaactgg tggactaata caaactgctt |
| 901 | tttaccactt gtaagatata tgggttgctc attcaaattt tataaaactg aattttatga |
| 961 | ttatattgta ctaattgaaa gatgttatcc acttgcttgt actgatgaaa tgtacttatc |
| 1021 | tactcaacct agtattatga tgcttacaag aaaatgtatt tttgtaccat gcaaacaaaa |
| 1081 | cagcaaaggt aaaaaacctt acaaaaaagt tagagtaaga ccaccttcac aaatgactac |
| 1141 | aggatggcat ttctcacaag acttagcaaa catgccactt gtagtactaa aaacttcagt |
| 1201 | atgcagcttt gacagatatt acacagacag tacagctaaa tcaaccacaa taggctttaa |
| 1261 | aacacttaac acacaaacat ttagatatca tgactggcag gaaccaccta caacaggata |
| 1321 | caaaccacaa aacctactat ggttttatgg agcagaaaac ggatcaccag tagaccccaa |
| 1381 | caacacaata gtatcaaacc taatatactt aggaggcaca ggaccttatg aaaaaggcac |
| 1441 | accaataaaa acaaacataa gcaattactt ttcagagcct aaactgtggg gaaatatatt |
| 1501 | tcacgatgat tatacatcag gaacatcacc cgtgtttgtt acaaacaaat caccatcaga |
| 1561 | aattaaaacc gcatggaaca ctataaaaga cttaactgtt aaagctagcg gtgtatttac |
| 1621 | attaagaaca attccactat ggctaccttg cagatacaac ccatttgcag acaaagcaac |
| 1681 | caacaacaaa atatggctag tttctataca ttcagaccac acagaatgga aaccaataga |
| 1741 | caatccatta ctacaacgaa cagaccttcc tttatggtta cttgtatggg gttggcaaga |
| 1801 | ttggcagaaa aaaaaccaac aaacttcaca acctgatatt aattatttaa cagtaatatc |
| 1861 | ttcaccatat atatcatgct acccaaaatt agattactat gtgttactag atgaaggatt |
| 1921 | ttgggagggt cactcaacat acatagagtc aattacagac tcagacaaaa aacactggta |
| 1981 | ccctaaaaat agatttcaaa tagaaacact taatctaata gctaacacag gtccaggaac |
| 2041 | tgtaaaacta agagaaaacc aagcagcaga aggtcacatg gtatatcgct ttaattttaa |
| 2101 | gcttggagga tgtcccgcac cgatggaaaa aatatgtgac cctagcaaac aatccaaata |
| 2161 | tcctattccc aataaccagc aacaaacaac ttcgttgcag agtccagaaa acccaattca |
| 2221 | aacctatctc tacgacttcg acgaaaggag gggcctactt acagaaagag ctacaaaaag |
| 2281 | aatcaaacaa gatcacacat ctgaaaaaac tgttttgcca tttacaggag cagcaacaga |
| 2341 | cctccccata ctccaaacaa catcacagga ggaaagctcc tcggaagaag aagaagagca |
| 2401 | acaagcggag aagaaactac tccagctccg aagaaagcag caccgactcc gggagcgaat |
| 2461 | cctccagcta ttagacatac aaaatacata ataaaacaaa gtactgtaaa aattgatatg |
| 2521 | tttggagata ctcatgtacc taaccgtaga atgaccccag aagaatttga acaagaacta |
| 2581 | attgtcgctg gtgtttttcg cagacctcct tgttactata taaaagatag acctacttat |
| 2641 | ccttatgtac caaaacctac tgatgaaaaa tgtatggtaa actttgactt aaactttcct |
| 2701 | taataaacta cgcctgcaaa ctttcactct cggtgtccat ttatataaga taaaacttaa |
| 2761 | ataaacatcc accactctcc caaatacgca ggcgcacaag ggggctccgc ccccttaaac |
| 2821 | ccccaagggg gctccgcccc cttaaacccc caagggggct ccgccccctt acacccccta |
| 2881 | ataaatattc aacaggaaaa ccacctaatt agaattgccg accacaaacc gtcacttact |
| 2941 | tctccttttt gcacttactt cctcttttac ttattattat tcattacatt aattaataat |
| 3001 | cactgtaatt ccggggagga gctaacaatc tatataacta actacacttc cgaatggctg |
| 3061 | agtttatgcc gccagacgga gacgggatca cttcagtgac tccaggctga acttgggcgg |
| 3121 | gagccgaagg tgagtgcaac caccgtagtc taggggcaat tcgggctagt tcagtatggc |
| 3181 | ggaacgggca agaaacttaa atattattat tttacagaaa caaatacaac cacctattag |
| 3241 | aacgttcaaa caaacaattt cagattggaa aaacttaatt gtccacgttc acgacaacat |
| 3301 | ttgcaactgc aataaaccat tagaacacac tattgatacc tgtatcacca atccagatga |
| 3361 | attaagatta aacaaatcta ctaagcagca actacaaaaa aaccttggta ccccagaaga |
| 3421 | agatacccaa gaagacgtta tcgatggctt cgcagatgga gagctagacg cccttttcgc |
| 3481 | ccaagataca gaagaagata ctgggtaaga aactattctc gaaagagaaa actatttaaa |
| 3541 | ataacaacca aagaatggca accaaaagtt ataagaaaga ctcatgtaaa gggcacctat |
| 3601 | cctttgtttc tttgtacaaa gcacagaatt aacaataata tgatacaata tttagactct |
| 3661 | atagctccag aacactatta cggaatggtt acgcaggtga agtacgcccc ctattgacgt |
| 3721 | caatgacggt aaatggcccg cctggcatta tgcccagtac atgaccttat gggactttcc |
| 3781 | tacttggcag tacatctacg tattagtcat cgctattacc atggtgatgc ggttttggca |
| 3841 | gtacatcaat gggcgtggat agcggtttga ctcacgggga tttccaagtc tccaccccat |
| 3901 | tgacgtcaat gggagtttgt tttggcacca aaatcaacgg gactttccaa aatgtcgtaa |
| 3961 | caactccgcc ccattgacgc aaatgggcgg taggcgtgta cggtgggagg tctatataag |
| 4021 | cagagctctc tggctaactg gatctacaaa aaagcagatc caccggtcgc caccatggtg |
| 4081 | ttcacactcg aagatttcgt tggggactgg cgacagacag ccggctacaa cctggaccaa |
| 4141 | gtccttgaac agggaggtgt gtccagtttg tttcagaatc tcggggtgtc cgtaactccg |
| 4201 | atccaaagga ttgtcctgag cggtgaaaat gggctgaaga tcgacatcca tgtcatcatc |
| 4261 | ccgtatgaag gtctgagcgg cgaccaaatg ggccagatcg aaaaaatttt taaggtggtg |
| 4321 | taccctgtgg atgatcatca ctttaaggtg atcctgcact atggcacact ggtaatcgac |
| 4381 | ggggttacgc cgaacatgat cgactatttc ggacggccgt atgaaggcat cgccgtgttc |
| 4441 | gacggcaaaa agatcactgt aacagggacc ctgtggaacg gcaacaaaat tatcgacgag |
| 4501 | cgcctgatca accccgacgg ctccctgctg ttccgagtaa ccatcaacgg agtgaccggc |
| 4561 | tggcggctgt gcgaacgcat tctggcgtaa taactgttca ataaaatatc tttattttca |
| 4621 | ttacatctgt gtgttggttt tttgtgtgca cctgctatat gaaaagattg gcagaaaaaa |
| 4681 | aaccaacaaa cttcacaacc tgatattaat tatttaacag taatatcttc accatatata |
| 4741 | tcatgctacc caaaattaga ttactatgtg ttactagatg aaggattttg ggagggtcac |
| 4801 | tcaacataca tagagtcaat tacagactca gacaaaaaac actggtaccc taaaaataga |
| 4861 | tttcaaatag aaacacttaa tctaatagct aacacaggtc caggaactgt aaaactaaga |
| 4921 | gaaaaccaag cagcagaagg tcacatggta tatcgcttta attttaagct tggaggatgt |
| 4981 | cccgcaccca tggaaaaaat atgtgaccct agcaaacaat ccaaatatcc tattcccaat |
| 5041 | aaccagcaac aaacaacttc gttgcagagt ccagaaaacc caattcaaac ctatctctac |
| 5101 | gacttcgacg aaaggagggg cctacttaca gaaagagcta caaaaagaat caaacaagat |
| 5161 | cacacatctg aaaaaactgt tttgccattt acaggagcag caacagacct ccccatactc |
| 5221 | caaacaacat cacaggagga aagctcctcg gaagaagaag aagagcaaca agcggagaag |
| 5281 | aaactactcc agctccgaag aaagcagcac cgactccggg agcgaatcct ccagctatta |
| 5341 | gacatacaaa atacataata aaacaaagta ctgtaaaaat tgatatgttt ggagatactc |
| 5401 | atgtacctaa ccgtagaatg accccagaag aatttgaaca agaactaatt gtcgctggtg |
| 5461 | tttttcgcag acctccttgt tactatataa aagatagacc tacttatcct tatgtaccaa |
| 5521 | aacctactga tgaaaaatgt atggtaaact ttgacttaaa ctttccttaa taaactacgc |
| 5581 | ctgcaaactt tcactctcgg tgtccattta tataagataa aacttaaata aacatccacc |
| 5641 | actctcccaa atacgcaggc gcacaagggg gctccgcccc cttaaacccc caagggggct |
| 5701 | ccgccccctt aaacccccaa gggggctccg cccccttaca ccc (SEQ ID NO: 8) |

Annotations:

| Putative Domain | Base range |
|---|---|
| Full length ORF1 | 524-2491 |
| Full length ORF2 | 342-632 |
| ORF2/2 | 342-628; 2201-2474 |
| ORF2/3 | 342-628; 2328-2703 |
| GC-rich region, or a portion thereof | 2767-2876 |
| 5′ UTR Conserved Domain, or a portion thereof | 242-312 |
| ORF1 with deletion | 3400-3684; 4663-5358 |
| Full length ORF2 | 3218-3508 |
| ORF2/2 | 3218-3504; 5068-5341 |
| ORF2/3 | 3218-3504; 5195-5570 |
| ORF3 | 5385-5570 |
| CMV promoter | 3824-4027 |
| Nano-Luciferase (nLuc) | 4075-4590 |
| SV40 poly A | 4600-4648 |
| GC-rich region, or a portion thereof | 5634-5743 |
| 5′ UTR Conserved Domain, or a portion thereof | 3118-3188 |

TABLE D4
Exemplary Ring19 CMV_nLuc5 tandem construct sequence

| | |
|---|---|
| Name | RING 19 WT-CMV_nLuc5 tandem construct |
| Genus/Clade | Betatorquevirus |
| Accession | N/A |

Full Sequence: 5743 bp

| | |
|---|---|
| 1 | cctaataaat attcaacagg aaaaccacct aattagaatt gccgaccaca aaccgtcact |
| 61 | tacttctcct ttttgcactt acttcctctt ttacttatta ttattcatta cattaattaa |
| 121 | taatcactgt aattccgggg aggagctaac aatctatata actaactaca cttccgaatg |
| 181 | gctgagttta tgccgccaga cggagacggg atcacttcag tgactccagg ctgaacttgg |
| 241 | gcgggagccg aaggtgagtg caaccaccgt agtctagggg caattcgggc tagttcagta |
| 301 | tggcggaacg ggcaagaaac ttaaatatta ttattttaca gatgcaaata caaccaccta |
| 361 | ttagaacctt caaacaaaca atttcagatt ggaaaaactt aattgtccac gttcacgaca |
| 421 | acatttgcaa ctgcaataaa ccattagaac acactattga tacctgtatc accaatccag |
| 481 | atgaattaag attaaacaaa tctactaaac aacaactaca aaaatgcctt ggtaccccag |
| 541 | aagaagatac ccaagaagac gttatcgatg gcttcgcaga tggagagcta gacgcccttt |
| 601 | tcgcccaaga tacagaagaa gatactgggt aagaaactat tctcgaaaga gaaaactatt |
| 661 | taaaataaca accaaagaat ggcaaccaaa agttataaga aagactcatg taaagggcac |
| 721 | ctatcctttg tttctttgta caaagcacag aattaacaat aatatgatac aatatttaga |
| 781 | ctctatagct ccagaacact attacggagg aggaggattt tcaataatgc aattttcctt |
| 841 | acaagcctta tatgaagaat ttataaaagc aaaaaactgg tggactaata caaactgctt |
| 901 | tttaccactt gtaagatata tgggttgctc attcaaattt tataaaactg aattttatga |
| 961 | ttatattgta ctaattgaaa gatgttatcc acttgcttgt actgatgaaa tgtacttatc |
| 1021 | tactcaacct agtattatga tgcttacaag aaaatgtatt tttgtaccat gcaaacaaaa |
| 1081 | cagcaaaggt aaaaaacctt acaaaaaagt tagagtaaga ccaccttcac aaatgactac |
| 1141 | aggatggcat ttctcacaag acttagcaaa catgccactt gtagtactaa aaacttcagt |
| 1201 | atgcagcttt gacagatatt acacagacag tacagctaaa tcaaccacaa taggctttaa |
| 1261 | aacacttaac acacaaacat ttagatatca tgactggcag gaaccaccta caacaggata |
| 1321 | caaaccacaa aacctactat ggttttatgg agcagaaaac ggatcaccag tagaccccaa |
| 1381 | caacacaata gtatcaaacc taatatactt aggaggcaca ggaccttatg aaaaaggcac |
| 1441 | accaataaaa acaaacataa gcaattactt ttcagagcct aaactgtggg gaaatatatt |
| 1501 | tcacgatgat tatacatcag gaacatcacc cgtgtttgtt acaaacaaat caccatcaga |
| 1561 | aattaaaacc gcatggaaca ctataaaaga cttaactgtt aaagctagcg gtgtatttac |
| 1621 | attaagaaca attccactat ggctaccttg cagatacaac ccatttgcag acaaagcaac |
| 1681 | caacaacaaa atatggctag tttctataca ttcagaccac acagaatgga aaccaataga |
| 1741 | caatccatta ctacaacgaa cagaccttcc tttatggtta cttgtatggg gttggcaaga |
| 1801 | ttggcagaaa aaaaaccaac aaacttcaca acctgatatt aattatttaa cagtaatatc |
| 1861 | ttcaccatat atatcatgct acccaaaatt agattactat gtgttactag atgaaggatt |
| 1921 | ttgggagggt cactcaacat acatagagtc aattacagac tcagacaaaa aacactggta |
| 1981 | ccctaaaaat agatttcaaa tagaaacact taatctaata gctaacacag gtccaggaac |
| 2041 | tgtaaaacta agagaaaacc aagcagcaga aggtcacatg gtatatcgct ttaattttaa |
| 2101 | gcttggagga tgtcccgcac cgatggaaaa aatatgtgac cctagcaaac aatccaaata |
| 2161 | tcctattccc aataaccagc aacaaacaac ttcgttgcag agtccagaaa acccaattca |
| 2221 | aacctatctc tacgacttcg acgaaaggag gggcctactt acagaaagag ctacaaaaag |
| 2281 | aatcaaacaa gatcacacat ctgaaaaaac tgttttgcca tttacaggag cagcaacaga |
| 2341 | cctccccata ctccaaacaa catcacagga ggaaagctcc tcggaagaag aagaagagca |
| 2401 | acaagcggag aagaaactac tccagctccg aagaaagcag caccgactcc gggagcgaat |
| 2461 | cctccagcta ttagacatac aaaatacata ataaaacaaa gtactgtaaa aattgatatg |
| 2521 | tttggagata ctcatgtacc taaccgtaga atgaccccag aagaatttga acaagaacta |
| 2581 | attgtcgctg gtgtttttcg cagacctcct tgttactata taaaagatag acctacttat |
| 2641 | ccttatgtac caaaacctac tgatgaaaaa tgtatggtaa actttgactt aaactttcct |
| 2701 | taataaacta cgcctgcaaa ctttcactct cggtgtccat ttatataaga taaaacttaa |
| 2761 | ataaacatcc accactctcc caaatacgca ggcgcacaag ggggctccgc ccccttaaac |
| 2821 | ccccaagggg gctccgcccc cttaaacccc caagggggct ccgccccctt acacccccta |
| 2881 | ataaatattc aacaggaaaa ccacctaatt agaattgccg accacaaacc gtcacttact |
| 2941 | tctccttttt gcacttactt cctcttttac ttattattat tcattacatt aattaataat |
| 3001 | cactgtaatt ccggggagga gctaacaatc tatataacta actacacttc cgaatggctg |
| 3061 | agtttatgcc gccagacgga gacgggatca cttcagtgac tccaggctga acttgggcgg |
| 3121 | gagccgaagg tgagtgcaac caccgtagtc taggggcaat tcgggctagt tcagtatggc |
| 3181 | ggaacgggca agaaacttaa atattattat tttacagaaa caaatacaac cacctattag |
| 3241 | aacgttcaaa caaacaattt cagattggaa aaacttaatt gtccacgttc acgacaacat |
| 3301 | ttgcaactgc aataaaccat tagaacacac tattgatacc tgtatcacca atccagatga |
| 3361 | attaagatta aacaaatcta ctaagcagca actacaaaaa aaccttggta ccccagaaga |
| 3421 | agatacccaa gaagacgtta tcgatggctt cgcagatgga gagctagacg cccttttcgc |
| 3481 | ccaagataca gaagaagata ctgggtaaga aactattctc gaaagagaaa actatttaaa |
| 3541 | ataacaacca aagaatggca accaaaagtt ataagaaaga ctcatgtaaa gggcacctat |
| 3601 | cctttgtttc tttgtacaaa gcacagaatt aacaataata tgatacaata tttagactct |
| 3661 | atagctccag aacactatta cggaggagga ggattttcaa taatgcaatt ttccttacaa |
| 3721 | gccttatatg aagaatttat aaaagcaaaa aactggtgga ctaatacaaa ctgcttttta |
| 3781 | ccacttgtaa gatatatggg ttgctcattc aaattttata aaactgaatt ttatgattat |
| 3841 | attgtactaa ttgaaagatg ttatccactt gcttgtactg atgaaatgta cttatctact |
| 3901 | caacctagta ttatgatgct tacaagaaaa tgtatttttg taccatgcaa acaaaacagc |
| 3961 | aaaggtaaaa aaccttacaa aaaaatggtt acgcaggtga agtacgcccc ctattgacgt |
| 4021 | caatgacggt aaatggcccg cctggcatta tgcccagtac atgaccttat gggactttcc |
| 4081 | tacttggcag tacatctacg tattagtcat cgctattacc atggtgatgc ggttttggca |
| 4141 | gtacatcaat gggcgtggat agcggtttga ctcacgggga tttccaagtc tccaccccat |
| 4201 | tgacgtcaat gggagtttgt tttggcacca aaatcaacgg gactttccaa aatgtcgtaa |
| 4261 | caactccgcc ccattgacgc aaatgggcgg taggcgtgta cggtgggagg tctatataag |
| 4321 | cagagctctc tggctaactg gatctacaaa aaagcagatc caccggtcgc caccatggtg |
| 4381 | ttcacactcg aagatttcgt tggggactgg cgacagacag ccggctacaa cctggaccaa |
| 4441 | gtccttgaac agggaggtgt gtccagtttg tttcagaatc tcggggtgtc cgtaactccg |
| 4501 | atccaaagga ttgtcctgag cggtgaaaat gggctgaaga tcgacatcca tgtcatcatc |
| 4561 | ccgtatgaag gtctgagcgg cgaccaaatg ggccagatcg aaaaaatttt taaggtggtg |
| 4621 | taccctgtgg atgatcatca ctttaaggtg atcctgcact atggcacact ggtaatcgac |
| 4681 | ggggttacgc cgaacatgat cgactatttc ggacggccgt atgaaggcat cgccgtgttc |
| 4741 | gacggcaaaa agatcactgt aacagggacc ctgtggaacg gcaacaaaat tatcgacgag |
| 4801 | cgcctgatca accccgacgg ctccctgctg ttccgagtaa ccatcaacgg agtgaccggc |
| 4861 | tggcggctgt gcgaacgcat tctggcgtaa taactgttca ataaaatatc tttattttca |
| 4921 | ttacatctgt gtgttggttt tttgtgtgca cctgctatat gaattaagct tggaggatgt |
| 4981 | cccgcaccca tggaaaaaat atgtgaccct agcaaacaat ccaaatatcc tattcccaat |
| 5041 | aaccagcaac aaacaacttc gttgcagagt ccagaaaacc caattcaaac ctatctctac |
| 5101 | gacttcgacg aaaggagggg cctacttaca gaaagagcta caaaaagaat caaacaagat |
| 5161 | cacacatctg aaaaaactgt tttgccattt acaggagcag caacagacct ccccatactc |
| 5221 | caaacaacat cacaggagga aagctcctcg gaagaagaag aagagcaaca agcggagaag |
| 5281 | aaactactcc agctccgaag aaagcagcac cgactccggg agcgaatcct ccagctatta |
| 5341 | gacatacaaa atacataata aaacaaagta ctgtaaaaat tgatatgttt ggagatactc |
| 5401 | atgtacctaa ccgtagaatg accccagaag aatttgaaca agaactaatt gtcgctggtg |
| 5461 | tttttcgcag acctccttgt tactatataa aagatagacc tacttatcct tatgtaccaa |
| 5521 | aacctactga tgaaaaatgt atggtaaact ttgacttaaa ctttccttaa taaactacgc |
| 5581 | ctgcaaactt tcactctcgg tgtccattta tataagataa aacttaaata aacatccacc |
| 5641 | actctcccaa atacgcaggc gcacaagggg gctccgcccc cttaaacccc caagggggct |
| 5701 | ccgccccctt aaacccccaa gggggctccg cccccttaca ccc (SEQ ID NO: 9) |

Annotations:

| Putative Domain | Base range |
|---|---|
| Full length ORF1 | 524-2491 |
| Full length ORF2 | 342-632 |
| ORF2/2 | 342-628; 2201-2474 |
| ORF2/3 | 342-628; 2328-2703 |
| GC-rich region, or a portion thereof | 2767-2876 |
| 5′ UTR Conserved Domain, or a portion thereof | 242-312 |
| ORF1 with deletion | 3400-3984; 4964-5358 |
| Full length ORF2 | 3218-3508 |
| ORF2/2 | 3218-3504; 5068-5341 |
| ORF2/3 | 3218-3504; 5195-5570 |
| ORF3 | 5385-5570 |
| CMV promoter | 4124-4327 |
| Nano-Luciferase (nLuc) | 4375-4890 |
| SV40 poly A | 4900-4948 |
| GC-rich region, or a portion thereof | 5634-5743 |
| 5′ UTR Conserved Domain, or a portion thereof | 3118-3188 |

A tandem plasmid comprising two copies of the wild-type Ring19 genome sequence was used as a negative control. As shown in FIG. 59B, 1e7 MOLT-4 cells were electroporated with 100 μg of one of the tandem plasmids. After four days, the cells were harvested. The cells were lysed in 0.5% Triton X-100, 50 mM Tris pH 8.0, 100 mM NaCl, followed by two rounds of freeze thaw, and then treatment with 100 U/ml of benzonase for 90 minutes at room temperature. The resultant solution was run through an iodixanol linear gradient, and DNase qPCR was used to quantify DNase-protected genomes comprising nLuc sequence, thereby determining a level of packaged Ring19 anellovector particles rescued.

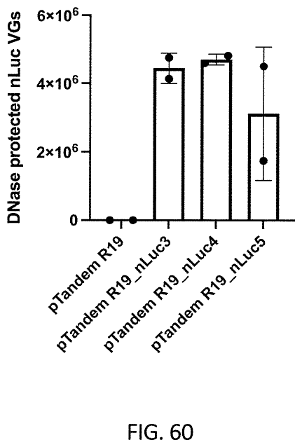

As shown in FIG. 60, each of the exemplary R19 nLuc anellovectors were successfully rescued, showing DNase-protected nLuc signal, whereas the negative control construct comprising two tandem copies of wild-type Ring19 genome did not show any detectable DNase-protected nLuc signal.

### Example 25. Ring19 Anellovectors Encoding a Variety of Exogenous Effectors

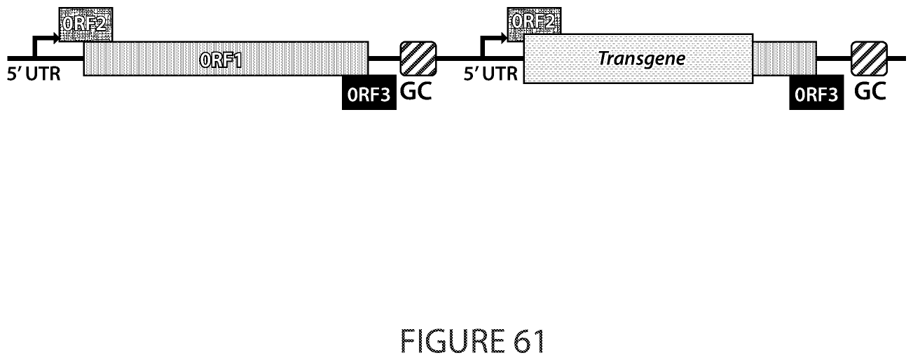

In this Example, the vectorization and rescue method described in Example 24 was utilized to produce a series of Ring19-based anellovectors carrying a number of different transgene cassettes comprising a promoter and a sequence encoding an exogenous effector. As shown in FIG. 61, the tandem nucleic acid construct used for these experiments was modeled after the R19_nLuc3, nLuc4, and nLuc5 constructs described in Example 24, in which the transgene cassette sequence replaces the regions extending across the Ring19 genome that were replaced by the R19_nLuc3, nLuc4, and nLuc5 cassettes. Promoters tested included SV40, min-hEF1a, Ubiquitin C, MSCV, SFFV, hPGK, INS84-eGFP, U1a-eGFP, CMV. Exemplary exogenous effectors tested included eGFP, mCherry, hGH, iCre, gLuc, and hEpo.

The tandem anellovector constructs were generated as follows. In brief, a portion of the Ring19 genome in the second copy of the tandem construct was hollowed out according to the limits identified from the nLuc3 construct (deletion after amino acid 56 of ORF2, 2/2, 2/3) and nLuc5 construct (deletion ending at amino acid 525 of ORF1). 1588 bp of genomic sequence was removed and replaced with an expression cassette carrying the SV40 promoter, eGFP coding sequence, and SV40polyA. The Methionine of ORF1 and ORF2, 2/2, 2/3 were changed to encode a Lysine (ATG to AAA) to knock out gene expression of these ORFs such that only the first wild type copy of the Ring19 genome is capable of ORF1 and ORF2 gene expression. The expression cassette was flanked by two BsaI sites. These sites were used for efficient exchange of payload and for insertion of a library of publicly available promoter and reporter sequences. As noted above, the following promoter sequences were tested: SV40, minimal hEF1a, UbC, MSCV, SFFV, hPGK, minimal CMV, INS84, and U1a. The following reporter sequences were tested: eGFP, mCherry, Epo, hGH, hGluc, and iCRE. All tandem constructs were cloned and propagated in a pUC57 standard backbone carrying the Kanamycin resistance marker.

The sequence of the exemplary tandem construct used to produce a Ring19 anellovector comprising an SV40-eGFP cassette is provided in Table D5 below. Selected silent mutations were introduced to remove internal BsaI restriction sites and facilitate cloning.

TABLE D5
Exemplary Ring19 SV40-eGFP tandem construct sequence

| | |
|---|---|
| Name | RING 19 WT-SV40-eGFP tandem construct |
| Genus/Clade | Betatorquevirus |
| Accession | N/A |

Full Sequence: 5669 bp

| | |
|---|---|
| 1 | cctaataaat attcaacagg aaaaccacct aattagaatt gccgaccaca aaccgtcact |
| 61 | tacttctcct ttttgcactt acttcctctt ttacttatta ttattcatta cattaattaa |
| 121 | taatcactgt aattccgggg aggagctaac aatctatata actaactaca cttccgaatg |
| 181 | gctgagttta tgccgccaga cggagacggg atcacttcag tgactccagg ctgaacttgg |
| 241 | gcgggagccg aaggtgagtg caaccaccgt agtctagggg caattcgggc tagttcagta |
| 301 | tggcggaacg ggcaagaaac ttaaatatta ttattttaca gatgcaaata caaccaccta |
| 361 | ttagaacctt caaacaaaca atttcagatt ggaaaaactt aattgtccac gttcacgaca |
| 421 | acatttgcaa ctgcaataaa ccattagaac acactattga tacctgtatc accaatccag |
| 481 | atgaattaag attaaacaaa tctactaaac aacaactaca aaaatgcctt ggtaccccag |
| 541 | aagaagatac ccaagaagac gttatcgatg gcttcgcaga tggagagcta gacgcccttt |
| 601 | tcgcccaaga tacagaagaa gatactgggt aagaaactat tctcgaaaga gaaaactatt |
| 661 | taaaataaca accaaagaat ggcaaccaaa agttataaga aagactcatg taaagggcac |
| 721 | ctatcctttg tttctttgta caaagcacag aattaacaat aatatgatac aatatttaga |
| 781 | ctctatagct ccagaacact attacggagg aggaggattt tcaataatgc aattttcctt |
| 841 | acaagcctta tatgaagaat ttataaaagc aaaaaactgg tggactaata caaactgctt |
| 901 | tttaccactt gtaagatata tgggttgctc attcaaattt tataaaactg aattttatga |
| 961 | ttatattgta ctaattgaaa gatgttatcc acttgcttgt actgatgaaa tgtacttatc |
| 1021 | tactcaacct agtattatga tgcttacaag aaaatgtatt tttgtaccat gcaaacaaaa |
| 1081 | cagcaaaggt aaaaaacctt acaaaaaagt tagagtaaga ccaccttcac aaatgactac |
| 1141 | aggatggcat ttctcacaag acttagcaaa catgccactt gtagtactaa aaacttcagt |
| 1201 | atgcagcttt gacagatatt acacagacag tacagctaaa tcaaccacaa taggctttaa |
| 1261 | aacacttaac acacaaacat ttagatatca tgactggcag gaaccaccta caacaggata |
| 1321 | caaaccacaa aacctactat ggttttatgg agcagaaaac ggatcaccag tagaccccaa |
| 1381 | caacacaata gtatcaaacc taatatactt aggaggcaca ggaccttatg aaaaaggcac |
| 1441 | accaataaaa acaaacataa gcaattactt ttcagagcct aaactgtggg gaaatatatt |
| 1501 | tcacgatgat tatacatcag gaacatcacc cgtgtttgtt acaaacaaat caccatcaga |
| 1561 | aattaaaacc gcatggaaca ctataaaaga cttaactgtt aaagctagcg gtgtatttac |
| 1621 | attaagaaca attccactat ggctaccttg cagatacaac ccatttgcag acaaagcaac |
| 1681 | caacaacaaa atatggctag tttctataca ttcagaccac acagaatgga aaccaataga |
| 1741 | caatccatta ctacaacgaa cagaccttcc tttatggtta cttgtatggg gttggcaaga |
| 1801 | ttggcagaaa aaaaaccaac aaacttcaca acctgatatt aattatttaa cagtaatatc |
| 1861 | ttcaccatat atatcatgct acccaaaatt agattactat gtgttactag atgaaggatt |
| 1921 | ttgggagggt cactcaacat acatagagtc aattacagac tcagacaaaa aacactggta |
| 1981 | ccctaaaaat agatttcaaa tagaaacact taatctaata gctaacacag gtccaggaac |
| 2041 | tgtaaaacta agagaaaacc aagcagcaga aggtcacatg gtatatcgct ttaattttaa |
| 2101 | gcttggagga tgtcccgcac cgatggaaaa aatatgtgac cctagcaaac aatccaaata |
| 2161 | tcctattccc aataaccagc aacaaacaac ttcgttgcag agtccagaaa acccaattca |
| 2221 | aacctatctc tacgacttcg acgaaaggag gggcctactt acagaaagag ctacaaaaag |
| 2281 | aatcaaacaa gatcacacat ctgaaaaaac tgttttgcca tttacaggag cagcaacaga |
| 2341 | cctccccata ctccaaacaa catcacagga ggaaagctcc tcggaagaag aagaagagca |
| 2401 | acaagcggag aagaaactac tccagctccg aagaaagcag caccgactcc gggagcgaat |
| 2461 | cctccagcta ttagacatac aaaatacata ataaaacaaa gtactgtaaa aattgatatg |
| 2521 | tttggagata ctcatgtacc taaccgtaga atgaccccag aagaatttga acaagaacta |
| 2581 | attgtcgctg gtgtttttcg cagacctcct tgttactata taaaagatag acctacttat |
| 2641 | ccttatgtac caaaacctac tgatgaaaaa tgtatggtaa actttgactt aaactttcct |
| 2701 | taataaacta cgcctgcaaa ctttcactct cggtgtccat ttatataaga taaaacttaa |
| 2761 | ataaacatcc accactctcc caaatacgca ggcgcacaag ggggctccgc ccccttaaac |
| 2821 | ccccaagggg gctccgcccc cttaaacccc caagggggct ccgccccctt acacccccta |
| 2881 | ataaatattc aacaggaaaa ccacctaatt agaattgccg accacaaacc gtcacttact |
| 2941 | tctccttttt gcacttactt cctcttttac ttattattat tcattacatt aattaataat |
| 3001 | cactgtaatt ccggggagga gctaacaatc tatataacta actacacttc cgaatggctg |
| 3061 | agtttatgcc gccagacgga gacgggatca cttcagtgac tccaggctga acttgggcgg |
| 3121 | gagccgaagg tgagtgcaac caccgtagtc taggggcaat tcgggctagt tcagtatggc |
| 3181 | ggaacgggca agaaacttaa atattattat tttacagaaa caaatacaac cacctattag |
| 3241 | aacgttcaaa caaacaattt cagattggaa aaacttaatt gtccacgttc acgacaacat |
| 3301 | ttgcaactgc aataaaccat tagaacacac tattgatacc tgtatcacca atccagatga |
| 3361 | attaagatta aacaaatcta ctaaatggct gatcgagtgt agccagatct gcgatctgca |
| 3421 | tctcaattag tcagcaacca tagtcccgcc cctaactccg cccatcccgc ccctaactcc |
| 3481 | gcccagttcc gcccattctc cgccccatcg ctgactaatt ttttttattt atgcagaggc |
| 3541 | cgaggccgcc tcggcctctg agctattcca gaagtagtga ggaggctttt ttggaggcct |
| 3601 | aggcttttgc agatctttgt cgatcctacc atccggatct acaaaaaagc agatccaccg |
| 3661 | gtcgccacca tggtgagcaa gggcgaggag ctgttcaccg gggtggtgcc catcctggtc |
| 3721 | gagctggacg gcgacgtaaa cggccacaag ttcagcgtgt ccggcgaggg cgagggcgat |
| 3781 | gccacctacg gcaagctgac cctgaagttc atctgcacca ccggcaagct gcccgtgccc |
| 3841 | tggcccaccc tcgtgaccac cctgacctac ggcgtgcagt gcttcagccg ctaccccgac |
| 3901 | cacatgaagc agcacgactt cttcaagtcc gccatgcccg aaggctacgt ccaggagcgc |
| 3961 | accatcttct tcaaggacga cggcaactac aagacccgcg ccgaggtgaa gttcgagggc |
| 4021 | gacaccctgg tgaaccgcat cgagctgaag ggcatcgact tcaaggagga cggcaacatc |
| 4081 | ctggggcaca agctggagta caactacaac agccacaacg tctatatcat ggccgacaag |
| 4141 | cagaagaacg gcatcaaggt gaacttcaag atccgccaca acatcgagga cggcagcgtg |
| 4201 | cagctcgccg accactacca gcagaacacc cccatcggcg acggccccgt gctgctgccc |
| 4261 | gacaaccact acctgagcac ccagtccgcc ctgagcaaag accccaacga gaagcgcgat |
| 4321 | cacatggtcc tgctggagtt cgtgaccgcc gccgggatca ctctcggcat ggacgagctg |
| 4381 | tacaagtaat aacttgtaca aagtggtacg cgtgaagttc gcttgcggcc gcttcgagca |
| 4441 | gacatgataa gatacattga tgagtttgga caaaccacaa ctagaatgca gtgaaaaaaa |
| 4501 | tgctttattt gtgaaattty tgatgctatt gctttatttg taaccattat aagctgcaat |
| 4561 | aaacaagtta acaacaacaa ttgcatcgag ttagtttgct ccagtaaagt tgtttataat |
| 4621 | aactactaaa tccgcatgtt acggaatttc ttattaattt ttttttcgta aggaacaacg |
| 4681 | gatcttaatg aatggccgca aatggtatgg aagctaatag cgcgggtgag agggtaatta |
| 4741 | gccatgttca ccaatataac gctatcaggc aattctataa gattccacat tgcatctatt |
| 4801 | tataaaatgt ctcaatggta tccgcaactt gtgatgtgtc tactatcctt aaatgcatat |
| 4861 | ctcgcttaat aacttctcaa tataatgaat taagcttgga ggatgtcccg cacccatgga |
| 4921 | aaaaatatgt gaccctagca aacaatccaa atatcctatt cccaataacc agcaacaaac |
| 4981 | aacttcgttg cagagtccag aaaacccaat tcaaacctat ctctacgact tcgacgaaag |
| 5041 | gaggggccta cttacagaaa gagctacaaa aagaatcaaa caagatcaca catctgaaaa |
| 5101 | aactgttttg ccatttacag gagcagcaac agacctcccc atactccaaa caacatcaca |
| 5161 | ggaggaaagc tcctcggaag aagaagaaga gcaacaagcg gagaagaaac tactccagct |
| 5221 | ccgaagaaag cagcaccgac tccgggagcg aatcctccag ctattagaca tacaaaatac |
| 5281 | ataataaaac aaagtactgt aaaaattgat atgtttggag atactcatgt acctaaccgt |
| 5341 | agaatgaccc cagaagaatt tgaacaagaa ctaattgtcg ctggtgtttt tcgcagacct |
| 5401 | ccttgttact atataaaaga tagacctact tatccttatg taccaaaacc tactgatgaa |
| 5461 | aaatgtatgg taaactttga cttaaacttt ccttaataaa ctacgcctgc aaactttcac |
| 5521 | tctcggtgtc catttatata agataaaact taaataaaca tccaccactc tcccaaatac |
| 5581 | gcaggcgcac aagggggctc cgccccctta aacccccaag ggggctccgc ccccttaaac |
| 5641 | ccccaagggg gctccgcccc cttacaccc (SEQ ID NO: 11) |

Annotations:

| Putative Domain | Base range |
|---|---|
| Full length ORF1 | 524-2491 |
| Full length ORF2 | 342-632 |
| ORF2/2 | 342-628; 2201-2474 |
| ORF2/3 | 342-628; 2328-2703 |
| GC-rich region, or a portion thereof | 2767-2876 |
| 5′ UTR Conserved Domain, or a portion thereof | 242-312 |
| Truncated ORF1 | 4890-5284 |
| Truncated ORF2 | 3218-3385 |
| ORF2/2 | 3218-3385; 4994-5267 |
| ORF2/3 | 3218-3385; 5121-5496 |
| ORF3 | 5311-5496 |
| SV40 promoter | 3417-3613 |
| SV40 ori | 3464-3599 |
| eGFP | 3670-4389 |
| SV40 poly A | 4448-4569 |
| GC-rich region, or a portion thereof | 5560-5669 |
| 5′ UTR Conserved Domain, or a portion thereof | 3118-3188 |

The eGFP polypeptide encoded by the construct above comprises the amino acid sequence:

(SEQ ID NO: 12)
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK.

For transfection of each vector-tandem plasmid, 1e7 MOLT-4 cells were pelleted and resuspended in 500 μL of electroporation buffer (5 mM KCl, 15 mM MgCl2, 15 mM HEPES, 150 mM Na2HPO4 pH7.2, 50 mM sodium succinate), and 50 ug of vector-plasmid was added to each suspension of 1e7 cells. The plasmid+cells suspension was distributed across five 2 mm electroporation cuvettes, 110 μL of suspension mixture per cuvette. Cuvettes containing the plasmid+cell suspension were subjected to the following electroporation condition using the NEPA21 transfection instrument:

| | | Poring | | | | | | Transfer | | | |
|---|---|---|---|---|---|---|---|---|---|---|---|
| | | Pulse | | D. | | | | Pulse | | D. | |
| | Length | Interval | | Rate | | | Length | Interval | | Rate | |
| V | (ms) | (ms) | No. | (%) | Polarity | V | (ms) | (ms) | No. | (%) | Polarity |
| 150 | 5 | 50 | 2 | 10 | + | 20 | 50 | 50 | 5 | 40 | +/− |

Pre-warmed media (300 uL) was added to each cuvette. The suspension was transferred to a 150 mL flask containing 25 mL of pre-warmed complete media (RPMI+10% FBS+1 mM sodium pyruvate) and incubated for 4 days at 37 C and 5% CO₂. Cells from day 4 cultures were pelleted and resuspended in 1 m lysis buffer (0.5% Triton, 50 mM Tris pH 8.0, 300 mM NaCl) and subjected to 3× freeze-thaw cycles. After freeze-thaw, 1 ml of benzonase buffer (2 mM MgCl2, 50 mM Tris pH 8.0, 200 U benzonase) was added to the lysate and incubated at room temperature for 90 min. Lysates were clarified by centrifugation at 10K RCF for 30 min. Lysates were loaded onto a linear iodixanol gradient and ultracentrifuged at 60000 RPM for 1 hour in a 70Ti rotor. Gradients were fractionated and viral particle content was determined by a DNase-protected qPCR titer assay.

To determine DNase-protected vector genome copies, 5 μL of each fraction was incubated with 20 U of DNase I (20 uL final reaction volume) at 37 C for 30 min. The reaction was then subjected to Proteinase K digestion (40 uL final volume) at 55 C for 30 min followed by a deactivation step at 95 C for 15 min. The sample was then diluted 1:10 in 0.05% pluronic buffer. DNA content was then determined using a probe-based qPCR reaction using a standard curve comprised of a serial dilution of plasmid of known concentration.

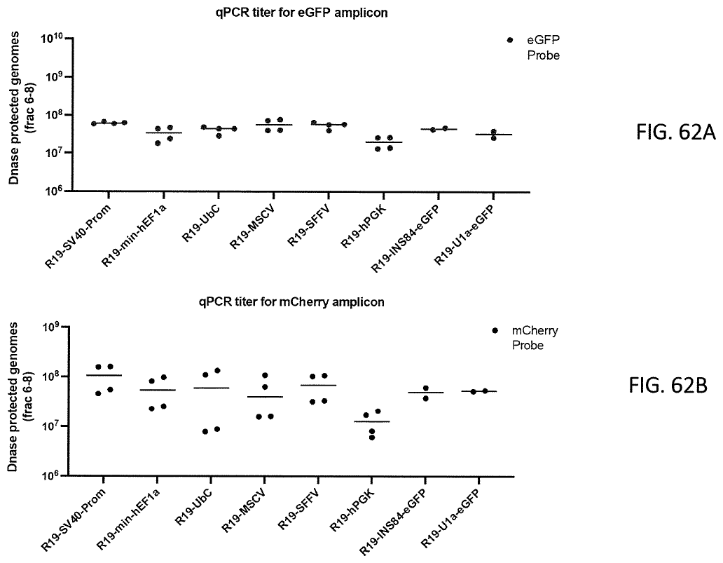

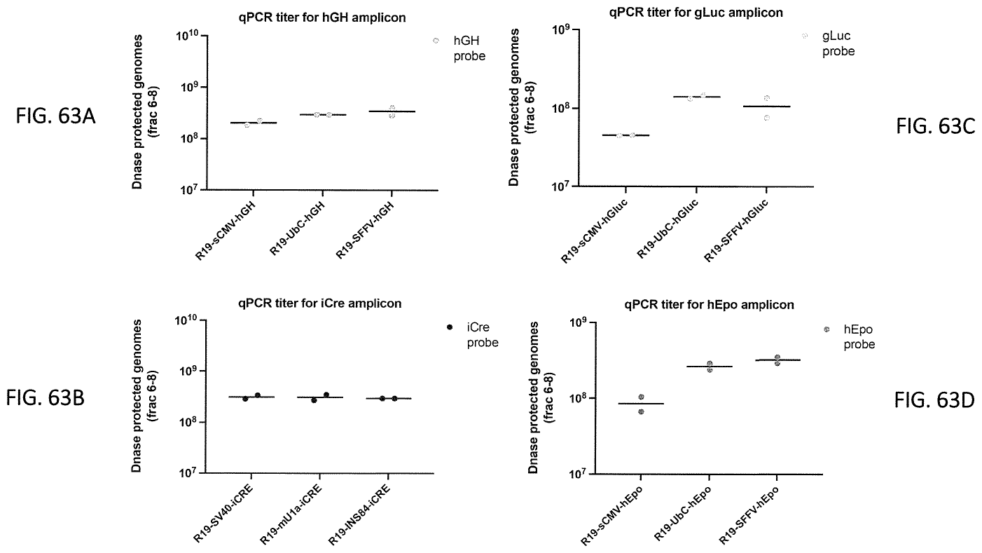

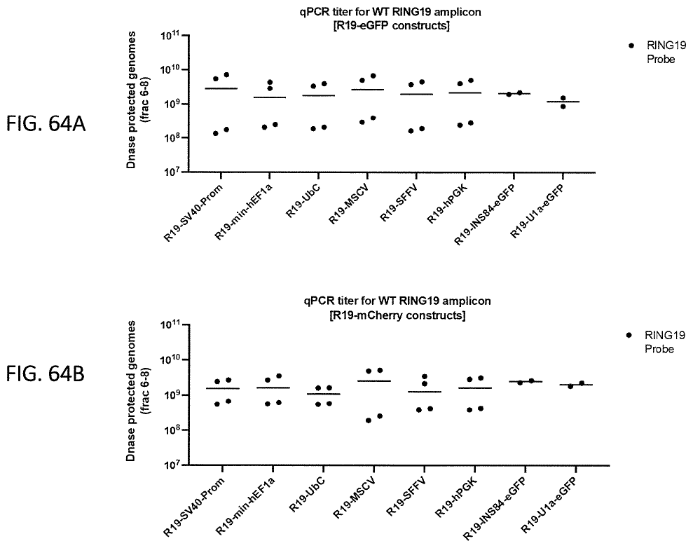

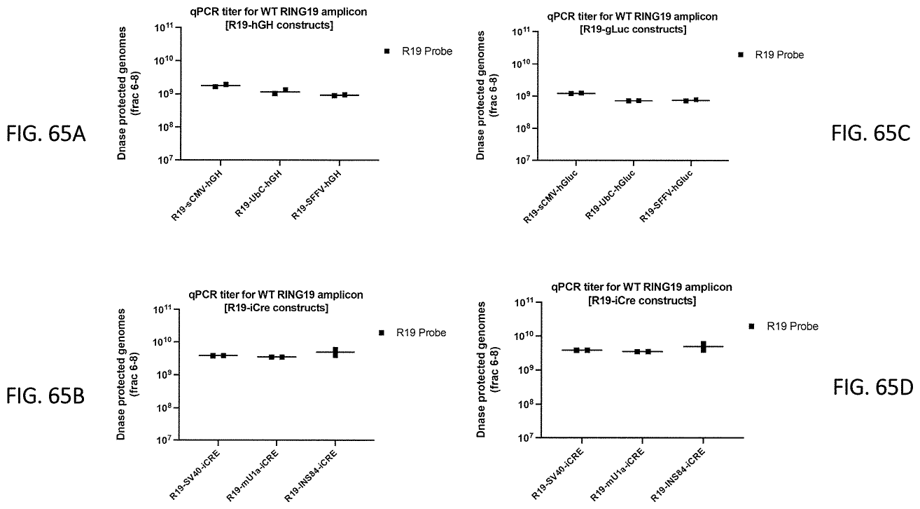

As shown in FIGS. 62A and 62B, Ring19 anellovector constructs carrying an eGFP cassette (FIG. 62A) or an mCherry cassette (FIG. 62B) under the control of the promoters indicated on the x-axes showed detectable levels of DNase-protected genomes, indicating successful rescue of packaged anellovector particles. Ring19 anellovector constructs carrying an hGH transgene, gLuc transgene, iCre transgene, or hEpo transgene under the control of a CMV, ubiquitin C, or SFFV promoter were also tested. As shown in FIGS. 63A-63D, packaged Ring19 anellovector particles were recovered for each of these constructs. FIGS. 64A-64B and 65A-65B show that the wild-type genome on the tandem plasmids also yielded Ring19 Anellovirus particles. Taken together, these data demonstrate successful vectorization and rescue of Ring19 anellovector particles carrying genetic elements encoding exogenous effectors.

What is claimed is:

1. An anellovector comprising:
(i) a proteinaceous exterior comprising an Anellovirus ORF1 protein as listed in Table A1, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.

2. An anellovector comprising:
(i) a proteinaceous exterior comprising an Anellovirus ORF1 protein as listed in Table A1, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

3. An anellovector comprising:
(i) a proteinaceous exterior comprising a polypeptide encoded by an Anellovirus ORF1 nucleic acid sequence as listed in Table N1, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.

4. An anellovector comprising:
(i) a proteinaceous exterior comprising a polypeptide encoded by an Anellovirus ORF1 nucleic acid sequence as listed in Table N1, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

5. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in Table N1, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an exogenous effector.

6. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in Table N1, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

7. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an exogenous effector, and wherein the genetic element has at least about 70%, 75%, 80%, 85%, 90%, 95%, 96%, 97%, 98%, 99%, or 100% sequence identity to an Anellovirus genome sequence as listed in Table N1.

8. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector), and wherein the genetic element has at least about 70%, 75%, 80%, 85%, 90%, 95%, 96%, 97%, 98%, 99%, or 100% sequence identity to an Anellovirus genome sequence as listed in Table N1;
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein) or genomic region (e.g., one or more of a TATA box, cap site, transcriptional start site, 5′ UTR, open reading frame (ORF), poly(A) signal, or GC-rich region, e.g., as described herein).

9. An isolated ORF1 molecule comprising the amino acid sequence of an ORF1 as listed in Table A1, or an amino acid sequence having at least about 70%, 75%, 80%, 85%, 90%, 95%, 96%, 97%, 98%, 99%, or 100% sequence identity thereto;
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

10. An isolated ORF1 molecule comprising the amino acid sequence of the jelly-roll domain of an ORF1 as listed in Table A1, or an amino acid sequence having at least about 70%, 75%, 80%, 85%, 90%, 95%, 96%, 97%, 98%, 99%, or 100% sequence identity thereto;
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

11. An isolated ORF2 molecule comprising the amino acid sequence of an ORF2 as listed in Table A1, or an amino acid sequence having at least about 70%, 75%, 80%, 85%, 90%, 95%, 96%, 97%, 98%, 99%, or 100% sequence identity thereto;
wherein the ORF2 molecule comprises at least one difference (e.g., a mutation, chemical modification, or epigenetic alteration) relative to a wild-type ORF2 protein (e.g., as described herein), e.g., an insertion, substitution, chemical or enzymatic modification, and/or deletion, e.g., a deletion of a domain.

12. An isolated nucleic acid molecule (e.g., a genetic element construct or a genetic element) comprising the nucleic acid sequence of a 5′ UTR conserved domain as listed in Table N1, or a nucleic acid sequence having at least 93%, 94%, 95%, 96%, 97%, 98%, or 99% sequence identity thereto.

13. An isolated nucleic acid molecule (e.g., a genetic element construct or a construct for providing an ORF1 molecule in trans, e.g., as described herein) comprising the nucleic acid sequence of an ORF1 gene as listed in Table N1, or a nucleic acid sequence having at least 70%, 75%, 80%, 85%, 90%, 95%, 96%, 97%, 98%, or 99% sequence identity thereto.

14. An isolated nucleic acid molecule (e.g., a genetic element construct or a construct for providing an ORF2 molecule in trans, e.g., as described herein) comprising the nucleic acid sequence of an ORF2 gene as listed in Table N1, or a nucleic acid sequence having at least 70%, 75%, 80%, 85%, 90%, 95%, 96%, 97%, 98%, or 99% sequence identity thereto.

15. An isolated nucleic acid molecule (e.g., a genetic element construct, a genetic element, or a construct for providing an ORF1 or ORF2 molecule in trans, e.g., as described herein) comprising an Anellovirus genome sequence as listed in Table N1, or a nucleic acid sequence having at least 50%, 60%, 70%, 75%, 80%, 85%, 90%, 95%, 96%, 97%, 98%, or 99% sequence identity thereto.

16. A genetic element comprising:
(a) a 5′ UTR conserved domain as listed in Table N1, or a nucleic acid sequence having at least 93%, 94%, 95%, 96%, 97%, 98%, 99%, or 100% sequence identity thereto, and
(b) a promoter element operably linked to a nucleic acid sequence (e.g., a DNA sequence) encoding an exogenous effector.

17. A method of manufacturing an anellovector composition, the method comprising:
(a) providing a cell, e.g., a host cell as described herein;
(b) introducing a nucleic acid molecule encoding an ORF1 polypeptide as listed in Table A1 (or an amino acid sequence having at least about 70%, 75%, 80%, 85%, 90%, 95%, 96%, 97%, 98%, or 99% sequence identity thereto) into the cell;
(c) introducing a genetic element construct into the cell (e.g., before, after, or simultaneously with (b)),
(d) incubating the cell under conditions that allow the cell to produce anellovector; and
(e) formulating the anellovectors, e.g., as a pharmaceutical composition suitable for administration to a subject,
thereby making the anellovector composition.

18. A method of manufacturing an anellovector composition, the method comprising:
(a) providing a cell, e.g., a host cell as described herein;
(b) introducing a nucleic acid molecule encoding an ORF1 polypeptide into the cell;
(c) introducing a genetic element construct into the cell as listed in Table N1 (or a nucleic acid sequence having at least about 70%, 75%, 80%, 85%, 90%, 95%, 96%, 97%, 98%, or 99% sequence identity thereto) (e.g., before, after, or simultaneously with (b)),
(d) incubating the cell under conditions that allow the cell to produce anellovector; and
(e) formulating the anellovectors, e.g., as a pharmaceutical composition suitable for administration to a subject,
thereby making the anellovector composition.

19. A method of making an anellovector, e.g., a synthetic anellovector, comprising:
(a) providing a host cell comprising:
(i) a nucleic acid molecule, e.g., a first nucleic acid molecule, comprising the nucleic acid sequence of a Anellovirus genome as listed in Table N1 (or a nucleic acid sequence having at least 70%, 75%, 80%, 85%, 90%, 95%, 96%, 97%, 98%, or 99% sequence identity thereto), and
(ii) a nucleic acid molecule, e.g., a second nucleic acid molecule, encoding one or more of an amino acid sequence chosen from ORF1, ORF2, ORF2/2, ORF2/3, ORF1/1, or ORF1/2, e.g., as listed in Table A1, or an amino acid sequence having at least 70% 75%, 80%, 85%, 90%, 95%, 96%, 97%, 98%, or 99% sequence identity thereto; and
(b) culturing the host cell under conditions suitable to make the anellovector.

20. A method of delivering an effector to an eye of a subject, the method comprising administering to the eye of the subject an anellovector of any of claims 1-8.

21. A method of modulating a biological function in an eye of a subject, the method comprising administering the anellovector of any of claims 1-8 to the subject.

22. A method of treating an eye disease or disorder in a subject in need thereof, the method comprising administering to the subject an anellovector of any of claims 1-8.

23. A method of delivering an effector (e.g., an exogenous effector or an endogenous effector, e.g., overexpressing an endogenous effector) to an eye of the subject (e.g., to a photoreceptor, retina, posterior eye cup (PEC), optic nerve, optic nerve head, intravitreal space, subretinal space, retinal ganglion, or retinal pigmented epithelium (RPE) of the subject), the method comprising administering to the eye of the subject (e.g., to a photoreceptor, retina, posterior eye cup (PEC), optic nerve, optice nerve head, intravitreal space, subretinal space, retinal ganglion, or retinal pigmented epithelium (RPE) of the subject) an Anelloviridae family vector (e.g., an anellovector).

24. A method of modulating, e.g., enhancing or inhibiting, a biological function (e.g., as described herein) in an eye of the subject (e.g., in a photoreceptor, retina, posterior eye cup (PEC), optic nerve, optic nerve head, intravitreal space, subretinal space, retinal ganglion, or retinal pigmented epithelium (RPE) of the subject), the method comprising administering the Anelloviridae family vector (e.g., the anellovector) or the pharmaceutical composition of any of the preceding embodiments to the eye of the subject (e.g., to a photoreceptor, retina, posterior eye cup (PEC), optic nerve, optic nerve head, intravitreal space, subretinal space, retinal ganglion, or retinal pigmented epithelium (RPE) of the subject).

25. A method of treating a disease or disorder (e.g., an eye disease or disorder) in a subject in need thereof, the method comprising administering to an eye of the subject (e.g., to a photoreceptor, retina, posterior eye cup (PEC), optic nerve, optic nerve head, intravitreal space, subretinal space, retinal gangkion, or retinal pigmented epithelium (RPE) of the subject) an Anelloviridae family vector (e.g., an anellovector) or pharmaceutical composition of any of the preceding embodiments.

26. An ocular delivery system comprising an Anelloviridae family vector (e.g., an anellovector, e.g., as described herein).

27. A genetic element comprising (e.g., in 5′ to 3′ order):
(i) nucleotides 1-71 of SEQ ID NO: 1, or a nucleic acid sequence having at least 93%, 94%, 95%, 96%, 97%, 98%, or 99% sequence identity thereto;
(ii) a 5′ portion of an ORF2 nucleic acid sequence;
(iii) a promoter element;
(iv) a nucleic acid sequence encoding an exogenous effector (e.g., a therapeutic exogenous effector); and
(v) a 3′ portion of an ORF1 nucleic acid sequence;
or a complement of (i)-(v);
wherein the genetic element does not encode a full-length ORF1 polypeptide or a full-length ORF2 polypeptide.

28. A genetic element comprising (e.g., in 5′ to 3′ order):
(i) nucleotides 1-71 of SEQ ID NO: 1, or a nucleic acid sequence having at least 93%, 94%, 95%, 96%, 97%, 98%, or 99% sequence identity thereto;
(ii) a 5′ portion of an ORF1 nucleic acid sequence;
(iii) a promoter element;
(iv) a nucleic acid sequence encoding an exogenous effector (e.g., a therapeutic exogenous effector); and
(v) a 3′ portion of an ORF1 nucleic acid sequence;
or a complement of (i)-(v);
wherein the genetic element does not encode a full-length ORF1 polypeptide


SEQUENCE LISTING (1016 sequences)

SEQ ID NO: 1 - DNA, 2876 bp, Betatorquevirus sp.
  source 1..2876: mol_type = genomic DNA
cgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttggg

SEQ ID NO: 2 - protein, 655 aa, Betatorquevirus sp.
MPWYPRRRYPRRRYRWLRRWRARRPFRPRYRRRYWVRNYSRKRKLFKITTKEWQPKVIRKTHVKGTYPLFLCTKHRINNNMIQYLDSIAPEHYYGGGGFSIMQFSLQALYEEFIKAKNWWTNTNCFLPLVRYMGCSFKFYKTEFYDYIVLIERCYPLACTDEMYLSTQPSIMMLTRKCIFVPCKQNSKGKKPYKKVRVRPPSQMTTGWHFSQDLANMPLVVLKTSVCSFDRYYTDSTAKSTTIGFKTLNTQTFRYHDWQEPPTTGYKPQNLLWFYGAENGSPVDPNNTIVSNLIYLGGTGPYEKGTPIKTNISNYFSEPKLWGNIFHDDYTSGTSPVFVTNKSPSEIKTAWNTIKDLTVKASGVFTLRTIPLWLPCRYNPFADKATNNKIWLVSIHSDHTEWKPIDNPLLQRTDLPLWLLVWGWQDWQKKNQQTSQPDINYLTVISSPYISCYPKLDYYVLLDEGFWEGHSTYIESITDSDKKHWYPKNRFQIETLNLIANTGPGTVKLRENQAAEGHMVYRFNFKLGGCPAPMEKICDPSKQSKYPIPNNQQQTTSLQSPENPIQTYLYDFDERRGLLTERATKRIKQDHTSEKTVLPFTGAATDLPILQTTSQEESSSEEEEEQQAEKKLLQLRRKQHRLRERILQLLDIQNT

SEQ ID NO: 3 - protein, 110 aa, Betatorquevirus sp.
MAERARNLNIIILQMQIQPPIRTFKQTISDWKNLIVHVHDNICNCNKPLEHTIDTCITNPDELRLNKSTKQQLQKCLGTPEEDTQEDVIDGFADGELDALFAQDTEEDTG

SEQ ID NO: 4 - protein, 61 aa, Betatorquevirus sp.
MFGDTHVPNRRMTPEEFEQELIVAGVFRRPPCYYIKDRPTYPYVPKPTDEKCMVNFDLNFP

SEQ ID NO: 5 - DNA, 2313 bp, Gyrovirus sp.
  source 1..2313: mol_type = genomic DNA
cgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 6 - DNA, 5752 bp, synthetic construct
  source 1..5752: mol_type = other DNA
cctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccc

SEQ ID NO: 7 - DNA, 5743 bp, synthetic construct
  source 1..5743: mol_type = other DNA
cctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagaaacaaatacaaccacctattagaacgttcaaacaaacaatttcagattggaaaaacttaattgtccacgttcacgacaacatttgcaactgcaataaaccattagaacacactattgatacctgtatcaccaatccagatgaattaagattaaacaaatctactaagatggttacgcaggtgaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactggatctacaaaaaagcagatccaccggtcgccaccatggtgttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataactgttcaataaaatatctttattttcattacatctgtgtgttggttttttgtgtgcacctgctatatgaaatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcacccatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccc

SEQ ID NO: 8 - DNA, 5743 bp, synthetic construct
  source 1..5743: mol_type = other DNA
cctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagaaacaaatacaaccacctattagaacgttcaaacaaacaatttcagattggaaaaacttaattgtccacgttcacgacaacatttgcaactgcaataaaccattagaacacactattgatacctgtatcaccaatccagatgaattaagattaaacaaatctactaagcagcaactacaaaaaaaccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaatggttacgcaggtgaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactggatctacaaaaaagcagatccaccggtcgccaccatggtgttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataactgttcaataaaatatctttattttcattacatctgtgtgttggttttttgtgtgcacctgctatatgaaaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcacccatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccc

SEQ ID NO: 9 - DNA, 5743 bp, synthetic construct
  source 1..5743: mol_type = other DNA
cctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagaaacaaatacaaccacctattagaacgttcaaacaaacaatttcagattggaaaaacttaattgtccacgttcacgacaacatttgcaactgcaataaaccattagaacacactattgatacctgtatcaccaatccagatgaattaagattaaacaaatctactaagcagcaactacaaaaaaaccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaaatggttacgcaggtgaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactggatctacaaaaaagcagatccaccggtcgccaccatggtgttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataactgttcaataaaatatctttattttcattacatctgtgtgttggttttttgtgtgcacctgctatatgaattaagcttggaggatgtcccgcacccatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccc

SEQ ID NO: 10 - protein, 171 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 11 - DNA, 5669 bp, synthetic construct
  source 1..5669: mol_type = other DNA
cctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagatgcaaatacaaccacctattagaaccttcaaacaaacaatttcagattggaaaaacttaattgtccacgttcacgacaacatttgcaactgcaataaaccattagaacacactattgatacctgtatcaccaatccagatgaattaagattaaacaaatctactaaacaacaactacaaaaatgccttggtaccccagaagaagatacccaagaagacgttatcgatggcttcgcagatggagagctagacgcccttttcgcccaagatacagaagaagatactgggtaagaaactattctcgaaagagaaaactatttaaaataacaaccaaagaatggcaaccaaaagttataagaaagactcatgtaaagggcacctatcctttgtttctttgtacaaagcacagaattaacaataatatgatacaatatttagactctatagctccagaacactattacggaggaggaggattttcaataatgcaattttccttacaagccttatatgaagaatttataaaagcaaaaaactggtggactaatacaaactgctttttaccacttgtaagatatatgggttgctcattcaaattttataaaactgaattttatgattatattgtactaattgaaagatgttatccacttgcttgtactgatgaaatgtacttatctactcaacctagtattatgatgcttacaagaaaatgtatttttgtaccatgcaaacaaaacagcaaaggtaaaaaaccttacaaaaaagttagagtaagaccaccttcacaaatgactacaggatggcatttctcacaagacttagcaaacatgccacttgtagtactaaaaacttcagtatgcagctttgacagatattacacagacagtacagctaaatcaaccacaataggctttaaaacacttaacacacaaacatttagatatcatgactggcaggaaccacctacaacaggatacaaaccacaaaacctactatggttttatggagcagaaaacggatcaccagtagaccccaacaacacaatagtatcaaacctaatatacttaggaggcacaggaccttatgaaaaaggcacaccaataaaaacaaacataagcaattacttttcagagcctaaactgtggggaaatatatttcacgatgattatacatcaggaacatcacccgtgtttgttacaaacaaatcaccatcagaaattaaaaccgcatggaacactataaaagacttaactgttaaagctagcggtgtatttacattaagaacaattccactatggctaccttgcagatacaacccatttgcagacaaagcaaccaacaacaaaatatggctagtttctatacattcagaccacacagaatggaaaccaatagacaatccattactacaacgaacagaccttcctttatggttacttgtatggggttggcaagattggcagaaaaaaaaccaacaaacttcacaacctgatattaattatttaacagtaatatcttcaccatatatatcatgctacccaaaattagattactatgtgttactagatgaaggattttgggagggtcactcaacatacatagagtcaattacagactcagacaaaaaacactggtaccctaaaaatagatttcaaatagaaacacttaatctaatagctaacacaggtccaggaactgtaaaactaagagaaaaccaagcagcagaaggtcacatggtatatcgctttaattttaagcttggaggatgtcccgcaccgatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccccctaataaatattcaacaggaaaaccacctaattagaattgccgaccacaaaccgtcacttacttctcctttttgcacttacttcctcttttacttattattattcattacattaattaataatcactgtaattccggggaggagctaacaatctatataactaactacacttccgaatggctgagtttatgccgccagacggagacgggatcacttcagtgactccaggctgaacttgggcgggagccgaaggtgagtgcaaccaccgtagtctaggggcaattcgggctagttcagtatggcggaacgggcaagaaacttaaatattattattttacagaaacaaatacaaccacctattagaacgttcaaacaaacaatttcagattggaaaaacttaattgtccacgttcacgacaacatttgcaactgcaataaaccattagaacacactattgatacctgtatcaccaatccagatgaattaagattaaacaaatctactaaatggctgatcgagtgtagccagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcagatctttgtcgatcctaccatccggatctacaaaaaagcagatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataacttgtacaaagtggtacgcgtgaagttcgcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcatcgagttagtttgctccagtaaagttgtttataataactactaaatccgcatgttacggaatttcttattaatttttttttcgtaaggaacaacggatcttaatgaatggccgcaaatggtatggaagctaatagcgcgggtgagagggtaattagccatgttcaccaatataacgctatcaggcaattctataagattccacattgcatctatttataaaatgtctcaatggtatccgcaacttgtgatgtgtctactatccttaaatgcatatctcgcttaataacttctcaatataatgaattaagcttggaggatgtcccgcacccatggaaaaaatatgtgaccctagcaaacaatccaaatatcctattcccaataaccagcaacaaacaacttcgttgcagagtccagaaaacccaattcaaacctatctctacgacttcgacgaaaggaggggcctacttacagaaagagctacaaaaagaatcaaacaagatcacacatctgaaaaaactgttttgccatttacaggagcagcaacagacctccccatactccaaacaacatcacaggaggaaagctcctcggaagaagaagaagagcaacaagcggagaagaaactactccagctccgaagaaagcagcaccgactccgggagcgaatcctccagctattagacatacaaaatacataataaaacaaagtactgtaaaaattgatatgtttggagatactcatgtacctaaccgtagaatgaccccagaagaatttgaacaagaactaattgtcgctggtgtttttcgcagacctccttgttactatataaaagatagacctacttatccttatgtaccaaaacctactgatgaaaaatgtatggtaaactttgacttaaactttccttaataaactacgcctgcaaactttcactctcggtgtccatttatataagataaaacttaaataaacatccaccactctcccaaatacgcaggcgcacaagggggctccgcccccttaaacccccaagggggctccgcccccttaaacccccaagggggctccgcccccttacaccc

SEQ ID NO: 12 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13-53 - skipped (41 entries)

SEQ ID NO: 54 - DNA, 2979 bp, Betatorquevirus sp.
  source 1..2979: mol_type = genomic DNA
taataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgcacttccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatgagcgactgctttaaaccaacatgctacaacaacaaaacaaagcaaactcactggattaataacctgcatttaacccacgacctgatctgcttctgcccaacaccaactagacacttattactagctttagcagaacaacaagaaacaattgaagtgtctaaacaagaaaaagaaaaaataacaagatgccttattactacagaagaagacggtacaactacagacgtcctagatggtatggacgaggttggattagacgcccttttcgcagaagatttcgaagaaaaagaagggtaagacctacttatactactattcctctaaagcaatggcaaccgccatataaaagaacatgctatataaaaggacaagactgtttaatatactatagcaacttaagactgggaatgaatagtacaatgtatgaaaaaagtattgtacctgtacattggccgggagggggttctttttctgtaagcatgttaactttagatgccttgtatgatatacataaactttgtagaaactggtggacatccacaaaccaagacttaccactagtaagatataaaggatgcaaaataacattttatcaaagcacatttacagactacatagtaagaatacatacagaactaccagctaacagtaacaaactaacatacccaaacacacatccactaatgatgatgatgtctaagtacaaacacattatacctagtagacaaacaagaagaaaaaagaaaccatacacaaaaatatttgtaaaaccacctccgcaatttgaaaacaaatggtactttgctacagacctctacaaaattccattactacaaatacactgcacagcatgcaacttacaaaacccatttgtaaaaccagacaaattatcaaacaatgttacattatggtcactaaacaccataagcatacaaaatagaaacatgtcagtggatcaaggacaatcatggccatttaaaatactaggaacacaaagcttttatttttacttttacaccggagcaaacctaccaggtgacacaacacaaataccagtagcagacctattaccactaacaaacccaagaataaacagaccaggacaatcactaaatgaggcaaaaattacagaccatattactttcacagaatacaaaaacaaatttacaaattattggggtaacccatttaataaacacattcaagaacacctagatatgatactatactcactaaaaagtccagaagcaataaaaaacgaatggacaacagaaaacatgaaatggaaccaattaaacaatgcaggaacaatggcattaacaccatttaacgagccaatattcacacaaatacaatataacccagatagagacacaggagaagacactcaattatacctactctctaacgctacaggaacaggatgggacccaccaggaattccagaattaatactagaaggatttccactatggttaatatattggggatttgcagactttcaaaaaaacctaaaaaaagtaacaaacatagacacaaattacatgttagtagcaaaaacaaaatttacacaaaaacctggcacattctacttagtaatactaaatgacacctttgtagaaggcaatagcccatatgaaaaacaacctttacctgaagacaacattaaatggtacccacaagtacaataccaattagaagcacaaaacaaactactacaaactgggccatttacaccaaacatacaaggacaactatcagacaatatatcaatgttttataaattttactttaaatggggaggaagcccaccaaaagcaattaatgttgaaaatcctgcccaccagattcaatatcccataccccgtaacgagcatgaaacaacttcgttacagagtccaggggaagccccagaatccatcttatactccttcgactatagacacgggaactacacaacaacagctttgtcacgaattagccaagactgggcacttaaagacactgtttctaaaattacagagccagatcgacagcaactgctcaaacaagccctcgaatgcctgcaaatctcggaagaaacgcaggagaaaaaagaaaaagaagtacagcagctcatcagcaacctcagacagcagcagcagctgtacagagagcgaataatatcattattaaaggaccaataacttttaactgtgtaaaaaaggtgaaattgtttgatgataaaccaaaaaaccgtagatttacacctgaggaatttgaaactgagttacaaatagcaaaatggttaaagagacccccaagatcctttgtaaatgatcctcccttttacccatggttaccacctgaacctgttgtaaactttaagcttaattttactgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacaccccc

SEQ ID NO: 55 - protein, 99 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEG

SEQ ID NO: 56 - protein, 203 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGFNIPYPVTSMKQLRYRVQGKPQNPSYTPSTIDTGTTQQQLCHELAKTGHLKTLFLKLQSQIDSNCSNKPSNACKSRKKRRRKKKKKYSSSSATSDSSSSCTESE

SEQ ID NO: 57 - protein, 219 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGARSTATAQTSPRMPANLGRNAGEKRKRSTAAHQQPQTAAAAVQRANNIIIKGPITFNCVKKVKLFDDKPKNRRFTPEEFETELQIAKWLKRPPRSFVNDPPFYPWLPPEPVVNFKLNFTE

SEQ ID NO: 58 - protein, 666 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 59 - protein, 148 aa, Betatorquevirus sp.
MPYYYRRRRYNYRRPRWYGRGWIRRPFRRRFRRKRRIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 60 - protein, 82 aa, Betatorquevirus sp.
MPYYYRRRRYNYRRPRWYGRGWIRRPFRRRFRRKRRSQIDSNCSNKPSNACKSRKKRRRKKKKKYSSSSATSDSSSSCTESE

SEQ ID NO: 61-67 - skipped (7 entries)

SEQ ID NO: 68 - protein, 25 aa, synthetic construct
  VARIANT 9: note = Any amino acid
  VARIANT 10: note = Any amino acid
  VARIANT 15: note = Any amino acid
  VARIANT 17: note = Any amino acid
  VARIANT 20: note = Any amino acid
  VARIANT 24: note = Any amino acid
LIJRQWQPXXIRRCXIXGYXPLIXC

SEQ ID NO: 69 - protein, 7 aa, synthetic construct
  VARIANT 3: note = Any amino acid
  VARIANT 4: note = Any amino acid
  VARIANT 6: note = Any amino acid
NYXXHXD

SEQ ID NO: 70 - protein, 9 aa, synthetic construct
  VARIANT 4: note = Any amino acid
  VARIANT 5: note = Any amino acid
FSLXXLYDZ

SEQ ID NO: 71 - protein, 18 aa, synthetic construct
  VARIANT 2: note = Any amino acid
  VARIANT 5: note = Any amino acid
  VARIANT 8: note = Any amino acid
  VARIANT 16: note = Any amino acid
NXWTXSNXDLDLCRYXGC

SEQ ID NO: 72 - protein, 16 aa, synthetic construct
  VARIANT 2: note = Any amino acid
  VARIANT 5: note = Any amino acid
  VARIANT 9: note = Any amino acid
  VARIANT 11: note = Any amino acid
  VARIANT 13: note = Any amino acid
TXPSXHPGXMXLXKHK

SEQ ID NO: 73 - protein, 10 aa, synthetic construct
  VARIANT 5: note = Any amino acid
  VARIANT 9: note = Any amino acid
IPSLXTRPXG

SEQ ID NO: 74 - protein, 18 aa, synthetic construct
  VARIANT 3: note = Any amino acid
  VARIANT 6: note = Any amino acid
  VARIANT 9: note = Any amino acid
  VARIANT 16: note = Any amino acid
RIXPPXLFXDKWYFQXDL

SEQ ID NO: 75 - protein, 8 aa, synthetic construct
  VARIANT 3: note = Any amino acid
  VARIANT 5: note = Any amino acid
LLXIXATA

SEQ ID NO: 76 - protein, 11 aa, synthetic construct
  VARIANT 2: note = Any amino acid
  VARIANT 3: note = Any amino acid
  VARIANT 6: note = Any amino acid
  VARIANT 9: note = Any amino acid
LXXPFXSPXTD

SEQ ID NO: 77 - protein, 14 aa, synthetic construct
  VARIANT 4: note = Any amino acid
  VARIANT 5: note = Any amino acid
  VARIANT 9: note = Any amino acid
  VARIANT 12: note = Any amino acid
YNPXXDKGXGNXIW

SEQ ID NO: 78 - protein, 8 aa, synthetic construct
  VARIANT 7: note = Any amino acid
CPYTZPXL

SEQ ID NO: 79 - protein, 8 aa, synthetic construct
  VARIANT 1: note = Any amino acid
  VARIANT 4: note = Any amino acid
  VARIANT 6: note = Any amino acid
XFGXGXMP

SEQ ID NO: 80 - protein, 8 aa, synthetic construct
  VARIANT 3: note = Any amino acid
  VARIANT 6: note = Any amino acid
  VARIANT 8: note = Any amino acid
HQXEVXEX

SEQ ID NO: 81 - protein, 12 aa, synthetic construct
  VARIANT 3: note = Any amino acid
  VARIANT 5: note = Any amino acid
  VARIANT 7: note = Any amino acid
KYXFXFXWGGNP

SEQ ID NO: 82 - protein, 8 aa, synthetic construct
  VARIANT 5: note = Any amino acid
HSWDXRRG

SEQ ID NO: 83 - protein, 7 aa, synthetic construct
  VARIANT 5: note = Any amino acid
AIKRXQQ

SEQ ID NO: 84 - protein, 8 aa, synthetic construct
  VARIANT 1: note = Any amino acid
  VARIANT 5: note = Any amino acid
  VARIANT 6: note = Any amino acid
XQZQXXLR

SEQ ID NO: 85 - protein, 9 aa, synthetic construct
  VARIANT 3: note = Any amino acid
  VARIANT 6: note = Any amino acid
  VARIANT 7: note = Any amino acid
PRXJQXXDP

SEQ ID NO: 86 - protein, 8 aa, synthetic construct
  VARIANT 5: note = Any amino acid
HSWDXRRG

SEQ ID NO: 87 - protein, 7 aa, synthetic construct
  VARIANT 5: note = Any amino acid
AIKRXQQ

SEQ ID NO: 88 - protein, 7 aa, synthetic construct
  VARIANT 4: note = Any amino acid
  VARIANT 5: note = Any amino acid
QZQXXLR

SEQ ID NO: 89 - protein, 7 aa, synthetic construct
  VARIANT 2: note = Any amino acid
KXKRRRR

SEQ ID NO: 90 - protein, 13 aa, synthetic construct
  VARIANT 3: note = Any amino acid
  VARIANT 6: note = Any amino acid
  VARIANT 7: note = Any amino acid
  VARIANT 10: note = Any amino acid
  VARIANT 11: note = Any amino acid
PIXSLXXYKXXTR

SEQ ID NO: 91 - protein, 12 aa, synthetic construct
  VARIANT 3: note = Any amino acid
  VARIANT 10: note = Any amino acid
LAXQLLKECXKN

SEQ ID NO: 92 - protein, 6 aa, synthetic construct
  VARIANT 4: note = Any amino acid
HLNXLA

SEQ ID NO: 93 - protein, 5 aa, synthetic construct
DRPPR

SEQ ID NO: 94 - protein, 9 aa, synthetic construct
  VARIANT 2: note = Any amino acid
  VARIANT 8: note = Any amino acid
DXPFYPWXP

SEQ ID NO: 95 - protein, 7 aa, synthetic construct
  VARIANT 2: note = Any amino acid
  VARIANT 6: note = Any amino acid
VXFKLXF

SEQ ID NO: 96 - protein, 15 aa, synthetic construct
  VARIANT 2: note = Any amino acid
  VARIANT 9: note = Any amino acid
  VARIANT 14: note = Any amino acid
WXPPVHBVXGIERXW

SEQ ID NO: 97 - protein, 6 aa, synthetic construct
  VARIANT 6: note = Any amino acid
AKRKLX

SEQ ID NO: 98 - protein, 10 aa, synthetic construct
  VARIANT 4: note = Any amino acid
  VARIANT 7: note = Any amino acid
  VARIANT 8: note = Any amino acid
PSSXDWXXEY

SEQ ID NO: 99 - protein, 5 aa, synthetic construct
DRPPR

SEQ ID NO: 100 - DNA, 2319 bp, Gyrovirus sp.
  source 1..2319: mol_type = genomic DNA
gaattccgagtggttactattccatcaccattctagcctgtacacagaaagtcaagatggacgaatcgctcgacttcgctcgcgattcgtcgaaggcggggggccggaggccccccggtggcccccctccaacgagtggagcacgtacaggggggtacgtcatccgtacaggggggtacgtcatccgtacaggggggtacgtcacaaagaggcgttcccgtacaggggggtacgtcacgcgtacaggggggtacgtcacagccaatcaaaagctgccacgttgcgaaagtgacgtttcgaaaatgggcggcgcaagcctctctatatattgagcgcacataccggtcggcagtaggtatacgcaaggcggtccgggtggatgcacgggaacggcggacaaccggccgctgggggcagtgaatcggcgcttagccgagaggggcaacctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatggcaagacgagctcgcagaccgagaggccgattttactccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctactatgactatccgcttccaaggggtcatctttctcacggaaggactcattctgcctaaaaacagcacagcggggggctatgcagaccacatgtacggggcgagagtcgccaagatctctgtgaacctgaaagagttcctgctagcctcaatgaacctgacatacgtgagcaaaatcggaggccccatcgccggtgagttgattgcggacgggtctaaatcacaagccgcggacaattggcctaattgctggctgccgctagataataacgtgccctccgctacaccatcggcatggtggagatgggccttaatgatgatgcagcccacggactcttgccggttctttaatcacccaaagcagatgaccctgcaagacatgggtcgcatgtttgggggctggcacctgttccgacacattgaaacccgctttcagctccttgccactaagaatgagggatccttcagccccgtggcgagtcttctctcccagggagagtacctcacgcgtcgggacgatgttaagtacagcagcgatcaccagaaccggtggcaaaaaggcggacaaccgatgacggggggcattgcttatgcgaccgggaaaatgagacccgacgagcaacagtaccctgctatgcccccagaccccccgatcatcaccgctactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaagggccagagatggcacacgctggtgccgctcggcacggagaccatcaccgacagctacatgtcagcacccgcatcagagctggacactaatttctttacgctttacgtagcgcaaggcacaaataagtcgcaacagtacaagttcggcacagctacatacgcgctaaaggagccggtaatgaagagcgatgcatgggcagtggtacgcgtccagtcggtctggcagctgggtaacaggcagaggccatacccatgggacgtcaactgggcgaacagcaccatgtactgggggacgcagccctgaaaaggggggggggctaaagccccccccccttaaacccccccctgggggggattcccccccagaccccccctttatatagcactcaataaacgcagaaaatagatttatcgcactatc

SEQ ID NO: 101 - protein, 8 aa, synthetic construct
  VARIANT 3: note = Any amino acid
  VARIANT 7: note = Any amino acid
NVXFKLXF

SEQ ID NO: 102 - protein, 25 aa, synthetic construct
  VARIANT 2: note = Any amino acid
  VARIANT 3: note = Any amino acid
  VARIANT 4: note = Any amino acid
  VARIANT 5: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 15: note = Any amino acid
  VARIANT 17: note = Any amino acid
  VARIANT 19: note = Any amino acid
  VARIANT 20: note = Any amino acid
  VARIANT 21: note = Any amino acid
JXXXXWQPXXXXXCXIXGXXXJWQP

SEQ ID NO: 103 - protein, 15 aa, synthetic construct
  VARIANT 2: note = Any amino acid
  VARIANT 4: note = Any amino acid
  VARIANT 5: note = Any amino acid
  VARIANT 6: note = Any amino acid
  VARIANT 8: note = Any amino acid
  VARIANT 9: note = Any amino acid
  VARIANT 10: note = Any amino acid
  VARIANT 11: note = Any amino acid
  VARIANT 13: note = Any amino acid
NXWXXXNXXXXLXRY

SEQ ID NO: 104 - protein, 8 aa, synthetic construct
  VARIANT 4: note = Any amino acid
  VARIANT 5: note = Any amino acid
  VARIANT 7: note = Any amino acid
YNPXXDXG

SEQ ID NO: 105 - DNA, 71 bp, synthetic construct
  source 1..71: mol_type = other DNA
cgggtgccgkaggtgagtttacacaccgmagtcaaggggcaattcgggctcrggactggccgggcyhtggg

SEQ ID NO: 106 - DNA, 71 bp, synthetic construct
  source 1..71: mol_type = other DNA
cgggtgccggaggtgagtttacacaccgcagtcaaggggcaattcgggctcgggactggccgggctwtggg

SEQ ID NO: 107 - DNA, 71 bp, Torque teno virus
  source 1..71: mol_type = genomic DNA
cgggtgccgtaggtgagtttacacaccgcagtcaaggggcaattcgggctcgggactggccgggctatggg

SEQ ID NO: 108 - DNA, 71 bp, Torque teno virus
  source 1..71: mol_type = genomic DNA
cgggtgccggaggtgagtttacacaccgcagtcaaggggcaattcgggctcgggactggccgggccctggg

SEQ ID NO: 109 - DNA, 71 bp, Torque teno virus
  source 1..71: mol_type = genomic DNA
cgggtgccggaggtgagtttacacaccgcagtcaaggggcaattcgggctcgggactggccgggctttggg

SEQ ID NO: 110 - DNA, 71 bp, Torque teno virus
  source 1..71: mol_type = genomic DNA
cgggtgccggaggtgagtttacacaccgcagtcaaggggcaattcgggctcgggactggccgggctatggg

SEQ ID NO: 111 - DNA, 71 bp, Torque teno virus
  source 1..71: mol_type = genomic DNA
cgggtgccggaggtgagtttacacaccgaagtcaaggggcaattcgggctcaggactggccgggctttggg

SEQ ID NO: 112 - DNA, 71 bp, synthetic construct
  source 1..71: mol_type = other DNA
cgggtgccggaggtgagtttacacaccgcagtcaaggggcaattcgggctcgggactggccgggcyhtggg

SEQ ID NO: 113 - DNA, 71 bp, Torque teno virus; identical to SEQ ID NO: 107
  source 1..71: mol_type = genomic DNA

SEQ ID NO: 114 - DNA, 70 bp, Torque teno virus
  source 1..70: mol_type = genomic DNA
cgggtgccggaggtgagtttacacaccgcagtcaaggggcaattcgggctcgggactggccgggcccggg

SEQ ID NO: 115 - DNA, 71 bp, Torque teno virus; identical to SEQ ID NO: 111
  source 1..71: mol_type = genomic DNA

SEQ ID NO: 116 - DNA, 69 bp, Torque teno virus
  source 1..69: mol_type = genomic DNA
cgggtgccggaggtgagtttacacaccgcagtcaaggggcaattcgggctcgggaggccgggccatggg

SEQ ID NO: 117 - DNA, 71 bp, Torque teno virus
  source 1..71: mol_type = genomic DNA
cgggtgccggaggtgagtttacacaccgcagtcaaggggcaattcgggctcgggactggccgggccccggg

SEQ ID NO: 118 - DNA, 71 bp, Torque teno virus; identical to SEQ ID NO: 110
  source 1..71: mol_type = genomic DNA

SEQ ID NO: 119 - DNA, 71 bp, Torque teno virus
  source 1..71: mol_type = genomic DNA
cgggtgccgaaggtgagtttacacaccgcagtcaaggggcaattcgggctcgggactggccgggctatggg

SEQ ID NO: 120 - DNA, 117 bp, synthetic construct
  source 1..117: mol_type = other DNA
  misc_difference 10: note = This base may be absent
  misc_difference 12: note = This base may be absent
  misc_difference 30: note = This base may be absent
  misc_difference 31: note = This base may be absent
  misc_difference 32: note = This base may be absent
  misc_difference 34: note = This base may be absent
  misc_difference 43: note = This base may be absent
  misc_difference 44: note = This base may be absent
  misc_difference 45: note = This base may be absent
  misc_difference 46: note = This base may be absent
  misc_difference 52: note = This base may be absent
  misc_difference 53: note = This base may be absent
  misc_difference 54: note = This base may be absent
  misc_difference 70: note = This base may be absent
  misc_difference 71: note = This base may be absent
  misc_difference 89: note = This base may be absent
  misc_difference 90: note = This base may be absent
  misc_difference 103: note = This base may be absent
cggcggsggsgcsscgcgctdcgcgcgcsgcccrsyrggggrdssmmwgcskcscccccccscgcgcatgcgcrcgggkcccccccccyvsggggggctccgcccccccggcccccc

SEQ ID NO: 121 - DNA, 169 bp, synthetic construct
  source 1..169: mol_type = other DNA
  misc_difference 20: note = a, c, t, g, unknown or other
  misc_difference 22: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 62: note = a, c, t, g, unknown or other
  misc_difference 64: note = a, c, t, g, unknown or other
  misc_difference 97: note = a, c, t, g, unknown or other
  misc_difference 98: note = a, c, t, g, unknown or other
gccgccgcggcggcggsggngnsgcgcgctdcgcgcgcsnnncrccrgggggnnnncwgcsncncccccccccgcgcatgcgcgggkcccccccccnncggggggctccgccccccggcccccccccgtgctaaacccaccgcgcatgcgcgaccacgcccccgccgcc

SEQ ID NO: 122 - DNA, 79 bp, synthetic construct
  source 1..79: mol_type = other DNA
  misc_difference 20: note = a, c, t, g, unknown or other
  misc_difference 22: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 62: note = a, c, t, g, unknown or other
  misc_difference 64: note = a, c, t, g, unknown or other
gccgccgcggcggcggsggngnsgcgcgctdcgcgcgcsnnncrccrgggggnnnncwgcsncncccccccccgcgcat

SEQ ID NO: 123 - DNA, 31 bp, synthetic construct
  source 1..31: mol_type = other DNA
  misc_difference 18: note = a, c, t, g, unknown or other
  misc_difference 19: note = a, c, t, g, unknown or other
gcgcgggkcccccccccnncggggggctccg

SEQ ID NO: 124 - DNA, 59 bp, synthetic construct
  source 1..59: mol_type = other DNA
ccccccggcccccccccgtgctaaacccaccgcgcatgcgcgaccacgcccccgccgcc

SEQ ID NO: 125 - DNA, 156 bp, Torque teno virus
  source 1..156: mol_type = genomic DNA
gcggcgggggggcggccgcgttcgcgcgccgcccaccagggggtgctgcgcgcccccccccgcgcatgcgcggggcccccccccgggggggctccgcccccccggcccccccccgtgctaaacccaccgcgcatgcgcgaccacgcccccgccgcc

SEQ ID NO: 126-128 - skipped (3 entries)

SEQ ID NO: 129 - DNA, 25 bp, Torque teno virus
  source 1..25: mol_type = genomic DNA
ttcgcgcgccgcccaccagggggtg

SEQ ID NO: 130 - skipped

SEQ ID NO: 131 - DNA, 17 bp, Torque teno virus
  source 1..17: mol_type = genomic DNA
cgcccccccccgcgcat

SEQ ID NO: 132 - DNA, 17 bp, Torque teno virus
  source 1..17: mol_type = genomic DNA
gcgcggggccccccccc

SEQ ID NO: 133 - DNA, 72 bp, Torque teno virus
  source 1..72: mol_type = genomic DNA
gggggggctccgcccccccggcccccccccgtgctaaacccaccgcgcatgcgcgaccacgcccccgccgcc

SEQ ID NO: 134 - DNA, 115 bp, Torque teno virus
  source 1..115: mol_type = genomic DNA
cggcggcggcggcgcgcgcgctgcgcgcgcgcgccgggggggcgccagcgcccccccccccgcgcatgcacgggtccccccccccacggggggctccgccccccggccccccccc

SEQ ID NO: 135 - DNA, 14 bp, Torque teno virus
  source 1..14: mol_type = genomic DNA
cggcggcggcggcg

SEQ ID NO: 136 - DNA, 17 bp, Torque teno virus
  source 1..17: mol_type = genomic DNA
cgcgcgctgcgcgcgcg

SEQ ID NO: 137 - DNA, 19 bp, Torque teno virus
  source 1..19: mol_type = genomic DNA
cgccgggggggcgccagcg

SEQ ID NO: 138 - DNA, 17 bp, Torque teno virus
  source 1..17: mol_type = genomic DNA
cccccccccccgcgcat

SEQ ID NO: 139 - DNA, 31 bp, Torque teno virus
  source 1..31: mol_type = genomic DNA
gcacgggtccccccccccacggggggctccg

SEQ ID NO: 140 - DNA, 17 bp, Torque teno virus
  source 1..17: mol_type = genomic DNA
ccccccggccccccccc

SEQ ID NO: 141 - DNA, 121 bp, Torque teno virus
  source 1..121: mol_type = genomic DNA
ccgtcggcgggggggccgcgcgctgcgcgcgcggcccccgggggaggcacagcctcccccccccgcgcgcatgcgcgcgggtccccccccctccggggggctccgccccccggcccccccc

SEQ ID NO: 142 - DNA, 37 bp, Torque teno virus
  source 1..37: mol_type = genomic DNA
ccgtcggcgggggggccgcgcgctgcgcgcgcggccc

SEQ ID NO: 143 - DNA, 84 bp, Torque teno virus
  source 1..84: mol_type = genomic DNA
ccgggggaggcacagcctcccccccccgcgcgcatgcgcgcgggtccccccccctccggggggctccgccccccggcccccccc

SEQ ID NO: 144 - DNA, 104 bp, Torque teno virus
  source 1..104: mol_type = genomic DNA
cggcggcggcgcgcgcgctacgcgcgcgcgccggggggctgccgccccccccccgcgcatgcgcggggcccccccccgcggggggctccgccccccggcccccc

SEQ ID NO: 145 - DNA, 11 bp, Torque teno virus
  source 1..11: mol_type = genomic DNA
cggcggcggcg

SEQ ID NO: 146 - DNA, 17 bp, Torque teno virus
  source 1..17: mol_type = genomic DNA
cgcgcgctacgcgcgcg

SEQ ID NO: 147 - DNA, 10 bp, Torque teno virus
  source 1..10: mol_type = genomic DNA
cgccgggggg

SEQ ID NO: 148 - skipped

SEQ ID NO: 149 - DNA, 15 bp, Torque teno virus
  source 1..15: mol_type = genomic DNA
cccccccccgcgcat

SEQ ID NO: 150 - DNA, 17 bp, Torque teno virus
  source 1..17: mol_type = genomic DNA
gcgcggggccccccccc

SEQ ID NO: 151 - DNA, 13 bp, Torque teno virus
  source 1..13: mol_type = genomic DNA
gcggggggctccg

SEQ ID NO: 152 - DNA, 14 bp, Torque teno virus
  source 1..14: mol_type = genomic DNA
ccccccggcccccc

SEQ ID NO: 153 - DNA, 122 bp, Torque teno virus
  source 1..122: mol_type = genomic DNA
gccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 154 - DNA, 19 bp, Torque teno virus
  source 1..19: mol_type = genomic DNA
gccgccgcggcggcggggg

SEQ ID NO: 155 - DNA, 41 bp, Torque teno virus
  source 1..41: mol_type = genomic DNA
gcggcgcgctgcgcgcgccgcccagtagggggagccatgcg

SEQ ID NO: 156 - DNA, 15 bp, Torque teno virus
  source 1..15: mol_type = genomic DNA
cccccccccgcgcat

SEQ ID NO: 157 - DNA, 17 bp, Torque teno virus
  source 1..17: mol_type = genomic DNA
gcgcggggccccccccc

SEQ ID NO: 158 - DNA, 13 bp, Torque teno virus
  source 1..13: mol_type = genomic DNA
gcggggggctccg

SEQ ID NO: 159 - DNA, 17 bp, Torque teno virus
  source 1..17: mol_type = genomic DNA
ccccccggccccccccg

SEQ ID NO: 160 - DNA, 36 bp, Torque teno virus
  source 1..36: mol_type = genomic DNA
cgcgctgcgcgcgccgcccagtagggggagccatgc

SEQ ID NO: 161 - DNA, 78 bp, Torque teno virus
  source 1..78: mol_type = genomic DNA
ccgccatcttaagtagttgaggcggacggtggcgtgagttcaaaggtcaccatcagccacacctactcaaaatggtgg

SEQ ID NO: 162 - DNA, 172 bp, Torque teno virus
  source 1..172: mol_type = genomic DNA
cttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcc

SEQ ID NO: 163 - DNA, 36 bp, synthetic construct; identical to SEQ ID NO: 160
  source 1..36: mol_type = other DNA

SEQ ID NO: 164 - DNA, 36 bp, synthetic construct
  source 1..36: mol_type = other DNA
gcgctdcgcgcgcgcgccggggggctgcgccccccc

SEQ ID NO: 165 - DNA, 36 bp, Torque teno virus
  source 1..36: mol_type = genomic DNA
gcgcttcgcgcgccgcccactagggggcgttgcgcg

SEQ ID NO: 166 - DNA, 36 bp, Torque teno virus
  source 1..36: mol_type = genomic DNA
gcgctgcgcgcgccgcccagtagggggcgcaatgcg

SEQ ID NO: 167 - DNA, 36 bp, Torque teno virus
  source 1..36: mol_type = genomic DNA
gcgctgcgcgcgcggcccccgggggaggcattgcct

SEQ ID NO: 168 - DNA, 36 bp, Torque teno virus
  source 1..36: mol_type = genomic DNA
gcgctgcgcgcgcgcgccgggggggcgccagcgccc

SEQ ID NO: 169 - DNA, 36 bp, Torque teno virus
  source 1..36: mol_type = genomic DNA
gcgcttcgcgcgcgcgccggggggctccgccccccc

SEQ ID NO: 170 - DNA, 36 bp, Torque teno virus
  source 1..36: mol_type = genomic DNA
gcgcttcgcgcgcgcgccggggggctgcgccccccc

SEQ ID NO: 171 - DNA, 36 bp, Torque teno virus
  source 1..36: mol_type = genomic DNA
gcgctacgcgcgcgcgccggggggctgcgccccccc

SEQ ID NO: 172 - DNA, 36 bp, Torque teno virus
  source 1..36: mol_type = genomic DNA
gcgctacgcgcgcgcgccggggggctctgccccccc

SEQ ID NO: 173 - protein, 96 aa, Betatorquevirus sp.
MQIQPPIRTFKQTISDWKNLIVHVHDNICNCNKPLEHTIDTCITNPDELRLNKSTKQQLQKCLGTPEEDTQEDVIDGFADGELDALFAQDTEEDTG

SEQ ID NO: 174-214 - skipped (41 entries)

SEQ ID NO: 215 - protein, 666 aa, Betatorquevirus sp.; identical to SEQ ID NO: 58

SEQ ID NO: 216 - protein, 38 aa, Betatorquevirus sp.
MPYYYRRRRYNYRRPRWYGRGWIRRPFRRRFRRKRRVR

SEQ ID NO: 217 - protein, 208 aa, Betatorquevirus sp.
PTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 218 - protein, 128 aa, Betatorquevirus sp.
ISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAG

SEQ ID NO: 219 - protein, 163 aa, Betatorquevirus sp.
TMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFK

SEQ ID NO: 220 - protein, 129 aa, Betatorquevirus sp.
WGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 221-226 - skipped (6 entries)

SEQ ID NO: 227 - protein, 220 aa, synthetic construct
  VARIANT 29: note = Any amino acid
  VARIANT 30: note = Any amino acid
  VARIANT 31: note = Any amino acid
  VARIANT 29..31: note = This region may encompass 0-3 residues
  VARIANT 100: note = Any amino acid
  VARIANT 125: note = Any amino acid
  VARIANT 126: note = Any amino acid
  VARIANT 127: note = Any amino acid
  VARIANT 128: note = Any amino acid
  VARIANT 129: note = Any amino acid
  VARIANT 125..129: note = This region may encompass 1-5 residues
  VARIANT 181: note = Any amino acid
  VARIANT 211: note = Any amino acid
LVLTQWQPNTVRRCYIRGYLPLIICGENXXXTTSRNYATHSDDTIQKGPFGGGMSTTTFSLRVLYDEYQRFMNRWTYSNEDLDLARYLGCKFTFYRHPDXDFIVQYNTNPPFKDTKLTAPSIHPXXXXXGMLMLSKRKILIPSLKTRPKGKHYVKVRIGPPKLFEDKWYTQSDLCDVPLVXLYATAADLQHPFGSPQTDNPCVTFQVLGSXYNKHLSISP

SEQ ID NO: 228 - protein, 172 aa, synthetic construct
  VARIANT 38: note = Any amino acid
  VARIANT 44: note = Any amino acid
  VARIANT 45: note = Any amino acid
  VARIANT 46: note = Any amino acid
  VARIANT 44..46: note = This region may encompass 0-3 residues
  VARIANT 77: note = Any amino acid
  VARIANT 79: note = Any amino acid
  VARIANT 98: note = Any amino acid
  VARIANT 99: note = Any amino acid
  VARIANT 100: note = Any amino acid
  VARIANT 101: note = Any amino acid
  VARIANT 98..101: note = This region may encompass 0-4 residues
SNFEFPGAYTDITYNPLTDKGVGNMVWIQYLTKPDTIXDKTQSXXXKCLIEDLPLWAALYGYVDFCEKETGDSAIIXNXGRVLIRCPYTKPPLYDKTXXXXNKGFVPYSTNFGNGKMPGGSGYVPIYWRARWYPTLFHQKEVLEDIVQSGPFAYKDEKPSTQLVMKYCFNFN

SEQ ID NO: 229 - protein, 258 aa, synthetic construct
  VARIANT 20: note = Any amino acid
  VARIANT 21: note = Any amino acid
  VARIANT 22: note = Any amino acid
  VARIANT 20..22: note = This region may encompass 0-3 residues
  VARIANT 25: note = Any amino acid
  VARIANT 78: note = Any amino acid
  VARIANT 89: note = Any amino acid
  VARIANT 91: note = Any amino acid
  VARIANT 95: note = Any amino acid
  VARIANT 96: note = Any amino acid
  VARIANT 97: note = Any amino acid
  VARIANT 98: note = Any amino acid
  VARIANT 95..98: note = This region may encompass 1-4 residues
  VARIANT 107: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 113: note = Any amino acid
  VARIANT 114: note = Any amino acid
  VARIANT 115: note = Any amino acid
  VARIANT 116: note = Any amino acid
  VARIANT 117: note = Any amino acid
  VARIANT 118: note = Any amino acid
  VARIANT 119: note = Any amino acid
  VARIANT 120: note = Any amino acid
  VARIANT 107..120: note = This region may encompass 2-14 residues
  VARIANT 129: note = Any amino acid
  VARIANT 139: note = Any amino acid
  VARIANT 140: note = Any amino acid
  VARIANT 141: note = Any amino acid
  VARIANT 142: note = Any amino acid
  VARIANT 143: note = Any amino acid
  VARIANT 144: note = Any amino acid
  VARIANT 145: note = Any amino acid
  VARIANT 146: note = Any amino acid
  VARIANT 147: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
  VARIANT 154: note = Any amino acid
  VARIANT 155: note = Any amino acid
  VARIANT 156: note = Any amino acid
  VARIANT 157: note = Any amino acid
  VARIANT 158: note = Any amino acid
  VARIANT 159: note = Any amino acid
  VARIANT 160: note = Any amino acid
  VARIANT 161: note = Any amino acid
  VARIANT 162: note = Any amino acid
  VARIANT 163: note = Any amino acid
  VARIANT 164: note = Any amino acid
  VARIANT 165: note = Any amino acid
  VARIANT 166: note = Any amino acid
  VARIANT 167: note = Any amino acid
  VARIANT 168: note = Any amino acid
  VARIANT 139..168: note = This region may encompass 0-30 residues
  VARIANT 201: note = Any amino acid
  VARIANT 202: note = Any amino acid
  VARIANT 203: note = Any amino acid
  VARIANT 204: note = Any amino acid
  VARIANT 201..204: note = This region may encompass 0-4 residues
  VARIANT 219: note = Any amino acid
  VARIANT 220: note = Any amino acid
  VARIANT 221: note = Any amino acid
  VARIANT 222: note = Any amino acid
  VARIANT 223: note = Any amino acid
  VARIANT 224: note = Any amino acid
  VARIANT 225: note = Any amino acid
  VARIANT 226: note = Any amino acid
  VARIANT 227: note = Any amino acid
  VARIANT 228: note = Any amino acid
  VARIANT 229: note = Any amino acid
  VARIANT 230: note = Any amino acid
  VARIANT 231: note = Any amino acid
  VARIANT 232: note = Any amino acid
  VARIANT 233: note = Any amino acid
  VARIANT 234: note = Any amino acid
  VARIANT 235: note = Any amino acid
  VARIANT 236: note = Any amino acid
  VARIANT 237: note = Any amino acid
  VARIANT 238: note = Any amino acid
  VARIANT 239: note = Any amino acid
  VARIANT 240: note = Any amino acid
  VARIANT 241: note = Any amino acid
  VARIANT 242: note = Any amino acid
  VARIANT 243: note = Any amino acid
  VARIANT 244: note = Any amino acid
  VARIANT 245: note = Any amino acid
  VARIANT 246: note = Any amino acid
  VARIANT 247: note = Any amino acid
  VARIANT 248: note = Any amino acid
  VARIANT 249: note = Any amino acid
  VARIANT 250: note = Any amino acid
  VARIANT 251: note = Any amino acid
  VARIANT 252: note = Any amino acid
  VARIANT 253: note = Any amino acid
  VARIANT 254: note = Any amino acid
  VARIANT 255: note = Any amino acid
  VARIANT 256: note = Any amino acid
  VARIANT 257: note = Any amino acid
  VARIANT 258: note = Any amino acid
  VARIANT 219..258: note = This region may encompass 0-40 residues
WGGNPISQQVVRNPCKDSGXXXSGXGRQPRSVQVVDPKYMGPEYTFHSWDWRRGLFGEKAIKRMSEQPTDDEIFTGGXPKRPRRDPPTXQXPEEXXXXQKESSSFRXXXXXXXXXXXXXXPWESSSQEXESESQEEEEXXXXXXXXXXXXXXXXXXXXXXXXXXXXXXEQTVQQQLRQQLREQRRLRVQLQLLFQQLLKTXXXXQAGLHINPLLLSQAXXXXXXXXXXXXXXXXXXXXXXXXXXXXXXXXXXXXXXXX

SEQ ID NO: 230 - protein, 214 aa, synthetic construct
  VARIANT 136: note = Any amino acid
  VARIANT 138: note = Any amino acid
  VARIANT 139: note = Any amino acid
  VARIANT 140: note = Any amino acid
  VARIANT 141: note = Any amino acid
  VARIANT 138..141: note = This region may encompass 1-4 residues
  VARIANT 179: note = Any amino acid
LKQWQPSTIRKCKIKGYLPLFQCGKGRISNNYTQYKESIVPHHEPGGGGWSIQQFTLGALYEEHLKLRNWWTKSNDGLPLVRYLGCTIKLYRSEDTDYIVTYQRCYPMTATKLTYLSTQPSRMLMNKHKIIVPSKXTXXXXNKKKKPYKKIFIKPPSQMQNKWYFQQDIANTPLLQLTXTACSLDRMYLSSDSISNNITFTSLNTNFFQNPNFQ

SEQ ID NO: 231 - protein, 187 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 1..10: note = This region may encompass 4-10 residues
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 38..45: note = This region may encompass 1-8 residues
  VARIANT 94: note = Any amino acid
  VARIANT 100: note = Any amino acid
  VARIANT 101: note = Any amino acid
  VARIANT 102: note = Any amino acid
  VARIANT 100..102: note = This region may encompass 1-3 residues
  VARIANT 112: note = Any amino acid
  VARIANT 114: note = Any amino acid
  VARIANT 115: note = Any amino acid
  VARIANT 114..115: note = This region may encompass 0-2 residues
  VARIANT 124: note = Any amino acid
  VARIANT 125: note = Any amino acid
  VARIANT 126: note = Any amino acid
  VARIANT 127: note = Any amino acid
  VARIANT 128: note = Any amino acid
  VARIANT 129: note = Any amino acid
  VARIANT 130: note = Any amino acid
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 139: note = Any amino acid
  VARIANT 124..139: note = This region may encompass 3-16 residues
  VARIANT 154: note = Any amino acid
XXXXXXXXXXTPLYFECRYNPFKDKGTGNKVYLVSNNXXXXXXXXTGWDPPTDPDLIIEGFPLWLLLWGWLDWQKKLGKIQNIDTDYILVIQSXYYIPPXXXKLPYYVPLDXDXXFLHGRSPYXXXXXXXXXXXXXXXXPSDKQHWHPKVRFQXETINNIALTGPGTPKLPNQKSIQAHMKYKFYFK

SEQ ID NO: 232 - protein, 163 aa, synthetic construct
  VARIANT 34: note = Any amino acid
  VARIANT 65: note = Any amino acid
  VARIANT 77: note = Any amino acid
  VARIANT 78: note = Any amino acid
  VARIANT 86: note = Any amino acid
  VARIANT 87: note = Any amino acid
  VARIANT 96: note = Any amino acid
  VARIANT 102: note = Any amino acid
  VARIANT 103: note = Any amino acid
  VARIANT 104: note = Any amino acid
  VARIANT 105: note = Any amino acid
  VARIANT 106: note = Any amino acid
  VARIANT 102..106: note = This region may encompass 0-5 residues
  VARIANT 125: note = Any amino acid
  VARIANT 135: note = Any amino acid
  VARIANT 138: note = Any amino acid
  VARIANT 139: note = Any amino acid
  VARIANT 140: note = Any amino acid
  VARIANT 141: note = Any amino acid
  VARIANT 142: note = Any amino acid
  VARIANT 143: note = Any amino acid
  VARIANT 144: note = Any amino acid
  VARIANT 145: note = Any amino acid
  VARIANT 146: note = Any amino acid
  VARIANT 147: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
  VARIANT 154: note = Any amino acid
  VARIANT 155: note = Any amino acid
  VARIANT 156: note = Any amino acid
  VARIANT 157: note = Any amino acid
  VARIANT 158: note = Any amino acid
  VARIANT 159: note = Any amino acid
  VARIANT 160: note = Any amino acid
  VARIANT 161: note = Any amino acid
  VARIANT 162: note = Any amino acid
  VARIANT 163: note = Any amino acid
  VARIANT 138..163: note = This region may encompass 0-26 residues
WGGCPAPMETITDPCKQPKYPIPNNLLQTTSLQXPTTPIETYLYKFDERRGLLTKKAAKRIKKDXTTETTLFTDTGXXTSTTLPTXXQTETTQEEXTSEEEXXXXXETLLQQLQQLRRKQKQLRXRILQLLQLLXLLXXXXXXXXXXXXXXXXXXXXXXXXXX

SEQ ID NO: 233 - protein, 203 aa, synthetic construct
  VARIANT 79: note = Any amino acid
  VARIANT 104: note = Any amino acid
  VARIANT 116: note = Any amino acid
  VARIANT 120: note = Any amino acid
  VARIANT 121: note = Any amino acid
  VARIANT 125: note = Any amino acid
  VARIANT 170: note = Any amino acid
TIPLKQWQPESIRKCKIKGYGTLVLGAEGRQFYCYTNEKDEYTPPKAPGGGGFGVELFSLEYLYEQWKARNNIWTKSNXYKDLCRYTGCKITFYRHPTTDFIVXYSRQPPFEIDKXTYMXXHPQXLLLRKHKKIILSKATNPKGKLKKKIKIKPPKQMLNKWFFQKQFAXYGLVQLQAAACBLRYPRLGCCNENRLITLYYLN

SEQ ID NO: 234 - protein, 162 aa, synthetic construct
  VARIANT 12: note = Any amino acid
  VARIANT 20: note = Any amino acid
  VARIANT 23: note = Any amino acid
  VARIANT 30: note = Any amino acid
  VARIANT 84: note = Any amino acid
  VARIANT 90: note = Any amino acid
  VARIANT 95: note = Any amino acid
  VARIANT 105: note = Any amino acid
  VARIANT 113: note = Any amino acid
  VARIANT 154: note = Any amino acid
  VARIANT 156: note = Any amino acid
LPIVVARYNPAXDTGKGNKXWLXSTLNGSXWAPPTTDKDLIIEGLPLWLALYGYWSYJKKVKKDKGILQSHMFVVKSPAIQPLXTATTQXTFYPXIDNSFIQGKXPYDEPJTXNQKKLWYPTLEHQQETINAIVESGPYVPKLDNQKNSTWELXYXYTFYFK

SEQ ID NO: 235 - protein, 177 aa, synthetic construct
  VARIANT 16: note = Any amino acid
  VARIANT 26: note = Any amino acid
  VARIANT 33: note = Any amino acid
  VARIANT 73: note = Any amino acid
  VARIANT 81: note = Any amino acid
  VARIANT 82: note = Any amino acid
  VARIANT 81..82: note = This region may encompass 0-2 residues
  VARIANT 90: note = Any amino acid
  VARIANT 94: note = Any amino acid
  VARIANT 119: note = Any amino acid
  VARIANT 120: note = Any amino acid
  VARIANT 121: note = Any amino acid
  VARIANT 122: note = Any amino acid
  VARIANT 123: note = Any amino acid
  VARIANT 124: note = Any amino acid
  VARIANT 119..124: note = This region may encompass 1-6 residues
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
  VARIANT 173: note = Any amino acid
  VARIANT 174: note = Any amino acid
  VARIANT 175: note = Any amino acid
  VARIANT 176: note = Any amino acid
  VARIANT 177: note = Any amino acid
  VARIANT 168..177: note = This region may encompass 1-10 residues
WGGPQIPDQPVEDPKXQGTYPVPDTXQQTIQIXNPLKQKPETMFHDWDYRRGIITSTALKRMQENLETDSSFXSDSEETPXXKKKKRLTXELPXPQEETEEIQSCLLSLCEESTCQEEXXXXXXENLQQLIHQQQQQQQQLKHNILKLLSDLKZKQRLLQLQTGILEXXXXXXXXXX

SEQ ID NO: 236-696 - skipped (461 entries)

SEQ ID NO: 697 - DNA, 20 bp, synthetic construct
  source 1..20: mol_type = other DNA
gaagcccaccaaaagcaatt

SEQ ID NO: 698 - DNA, 21 bp, synthetic construct
  source 1..21: mol_type = other DNA
agttcccgtgtctatagtcga

SEQ ID NO: 699 - DNA, 22 bp, synthetic construct
  source 1..22: mol_type = other DNA
acttcgttacagagtccagggg

SEQ ID NO: 700 - DNA, 20 bp, synthetic construct
  source 1..20: mol_type = other DNA
agcaacaggtaatggaggac

SEQ ID NO: 701 - DNA, 20 bp, synthetic construct
  source 1..20: mol_type = other DNA
tggaagctggggtctttaac

SEQ ID NO: 702 - DNA, 22 bp, synthetic construct
  source 1..22: mol_type = other DNA
tctaccttaggtgcaaagggcc

SEQ ID NO: 703-800 - skipped (98 entries)

SEQ ID NO: 801 - DNA, 156 bp, Torque teno virus; identical to SEQ ID NO: 125
  source 1..156: mol_type = genomic DNA

SEQ ID NO: 802 - DNA, 150 bp, Torque teno virus
  source 1..150: mol_type = genomic DNA
ccgagcgttagcgaggagtgcgaccctaccccctgggcccacttcttcggagccgcgcgctacgccttcggctgcgcgcggcacctcagacccccgctcgtgctgacacgcttgcgcgtgtcagaccacttcgggctcgcgggggtcggg

SEQ ID NO: 803 - DNA, 122 bp, Torque teno virus; identical to SEQ ID NO: 153
  source 1..122: mol_type = genomic DNA

SEQ ID NO: 804 - DNA, 111 bp, Torque teno virus
  source 1..111: mol_type = genomic DNA
cggcccagcggcggcgcgcgcgcttcgcgcgcgcgccggggggctccgcccccccccgcgcatgcgcggggcccccccccgcggggggctccgccccccggtccccccccg

SEQ ID NO: 805 - DNA, 115 bp, Torque teno virus
  source 1..115: mol_type = genomic DNA
cggccgtgcggcggcgcgcgcgcttcgcgcgcgcgccgggggctgccgcccccccccgcgcatgcgcgcggggcccccccccgcggggggctccgccccccggcccccccccccg

SEQ ID NO: 806 - DNA, 104 bp, Torque teno virus; identical to SEQ ID NO: 144
  source 1..104: mol_type = genomic DNA

SEQ ID NO: 807 - DNA, 108 bp, Torque teno virus
  source 1..108: mol_type = genomic DNA
ggcggcggcgcgcgcgctacgcgcgcgcgccggggagctctgcccccccccgcgcatgcgcgcgggtcccccccccgcggggggctccgccccccggtcccccccccg

SEQ ID NO: 808-809 - skipped (2 entries)

SEQ ID NO: 810 - DNA, 71 bp, synthetic construct
  source 1..71: mol_type = other DNA
cgggtgccggaggtgagtttacacaccgcagtcaaggggcaattcgggctcgggactggccgggcymtggg

SEQ ID NO: 811 - DNA, 71 bp, Torque teno virus; identical to SEQ ID NO: 107
  source 1..71: mol_type = genomic DNA

SEQ ID NO: 812 - DNA, 70 bp, Torque teno virus; identical to SEQ ID NO: 114
  source 1..70: mol_type = genomic DNA

SEQ ID NO: 813 - DNA, 71 bp, Torque teno virus; identical to SEQ ID NO: 111
  source 1..71: mol_type = genomic DNA

SEQ ID NO: 814 - DNA, 69 bp, Torque teno virus; identical to SEQ ID NO: 116
  source 1..69: mol_type = genomic DNA

SEQ ID NO: 815 - DNA, 71 bp, Torque teno virus; identical to SEQ ID NO: 117
  source 1..71: mol_type = genomic DNA

SEQ ID NO: 816 - DNA, 71 bp, Torque teno virus; identical to SEQ ID NO: 110
  source 1..71: mol_type = genomic DNA

SEQ ID NO: 817 - DNA, 71 bp, Torque teno virus; identical to SEQ ID NO: 119
  source 1..71: mol_type = genomic DNA

SEQ ID NO: 818 - DNA, 36 bp, synthetic construct
  source 1..36: mol_type = other DNA
gcgctkcgcgcgcgcgccggggggctgcgccccccc

SEQ ID NO: 819 - DNA, 36 bp, Torque teno virus; identical to SEQ ID NO: 165
  source 1..36: mol_type = genomic DNA

SEQ ID NO: 820 - DNA, 36 bp, Torque teno virus; identical to SEQ ID NO: 166
  source 1..36: mol_type = genomic DNA

SEQ ID NO: 821 - DNA, 36 bp, Torque teno virus; identical to SEQ ID NO: 167
  source 1..36: mol_type = genomic DNA

SEQ ID NO: 822 - DNA, 36 bp, Torque teno virus; identical to SEQ ID NO: 168
  source 1..36: mol_type = genomic DNA

SEQ ID NO: 823 - DNA, 36 bp, Torque teno virus; identical to SEQ ID NO: 169
  source 1..36: mol_type = genomic DNA

SEQ ID NO: 824 - DNA, 36 bp, Torque teno virus; identical to SEQ ID NO: 169
  source 1..36: mol_type = genomic DNA

SEQ ID NO: 825 - DNA, 36 bp, Torque teno virus; identical to SEQ ID NO: 170
  source 1..36: mol_type = genomic DNA

SEQ ID NO: 826 - DNA, 36 bp, Torque teno virus; identical to SEQ ID NO: 171
  source 1..36: mol_type = genomic DNA

SEQ ID NO: 827 - DNA, 36 bp, Torque teno virus; identical to SEQ ID NO: 172
  source 1..36: mol_type = genomic DNA

SEQ ID NO: 828 - protein, 727 aa, synthetic construct
  VARIANT 36: note = Any amino acid
  VARIANT 170: note = Any amino acid
  VARIANT 246: note = Any amino acid
  VARIANT 276: note = Any amino acid
  VARIANT 361: note = Any amino acid
  VARIANT 371: note = Any amino acid
  VARIANT 436: note = Any amino acid
  VARIANT 685: note = Any amino acid
MAWGWWRRRRMAWRRWRRRRRRRRRRRRRRRRRRPXRRRRRRRRVRRRRRGRWRRRRYRRWRRRRRRRRRKKLVLTQWQPNTVRRCYIRCRGYLPLIICGENTTSRNYATHSDDTPQGPFGGGMSTTTFSLRVLYDEYQRFMNRWTYSNEDLDLARYLGCKFTFYRHPDXDFIVQYNTNPPFKDTKLTAPSIHPGMLMLSKRKILIPSLKTRPKGKHYVKVRIGPPKLFEDKWYTQSDLCDVPLVXLYATAADLQHPFGSPQTDNPCVTFQVLGSXKYNKHLSISPTNDNLKHLLNNLYTSGYNTFQTIAQLKPTIDAKNGTTNTTNTTTTGTTFTTTKNDSWYGGNTYNPNIKKLKKIRXYFKKLTKKLXPTYTTPTDYLEYHLGIYSPIFLSPGRSNFEFPGAYTDITYNPLTDKGVGNMVWYIQYLTKPDTIXDKTQSKCLIEDLPLWAALYGYVDFCEKETGDQDIETNGRVLIRCPYTKPPLYDKTDPNKGFVPYSTNFGNGKMPGGSGYVPIYWRARWYPTLFHQKEVLEDIVQSGPPFAYKDDLKSATLTAKYKFKFKWGGNPISQQVVRNPCKDSGSSPPGRGPRSVQVVDPKYMGPEYTFHSWDWRRGLFGEKAIKRMSEQPTDDELFPAGPKRPRRDPPEEQEESSLFLRRRPPWEESSQEESSSEEEEQEEQTXQQQLRQQLREQRQLRVQLQLLFQQLLKTQAGLHINPLLLSQA

SEQ ID NO: 829 - protein, 11 aa, synthetic construct
  VARIANT 4: note = Any amino acid
  VARIANT 5: note = Any amino acid
  VARIANT 7: note = Any amino acid
  VARIANT 9: note = Any amino acid
  VARIANT 10: note = Any amino acid
YNPXXDXGXXN

SEQ ID NO: 830-850 - skipped (21 entries)

SEQ ID NO: 851 - protein, 20 aa, Torque teno midi virus
LRYNPAADQGPGNIVTIIDL

SEQ ID NO: 852-948 - skipped (97 entries)

SEQ ID NO: 949 - protein, 21 aa, synthetic construct
  VARIANT 1: note = W or F
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 10: note = Any amino acid
  VARIANT 11: note = Any amino acid
  VARIANT 12: note = Any amino acid
  VARIANT 14: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
XXXXXXXXHXXXCXCXXXXXH

SEQ ID NO: 950 - protein, 203 aa, Alphatorquevirus sp.
KIIIRQWQPNYTRRCNIVGYMPLLICGENTVATNYATHSDDSYYPGPFGGGMTTDKFTLRILYDEYKRFMNYWTSSNEDLDLCRYLGCTLYVFRHPEVDFIIIINTSPPFLDTEITGPSIHPGMMALNKRSRWIPSIKNRPGRKHYIKIKVGAPRMFTDKWYPQTDLCDMTLLTIFASAADMQYPFGSPLTDTIVVSFQVLQS

SEQ ID NO: 951 - protein, 203 aa, Torque teno virus
KIIIRQWQPNYRRRCNIVGYLPILICGGNTVSRNYATHSDDTNYPGPFGGGMTTDKFSLRILYDEYKRFMNYWTASNEDLDLCRYLGCTFYFFRHPEVDFIIKINTMPPFLDTTITAPSIHPGLMALDKRARWIPSLKNRPGKKHYIKIRVGAPKMFTDKWYPQTDLCDMTLLTIYATAADMQYPFGSPLTDTVVVNSQVLQS

SEQ ID NO: 952 - protein, 203 aa, Torque teno virus
KIIIKQWQPNFIRRCYIIGCLPLVFCGENTTAQNYATHSDDMISKGPYGGGMTTTKFTLRILYDEFTRFMNFWTVSNEDLDLCRYVGCKLIFFKHPTVDFMVQINTQPPFLDTSLTAASIHPGIMMLSKRRILIPSLKTRPSRKHRVVVRVGAPRLFQDKWYPQSDLCDTVLLSIFATARDLQYPFGSPLTDNPCVNFQILGP

SEQ ID NO: 953 - protein, 203 aa, Torque teno virus
KIIIKQWQPNFIRRCYIIGYLPLIFCGENTTANNFATHSDDMIAKGPWGGGMTTTKFTLRILYDEFTRFMNFWTVSNEDLDLCRYVSCKLIFFKHPTVDFIVRINTEPPFLDTNLTAAQIHPGIMMLSKKHILIPSLKTRPSRKHRVVVRVGPPRLFQDKWYPQSDLCDTVLLSVFATACDLQYPFGSPLTDNPCVNFQILGH

SEQ ID NO: 954 - protein, 203 aa, Torque teno virus
KLIIKQWQPNFIRHCYIIGYMPLIICGENTFSHNYATHSDDMLSTGPYGGGMTTTKFTLRILFDEYQRHLNFWTVSNQDLDLARYLGTKIIFFRHPTVDFVVQIHTQPPFQDTEITAPSIHPGMLILSKKHILIPSLKTRPSKKHYVKVRVGAPRLFQDKWYPQSELCDVTLLVIYATACDLQYPFGSPQTDNVCVNFQILGQ

SEQ ID NO: 955 - protein, 204 aa, Torque teno virus
TLILRQWQPDCIRHCKIIGWMPLITCGKGSTQFNYITHADDITPRGASYGGNFTNMTFSLEAIYEQFLYHRNRWSASNHDLELCRYKGTTLKLYRHPEVDYIVTYSRTGPFEISHMTYLSTHPMLMLLNKHHIVVPSLKTKPRGRKAIKVRIRPPKLMNNKWYFTRDFCNIGLFQLWATGLELRNPWLRMSTLSPCIGFNVLKN

SEQ ID NO: 956 - protein, 204 aa, Torque teno virus
TLMLRQWQPDVIRHCKITGRMPLIICGKGSTQFNYITHADDITPRGASYGGNFTNMTFSLEAIYEQFLYHRNRWSASNHDLELCRYKGTTLKLYRHPDVDYIVTYSRTGPFEISHMTYLSTHPLLMLLNKHHIVVPSLKTKPRGRKAIKVRIRPPKLMNNKWYFTRDFCNIGLFQLWATGLELRNPWLRMSTLSPCIGFNVLKN

SEQ ID NO: 957 - protein, 203 aa, Torque teno virus
KLMLTQWNPQTVRKCVIRGFLPLFFCGQGAYHRNFTDHYDDVFPKGPSGGGHGSMVFNLSFLYQEFKKHHNKWSRSNLDFDLVRYKGTVIKLYRHQDFDYIVWISRTPPFQESLLTVMTHQPSVMLQAKKCIIVKSYRTHPGGKPYVTAKVRPPRLLTDKWYFQSDFCNVPLFSLQFALAELRFPICSPQTDTNCINFLVLDD

SEQ ID NO: 958 - protein, 203 aa, Torque teno virus
KLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDTADLTLFNIMAVEADLRFPFCSPQTDNTCISFQVLSS

SEQ ID NO: 959 - protein, 204 aa, Alphatorquevirus sp.
RLMLTQWSPQTVRNCSIRGIVPMVICGHTKAGRNYAIHSEDFTTQIQPFGGSFSTTTWSLKVLWDEHQKFQNRWSYPNTQLDLARYRGVTFWFYRDQKTDYIVQWSRNPPFKLNKYSSAMYHPGMMMQAKRKLVVPSFQTRPKGKKRYRVTIKPPNMFADKWYTQEDLCPVPLVQIVVSAASLLHPFCPPQTNNPCITFQVLKD

SEQ ID NO: 960 - protein, 205 aa, Torque teno virus
KLMLTQWHPNTMRRCLIKGIVPLVICGHTRWNYNYALHSKDYTEEGRYPHGGALSTTTWSLKVLYDEHLKHHDFWGYPNNQLDLARYKGAKFTFYRHKKTDFIIFFNRKPPFKLNKYSCASYHPGMLMQQRHKILLPSYETKPKGRPKITVRIKPPTLLEDKWYTQQDLCDVNLLQLVVTAADFRHPLCSPQTNTPTTTFQVLKD

SEQ ID NO: 961 - protein, 204 aa, Torque teno virus
RLILRQWQPATRRRCTITGYLPIVFCGHTRGNKNYALHSDDYTPQGQPFGGALSTTSFSLKVLFDQHQRGLNKWSFPNDQLDLARYRGCKFIFYRTKQTDWVGQYDISEPYKLDKYSCPNYHPGNMIKAKHKFLIPSYDTNPRGRQKIIVKIPPPDLFVDKWYTQEDLCSVNLVSLAVSAASFLHPFGSPQTDNPCYTFQVLKE

SEQ ID NO: 962 - protein, 204 aa, Torque teno virus
KLILTQWNPAIVKRCNIKGGLPIIICGEPRAAFNYGYHMEDYTPQPFPFGGGMSTVTFSLKALYDQYLKHQNRWTFSNDQLDLARYRGCKLRFYRSPVCDFIVHYNLIPPLKMNQFTSPNTHPGLLMLSKHKIIIPSFQTRPGGRRFVKIRLNPPKLFEDKWYTQQDLCKVPLVSITATAADLRYPFCSPQTNNPCTTFQVLRK

SEQ ID NO: 963 - protein, 203 aa, Torque teno virus
KIMLTQWNPQTTRKCIIRGMMPVLWAGMGTGGRNYAVRSDDYVVNKGFGGSFATETFSLKVLYDQFQRGFNRWSHTNEDLDLARYRGCRWTFYRHKDTDFIVYFTNNPPMKTNQFSAPLTTPGMLMRSKYKVLIPSFQTRPKGRKTVTVKIRPPKLFQDKWYTQQDLCSVPLVQLNVTAADFTHPFGSPLTETPCVEFQVLGD

SEQ ID NO: 964 - protein, 204 aa, Torque teno mini virus
KLNIKEWQPITIRKTKVKGLYPAFLCNDQRIGNNAIQYLDSIAPHHFPGGGGFGIIQFTLQGLYEQFIKATNWWTQTNCSLPLIRYNFCKLKFYRTANVDYVVKIIRCYPLKATHDLYMQTQPSILMRDKHSILIPCMKNGRNRKTYKTIKVRPPTQMTNGWFFQKDLCNFPLLVILATAASFDRYYTNSKAKSTTIGFISLNT

SEQ ID NO: 965 - protein, 204 aa, Torque teno mini virus
KIRISEWQPTKIIKSKVKGIYPCFLSNHKRLSNNFVQWIDSTTAHLMPGGGGFGIIQFTLNGLYELFCKAQNWWTKSNCNLPLVRFCGTTLKFWAAENYDYVVHIQRCYPMCCTDLMYMSCQPFIMMMTKNTIFVPCTKNKPRAKRYKKIFVKPPAQMTTQWYFQSQLAKTGLIIIRTAACSLDRIYTSSTASSTTIGLVSLHT

SEQ ID NO: 966 - protein, 204 aa, Torque teno mini virus
TIRLRQWNPRTTRLCKIKGHIPLIICGRDRQIFNWMQYYDSIGPVEQSGGGGWSSIVFSLGALYQQFKRLMNWWTKDNDGLPLVQYRGCKFKFYKSWDCDYIVTAQTCPPMTDTEYKHLDSHPYRQLMNKRPIIVPNLVRKPSKKTYKIKRFPPPSLLQNKWYFQQDFCATNLLMLTTSAASFDQFWLPNDEISQVITFHSLNT

SEQ ID NO: 967 - protein, 204 aa, Torque teno mini virus
KIILKQFQPQTIKRVKIKRLECLFHCNAKKIYFNLQMYETSTVPPHLPGGGGWSCKVFTLNALYDAFKHCRNVWTGSNHNLPLVRYMGCKFTLYQSETQDYVFKYQNHYPMVSTIESYNACQPSMLMMDNRTKKIPSKKTKHKRKPYTIVKIRPPAQMQNKWYFTHDTANTPLLLIYSAACSFDNYYISTDSESTNISIPLLRT

SEQ ID NO: 968 - protein, 204 aa, Torque teno mini virus
TITVKQFQPPNVRKCKIKGLMNLIYFNQKRLIFNSVMYENSLVPEEHPGGGGFSVIKLSLETMYDSHLRCHNWWTTSNEDLPLVRYSGMTVKLYQSKYTDYVFKYQNYLPGTSTYLTYPACQPSMMMMAKNSVIVPSLETKRRRKPYKKIHIKPTSQLQTKWYFQTDLNKTPLAIFYTAACSLTSYYLSPDWESNNISILHLNT

SEQ ID NO: 969 - protein, 204 aa, Torque teno mini virus
TIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 970 - protein, 204 aa, Torque teno mini virus
KLTLKEWQPKTIRKSCIKGLHCLFIVTEDTISRNYRMYEHSFTGEHWPSGGGFSVTKYTLDGLYEQHQLDRNWWTKPNTNMPLVRYLGCKITFYQSWEVDYVCNINLTWPMVATNLLYLSCHPNFMMMNHKAIFVPSKITKRLRRGKKTVRLRPPHQMINKWYFAKELAKTGLVMLTAAAASFDHYYIGSDKLSNNCTFTSLNP

SEQ ID NO: 971 - protein, 204 aa, Torque teno midi virus
KIPIYQWQPDSIRKCKIKGVGTLVLGAHGKQFVCYTDVETRAPPPKAPGGGGFGCKQFSLQYLYEEYRFRNNIWTHTNINLDLVRYLRAAFTFYRHPDIDFIINYDRQPPFYLDKFTYPLCHPQNLLLGKHKIILLSKASKPNGKVKKRIIIKPPKQMITKWFFQEQFTTQPLLSLRAAAASFQYPHIGCCMPNRVVTFSALNP

SEQ ID NO: 972 - protein, 204 aa, Torque teno midi virus
SLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNH

SEQ ID NO: 973 - protein, 204 aa, Torque teno midi virus
TLIVRQWQPDSIVLCKIKGYDSIIWGAEGTQFQCSTHEMYEYTRQKYPGGGGFGVQLYSLEYLYDQWKLRNNIWTKTNQLKDLCRYLKCVMTFYRHQHIDFVIVYERQPPFEIDKLTYMKYHPYMLLQRKHKIILPSQTTNPRGKLKKKKTIKPPKQMLSKWFFQQQFAKYDLLLIAAAACSLRYPRIGCCNENRMITLYCLNT

SEQ ID NO: 974 - protein, 204 aa, Torque teno midi virus
KITIKQWQPDSVKKCKIKGYSTLVMGAQGKQYNCYTNQASDYVQPKAPQGGGFGCEVFNLKWLYQEYTAHRNIWTKTNEYTDLCRYTGAQIILYRHPDVDFIVSWDNQPPFLLNKYTYPELQPQNLLLARRKRIILSQKSNPKGKLRIKLRIPPPKQMITKWFFQRDFCDVNLFKLCASAASFRYPGISHGAQSTIFSAYALNT

SEQ ID NO: 975 - protein, 204 aa, Torque teno midi virus
FLKLVQWQPDRIRKCTIKGRGTLVMGAHGRQMICFTNVKDRLTPSRAPAGGGFGVERITLAHLYEEWKARNNVWTATNENTDLGRYTGCKISFYRHLDTDFIIKYSRNPPFNLEKYSYMYMQPQELLLGKHKRILLSKKTNPKGKLKTTLKIGPPKLLTNKWLLQSELAKQDLVEISAAAASFTYPTIGCCNENRILNLYSINT

SEQ ID NO: 976 - protein, 24 aa, Alphatorquevirus sp.
PYTDITYNPFSDRGEGNMLWIDWL

SEQ ID NO: 977 - protein, 24 aa, Torque teno virus
AYTDIKYNPFTDRGEGNMLWIDWL

SEQ ID NO: 978 - protein, 24 aa, Torque teno virus
AYSDIIYNPLTDKGTGNIIWIDYL

SEQ ID NO: 979 - protein, 24 aa, Torque teno virus
AYTDVIYNPFTDKGTGNMVWIDYL

SEQ ID NO: 980 - protein, 24 aa, Torque teno virus
AYQDIIYNPFTDKGIGNIVWIDWL

SEQ ID NO: 981 - protein, 24 aa, Torque teno virus
PYTHVRYNPLVDKGFGNRIYIQWC

SEQ ID NO: 982 - protein, 24 aa, Torque teno virus
PYTHVRYNPLVDKGFGNRIYIQWC

SEQ ID NO: 983 - protein, 24 aa, Torque teno virus
AYTEVSYNPWTDEGTGNVVCLQYL

SEQ ID NO: 984 - protein, 24 aa, Torque teno virus
LYTEIIYNPYTDKGTGNKVWMDPL

SEQ ID NO: 985 - protein, 24 aa, Alphatorquevirus sp.
AYQDVTYNPNCDRAVGNVVWFQYS

SEQ ID NO: 986 - protein, 24 aa, Torque teno virus
AYQDVTYNPNCDRGVNNRVWVQPL

SEQ ID NO: 987 - protein, 24 aa, Torque teno virus
AYQDVTYNPLMDKGVGNHIWFQYN

SEQ ID NO: 988 - protein, 24 aa, Torque teno virus
AYVDTTYNPLMDKGKGNKIWFQYL

SEQ ID NO: 989 - protein, 24 aa, Torque teno virus
AYRDIVYNPFLDKAQGNMVWFQYH

SEQ ID NO: 990 - protein, 21 aa, Torque teno mini virus
ECRYNPFNDKSTGNNTFFVSN

SEQ ID NO: 991 - protein, 21 aa, Torque teno mini virus
ECRYNPLADKGKGNKIYLISI

SEQ ID NO: 992 - protein, 21 aa, Torque teno mini virus
TCRYNPLKDTGEGNQVFFKST

SEQ ID NO: 993 - protein, 21 aa, Torque teno mini virus
TIRYSPNRDTGENNICYFKPI

SEQ ID NO: 994 - protein, 21 aa, Torque teno mini virus
TTRYNPNRDEGIQNEMYLVNV

SEQ ID NO: 995 - protein, 21 aa, Torque teno mini virus
QIQYNPDRDTGEDTQLYLLSN

SEQ ID NO: 996 - protein, 21 aa, Torque teno mini virus
DIRYAPDRDTGVGNKVYLKSV

SEQ ID NO: 997 - protein, 20 aa, Torque teno midi virus
CRYNPAIDTGKGNTIWLHSN

SEQ ID NO: 998 - protein, 20 aa, Torque teno midi virus
AIYNPVKDNGDGNMIYLVST

SEQ ID NO: 999 - protein, 20 aa, Torque teno midi virus
GRYNPAIDDGKGNKIYLQTI

SEQ ID NO: 1000 - protein, 20 aa, Torque teno midi virus
LRYNPHEDTGHGNEIYLTST

SEQ ID NO: 1001 - protein, 5 aa, synthetic construct
PFYPW

SEQ ID NO: 1002 - protein, 449 aa, Gyrovirus sp.
MARRARRPRGRFYSFRRGRWHHLKRLRRRYKFRHRRRQRYRRRAFRKAFHNPRPGTYSVRLPNPQSTMTIRFQGVIFLTEGLILPKNSTAGGYADHMYGARVAKISVNLKEFLLASMNLTYVSKIGGPIAGELIADGSKSQAADNWPNCWLPLDNNVPSATPSAWWRWALMMMQPTDSCRFFNHPKQMTLQDMGRMFGGWHLFRHIETRFQLLATKNEGSFSPVASLLSQGEYLTRRDDVKYSSDHQNRWQKGGQPMTGGIAYATGKMRPDEQQYPAMPPDPPIITATTAQGTQVRCMNSTQAWWSWDTYMSFATLTALGAQWSFPPGQRSVSRRSFNHHKARGAGDPKGQRWHTLVPLGTETITDSYMSAPASELDTNFFTLYVAQGTNKSQQYKFGTATYALKEPVMKSDAWAVVRVQSVWQLGNRQRPYPWDVNWANSTMYWGTQP

SEQ ID NO: 1003 - protein, 216 aa, Gyrovirus sp.
MHGNGGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQPTPNRKKAYKTVRWQDELADREADFTPSEEDGGTTSSDFDEDINFDIGGDSGIVDELLGRPFTTPAPVRIV

SEQ ID NO: 1004 - protein, 121 aa, Gyrovirus sp.
MNALQEDTPPGPSTVFRPPTSSRPLETPHCREIRIGIAGITITLSLCGCANARAPTLRSATADNSESTGFKNVPDLRTDQPKPPSKKRSCDPSEYRVSELKESLITTTPSRPRTAKRRIRL

SEQ ID NO: 1005 - protein, 22 aa, synthetic construct
  VARIANT 13: note = E, T, I, F, V, K, Q, D, H, or P
  VARIANT 16: note = M, I, R, V, K, H, N, Q, E, or T
  VARIANT 17: note = L, I, V, T, C, or M
YDIRYNPLTDKGXGNXXWLQSL

SEQ ID NO: 1006 - DNA, 64 bp, synthetic construct
  source 1..64: mol_type = other DNA
acagctcttcaaggcgtctcacctaataaatattcaacaggaaaaccacctaatttaaattgcc

SEQ ID NO: 1007 - DNA, 43 bp, synthetic construct
  source 1..43: mol_type = other DNA
acagctcttcagtgcgtctcatagggggtgtaagggggcgtag

SEQ ID NO: 1008 - DNA, 66 bp, synthetic construct
  source 1..66: mol_type = other DNA
acagctcttcaaggcgtctcaatggtaataaatattcaacaggaaaaccacctaatttaaattgcc

SEQ ID NO: 1009 - DNA, 45 bp, synthetic construct
  source 1..45: mol_type = other DNA
acagctcttcagtgcgtctcattcagggggtgtaagggggcgtag

SEQ ID NO: 1010 - protein, 21 aa, synthetic construct
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 10: note = Any amino acid
  VARIANT 11: note = Any amino acid
  VARIANT 12: note = Any amino acid
  VARIANT 14: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
WXXXXXXXHXXXCXCXXXXXH

SEQ ID NO: 1011 - DNA, 20 bp, synthetic construct
  source 1..20: mol_type = other DNA
ccgactacttgaagctgtcc

SEQ ID NO: 1012 - DNA, 20 bp, synthetic construct
  source 1..20: mol_type = other DNA
cgcagcttcaccttgtagat

SEQ ID NO: 1013 - DNA, 20 bp, synthetic construct
  source 1..20: mol_type = other DNA
tgatgaacttcgaggacggc

SEQ ID NO: 1014 - DNA, 20 bp, synthetic construct
  source 1..20: mol_type = other DNA
ggattttgggagggtcactc

SEQ ID NO: 1015 - DNA, 20 bp, synthetic construct
  source 1..20: mol_type = other DNA
tacagttcctggacctgtgt

SEQ ID NO: 1016 - DNA, 27 bp, synthetic construct
  source 1..27: mol_type = other DNA
acactggtaccctaaaaatagatttca